SEQUENCE LISTING (247 sequences)

SEQ ID NO: 1 - protein, 253 aa, Homo sapiens
MHSWERLAVLVLLGAAACAAPPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 2 - protein, 234 aa, Homo sapiens
APPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 3 - protein, 228 aa, Homo sapiens
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 4 - protein, 6 aa, synthetic construct
  REGION 1..6: note = Synthetic
APPRGR

SEQ ID NO: 5 - protein, 7 aa, synthetic construct
  REGION 1..7: note = Synthetic
ILGGREA

SEQ ID NO: 6 - protein, 29 aa, synthetic construct
  REGION 1..29: note = Synthetic
ILGGREAGPGPGAKFVAAAWTLKAAAKKC

SEQ ID NO: 7 - protein, 728 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 8 - protein, 253 aa, Macaca fascicularis
MHSWEHLAVLVLLGVAACAAQPRGRILGGREAEAHARPYMASVQVNGEHLCGGVLVAEQWVLSAAHCLEDAADGKVQVLLGAHSLSQPEPSKRLYDVLRAVPHPDSRPDTIDHDLLLLQLSEKATLGPAVRPLPWQRVDRDVEPGTLCDVAGWGIVSHAGRRPDRLQHVLLPVLDRATCNRRTHHDGAITQRMMCAESNRRDSCKGDSGGPLVCGGVLEGVVTSGSRVCGNRKKPGIYTRVASYAAWIDSVLA

SEQ ID NO: 9 - protein, 261 aa, Canis lupus
MAPRLGPVPLVPLVLLGAALCAAQPRGRILGGSEAESHARPYMASVQVDGKHVCGGFLVSERWVLSAAHCLEDVADGKVRVLLGAHSLSQPEPSKRWYDVLRAVPHPDSRRDTIEHDLLLLQLSEDAELGPAVQPLRWQREDRDVAAGTRCDVAGWGVVSHTGRRPDRLQHLILPVLDRATCNLRTYHDGTITERMMCAESNRRDSCKGDSGGPLVCGGVAEAVVTSGSRVCGNRKKPGIYTRVASYAAWIDGVMAGGAAA

SEQ ID NO: 10 - protein, 263 aa, Rattus rattus
MHSSVYLVALVVLEAAVCVAQPRGRILGGQEAMAHARPYMASVQVNGTHVCGGTLVDEQWVLSAAHCMDGVTKDEVVQVLLGAHSLSSPEPYKHLYDVQSVVLHPGSRPDSVEDDLMLFKLSHNASLGPHVRPLPLQREDREVKPGTLCDVAGWGVVTHAGRRPDVLQQLTVSIMDRNTCNLRTYHDGAITKNMMCAESNRRDTCRGDSGGPLVCGDAVEAVVTWGSRVCGNRRKPGVFTRVATYVPWIENVLSGNVSVNVTA

SEQ ID NO: 11 - protein, 259 aa, Mus musculus
MHSSVYFVALVILGAAVCAAQPRGRILGGQEAAAHARPYMASVQVNGTHVCGGTLLDEQWVLSAAHCMDGVTDDDSVQVLLGAHSLSAPEPYKRWYDVQSVVPHPGSRPDSLEDDLILFKLSQNASLGPHVRPLPLQYEDKEVEPGTLCDVAGWGVVTHAGRRPDVLHQLRVSIMNRTTCNLRTYHDGVVTINMMCAESNRRDTCRGDSGSPLVCGDAVEGVVTWGSRVCGNGKKPGVYTRVSSYRMWIENITNGNMTS

SEQ ID NO: 12 - protein, 121 aa, synthetic construct
  REGION 1..121: note = Synthetic
QITLKESGPGILQSSQTLSLTCSFSGISLTTSGMGVSWIRQPSGKGLEWLAHIYWDDEKHYHPSLKSRLTISKDASRNQVFFRILSVDTADTATYYCALRYYGYRSFMDYWGQGTSVTVSS

SEQ ID NO: 13 - protein, 121 aa, synthetic construct
  REGION 1..121: note = Synthetic
QITLKESGPGILQSSQTLSLTCSFSGVSLTTSGMGVSWIRQPSGKGLEWLAHIYWDDEKHYHPSLKSRLTISKDASRNQVFFRILSVDTADTATYYCALRYYGYRSFMDYWGQGTSVTVSS

SEQ ID NO: 14 - protein, 121 aa, synthetic construct
  REGION 1..121: note = Synthetic
QVTLKESGPGILQSSQTLSLTCSFSGISLNISGMGVSWIRQPSGKGLEWLAHIYWDDEKHYNPSLKRRLTISKDASRNQVFFRISSVDSADTATYYCALRYYGYGSIMDYWGHGTSVTVSS

SEQ ID NO: 15 - protein, 121 aa, synthetic construct
  REGION 1..121: note = Synthetic
QVTLKESGPGILQSSQTLSLTCSFSGISLNTSIMGVSWIRQPSGKGLEWLAHIYWDDEKHYNPSLKSRLTISKDASRNQVFLKIISVDTADTATYYCALRYYGYNYVMHYWGQGTSVTVSS

SEQ ID NO: 16 - protein, 121 aa, synthetic construct
  REGION 1..121: note = Synthetic
QVTLKESGPGILQSSQTLSLTCSFSGISLSSSGMGVSWIRQPSGKGLEWLAHIYWDDEKHYNPSLKSRLTISKDASRNQIFLKIISVDTADTATYYCALRYYGYNYVMHYWGQGTSVTVSS

SEQ ID NO: 17 - protein, 121 aa, synthetic construct
  REGION 1..121: note = Synthetic
QVTLKESGPGILQSSQTLSLTCSFSGVSLSSSGMGVSWIRQPSGKGLEWLAHIYWDDEKHYNPSLKSRLTISKGASRNQVFLKIISVDTADTATYYCALRYYGYNSIMHYWGQGASVTVSS

SEQ ID NO: 18 - protein, 113 aa, synthetic construct
  REGION 1..113: note = Synthetic
DVLMTQSPLSLPVSLGDQASIFCRSNQSIVHSNGNTYFEWYLQKPGQSPKLLIYKVSNRFSGVPDRFSGSGSGTDFTLRISRVEAEDLGVYYCFQGSHVPPTFGGGTKLEIKR

SEQ ID NO: 19 - protein, 113 aa, synthetic construct
  REGION 1..113: note = Synthetic
DVLMTQSPLSLPVSLGDQASIFCRSNQSIVHSNGNTYFEWYLQKPGQSPKLLIYKVSNRFSGVPDRFSGSGSGTDFTLRISRVEAEDLGIYYCFQGSHVPPTFGGGTKLEIKR

SEQ ID NO: 20 - protein, 113 aa, synthetic construct
  REGION 1..113: note = Synthetic
DVLMTQTPLSLPVSLGDQASISCRSSQSIVHSNGNTYFEWYLQKPGQSPKLLIYKVSNRFSGVPDRFSGSGSGTDFTLKISRVEAEDLGVYYCFQGSHVPPTFGGGTKLEIKR

SEQ ID NO: 21 - protein, 113 aa, synthetic construct
  REGION 1..113: note = Synthetic
DVLMTQTPLSLPVSLGDQASISCRSSQSIVHSNGNTYLEWYLQKPGQSPKLLIYKVSNRFSGVPDRFSGSGSGTDFTLKISRVEADDLGVYYCFQGSHVPPTFGGGTKLEIKR

SEQ ID NO: 22 - protein, 113 aa, synthetic construct
  REGION 1..113: note = Synthetic
DVLMTQTPLSLPVSLGDQASISCRSSQSILHSNGNTYFEWYLQKPGQSPKLLIYKVSNRFSGVPDRFSGSGSGTDFTLKISRVEAEDLGVYYCFQGSHVPPTFGGGTKLEIKR

SEQ ID NO: 23 - protein, 113 aa, synthetic construct
  REGION 1..113: note = Synthetic
DVLMTQTPLSLPVSLGDQASISCRSSESIVHSNGNTYLEWYLQKPGQSPKLLIYKVYNRFSGVPDRFSGSGSGTDFTLKISRVEAEDLGVYYCFQGSHVPPTFGGGTKLEIKR

SEQ ID NO: 24 - protein, 30 aa, synthetic construct
  REGION 1..30: note = Synthetic
QITLKESGPGILQSSQTLSLTCSFSGISLT

SEQ ID NO: 25 - protein, 7 aa, synthetic construct
  REGION 1..7: note = Synthetic
TSGMGVS

SEQ ID NO: 26 - protein, 14 aa, synthetic construct
  REGION 1..14: note = Synthetic
WIRQPSGKGLEWLA

SEQ ID NO: 27 - protein, 16 aa, synthetic construct
  REGION 1..16: note = Synthetic
HIYWDDEKHYHPSLKS

SEQ ID NO: 28 - protein, 32 aa, synthetic construct
  REGION 1..32: note = Synthetic
RLTISKDASRNQVFFRILSVDTADTATYYCAL

SEQ ID NO: 29 - protein, 11 aa, synthetic construct
  REGION 1..11: note = Synthetic
RYYGYRSFMDY

SEQ ID NO: 30 - protein, 11 aa, synthetic construct
  REGION 1..11: note = Synthetic
WGQGTSVTVSS

SEQ ID NO: 31 - protein, 30 aa, synthetic construct
  REGION 1..30: note = Synthetic
QITLKESGPGILQSSQTLSLTCSFSGVSLT

SEQ ID NO: 32 - protein, 30 aa, synthetic construct
  REGION 1..30: note = Synthetic
QVTLKESGPGILQSSQTLSLTCSFSGISLN

SEQ ID NO: 33 - protein, 7 aa, synthetic construct
  REGION 1..7: note = Synthetic
ISGMGVS

SEQ ID NO: 34 - protein, 16 aa, synthetic construct
  REGION 1..16: note = Synthetic
HIYWDDEKHYNPSLKR

SEQ ID NO: 35 - protein, 32 aa, synthetic construct
  REGION 1..32: note = Synthetic
RLTISKDASRNQVFFRISSVDSADTATYYCAL

SEQ ID NO: 36 - protein, 11 aa, synthetic construct
  REGION 1..11: note = Synthetic
RYYGYGSIMDY

SEQ ID NO: 37 - protein, 11 aa, synthetic construct
  REGION 1..11: note = Synthetic
WGHGTSVTVSS

SEQ ID NO: 38 - protein, 7 aa, synthetic construct
  REGION 1..7: note = Synthetic
TSIMGVS

SEQ ID NO: 39 - protein, 16 aa, synthetic construct
  REGION 1..16: note = Synthetic
HIYWDDEKHYNPSLKS

SEQ ID NO: 40 - protein, 32 aa, synthetic construct
  REGION 1..32: note = Synthetic
RLTISKDASRNQVFLKIISVDTADTATYYCAL

SEQ ID NO: 41 - protein, 11 aa, synthetic construct
  REGION 1..11: note = Synthetic
RYYGYNYVMHY

SEQ ID NO: 42 - protein, 30 aa, synthetic construct
  REGION 1..30: note = Synthetic
QVTLKESGPGILQSSQTLSLTCSFSGISLS

SEQ ID NO: 43 - protein, 7 aa, synthetic construct
  REGION 1..7: note = Synthetic
SSGMGVS

SEQ ID NO: 44 - protein, 32 aa, synthetic construct
  REGION 1..32: note = Synthetic
RLTISKDASRNQIFLKIISVDTADTATYYCAL

SEQ ID NO: 45 - protein, 30 aa, synthetic construct
  REGION 1..30: note = Synthetic
QVTLKESGPGILQSSQTLSLTCSFSGVSLS

SEQ ID NO: 46 - protein, 32 aa, synthetic construct
  REGION 1..32: note = Synthetic
RLTISKGASRNQVFLKIISVDTADTATYYCAL

SEQ ID NO: 47 - protein, 11 aa, synthetic construct
  REGION 1..11: note = Synthetic
RYYGYNSIMHY

SEQ ID NO: 48 - protein, 11 aa, synthetic construct
  REGION 1..11: note = Synthetic
WGQGASVTVSS

SEQ ID NO: 49 - protein, 23 aa, synthetic construct
  REGION 1..23: note = Synthetic
DVLMTQSPLSLPVSLGDQASIFC

SEQ ID NO: 50 - protein, 16 aa, synthetic construct
  REGION 1..16: note = Synthetic
RSNQSIVHSNGNTYFE

SEQ ID NO: 51 - protein, 15 aa, synthetic construct
  REGION 1..15: note = Synthetic
WYLQKPGQSPKLLIY

SEQ ID NO: 52 - protein, 7 aa, synthetic construct
  REGION 1..7: note = Synthetic
KVSNRFS

SEQ ID NO: 53 - protein, 32 aa, synthetic construct
  REGION 1..32: note = Synthetic
GVPDRFSGSGSGTDFTLRISRVEAEDLGVYYC

SEQ ID NO: 54 - protein, 9 aa, synthetic construct
  REGION 1..9: note = Synthetic
FQGSHVPPT

SEQ ID NO: 55 - protein, 11 aa, synthetic construct
  REGION 1..11: note = Synthetic
FGGGTKLEIKR

SEQ ID NO: 56 - protein, 32 aa, synthetic construct
  REGION 1..32: note = Synthetic
GVPDRFSGSGSGTDFTLRISRVEAEDLGIYYC

SEQ ID NO: 57 - protein, 23 aa, synthetic construct
  REGION 1..23: note = Synthetic
DVLMTQTPLSLPVSLGDQASISC

SEQ ID NO: 58 - protein, 16 aa, synthetic construct
  REGION 1..16: note = Synthetic
RSSQSIVHSNGNTYFE

SEQ ID NO: 59 - protein, 32 aa, synthetic construct
  REGION 1..32: note = Synthetic
GVPDRFSGSGSGTDFTLKISRVEAEDLGVYYC

SEQ ID NO: 60 - protein, 16 aa, synthetic construct
  REGION 1..16: note = Synthetic
RSSQSIVHSNGNTYLE

SEQ ID NO: 61 - protein, 32 aa, synthetic construct
  REGION 1..32: note = Synthetic
GVPDRFSGSGSGTDFTLKISRVEADDLGVYYC

SEQ ID NO: 62 - protein, 16 aa, synthetic construct
  REGION 1..16: note = Synthetic
RSSQSILHSNGNTYFE

SEQ ID NO: 63 - protein, 16 aa, synthetic construct
  REGION 1..16: note = Synthetic
RSSESIVHSNGNTYLE

SEQ ID NO: 64 - protein, 7 aa, synthetic construct
  REGION 1..7: note = Synthetic
KVYNRFS

SEQ ID NO: 65 - protein, 7 aa, synthetic construct
  REGION 1..7: note = Synthetic
  VARIANT 1: note = Wherein Xaa at position 1 is T or I or S
  VARIANT 3: note = Wherein Xaa at position 3 is G or I
XSXMGVS

SEQ ID NO: 66 - protein, 16 aa, synthetic construct
  REGION 1..16: note = Synthetic
  VARIANT 11: note = Wherein Xaa at position 11 is H or N
  VARIANT 16: note = Wherein Xaa at position 16 is S or R
HIYWDDEKHYXPSLKX

SEQ ID NO: 67 - protein, 11 aa, synthetic construct
  REGION 1..11: note = Synthetic
  VARIANT 6: note = Wherein Xaa at position 6 is R or G or N
  VARIANT 7: note = Wherein Xaa at position 7 is S or Y
  VARIANT 8: note = Wherein Xaa at position 8 is F or I or V
  VARIANT 10: note = Wherein Xaa at position 10 is D or H
RYYGYXXXMXY

SEQ ID NO: 68 - protein, 16 aa, synthetic construct
  REGION 1..16: note = Synthetic
  VARIANT 3: note = Wherein Xaa at position 3 is N or S
  VARIANT 4: note = Wherein Xaa at position 4 is Q or E
  VARIANT 7: note = Wherein Xaa at position 7 is V or L
  VARIANT 15: note = Wherein Xaa at position 15 is F or L
RSXXSIXHSNGNTYXE

SEQ ID NO: 69 - protein, 7 aa, synthetic construct
  REGION 1..7: note = Synthetic
  VARIANT 3: note = Wherein Xaa at position 3 is S or Y
KVXNRFS

SEQ ID NO: 70 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 363 bp, synthetic construct
  misc_feature 1..363: note = Synthetic
  source 1..363: mol_type = other DNA
cagattactctgaaagagtctggccctgggatattgcagtcctcccagaccctcagtctgacttgttctttctctgggatttcactgactacttctggtatgggtgtgagctggattcgtcagccttcaggaaagggtctggaatggctggcacacatttattgggatgatgagaaacactatcatccatccctgaagagccggctcacaatctccaaggatgcctccagaaaccaggttttcttcaggatccttagtgtggacactgcagatactgccacatactactgtgctctccgttactacggttataggtcttttatggactactggggtcaaggaacctcagtcaccgtctcctca

SEQ ID NO: 74 - DNA, 363 bp, synthetic construct
  misc_feature 1..363: note = Synthetic
  source 1..363: mol_type = other DNA
cagattactctgaaagagtctggccctgggatattgcagtcctcccagaccctcagtctgacttgttctttctctggggtttcactgactacttctggtatgggtgtgagctggattcgtcagccttcaggaaagggtctggaatggctggcacacatttattgggatgatgagaaacactatcatccatccctgaagagccggctcacaatctccaaggatgcctccagaaaccaggttttcttcaggatccttagtgtggacactgcagatactgccacatattactgtgctctccgttactacggttataggtcttttatggactattggggtcaaggaacctcagtcaccgtctcctca

SEQ ID NO: 75 - DNA, 363 bp, synthetic construct
  misc_feature 1..363: note = Synthetic
  source 1..363: mol_type = other DNA
caggttactctgaaagagtctggccctgggatattgcagtcctcccagaccctcagtctgacttgttctttctctgggatttcactgaatatttccggtatgggtgtgagctggattcgtcagccttcaggaaagggtctggagtggctggcacacatttactgggatgatgaaaaacactataatccatccctgaagagacggctcactatctccaaggatgcctccagaaaccaggttttcttcaggatcagtagtgtggactctgcagatactgccacatactactgtgcgctccgttactacggttatggttctattatggactattggggtcatggaacctcagtcaccgtctcctca

SEQ ID NO: 76 - DNA, 363 bp, synthetic construct
  misc_feature 1..363: note = Synthetic
  source 1..363: mol_type = other DNA
caggttactctgaaagagtctggccctgggatattgcagtcctcccagaccctcagtctgacttgttctttctctgggatttcattgaatacttctattatgggtgtgagctggattcgtcagccttcaggaaagggtctggagtggctggcacacatttactgggatgatgagaaacactataacccatccctgaagagccgactcacaatctccaaggatgcctccagaaaccaggtattcctcaagatcattagtgtggacactgcagatactgccacatactactgtgctctccgttactacggttataactatgttatgcactactggggtcaaggaacctcagtcaccgtctcctca

SEQ ID NO: 77 - DNA, 363 bp, synthetic construct
  misc_feature 1..363: note = Synthetic
  source 1..363: mol_type = other DNA
caggttactctgaaagagtctggccctgggatattgcagtcctcccagaccctcagtctgacttgttctttctctgggatttcactgagttcttctggtatgggtgtgagctggattcgtcagccttcaggaaagggtctggagtggctggcacacatttactgggatgatgagaaacactataacccatccctgaagagccggctcacaatctccaaggatgcctccagaaaccagatattcctcaagatcattagtgtggacactgcagatactgccacatattattgtgctctccgttactacggttataactatgttatgcactactggggtcaaggaacctcagtcaccgtctcctca

SEQ ID NO: 78 - DNA, 363 bp, synthetic construct
  misc_feature 1..363: note = Synthetic
  source 1..363: mol_type = other DNA
caggttactctgaaagagtctggccctgggatattgcagtcctcccagaccctcagtctgacttgttctttctctggggtttcactgagttcttctggtatgggtgtgagctggattcgtcagccttcaggaaagggtctggagtggctggcacacatttactgggatgatgagaaacactataacccatccctgaagagccggctcacaatctccaagggtgcctccagaaaccaggtcttcctcaagatcattagtgtggacactgcagatactgccacatactactgtgctctccgttactacggttataactctattatgcactactggggtcaaggagcctcagtcaccgtctcctca

SEQ ID NO: 79 - DNA, 339 bp, synthetic construct
  misc_feature 1..339: note = Synthetic
  source 1..339: mol_type = other DNA
gatgttttgatgacccaatctccactctccctgcctgtcagtcttggagatcaagcctccatcttttgcagatctaatcagagcattgtacatagtaatggaaacacctatttcgaatggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggagtttattactgctttcaaggttcacatgttcctccgacgttcggtggaggcaccaagctggaaatcaaacgg

SEQ ID NO: 80 - DNA, 339 bp, synthetic construct
  misc_feature 1..339: note = Synthetic
  source 1..339: mol_type = other DNA
gatgttttgatgacccaatctccactctccctgcctgtcagtcttggagatcaagcctccatcttttgcagatctaatcagagcattgttcatagtaatggaaacacctatttcgaatggtacctgcagaaaccaggccagtctccaaagctcctgatctacaaagtttccaaccgattttctggggtcccagacaggttcagtggcagtggatcagggacagatttcacactcaggatcagcagagtggaggctgaggatctgggaatttattactgctttcaaggttcacatgttcctccgacgttcggtggaggcaccaagctggaaatcaaacgg

SEQ ID NO: 81 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattgttcatagtaatggaaatacctattttgaatggtacctccagaaaccaggccagtctccaaagctcctgatctacaaggtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggagatcaaacgg

SEQ ID NO: 82 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacagattcagtggcagtggatcagggacagatttcacactcaagatcagcagagtggaggctgatgatctgggagtttattactgctttcaaggttcacatgttcctccgacgttcggtggaggcaccaagctggaaatcaaacgg

SEQ ID NO: 83 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattctacatagtaatggaaacacctattttgaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaacgg

SEQ ID NO: 84 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtgagagcattgtacatagtaatggaaacacctatttagaatggtacctgcagaaaccaggccagtctccaaagctcctgatctacaaagtttacaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggagatcaaacgg

SEQ ID NO: 85 - protein, 120 aa, synthetic construct
  REGION 1..120: note = Synthetic
EVKLVESGGGLVQPGGSLKLSCATSGFTFSDYGMAWVRQAPGKGPEWVAFISNLAYSFYYVDIVMGRFTISRENAKNTLYLEMSSLRSEDTAMYYCARVGLYGNFFMDYWGQGTSVTVSS

SEQ ID NO: 86 - protein, 120 aa, synthetic construct
  REGION 1..120: note = Synthetic
EVQLVESGGGLVQPKGSLKLSCAASGFSFNTYAMNWVRQAPGKGLEWVARIRSKSNNYATHYADSVKDRFTISRDDSESMLYLQMNNLKTEDTAMYYCVRQGYYWYFDVWGTGTTVTVSS

SEQ ID NO: 87 - protein, 120 aa, synthetic construct
  REGION 1..120: note = Synthetic
EVQLVESGGGLVQPKGSLKLSCAASGFSFNTYAMNWVRQAPGKGLEWVARIRSKSNNYATYYADSVKDRFTISRDDSESMLYLQMNNLKTEDTAMYYCVRHGYYWYFDVWGTGTTVTVSS

SEQ ID NO: 88 - protein, 120 aa, synthetic construct
  REGION 1..120: note = Synthetic
EVQVVESGGGLVRPKGSLKLSCAASGFSFNTYAMNWVRQAPGKGLEWVARIRSKSNNYATYYADSVKDRFTISRDDSESMLSLQMNNLKTEDTAMYYCVRQGYYWYFDVWGTGTTVTVSS

SEQ ID NO: 89 - protein, 108 aa, synthetic construct
  REGION 1..108: note = Synthetic
DIQMNQSPSSLSASLGDTITITCHASQNINVWLSWYQQKPGNIPELLIYKASNLHTGVPSRFSGNRSGTSFTLTISSLQPEDIGTYFCQQGQSYPLTFGAGTKLELRR

SEQ ID NO: 90 - protein, 108 aa, synthetic construct
  REGION 1..108: note = Synthetic
DIQMNQSPSSLSASLGDTITITCHASQNINVWLSWYQQKPGNIPELLIYKASNLHTGVPSRFSGNRSGTSFTLTISSLQPEDIGTYFCQQGQSYPLTFGAGTKLEIKR

SEQ ID NO: 91 - protein, 113 aa, synthetic construct
  REGION 1..113: note = Synthetic
DVLMTQTPLSLPVSLGDQASISCRSSQSIVHSNGNTYLEWYLQKPGQSPKLLIYTVSNRFSGVPDRFSGSGSGTDFTLKISRVEAEDLGVYYCFQGSHVPWTFGGGTKLEIKR

SEQ ID NO: 92 - protein, 113 aa, synthetic construct
  REGION 1..113: note = Synthetic
DVLMTQTPLSLPVSLGDQASISCRSSQSIVHSDGNTYLEWYLQKPGQSPKLLIYRVSNRFSGVPDRFSGSGSGTDFTLKISRVEAEDLGVYYCFQGSHVPYTFGGGTKLEIKR

SEQ ID NO: 93 - protein, 113 aa, synthetic construct
  REGION 1..113: note = Synthetic
DVLMTQTPLSLPVSLGDQASISCRSSQSIVHSNGNTYLEWYLQKPGQSPKLLIYTVSNRFSGVPDRFRGSGSGTDFTLKISRVEAEDLGVYYCFQGSHVPWTFGGGTKLEIKR

SEQ ID NO: 94 - protein, 30 aa, synthetic construct
  REGION 1..30: note = Synthetic
EVKLVESGGGLVQPGGSLKLSCATSGFTFS

SEQ ID NO: 95 - protein, 5 aa, synthetic construct
  REGION 1..5: note = Synthetic
DYGMA

SEQ ID NO: 96 - protein, 14 aa, synthetic construct
  REGION 1..14: note = Synthetic
WVRQAPGKGPEWVA

SEQ ID NO: 97 - protein, 17 aa, synthetic construct
  REGION 1..17: note = Synthetic
FISNLAYSFYYVDIVMG

SEQ ID NO: 98 - protein, 32 aa, synthetic construct
  REGION 1..32: note = Synthetic
RFTISRENAKNTLYLEMSSLRSEDTAMYYCAR

SEQ ID NO: 99 - protein, 11 aa, synthetic construct
  REGION 1..11: note = Synthetic
VGLYGNFFMDY

SEQ ID NO: 100 - protein, 30 aa, synthetic construct
  REGION 1..30: note = Synthetic
EVQLVESGGGLVQPKGSLKLSCAASGFSFN

SEQ ID NO: 101 - protein, 5 aa, synthetic construct
  REGION 1..5: note = Synthetic
TYAMN

SEQ ID NO: 102 - protein, 14 aa, synthetic construct
  REGION 1..14: note = Synthetic
WVRQAPGKGLEWVA

SEQ ID NO: 103 - protein, 19 aa, synthetic construct
  REGION 1..19: note = Synthetic
RIRSKSNNYATHYADSVKD

SEQ ID NO: 104 - protein, 32 aa, synthetic construct
  REGION 1..32: note = Synthetic
RFTISRDDSESMLYLQMNNLKTEDTAMYYCVR

SEQ ID NO: 105 - protein, 9 aa, synthetic construct
  REGION 1..9: note = Synthetic
QGYYWYFDV

SEQ ID NO: 106 - protein, 11 aa, synthetic construct
  REGION 1..11: note = Synthetic
WGTGTTVTVSS

SEQ ID NO: 107 - protein, 19 aa, synthetic construct
  REGION 1..19: note = Synthetic
RIRSKSNNYATYYADSVKD

SEQ ID NO: 108 - protein, 9 aa, synthetic construct
  REGION 1..9: note = Synthetic
HGYYWYFDV

SEQ ID NO: 109 - protein, 30 aa, synthetic construct
  REGION 1..30: note = Synthetic
EVQVVESGGGLVRPKGSLKLSCAASGFSFN

SEQ ID NO: 110 - protein, 23 aa, synthetic construct
  REGION 1..23: note = Synthetic
DIQMNQSPSSLSASLGDTITITC

SEQ ID NO: 111 - protein, 11 aa, synthetic construct
  REGION 1..11: note = Synthetic
HASQNINVWLS

SEQ ID NO: 112 - protein, 15 aa, synthetic construct
  REGION 1..15: note = Synthetic
WYQQKPGNIPELLIY

SEQ ID NO: 113 - protein, 7 aa, synthetic construct
  REGION 1..7: note = Synthetic
KASNLHT

SEQ ID NO: 114 - protein, 32 aa, synthetic construct
  REGION 1..32: note = Synthetic
GVPSRFSGNRSGTSFTLTISSLQPEDIGTYFC

SEQ ID NO: 115 - protein, 9 aa, synthetic construct
  REGION 1..9: note = Synthetic
QQGQSYPLT

SEQ ID NO: 116 - protein, 11 aa, synthetic construct
  REGION 1..11: note = Synthetic
FGAGTKLELRR

SEQ ID NO: 117 - protein, 11 aa, synthetic construct
  REGION 1..11: note = Synthetic
FGAGTKLEIKR

SEQ ID NO: 118 - protein, 23 aa, synthetic construct
  REGION 1..23: note = Synthetic
DVLMTQTPLSLPVSLGDQASISC

SEQ ID NO: 119 - protein, 7 aa, synthetic construct
  REGION 1..7: note = Synthetic
TVSNRFS

SEQ ID NO: 120 - protein, 32 aa, synthetic construct; identical to SEQ ID NO: 59
  REGION 1..32: note = Synthetic

SEQ ID NO: 121 - protein, 9 aa, synthetic construct
  REGION 1..9: note = Synthetic
FQGSHVPWT

SEQ ID NO: 122 - protein, 11 aa, synthetic construct
  REGION 1..11: note = Synthetic
FGGGTKLEIKR

SEQ ID NO: 123 - protein, 16 aa, synthetic construct
  REGION 1..16: note = Synthetic
RSSQSIVHSDGNTYLE

SEQ ID NO: 124 - protein, 7 aa, synthetic construct
  REGION 1..7: note = Synthetic
RVSNRFS

SEQ ID NO: 125 - protein, 9 aa, synthetic construct
  REGION 1..9: note = Synthetic
FQGSHVPYT

SEQ ID NO: 126 - protein, 32 aa, synthetic construct
  REGION 1..32: note = Synthetic
GVPDRFRGSGSGTDFTLKISRVEAEDLGVYYC

SEQ ID NO: 127 - DNA, 360 bp, synthetic construct
  misc_feature 1..360: note = Synthetic
  source 1..360: mol_type = other DNA
gaggtgaagctggtggagtctgggggaggcttagtgcagcctggagggtccctgaaactctcctgtgcaacctctggattcactttcagtgactacggaatggcgtgggttcgacaggctccagggaaggggcctgagtgggtagcattcattagtaatttggcatatagtttctactatgtagacattgtgatgggccgattcaccatctctagagagaatgccaagaacaccctgtacctggaaatgagcagtctgaggtctgaggacacggccatgtattactgtgcaagagtggggctctatggtaacttttttatggactactggggtcaaggaacctcagtcaccgtctcctca

SEQ ID NO: 128 - DNA, 360 bp, synthetic construct
  misc_feature 1..360: note = Synthetic
  source 1..360: mol_type = other DNA
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattatgcaacacattatgccgattcagtgaaagacagattcaccatctccagagatgattcagaaagcatgctctatctgcaaatgaacaacttgaaaactgaggacacagccatgtattactgtgtgagacagggttactactggtacttcgatgtctggggcacagggaccacggtcaccgtctcctca

SEQ ID NO: 129 - DNA, 360 bp, synthetic construct
  misc_feature 1..360: note = Synthetic
  source 1..360: mol_type = other DNA
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattatgcaacatattatgccgattcagtgaaagacagattcaccatctccagagatgattcagaaagcatgctctatctgcaaatgaacaacttgaaaactgaggacacagccatgtattactgtgtgagacatggttactactggtacttcgatgtctggggcacagggaccacggtcaccgtctcctca

SEQ ID NO: 130 - DNA, 360 bp, synthetic construct
  misc_feature 1..360: note = Synthetic
  source 1..360: mol_type = other DNA
gaggtgcaggttgttgagtctggtggaggattggtgcggcctaaagggtcattgaaactctcatgtgcagcctctggattcagcttcaatacctacgccatgaactgggtccgccaggctccaggaaagggtttggaatgggttgctcgcataagaagtaaaagtaataattatgcaacatattatgccgattcagtgaaagacagattcaccatctccagagatgattcagaaagcatgctctctctgcaaatgaacaacttgaaaactgaggacacagccatgtattactgtgtgagacagggttactactggtacttcgatgtctggggcacagggaccacggtcaccgtctcctca

SEQ ID NO: 131 - DNA, 324 bp, synthetic construct
  misc_feature 1..324: note = Synthetic
  source 1..324: mol_type = other DNA
gacatccagatgaaccagtctccatccagtctgtctgcatcccttggagacacaattaccatcacttgccatgccagtcagaacattaatgtttggttaagctggtaccagcagaaaccaggaaatattcctgaacttttgatctataaggcttccaacttgcacacaggcgtcccttctaggtttagtggcaatagatctggaacaagtttcacattaaccatcagcagcctgcagcctgaagacattggcacttacttctgtcaacagggtcaaagttatccgctcacgttcggtgctgggaccaagctggagctgagacgg

SEQ ID NO: 132 - DNA, 324 bp, synthetic construct
  misc_feature 1..324: note = Synthetic
  source 1..324: mol_type = other DNA
gacatccagatgaaccagtctccatccagtctgtctgcatcccttggagacacaattaccatcacttgccatgccagtcagaacattaatgtttggttaagctggtaccagcagaaaccaggaaatattcctgaacttttgatctataaggcttccaacttgcacacaggcgtcccttctaggtttagtggcaatagatctggaacaagtttcacattaaccatcagcagcctgcagcctgaagacattggcacttacttctgtcaacagggtcaaagttatccgctcacgttcggtgcggggaccaagctggaaataaaacgg

SEQ ID NO: 133 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacacagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaacgg

SEQ ID NO: 134 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattgtacatagtgatggaaacacctatttagaatggtacctgcagaaaccaggccagtctccaaagctcctgatctacagagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggaggcaccaagctggaaatcaaacgg

SEQ ID NO: 135 - DNA, 339 bp, synthetic construct
  misc_feature 1..339: note = Synthetic
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacacagtttccaaccgattttctggggtcccagacaggttccgtggcagtggatcagggacagatttcacactcaagatcagcagagtggaggctgaggatctgggagtttattactgctttcaaggttcacatgttccgtggacgttcggtggaggcaccaagctggaaatcaaacgg

SEQ ID NO: 136 - protein, 116 aa, synthetic construct
  REGION 1..116: note = Synthetic
EVKLEESGGGLVQPGGSMKLSCVASGFTFGNYWMSWVRQSPEKGLEWVAEIRLKSDNYATHYAESVKGKFTISRDDSKSRLYLQMNSLRGEDTGLYYCTNAWFASWGQGTLVTVSA

SEQ ID NO: 137 - protein, 116 aa, synthetic construct
  REGION 1..116: note = Synthetic
EVKLEESGGGLVQPGGSMKLSCVASGFTFGSYWMSWVRQSPEKGLEWVAEIRLKSDNYAAHYAESVKGKFTISRDDSKSRLYLQMNSLRGEDTGIYYCTNAWFASWGQGTLVTVSA

SEQ ID NO: 138 - protein, 116 aa, synthetic construct
  REGION 1..116: note = Synthetic
EVKLEESGGGLVQPGGSMKLSCVASGFTFSTYWMSWVRQSPEKGLEWVAEIRLKSDNYATHYTESVKGKFTISRDDSKSRLYLQMNSLRVEDTGIYYCTNAWFAYWGQGTLVTVSA

SEQ ID NO: 139 - protein, 116 aa, synthetic construct
  REGION 1..116: note = Synthetic
EVKLEESGGGLVQPGGSMKLSCIASGFTFSTYWMSWVRQSPEKGLEWVAEIRLKSENYATYYAESVKGKFIISRDDSKSRLYLQMNSLRAEDTGIYYCTNAWFANWGQGTLVTVSA

SEQ ID NO: 140 - protein, 115 aa, synthetic construct
  REGION 1..115: note = Synthetic
DVQLQESGPGLVKPSQSLSLTCTVTGYSITSDYAWNWIRQFPGNKLEWMGYISYIGGIGYNPSLKSRISITRDTSKNQFFLHLNSVTTGDTATYYCARNGAMDFWGQGISVTVSS

SEQ ID NO: 141 - protein, 115 aa, synthetic construct
  REGION 1..115: note = Synthetic
DVQLQESGPGLVKPSQSLSLTCTVTGYSITSDYAWNWIRQFPGNKLEWMGYISYSGSTGYSPSLKSRISITRDTSKNQFFLHLNSVTTGDTATYYCARNGAMDYWGQGISVTVSS

SEQ ID NO: 142 - protein, 114 aa, synthetic construct
  REGION 1..114: note = Synthetic
DIVMSQSPSSLAVSVGEKVTMSCMSSQSLLYSKDQKNYLAWYQQKPGQSPKLLIYWASTRESGVPDRFTGSGSGTDFTLTISSVKAEDLAVYYCLQYYTYPYTFGGGTKLEIKR

SEQ ID NO: 143 - protein, 114 aa, synthetic construct
  REGION 1..114: note = Synthetic
DIVMSQSPSSLTVSVGEKVTMSCMSSQSLLYSKDQKNYLAWYQQKPGQSPTLLIYWASTRESGVPDRFTGSGSGTDFTLTISSVKAEDLAVYYCLQYYTYPYTFGGGTKLEIKR

SEQ ID NO: 144 - protein, 114 aa, synthetic construct
  REGION 1..114: note = Synthetic
DIVMSQSPSSLAVSVGEKFTMSCKSSQSLLYSRDQKNYLAWYQQQPGQSPKLLIYWASTRESGVPDRFTGSGSGTDFTLTISSVKTEDLAVYYCLQYYSYPYTFGGGTKLEIKR

SEQ ID NO: 145 - protein, 114 aa, synthetic construct
  REGION 1..114: note = Synthetic
DIVMSQSPSSLVVSVGEKVTMSCKSSQNLLYSRDQKNYLAWYQQKPGQSPNLLIYWASTRESGVPDRFTGSGSGTDFSLTISSVKAEDLAVYYCLQYYSYPYTFGGGTKLEMKR

SEQ ID NO: 146 - protein, 112 aa, synthetic construct
  REGION 1..112: note = Synthetic
DIVLTQSPASLAVSLGQRATISCKASQSVDYDGDSYMNWYQQKPGQPPKLLIYDASNLESGIPARFSGSGSGTDFTLNIHPVEEEDAATYYCQQSNEAPWTFGGGTKLEIKR

SEQ ID NO: 147 - protein, 110 aa, synthetic construct
  REGION 1..110: note = Synthetic
DIVLTQSPASLAVSLGQTISCKASQSVDYDGDSYMNWYQQKPGQPPKLLIYDASTLESGIPARFSGSGSGTDFTLNIHPVEEEDAATYYCQQNYEAPWTFGGGTKLEIKR

SEQ ID NO: 148 - protein, 30 aa, synthetic construct
  REGION 1..30: note = Synthetic
EVKLEESGGGLVQPGGSMKLSCVASGFTFG

SEQ ID NO: 149 - protein, 5 aa, synthetic construct
  REGION 1..5: note = Synthetic
NYWMS

SEQ ID NO: 150 - protein, 14 aa, synthetic construct
  REGION 1..14: note = Synthetic
WVRQSPEKGLEWVA

SEQ ID NO: 151 - protein, 19 aa, synthetic construct
  REGION 1..19: note = Synthetic
EIRLKSDNYATHYAESVKG

SEQ ID NO: 152 - protein, 32 aa, synthetic construct
  REGION 1..32: note = Synthetic
KFTISRDDSKSRLYLQMNSLRGEDTGLYYCTN

SEQ ID NO: 153 - protein, 5 aa, synthetic construct
  REGION 1..5: note = Synthetic
AWFAS

SEQ ID NO: 154 - protein, 11 aa, synthetic construct
  REGION 1..11: note = Synthetic
WGQGTLVTVSA

SEQ ID NO: 155 - protein, 5 aa, synthetic construct
  REGION 1..5: note = Synthetic
SYWMS

SEQ ID NO: 156 - protein, 19 aa, synthetic construct
  REGION 1..19: note = Synthetic
EIRLKSDNYAAHYAESVKG

SEQ ID NO: 157 - protein, 32 aa, synthetic construct
  REGION 1..32: note = Synthetic
KFTISRDDSKSRLYLQMNSLRGEDTGIYYCTN

SEQ ID NO: 158 - protein, 5 aa, synthetic construct
  REGION 1..5: note = Synthetic
TYWMS

SEQ ID NO: 159 - protein, 19 aa, synthetic construct
  REGION 1..19: note = Synthetic
EIRLKSDNYATHYTESVKG

SEQ ID NO: 160 - protein, 32 aa, synthetic construct
  REGION 1..32: note = Synthetic
KFTISRDDSKSRLYLQMNSLRVEDTGIYYCTN

SEQ ID NO: 161 - protein, 5 aa, synthetic construct
  REGION 1..5: note = Synthetic
AWFAY

SEQ ID NO: 162 - protein, 30 aa, synthetic construct
  REGION 1..30: note = Synthetic
EVKLEESGGGLVQPGGSMKLSCIASGFTFS

SEQ ID NO: 163 - protein, 19 aa, synthetic construct
  REGION 1..19: note = Synthetic
EIRLKSENYATYYAESVKG

SEQ ID NO: 164 - protein, 32 aa, synthetic construct
  REGION 1..32: note = Synthetic
KFIISRDDSKSRLYLQMNSLRAEDTGIYYCTN

SEQ ID NO: 165 - protein, 5 aa, synthetic construct
  REGION 1..5: note = Synthetic
AWFAN

SEQ ID NO: 166 - protein, 30 aa, synthetic construct
  REGION 1..30: note = Synthetic
DVQLQESGPGLVKPSQSLSLTCTVTGYSIT

SEQ ID NO: 167 - protein, 6 aa, synthetic construct
  REGION 1..6: note = Synthetic
SDYAWN

SEQ ID NO: 168 - protein, 14 aa, synthetic construct
  REGION 1..14: note = Synthetic
WIRQFPGNKLEWMG

SEQ ID NO: 169 - protein, 16 aa, synthetic construct
  REGION 1..16: note = Synthetic
YISYIGGIGYNPSLKS

SEQ ID NO: 170 - protein, 32 aa, synthetic construct
  REGION 1..32: note = Synthetic
RISITRDTSKNQFFLHLNSVTTGDTATYYCAR

SEQ ID NO: 171 - protein, 6 aa, synthetic construct
  REGION 1..6: note = Synthetic
NGAMDF

SEQ ID NO: 172 - protein, 11 aa, synthetic construct
  REGION 1..11: note = Synthetic
WGQGISVTVSS

SEQ ID NO: 173 - protein, 16 aa, synthetic construct
  REGION 1..16: note = Synthetic
YISYSGSTGYSPSLKS

SEQ ID NO: 174 - protein, 6 aa, synthetic construct
  REGION 1..6: note = Synthetic
NGAMDY

SEQ ID NO: 175 - protein, 23 aa, synthetic construct
  REGION 1..23: note = Synthetic
DIVMSQSPSSLAVSVGEKVTMSC

SEQ ID NO: 176 - protein, 17 aa, synthetic construct
  REGION 1..17: note = Synthetic
MSSQSLLYSKDQKNYLA

SEQ ID NO: 177 - protein, 15 aa, synthetic construct
  REGION 1..15: note = Synthetic
WYQQKPGQSPKLLIY

SEQ ID NO: 178 - protein, 7 aa, synthetic construct
  REGION 1..7: note = Synthetic
WASTRES

SEQ ID NO: 179 - protein, 32 aa, synthetic construct
  REGION 1..32: note = Synthetic
GVPDRFTGSGSGTDFTLTISSVKAEDLAVYYC

SEQ ID NO: 180 - protein, 9 aa, synthetic construct
  REGION 1..9: note = Synthetic
LQYYTYPYT

SEQ ID NO: 181 - protein, 23 aa, synthetic construct
  REGION 1..23: note = Synthetic
DIVMSQSPSSLTVSVGEKVTMSC

SEQ ID NO: 182 - protein, 15 aa, synthetic construct
  REGION 1..15: note = Synthetic
WYQQKPGQSPTLLIY

SEQ ID NO: 183 - protein, 23 aa, synthetic construct
  REGION 1..23: note = Synthetic
DIVMSQSPSSLAVSVGEKFTMSC

SEQ ID NO: 184 - protein, 17 aa, synthetic construct
  REGION 1..17: note = Synthetic
KSSQSLLYSRDQKNYLA

SEQ ID NO: 185 - protein, 15 aa, synthetic construct
  REGION 1..15: note = Synthetic
WYQQQPGQSPKLLIY

SEQ ID NO: 186 - protein, 32 aa, synthetic construct
  REGION 1..32: note = Synthetic
GVPDRFTGSGSGTDFTLTISSVKTEDLAVYYC

SEQ ID NO: 187 - protein, 9 aa, synthetic construct
  REGION 1..9: note = Synthetic
LQYYSYPYT

SEQ ID NO: 188 - protein, 23 aa, synthetic construct
  REGION 1..23: note = Synthetic
DIVMSQSPSSLVVSVGEKVTMSC

SEQ ID NO: 189 - protein, 17 aa, synthetic construct
  REGION 1..17: note = Synthetic
KSSQNLLYSRDQKNYLA

SEQ ID NO: 190 - protein, 15 aa, synthetic construct
  REGION 1..15: note = Synthetic
WYQQKPGQSPNLLIY

SEQ ID NO: 191 - protein, 32 aa, synthetic construct
  REGION 1..32: note = Synthetic
GVPDRFTGSGSGTDFSLTISSVKAEDLAVYYC

SEQ ID NO: 192 - protein, 11 aa, synthetic construct
  REGION 1..11: note = Synthetic
FGGGTKLEMKR

SEQ ID NO: 193 - protein, 23 aa, synthetic construct
  REGION 1..23: note = Synthetic
DIVLTQSPASLAVSLGQRATISC

SEQ ID NO: 194 - protein, 15 aa, synthetic construct
  REGION 1..15: note = Synthetic
KASQSVDYDGDSYMN

SEQ ID NO: 195 - protein, 15 aa, synthetic construct
  REGION 1..15: note = Synthetic
WYQQKPGQPPKLLIY

SEQ ID NO: 196 - protein, 7 aa, synthetic construct
  REGION 1..7: note = Synthetic
DASNLES

SEQ ID NO: 197 - protein, 32 aa, synthetic construct
  REGION 1..32: note = Synthetic
GIPARFSGSGSGTDFTLNIHPVEEEDAATYYC

SEQ ID NO: 198 - protein, 9 aa, synthetic construct
  REGION 1..9: note = Synthetic
QQSNEAPWT

SEQ ID NO: 199 - protein, 7 aa, synthetic construct
  REGION 1..7: note = Synthetic
DASTLES

SEQ ID NO: 200 - protein, 9 aa, synthetic construct
  REGION 1..9: note = Synthetic
QQNYEAPWT

SEQ ID NO: 201 - protein, 5 aa, synthetic construct
  REGION 1..5: note = Synthetic
  VARIANT 1: note = Wherein Xaa at position 1 is N or S or T
XYWMS

SEQ ID NO: 202 - protein, 19 aa, synthetic construct
  REGION 1..19: note = Synthetic
  VARIANT 7: note = Wherein Xaa at position 7 is D or E
  VARIANT 11: note = Wherein Xaa at position 11 is T or A
  VARIANT 12: note = Wherein Xaa at position 12 is H or Y
  VARIANT 14: note = Wherein Xaa at position 14 is A or T
EIRLKSXNYAXXYXESVKG

SEQ ID NO: 203 - protein, 5 aa, synthetic construct
  REGION 1..5: note = Synthetic
  VARIANT 5: note = Wherein Xaa at position 5 is S or Y or N
AWFAX

SEQ ID NO: 204 - protein, 17 aa, synthetic construct
  REGION 1..17: note = Synthetic
  VARIANT 1: note = Wherein Xaa at position 1 is M or K
  VARIANT 5: note = Wherein Xaa at position 5 is S or N
  VARIANT 10: note = Wherein Xaa at position 10 is K or R
XSSQXLLYSXDQKNYLA

SEQ ID NO: 205 - protein, 9 aa, synthetic construct
  REGION 1..9: note = Synthetic
  VARIANT 5: note = Wherein Xaa at position 5 is T or S
LQYYXYPYT

SEQ ID NO: 206 - DNA, 348 bp, synthetic construct
  misc_feature 1..348: note = Synthetic
  source 1..348: mol_type = other DNA
gaagtgaagcttgaggagtctggaggaggcctggtgcaacctggaggatccatgaaactctcctgtgtagcctctggatttactttcggtaactactggatgtcttgggtccgccagtctccagagaaggggcttgagtgggttgctgaaattagattgaaatctgataattatgcaacacattatgcggagtctgtgaaagggaagttcaccatctcaagagatgattccaaaagtcgtctctacctgcaaatgaacagcttaagaggtgaagacactggactttattactgtacgaatgcctggtttgcttcctggggccaagggactctggtcactgtctctgca

SEQ ID NO: 207 - DNA, 348 bp, synthetic construct
  misc_feature 1..348: note = Synthetic
  source 1..348: mol_type = other DNA
gaagtgaagcttgaggagtctggaggaggcttggtgcaacctggaggatccatgaaactctcctgtgttgcctctggatttactttcggtagctactggatgtcttgggtccgccagtctccagagaaggggcttgagtgggttgctgaaattagattgaaatctgataattatgcagcacattatgcggagtctgtgaaagggaagttcaccatctcaagagatgattccaaaagtcgtctctacctgcaaatgaacagcttaagaggcgaagacactggaatttattactgtacgaatgcctggtttgcttcctggggccaagggactctggtcactgtttctgca

SEQ ID NO: 208 - DNA, 348 bp, synthetic construct
  misc_feature 1..348: note = Synthetic
  source 1..348: mol_type = other DNA
gaagtgaagcttgaggagtctggaggaggcttggtgcaacctggaggatccatgaaactctcctgtgttgcctctggatttactttcagcacttattggatgtcttgggtccgccagtctccagagaaggggcttgagtgggttgctgaaattagattgaaatctgataattatgcaacacattatacggagtctgtgaaagggaagttcaccatctcaagagatgattccaaaagtcgtctctacctgcaaatgaacagtttaagagttgaagacactggaatttattattgtacgaatgcctggtttgcttactggggccaagggactctggtcactgtctctgca

SEQ ID NO: 209 - DNA, 348 bp, synthetic construct
  misc_feature 1..348: note = Synthetic
  source 1..348: mol_type = other DNA
gaagtgaagcttgaggagtctggaggaggcttggtgcaacctggaggatccatgaaactctcctgtattgcctctggatttactttcagtacctactggatgtcttgggtccgccagtctccagagaaggggcttgagtgggttgctgaaattagattgaaatctgaaaattatgcaacatattatgcggagtctgtgaaagggaagttcatcatctcaagagatgattccaaaagtcgtctctacctgcaaatgaacagcttaagagctgaagacactggaatttattactgtacgaatgcctggtttgctaactggggccaagggactctggtcactgtctctgca

SEQ ID NO: 210 - DNA, 345 bp, synthetic construct
  misc_feature 1..345: note = Synthetic
  source 1..345: mol_type = other DNA
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacattggtggcattggctacaacccatctctcaaaagtcgaatctctatcactcgagacacatccaagaaccagttcttcctgcacttgaattctgtgactactggggacacagccacatattactgtgcaagaaacggggctatggacttctggggtcaaggaatctcagtcaccgtctcctca

SEQ ID NO: 211 - DNA, 345 bp, synthetic construct
  misc_feature 1..345: note = Synthetic
  source 1..345: mol_type = other DNA
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagttacagtggtagcactggctatagcccatctctcaaaagtcgaatctctatcactcgagacacatccaagaaccagttcttcctgcacttgaattctgtgactactggagacacagccacatattactgtgcacgaaacggggctatggactactggggtcaaggaatctcagtcaccgtctcctca

SEQ ID NO: 212 - DNA, 342 bp, synthetic construct
  misc_feature 1..342: note = Synthetic
  source 1..342: mol_type = other DNA
gacattgtgatgtcacagtctccatcctccctagctgtgtcagttggagagaaggttactatgagctgcatgtccagtcagagccttttatatagtaaagatcaaaagaactacttggcctggtaccaacagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtctgcaatattatacctatccgtacacgttcggaggggggaccaagctggaaataaaacgg

SEQ ID NO: 213 - DNA, 342 bp, synthetic construct
  misc_feature 1..342: note = Synthetic
  source 1..342: mol_type = other DNA
gacattgtgatgtcacagtctccatcctccctaactgtgtcagttggagagaaggttactatgagctgcatgtccagtcagagccttttatatagtaaagatcaaaagaactacttggcctggtaccaacagaaaccagggcagtctcctacactgctgatttactgggcatccactagggaatctggggtccctgatcgcttcacaggcagtggatctgggacagatttcactctcaccatcagcagtgtgaaggctgaagacctggcagtttattactgtctgcaatattatacctatccgtacacgttcggaggggggaccaagctggaaataaaacgg

SEQ ID NO: 214 - DNA, 342 bp, synthetic construct
  misc_feature 1..342: note = Synthetic
  source 1..342: mol_type = other DNA
gacattgtgatgtcacagtctccatcctccctagctgtgtcagttggagagaagtttactatgagctgcaagtccagtcagagccttttatatagtcgcgatcaaaagaactacttggcctggtaccagcagcaaccagggcagtctcctaaacttctgatttactgggcatccactagggagtctggggtccctgatcgcttcacaggcagtggatctgggacagatttcactctcaccatcagcagtgtgaagactgaagacctggcagtttattactgtctccaatattatagctatccgtacactttcggaggggggaccaagctggaaataaaacgg

SEQ ID NO: 215 - DNA, 342 bp, synthetic construct
  misc_feature 1..342: note = Synthetic
  source 1..342: mol_type = other DNA
gacattgtgatgtcacagtctccatcctccctagttgtgtcagttggagagaaggttactatgagctgtaagtccagtcagaaccttttatatagtagggatcaaaagaactacttggcctggtaccagcagaaaccagggcagtctcctaacttgctgatttactgggcatccactagggaatctggggtccctgatcgcttcacaggcagtggatctgggacagatttctctctcaccatcagcagtgtgaaggctgaagacctggcagtttattactgtctccaatattatagctatccgtacacgttcggaggggggaccaagctggaaatgaaacgg

SEQ ID NO: 216 - DNA, 336 bp, synthetic construct
  misc_feature 1..336: note = Synthetic
  source 1..336: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgatgcatccaatctagaatctgggatcccagccaggtttagtggcagtgggtctgggacagacttcaccctcaacatccatcctgtggaggaggaggatgctgcaacctattactgtcagcaaagtaatgaggctccgtggacgttcggtggaggcaccaagctggaaatcaaacgg

SEQ ID NO: 217 - DNA, 336 bp, synthetic construct
  misc_feature 1..336: note = Synthetic
  source 1..336: mol_type = other DNA
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatttatgatgcttccactctagaatctgggatcccagccaggtttagtggcagtgggtctgggacagacttcaccctcaacatccatcctgtggaggaggaggatgctgcaacctattactgtcagcaaaattatgaggctccgtggacgttcggtggaggcaccaagctggaaatcaaacgg

SEQ ID NO: 218 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 219 - protein, 106 aa, Mus musculus
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 220 - protein, 114 aa, synthetic construct
  REGION 1..114: note = Synthetic
QVQLVQSGAEVKKPGASVKVSCKASGYTFTTDDINWVRQAPGQGLEWIGWIYPRDDRTKYNDKFKDKATLTVDTSSNTAYMELSSLRSEDTAVYYCSSLEDTYWGQGTLVTVSS

SEQ ID NO: 221 - protein, 113 aa, synthetic construct
  REGION 1..113: note = Synthetic
DIVMTQSPDSLAVSLGERATINCKSSQSLLASRTRKNYLAWYQQKPGQPPKLLIYWASTRESGVPDRFSGSGSGTDFTLTISSLQAEDVAVYYCKQSYNLYTFGQGTKVEIKR

SEQ ID NO: 222 - protein, 114 aa, synthetic construct
  REGION 1..114: note = Synthetic
QVQLVQSGAEVKKPGASVKVSCKASGYTFTTDDINWVRQAPGQGLEWIGWIYPRDDRTKYNDKFKDRATLTVDTSSNTAYMELSSLRSEDTAVYYCSSLEDTYWGQGTLVTVSS

SEQ ID NO: 223 - protein, 116 aa, synthetic construct
  REGION 1..116: note = Synthetic
QIQLVQSGSELKKPGASVKVSCKASGYIFTSYGMSWVRQAPGKGLKWMGWINTYSGVPTYADDFKGRFVFSLDTSVRTPYLQISSLKAEDTAVYFCARGGEAMDYWGQGTLVTVSS

SEQ ID NO: 224 - protein, 113 aa, synthetic construct
  REGION 1..113: note = Synthetic
DVLMTQTPLSLSVTPGQPASISCKSSQSLLDSDAKTYLNWLLQRPGQSPKRLIYLVSKLDSGVPDRFSGSGSGTDFTLKISRVEAEDVGVYYCWQGTHFPWTFGQGTKVEIKR

SEQ ID NO: 225 - protein, 116 aa, synthetic construct
  REGION 1..116: note = Synthetic
QIQLVQSGSELKKPGASVKVSCKASGYIFTSYGMSWVRQAPGKGLKWMGWINTYSGVPTYADDFKGRFVFSLDTSVRTPYLQISSLKAEDTATYFCARGGEAMDYWGQGTLVTVSS

SEQ ID NO: 226 - protein, 113 aa, synthetic construct
  REGION 1..113: note = Synthetic
QVQLVQSGAEVKKPGASVKVSCKASGYTFTGKWIEWVRQAPGQGLEWIGEILPGTGSTNYAQKFQGRATFTADSSTSTAYMELSSLRSEDTAVYYCLRSEDVWGQGTLVTVSS

SEQ ID NO: 227 - protein, 113 aa, synthetic construct
  REGION 1..113: note = Synthetic
DIVMTQSPDSLAVSLGERATINCKSSQSLLASRTRKNYLAWYQQKPGQPPKLLIYWASTRESGVPDRFSGSGSGTDFTLTISSLQAEDVAVYYCKQSYNIPTFGQGTKVEIKR

SEQ ID NO: 228 - protein, 113 aa, synthetic construct
  REGION 1..113: note = Synthetic
QVQLVQSGAEVKKPGASVKVSCKASGYTFTGKWIEWVRQAPGQGLEWIGEILPGTGSTNYNEKFKGRATFTADSSTSTAYMELSSLRSEDTAVYYCLRSEDVWGQGTLVTVSS

SEQ ID NO: 229 - protein, 5 aa, synthetic construct
  REGION 1..5: note = Synthetic
TDDIN

SEQ ID NO: 230 - protein, 5 aa, synthetic construct
  REGION 1..5: note = Synthetic
SYGMS

SEQ ID NO: 231 - protein, 5 aa, synthetic construct
  REGION 1..5: note = Synthetic
GKWIE

SEQ ID NO: 232 - protein, 17 aa, synthetic construct
  REGION 1..17: note = Synthetic
WIYPRDDRTKYNDKFKD

SEQ ID NO: 233 - protein, 17 aa, synthetic construct
  REGION 1..17: note = Synthetic
WINTYSGVPTYADDFKG

SEQ ID NO: 234 - protein, 17 aa, synthetic construct
  REGION 1..17: note = Synthetic
EILPGTGSTNYAQKFQG

SEQ ID NO: 235 - protein, 17 aa, synthetic construct
  REGION 1..17: note = Synthetic
EILPGTGSTNYNEKFKG

SEQ ID NO: 236 - protein, 5 aa, synthetic construct
  REGION 1..5: note = Synthetic
LEDTY

SEQ ID NO: 237 - protein, 7 aa, synthetic construct
  REGION 1..7: note = Synthetic
GGEAMDY

SEQ ID NO: 238 - protein, 4 aa, synthetic construct
  REGION 1..4: note = Synthetic
SEDV

SEQ ID NO: 239 - protein, 17 aa, synthetic construct
  REGION 1..17: note = Synthetic
KSSQSLLASRTRKNYLA

SEQ ID NO: 240 - protein, 16 aa, synthetic construct
  REGION 1..16: note = Synthetic
KSSQSLLDSDAKTYLN

SEQ ID NO: 241 - protein, 7 aa, synthetic construct
  REGION 1..7: note = Synthetic
LVSKLDS

SEQ ID NO: 242 - protein, 8 aa, synthetic construct
  REGION 1..8: note = Synthetic
KQSYNLYT

SEQ ID NO: 243 - protein, 9 aa, synthetic construct
  REGION 1..9: note = Synthetic
WQGTHFPWT

SEQ ID NO: 244 - protein, 8 aa, synthetic construct
  REGION 1..8: note = Synthetic
KQSYNIPT

SEQ ID NO: 245 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 246 - protein, 32 aa, synthetic construct
  REGION 1..32: note = Synthetic
RFTISRDDSESMLSLQMNNLKTEDTAMYYCVR

SEQ ID NO: 247 - protein, 30 aa, synthetic construct
  REGION 1..30: note = Synthetic
EVKLEESGGGLVQPGGSMKLSCVASGFTFS

(19) United States
(12) Patent Application Publication
(10) Pub. No.: US 2025/0179160 A1
(43) Pub. Date: Jun. 5, 2025

(54) MONOCLONAL ANTIBODIES, COMPOSITIONS AND METHODS FOR DETECTING COMPLEMENT FACTOR D

(71) Applicant: Omeros Corporation, Seattle, WA (US)

(72) Inventors: William Jason Cummings, Kirkland, WA (US); Jeremy A. Freeman, Shoreline, WA (US); Yi Li, Seattle, WA (US); Kathleen A. Shaffer, Rosharon, TX (US); Munehisa Yabuki, Seattle, WA (US)

(21) Appl. No.: 18/892,252

(22) Filed: Sep. 20, 2024

Related U.S. Application Data
(62) Division of application No. 17/404,323, filed on Aug. 17, 2021, now Pat. No. 12,129,290.
(60) Provisional application No. 63/066,942, filed on Aug. 18, 2020.
(60) Provisional application No. 63/066,948, filed on Aug. 18, 2020.
(60) Provisional application No. 63/197,833, filed on Jun. 7, 2021.

(51) Int. Cl. C07K 16/18; G01N 33/573

(52) U.S. Cl. CPC C07K 16/18; G01N 33/573; C07K 2317/34; C07K 2317/92; G01N 2333/96433

(57) ABSTRACT

Disclosed herein are monoclonal antibodies that specifically bind to human mature Factor D and that do not bind to human Pro-Factor D, monoclonal antibodies that specifically bind to human Pro-Factor D and do not bind to human mature Factor D, and monoclonal antibodies that bind to both human mature Factor D and human Pro-Factor D. Also disclosed are methods of using the monoclonal antibodies, and compositions comprising the same, for detection of the mature and/or the pro-form of Factor D in biological samples, to determine the status of the Alternative Pathway of Complement (APC) in a mammalian subject, or to determine the status of Factor D after treatment with a MASP-3 inhibitory agent which inhibits the conversion of Pro-Factor D to mature Factor D.

### CROSS-REFERENCE TO RELATED APPLICATIONS

This application is a divisional application of pending U.S. patent application Ser. No. 17/404,323, which claims the benefit of U.S. Provisional Application No. 63/066,942 filed Aug. 18, 2020, now expired, U.S. Provisional Application No. 63/066,948, filed Aug. 18, 2020, now expired, and U.S. Provisional Application No. 63/197,833 filed Jun. 7, 2021, now expired, which are hereby incorporated by reference in their entirety.

### FIELD OF THE INVENTION

The present invention relates to monoclonal antibodies and compositions comprising such antibodies for use in detecting the presence and amount of mature Factor D and Pro-Factor D.

### STATEMENT REGARDING SEQUENCE LISTING

The sequence listing associated with this application is provided in text format in lieu of a paper copy and is hereby incorporated by reference into the specification. The name of the XML file containing the sequence listing is: MP_1_0316_US2_Sequence Listing_20240920_ST26. The XML file is 331,128 bytes; was created on Sep. 20, 2024 and is being submitted via the Patent Center with the filing of the specification.

### BACKGROUND

The complement system supports innate host defense against pathogens, dysregulated and unabated complement activity can also function as a major driver of autoimmune disease, causing unchecked propagation of inflammation and tissue destruction. However, dysregulated and unabated complement activity can also function as a major driver of disease, causing unchecked propagation of inflammation and tissue destruction. The alternative pathway of complement (APC) is typically described as a downstream amplifier of complement activity, increasing the host immune response following activation of complement via the classical and lectin pathways. However, the ability of the APC to create a positive feedback loop of protease complexes with activity that drives the formation of new complexes of the same type is unique within the complement pathways (Lachmann P. J, Adv Immunol 104:115-49, 2009).

Complement Factor D (CFD) is a serine protease that is essential for activation of the APC. Factor D cleaves factor B bound to C3b, generating the C3b/Bb enzyme which is the active component of the alternative pathway C3/C5 convertases. While CFD is expressed as an inactive zymogen (referred to herein as “Pro-Factor D”), it circulates in plasma predominantly as a cleaved, mature serine protease (referred to herein as “mature Factor D”). As described in WO2013/180834 and WO2013/192240, it has recently been determined that MASP-3 is responsible for the conversion of complement factor D (CFD) from the zymogen form of the protein (Pro-Factor D) to the active form (mature Factor D), thus placing the MASP-3 protein at a key upstream regulatory step for the APC. As further described in WO2018/026722, hereby incorporated herein by reference, numerous high affinity anti-MASP-3 inhibitory antibodies have been generated that bind the serine protease domain of MASP-3 and inhibit its catalytic activity.

A current problem in the area of complement research is that anti-Factor D antibodies in commercially available test kits do not differentiate between Pro-Factor D and the active form (mature Factor D). In a wild-type animal or human plasma, the large majority of systemic CFD has already been processed to the mature form by in vivo MASP-3 activity, making in vitro assessment of APC inhibition by MASP-3 inhibitors using traditional assays impossible. Therefore, a need exists for detection reagents and assays for measuring the presence and amount of Pro-Factor D and/or mature Factor D in a biological sample for use as a biomarker of APC status and thereby allowing for in vitro assessment of APC inhibition by MASP-3 inhibitors.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present invention addresses the need for detection reagents and assays for measuring the presence and amount of Pro-Factor D and/or mature Factor D in a biological sample.

In one aspect, the present disclosure provides an isolated antibody, or antigen binding fragment thereof, that specifically binds to an epitope in the amino-terminal region of human mature Factor D, wherein the epitope comprises or consists of the amino acid sequence ILGGREA (SEQ ID NO:5). In one embodiment, the isolated antibody or fragment thereof specifically binds human mature Factor D (SEQ ID NO:3) and does not bind to human Pro-Factor D (SEQ ID NO:2). In one embodiment, the antibody is a monoclonal antibody. In one embodiment, the present disclosure provides a nucleic acid molecule encoding the CDRs of a heavy chain variable region and/or the CDRS of a light chain variable region of an antibody, or fragment thereof, that specifically binds human mature Factor D.

In another aspect, the present disclosure provides an isolated antibody, or antigen binding fragment thereof, that specifically binds to an epitope on the activation (“Pro”) peptide of human Factor D, wherein the epitope comprises or consists of “APPRGR” (SEQ ID NO: 4). In one embodiment, the antibody specifically binds to human Pro-Factor D (SEQ ID NO: 2) and does not bind to mature Factor D (SEQ ID NO:3). In one embodiment, the antibody is a monoclonal antibody. In one embodiment, the present disclosure provides a nucleic acid molecule encoding the CDRs of a heavy chain variable region and/or the CDRs of a light chain variable region of an antibody, or fragment thereof, that specifically binds human Pro-Factor D.

In another aspect, the present disclosure provides a kit for detecting the presence or amount of mature factor D and/or Pro-Factor D in a test sample, said kit comprising (a) at least one container, and (b) at least one antibody, or fragment thereof, that specifically binds human mature Factor D and/or Pro-Factor D.

In another aspect, the present disclosure provides an isolated antibody or antigen-binding fragment thereof that binds to an epitope shared by human mature Factor D and human Pro-Factor D, wherein the antibody comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

In another aspect, the present disclosure provides a method of determining the presence or amount of mature Factor D in a test sample, the method comprising: (a) contacting a test sample with a mature Factor D-specific monoclonal antibody or antigen-binding fragment thereof, in an in vitro immunoassay; and (b) detecting the presence or absence or amount of the antibody or fragment thereof bound to mature Factor D, wherein the presence of binding indicates the presence or amount of mature Factor D in the sample; wherein the anti-human mature Factor D-specific antibody or antigen binding fragment thereof binds to an epitope in the N-terminal region of mature Factor D, set forth as amino acids ILGGREA (SEQ ID NO:5).

In another aspect, the present disclosure provides a method of determining the presence or amount of Pro-Factor D in a test sample, the method comprising: (a) contacting a test sample with an anti-human Pro-Factor D-specific monoclonal antibody or antigen-binding fragment thereof, in an in vitro immunoassay; and (b) detecting the presence or amount of the antibody or fragment thereof bound to Pro-Factor D, wherein the presence of binding indicates the presence or amount of Pro-Factor D in the sample; wherein the anti-human mature Pro-Factor D-specific antibody or antigen binding fragment thereof specifically binds to an epitope in the activation (“Pro”) peptide of human Factor D, set forth as “APPRGR” (SEQ ID NO:4).

In another aspect, the present disclosure provides a method of assessing the extent of alternative pathway complement (APC) activation in a test sample comprising: (a) providing a test sample; (b) performing an immunoassay comprising at least one of: (i) capturing and detecting mature Factor D in the test sample, wherein mature Factor D is either captured or detected with a mature Factor D-specific monoclonal antibody or fragment thereof that specifically binds to an epitope in “ILGGREA” (SEQ ID NO:5) present in mature Factor D, but does not bind to Pro-Factor D; and/or (ii) capturing and detecting Pro-Factor D in the test sample, wherein Pro-Factor D is either captured or detected with a Pro-Factor D-specific monoclonal antibody or fragment thereof that specifically binds to an epitope on the activation (“Pro”) peptide “APPRGR” (SEQ ID NO:4) present in Pro-Factor D, but does not bind to mature Factor D; and (c) comparing the level of mature Factor D detected in accordance with (b) (i) with a predetermined level or control sample and/or comparing the level of Pro-Factor D detected in accordance with (b (ii) with a predetermined level or control sample, wherein the level of mature Factor D and/or Pro-Factor D detected in the test sample is indicative of the extent of alternative pathway complement activation.

In another aspect, the present disclosure provides a method for monitoring the efficacy of treatment with a MASP-3 inhibitory antibody in a mammalian subject, the method comprising: (a) administering a dose of a MASP-3 inhibitory antibody to a mammalian subject at a first point in time; (b) assessing a first concentration of mature Factor D and/or Pro-Factor D in a biological sample obtained from the subject after step (a); (c) treating the subject with the MASP-3 inhibitory antibody at a second point in time; (d) assessing a second concentration of mature Factor D and/or Pro-Factor D in a biological sample obtained from the subject after step (c); and (e) comparing the level of mature Factor D and/or Pro-Factor D assessed in step (b) with the level of mature Factor D and/or Pro-Factor D assessed in step (d) to determine the efficacy of the MASP-3 inhibitory antibody in the mammalian subject.

In another aspect, the present disclosure provides a method of treating a mammalian subject suffering from, or at risk of developing an alternative-pathway disease or disorder, comprising administering a MASP-3 inhibitory antibody to the subject if the subject is determined to have: (i) a lower or decreased level of Pro-Factor D in one or more samples taken from the subject compared to a predetermined Pro-Factor D level or compared to the Pro-Factor D level in one or more control samples: and/or (ii) a higher or increased level of mature Factor D in one or more samples taken from the subject compared to a predetermined mature Factor D level or compared to the mature Factor D level in one or more control samples.

In another aspect, the present disclosure provides a pharmaceutical composition comprising a MASP-3 inhibitory antibody in an aqueous solution comprising a buffer system having a pH of 6.0±5%, 20±5% mM histidine, 100±5% mg/mL sucrose, and 0.035%±5%, polysorbate 80 wherein said MASP-3 inhibitory antibody is included at a concentration of 110 mg/mL±5%, and wherein said MASP-3 inhibitory antibody comprises a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT).

In another aspect, the present disclosure provides an article of manufacture containing a pharmaceutical composition comprising a MASP-3 inhibitory antibody, wherein the MASP-3 inhibitory antibody is in a unit dosage form of from 10 mg to 1000 mg suitable for therapeutic administration to a human subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating the classical, lectin and alternative complement pathways.

FIG. 2 provides the amino acid sequences of (i) human full-length Factor D (SEQ ID NO:1), including the signal sequence aa 1-19 (shown in italic) and the activation (pro) peptide underlined; (ii) human Pro-Factor D (SEQ ID NO:2), with the pro-peptide underlined; and (iii) human mature Factor D (SEQ ID NO:3).

FIG. 3 provides an alignment of the amino acid sequences of pro-Factor D from various species.

FIG. 4 graphically illustrates the titration of anti-serum from representative mouse #2 after immunization with a peptide corresponding to the N-terminus of mature human factor D, as described in Example 1.

FIG. 5 graphically illustrates the results of a capture ELISA assay in which hybridoma supernatants were screened for binding to human mature-Factor D or human Pro-Factor D when captured by a polyclonal anti-Factor D antibody AF1824 (R&D Systems), as described in Example 1.

FIG. 6A graphically illustrates the results of an ELISA assay with coated polyclonal goat anti-human CFD 1824 and detected with hybridoma supernatant 14A11 present in each condition described in Example 1. As shown in FIG. 6A, hybridoma supernatant 14A11 is capable of selectively detecting recombinant mature complement factor D and does not detect recombinant pro factor D, as described in Example 1.

FIG. 6B graphically illustrates the results of an ELISA assay with coated polyclonal goat anti-human CFD 1824 and detected with hybridoma supernatant 6G6 present in each condition described in Example 1. As shown in FIG. 6B, hybridoma supernatant 6G6 is capable of selectively detecting recombinant mature complement factor D and does not detect recombinant pro factor D, as described in Example 1.

FIG. 6C graphically illustrates the results of an ELISA assay with coated polyclonal goat anti-human CFD 1824 and detected with monoclonal antibody mAb1824 (R&D Systems) present in each condition described in Example 1. As shown in FIG. 6C, monoclonal antibody 1824 (R&D Systems) detects both recombinant mature CFD and recombinant active CFD and therefore is not capable of selectively detecting mature CFD as compared to pro CFD, as described in Example 1.

FIG. 7A shows an amino acid alignment of the heavy chain variable region (VH) sequences for the anti-human mature-Factor D-specific clones: 6G6_VH (SEQ ID NO:12), 14A11_VH (SEQ ID NO:13), 27B3_VH (SEQ ID NO:14), 58F5_VH (SEQ ID NO:15), 49G3_VH (SEQ ID NO:16), and 10G1_VH (SEQ ID NO:17), as described in Example 2.

FIG. 7B shows an amino acid alignment of the light chain variable region (VL) sequences for the anti-human mature-Factor D-specific clones: 6G6_VK (SEQ ID NO:18), 14A11_VK: (SEQ ID NO:19), 27B3_VK: (SEQ ID NO:20), 58F5_VK: (SEQ ID NO:21), 49G3_VK: (SEQ ID NO:22), and 10G1_VK: (SEQ ID NO:23), as described in Example 2.

FIG. 8 graphically illustrates the detection (or lack thereof) of recombinant human pro-Factor D or mature-Factor D with numerous candidate anti-human mature-Factor-D-specific antibodies. As shown in FIG. 8, all the purified antibodies tested, namely 6G6, 14A11, 10G1, 49G3, 27B3 and 58F5, were found to be specific for the mature form of Factor D, as described in Example 3.

FIG. 9 graphically illustrates a titration of the serum of a representative mouse #1189 after immunization with human mature Factor D in the presence of recombinant mature Factor D or recombinant pro-Factor D. As shown in FIG. 9, the serum from representative mouse #1189 contains antibodies capable of binding to both mature Factor D and pro-Factor D, as described in Example 4.

FIG. 10A shows an amino acid alignment of the heavy chain variable region (VH) sequences for the anti-human Factor D clones: 3C5_VH (SEQ ID NO:85), 30H2_VH (SEQ ID NO:85), 11H1_VH (SEQ ID NO:86), 12H10_VH (SEQ ID NO:87), and 7H2_VH (SEQ ID NO:88), as described in Example 5.

FIG. 10B shows an amino acid alignment of the light chain variable region (VL) sequences for the anti-human Factor D clones: 3C5_VL (SEQ ID NO:89), 30H2_VL (SEQ ID NO: 90), 11H1_VL (SEQ ID NO:91), 12H10_VL (SEQ ID NO:92) and 7H2_VL (SEQ ID NO: 93), as described in Example 5.

FIG. 11A graphically illustrates the binding of recombinant human pro-Factor D or mature-Factor D with candidate anti-human Factor D antibody 3C5, demonstrating that antibody 3C5 binds to both human pro-Factor D and mature-Factor D, as described in Example 5.

FIG. 11B graphically illustrates the binding of recombinant human pro-Factor D or mature-Factor D with candidate anti-human Factor D antibody 12H10, demonstrating that antibody 12H10 binds to both human pro-Factor D and mature-Factor D, as described in Example 5.

FIG. 12A graphically illustrates the results of an ELISA assay in which the recombinant anti-Factor D antibody 3C5 was coated onto the ELISA plate and allowed to capture recombinant human and cynomolgus mature and pro-Factor D (huMat CFD, cy Mat CFD, huProCFD and cyPro CFD). Also captured was Factor D-depleted human serum (CFD Dpl serum) and a sample of pooled normal cynomolgus plasma (NCP). Detection of captured Factor D was done with a mouse IgG2a Fc version of anti-human mature Factor D-specific mAb 14A11, as described in Example 6.

FIG. 12B graphically illustrates the results of an ELISA assay in which the recombinant anti-Factor D antibody 12H10 was coated onto the ELISA plate and allowed to capture recombinant human and cynomolgus mature and pro-Factor D (huMat CFD, cy Mat CFD, huProCFD and cyPro CFD). Also captured was Factor D-depleted human serum (CFD Dpl serum) and a sample of pooled normal cynomolgus plasma (NCP). Detection of captured Factor D was done with a mouse IgG2a Fc version of anti-human mature Factor D-specific mAb 14A11, as described in Example 6.

FIG. 13 graphically illustrates the detection of human and cynomolgus monkey mature Factor D and Pro-Factor D with a combination of capture antibody 3C5 (anti-human/cyno Factor D) and detection antibody 14A11 (anti-human/cyno mature-Factor D-specific) in an ELISA assay, as described in Example 6.

FIG. 14 graphically illustrates a titration of the serum of a representative mouse #2 after immunization with a synthetic peptide corresponding to amino acid residues 26-32 of human complement factor D: “ILGGREA” (SEQ ID NO:5) in the presence of recombinant mature Factor D or recombinant pro-Factor D. As shown in FIG. 14, the serum from representative mouse #2 contains antibodies capable of selectively binding to mature Factor D as compared to pro-Factor D, as described in Example 7.

FIG. 15 graphically illustrates the results of a capture ELISA assay in which hybridoma supernatants were screened for binding to human Pro-Factor D or human mature-Factor D when captured by a polyclonal anti-Factor D antibody AF1824 (R&D Systems), as described in Example 7.

FIG. 16A shows an amino acid alignment of the heavy chain variable region (VH) sequences for the anti-human Pro-Factor D-specific clones: 18F5_VH (SEQ ID NO: 136), 1F9_VH (SEQ ID NO:137), 2A4_VH (SEQ ID NO:138), 20A1_VH (SEQ ID NO: 139), 13A10_VH (SEQ ID NO:140) and 21H1_VH (SEQ ID NO:141), as described in Example 8.

FIG. 16B shows an amino acid alignment of the light chain variable region (VL) sequences for the anti-human Pro-Factor D-specific clones: 18F5_VK (SEQ ID NO:142), 1F9_VK (SEQ ID NO:143), 2A4_VK (SEQ ID NO:144), 20A1_VK (SEQ ID NO:145), 13A10_VK (SEQ ID NO:146), and 21H1_VK (SEQ ID NO:147), as described in Example 8.

FIG. 17A graphically illustrates the detection of recombinant human Pro-Factor D with numerous candidate anti-human Pro Factor-D-specific antibodies. As shown in FIG. 17A, all the purified antibodies tested, namely, 18F5, 1F9, 2A4, 20A1, 13A10, and 21H1, were capable of detecting the pro form of Factor D, as described in Example 9.

FIG. 17B graphically illustrates the detection of recombinant human mature-Factor D with numerous candidate anti-human Pro Factor-D-specific antibodies. As shown in FIG. 17B, none of the purified antibodies tested, namely, 18F5, 1F9, 2A4, 20A1, 13A10, and 21H1, were capable of detecting the mature form of Factor D, as described in Example 9.

FIG. 18 graphically illustrates the detection of Pro-Factor D and mature Factor D in an ELISA assay with anti-Pro-Factor D antibody 21H1 as the coating antibody and goat polyclonal anti-Factor D antibody AF1824 (R&D Systems) as the detection antibody, as described in Example 9.

FIG. 19 graphically illustrates the detection of Pro-Factor D and mature Factor D in normal human plasma (NHP), normal human serum (NHS) or Factor-D-depleted serum (Df-Dpl serum) an ELISA assay with anti-Pro-Factor D antibody 21H1 as the coating antibody and goat polyclonal anti-Factor D antibody AF1824 (R&D Systems) as the detection antibody, as described in Example 9.

FIG. 20 graphically illustrates the amount of Pro-Factor D present in Normal Human Serum (NHS), C1q-Depleted Serum (C1q-Dpl), Factor D-Depleted Serum (Df-Dpl) and 3MC-syndrome patient serum_as determined in an ELISA assay with anti-Pro-Factor D antibody 21H1 as the coating antibody and goat polyclonal anti-Factor D antibody AF1824 (R&D Systems) as the detection antibody, as described in Example 9.

FIG. 21A graphically illustrates the amount of mature Factor D in a cynomolgus monkey over a time period of 912 hours post treatment with representative anti-MASP-3 mAb13B1, as described in Example 11.

FIG. 21B graphically illustrates the standard curve as determined from a 4-parameter logistics curve of cynomolgus recombinant mature Factor D dilutions, as described in Example 11.

FIG. 22A graphically illustrates the concentration of mature Factor D in monkeys over a time period of 1344 hours after subcutaneous (SC) or intravenous (IV) administration of anti-MASP-3 mAb13B1, as measured in an ELISA assay with mature Factor D-specific antibody 14A11, as described in Example 12.

FIG. 22B graphically illustrates the ex vivo alternative pathway activity (% baseline) over a time period of 1344 hours after administration anti-MASP-3 mAb13B1, as determined in a Factor Ba assay, as described in Example 12.

FIG. 23 graphically illustrates the PD-PD relationship of anti-MASP-3 mAb13B1 effects on ex vivo alternative pathway activity and mature Factor D concentration following a single intravenous bolus or subcutaneous administration in monkey, as described in Example 12.

FIG. 24 graphically illustrates the PK-PD relationship of the dosage of anti-MASP-3 mAb13B1 and the effect on mature Factor D (% baseline), as described in Example 12.

FIG. 25A graphically illustrates the concentration of mAb13B1 in serum of subjects over a time period of up to 84 days after intravenous (IV) administration of mAb13B1, as determined by ELISA, as described in Example 13.

FIG. 25B graphically illustrates the levels of mature Factor D in subjects over a time period of 84 days after intravenous (IV) administration of anti-MASP-3 mAb 13B1, as determined in an ELISA assay with mature Factor D-specific antibody 14A11 used as a detection antibody, as described in Example 13.

### DESCRIPTION OF THE SEQUENCE LISTING

SEQ ID NO:1 human full-length Factor D amino acid sequence (including the signal sequence)
SEQ ID NO:2 human pro-Factor D amino acid sequence (without signal sequence) SEQ ID NO:3: human mature Factor D amino acid sequence
SEQ ID NO:4 human pro peptide “APPRGR”, corresponding to residues 20-25 of human full-length Factor D
SEQ ID NO:5 human Mature Factor D N-terminal peptide (“ILGGREA”), corresponding to residues 1-7 of human mature Factor D.
SEQ ID NO:6 synthetic ILGGREA peptide-KLH conjugate “ILGGREAGPGPGAKFVAAAWTLKAAAKKC”
SEQ ID NO:7: human MASP-3 protein
SEQ ID NO:8: macaca full length Factor D
SEQ ID NO:9: canis full-length Factor D
SEQ ID NO:10: rattus full-length Factor D
SEQ ID NO:11: mus full-length Factor D
   Anti-Human Mature Factor D-Specific mAbs: VH Chains
SEQ ID NO:12: mAb clone 6G6 VH amino acid sequence
SEQ ID NO:13: mAb clone 14A11 VH amino acid sequence
SEQ ID NO:14: mAb clone 27B3 VH amino acid sequence
SEQ ID NO:15: mAb clone 58F5 VH amino acid sequence
SEQ ID NO:16: mAb clone 49G3 VH amino acid sequence
SEQ ID NO:17: mAb clone 10G1 VH amino acid sequence
   Anti-Human Mature Factor D-Specific mAbs: VL Chains
SEQ ID NO:18: mAb clone 6G6 VL amino acid sequence
SEQ ID NO:19: mAb clone 14A11 VL amino acid sequence
SEQ ID NO:20: mAb clone 27B3 VL amino acid sequence
SEQ ID NO:21: mAb clone 58F5 VL amino acid sequence
SEQ ID NO:22: mAb clone 49G3 VL amino acid sequence
SEQ ID NO:23: mAb clone 10G1 VL amino acid sequence
SEQ ID NOs: 24-48: heavy chain FRs and CDRs from mouse anti-human mature Factor D-specific mAbs
SEQ ID NOs: 49-64 light chain FRs and CDRs from mouse anti-human mature Factor D-specific mAbs
SEQ ID NOs: 65-69: CDR consensus sequences from mouse anti-human mature Factor D-specific mAbs
SEQ ID NO:70: human IgG4 constant region
SEQ ID NO:71: human IgG4 constant region with S228P mutation
SEQ ID NO:72: human IgK constant region
SEQ ID NO:73: nucleic acid encoding 6G6 HC variable region
SEQ ID NO:74: nucleic acid encoding 14A11 HC variable region
SEQ ID NO:75: nucleic acid encoding 27B3 HC variable region
SEQ ID NO:76: nucleic acid encoding 58F5 HC variable region
SEQ ID NO:77: nucleic acid encoding 49G3 HC variable region
SEQ ID NO:78: nucleic acid encoding 10G1 HC variable region
SEQ ID NO:79: nucleic acid encoding 6G6 LC variable region
SEQ ID NO:80: nucleic acid encoding 14A11 LC variable region
SEQ ID NO:81: nucleic acid encoding 27B3 LC variable region
SEQ ID NO:82: nucleic acid encoding 58F5 LC variable region
SEQ ID NO:83: nucleic acid encoding 49G3 LC variable region
SEQ ID NO:84: nucleic acid encoding 10G1 LC variable region
   Anti-Human Factor D (c-Term) mAbs: VH Chains
SEQ ID NO:85: mAb clone 3C5 VH amino acid sequence
SEQ ID NO:86: mAb clone 11H1 VH amino acid sequence
SEQ ID NO:87: mAb clone 12H10 VH amino acid sequence
SEQ ID NO:88: mAb clone 7H2 VH amino acid sequence
   Anti-Human Factor D (c-Term) mAbs: VL Chains
SEQ ID NO:89: mAb clone 3C5 VL amino acid sequence
SEQ ID NO:90: mAb clone 30H2 VL amino acid sequence
SEQ ID NO:91: mAb clone 11H1 VL amino acid sequence
SEQ ID NO:92: mAb clone 12H10 VL amino acid sequence
SEQ ID NO:93: mAb clone 7H2 VL amino acid sequence
SEQ ID NOs: 94-109: heavy chain FRs and CDRs from mouse anti-human Factor D mAbs that bind an epitope shared by mature Factor D and Pro-Factor D
SEQ ID NOs: 110-126: light chain FRs and CDRs from mouse anti-human Factor D mAbs that bind an epitope shared by mature Factor D and Pro-Factor D
SEQ ID NO:127: nucleic acid encoding 3C5 HC and 30H2 variable region
SEQ ID NO:128: nucleic acid encoding 11H1 VH variable region
SEQ ID NO:129: nucleic acid encoding 12H10 VH variable region
SEQ ID NO:130: nucleic acid encoding 7H2 VH variable region
SEQ ID NO:131: nucleic acid encoding 3C5 VL variable region
SEQ ID NO:132: nucleic acid encoding 30H2 VL variable region
SEQ ID NO:133: nucleic acid encoding 11H1 VL variable region
SEQ ID NO:134: nucleic acid encoding 12H10 VL variable region
SEQ ID NO:135: nucleic acid encoding 7H2 VL variable region
   Anti-Human Pro-Factor D-Specific mAbs: VH Chains
SEQ ID NO:136: mAb clone 18F5 VH amino acid sequence
SEQ ID NO:137: mAb clone 1F9 VH amino acid sequence
SEQ ID NO:138: mAb clone 2A4 VH amino acid sequence
SEQ ID NO:139: mAb clone 20A1 VH amino acid sequence
SEQ ID NO:140: mAb clone 13A10 VH amino acid sequence
SEQ ID NO:141: mAb clone 21H1VH amino acid sequence
   Anti-Human Pro-Factor D-Specific mAbs: VL Chains
SEQ ID NO:142: mAb clone 18F5 VL amino acid sequence
SEQ ID NO:143: mAb clone 1F9 VL amino acid sequence
SEQ ID NO:144: mAb clone 2A4 VL amino acid sequence
SEQ ID NO:145: mAb clone 20A1 VL amino acid sequence
SEQ ID NO:146: mAb clone 13A10 VL amino acid sequence
SEQ ID NO:147: mAb clone 21H1 VL amino acid sequence
SEQ ID NOs: 148-174: heavy chain FRs and CDRs from mouse anti-human Pro-Factor D-specific mAbs
SEQ ID NOs: 175-200: light chain FRs and CDRs from mouse anti-human Pro-Factor D-specific mAbs
SEQ ID NO:201-205: CDR consensus sequences from mouse anti-human Pro-Factor D-specific mAbs
SEQ ID NO:206 nucleic acid encoding 18F5 HC variable region
SEQ ID NO:207 nucleic acid encoding 1F9 HC variable region
SEQ ID NO:208: nucleic acid encoding 2A4 HC variable region
SEQ ID NO:209: nucleic acid encoding 20A1 HC variable region
SEQ ID NO:210: nucleic acid encoding 13A10 HC variable region
SEQ ID NO:211: nucleic acid encoding 21H1 HC variable region
SEQ ID NO:212 nucleic acid encoding 18F5 LC variable region
SEQ ID NO:213 nucleic acid encoding 1F9 LC variable region
SEQ ID NO:214: nucleic acid encoding 2A4 LC variable region
SEQ ID NO:215: nucleic acid encoding 20A1 LC variable region
SEQ ID NO:216: nucleic acid encoding 13A10 LC variable region
SEQ ID NO:217: nucleic acid encoding 21H1 LC variable region
SEQ ID NO:218: mouse IgG2a constant region
SEQ ID NO:219: mouse kappa light chain constant region
   Anti-Human MASP-3 Inhibitory mAbs
SEQ ID NO:220: h4D5_VH-14_VH
SEQ ID NO:221: h4D5_VL-1-NA
SEQ ID NO:222: h4D5_VH-19
SEQ ID NO:223: h10D12_VH-45
SEQ ID NO:224: h10D12_VL-21-GA
SEQ ID NO:225: h10D12_VH-49
SEQ ID NO:226: h13B1_VH-9
SEQ ID NO:227: h13B1_VL-1-NA
SEQ ID NO:228: h13B1_VH-10
SEQ ID NO:229:h4D5: 14_1 NA HC-CDR1
SEQ ID NO:230: h10D12-45-21-GA HC-CDR1
SEQ ID NO:231: h13B1-9-1-NA HC-CDR1
SEQ ID NO:232: h4D5: 14_1 NA HC-CDR2
SEQ ID NO:233: h10D12-45-21-GA HC-CDR2
SEQ ID NO:234: h13B1-9-1-NA HC-CDR2
SEQ ID NO:235: h13B1-10-1-NA: HC-CDR2
SEQ ID NO:236: h4D5: 14_1 NA HC-CDR3
SEQ ID NO:237: h10D12-45-21-GA HC-CDR3
SEQ ID NO:238: h13B1-9-1-NA HC-CDR3
SEQ ID NO:239: h4D5: 14_1 NA LC-CDR1
SEQ ID NO:240: h10D12-45-21-GA LC-CDR1
SEQ ID NO:241: h10D12-45-21-GA LC-CDR2
SEQ ID NO:242: h4D5: 14_1 NA LC-CDR3
SEQ ID NO:243: h10D12-45-21-GA LC-CDR3
SEQ ID NO:244: h13B1-9-1-NA LC-CDR3
SEQ ID NO:245: human IgG4 constant region with S228P and X mutation
SEQ ID NO:246: mAb clone 7H2 HC_FR3 amino acid sequence
SEQ ID NO:247: mAb clone 2A4 HC_FR1 amino acid sequence

### DETAILED DESCRIPTION

As described in Examples 1-3, monoclonal antibodies have been generated that specifically bind to the N-terminal region of human mature Factor D and that do not bind to Pro-Factor D. As further described in Examples 8-9, monoclonal antibodies have been generated that specifically bind to the Pro-peptide of Pro-Factor D and do not bind to mature Factor D. The mature-Factor D-specific monoclonal antibodies and the Pro-Factor D-specific antibodies are useful for detection of the mature and/or the pro-form of Factor D in biological samples and may be used to determine the status of the Alternative Pathway of Complement (APC) in a mammalian subject. As further described in Examples 10-12, the mature-Factor D specific monoclonal antibodies may also be used to determine the status of Factor D after treatment with a MASP-3 inhibitory agent which inhibits the conversion of Pro-Factor D to mature Factor D. Accordingly, in one embodiment, the present invention is directed to monoclonal antibodies that specifically bind to the N-terminal region of human mature Factor D and the use of these antibodies in methods of detecting the presence or amount of mature Factor D in a biological sample. In another embodiment, the present invention is directed to monoclonal antibodies that specifically bind to the activation (pro) peptide of Pro-Factor D and the use of these antibodies in methods of detecting the presence or amount of Pro-Factor D in a biological sample. In another embodiment, the present invention is directed to the use of mature-Factor-D specific monoclonal antibodies and/or the use of Pro-Factor-D-specific monoclonal antibodies to measure the presence or amount of mature-Factor D and/or Pro-Factor D in a mammalian subject before and after treatment with a MASP-3 inhibitory agent, such as a high affinity MASP-3 inhibitory antibody, wherein the MASP-3 inhibitory antibody is capable of inhibiting the conversion of Pro-Factor D to mature Factor D and thereby inhibit the APC.

### I. Definitions

Unless specifically defined herein, all terms used herein have the same meaning as would be understood by those of ordinary skill in the art of the present invention. The following definitions are provided in order to provide clarity with respect to the terms as they are used in the specification and claims to describe the present invention.

The terms “antibody” and “immunoglobulin” are used interchangeably herein. These terms are well understood by those in the field and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

As used herein, the term “antibody” encompasses antibodies and antibody fragments thereof, derived from any antibody-producing mammal (e.g., mouse, rat, rabbit, and primate including human), or from a hybridoma, phage selection, recombinant expression or transgenic animals (or other methods of producing antibodies or antibody fragments), that specifically bind to an antigen, such as human Pro-Factor D set forth as SEQ ID NO:2 (e.g., an epitope in the Pro Peptide “APPRGR” set forth as SEQ ID NO:4), or human mature Factor D, set forth as SEQ ID NO:3 (e.g., an epitope at the N-terminus of mature Factor D comprising or consisting of “ILGGREA,” set forth as SEQ ID NO:5), or that bind to an epitope shared by human Pro-Factor D and human mature Factor D (e.g., an epitope in the C-terminal region of Factor D (e.g., amino acids 8 to 228 of SEQ ID NO:3). It is not intended that the term “antibody” be limited as regards to the source of the antibody or manner in which it is made (e.g., by hybridoma, phage selection, recombinant expression, transgenic animal, peptide synthesis, etc). Exemplary antibodies include polyclonal, monoclonal and recombinant antibodies; multispecific antibodies (e.g., bispecific antibodies); humanized antibodies; fully human antibodies, murine antibodies; chimeric, mouse-human, mouse-primate, primate-human monoclonal antibodies; and anti-idiotype antibodies, and may be any intact molecule or fragment thereof. As used herein, the term “antibody” encompasses not only intact polyclonal or monoclonal antibodies, but also fragments thereof (such as dAb, Fab, Fab′, F(ab′)₂, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity.

As used herein, the term “antigen-binding fragment” refers to a polypeptide fragment that contains at least one CDR of an immunoglobulin heavy and/or light chains that specifically binds to an antigen such as human Pro-Factor D set forth as SEQ ID NO:2 (e.g., an epitope in the Pro Peptide “APPRGR” set forth as SEQ ID NO:4), or human mature Factor D, set forth as SEQ ID NO:3 (e.g., an epitope at the N-terminus of mature Factor D comprising or consisting of “ILGGREA,” set forth as SEQ ID NO:5), or an epitope shared by human Pro-Factor D and human mature Factor D (e.g., an epitope in the C-terminal region of Factor D (e.g., amino acids 8 to 228 of SEQ ID NO:3). In this regard, an antigen-binding fragment of the herein described antibodies may comprise 1, 2, 3, 4, 5, or all 6 CDRs of a VH and VL sequence, such as 1, 2, 3, 4, 5, or 6 CRS of a VH and VL sequence from the disclosed anti-human Factor D antibodies set forth herein.

As used herein the term “anti-Factor D monoclonal antibodies” refers to a homogenous antibody population, wherein the monoclonal antibody is comprised of amino acids that are involved in the selective binding of an epitope on human Factor D, such as human Pro-Factor D set forth as SEQ ID NO:2 (e.g., an epitope in the Pro Peptide “APPRGR” set forth as SEQ ID NO:4), or that specifically bind to human mature Factor D, set forth as SEQ ID NO:3 (e.g., an epitope at the N-terminus of mature Factor D comprising or consisting of “ILGGREA,” set forth as SEQ ID NO:5), or that bind to an epitope shared by human Pro-Factor D and human mature Factor D (e.g., an epitope in the C-terminal region of Factor D (e.g., amino acids 8 to 228 of SEQ ID NO:3). The term “monoclonal antibody” encompasses not only intact monoclonal antibodies and full-length monoclonal antibodies, but also fragments thereof (such as Fab, Fab′, F(ab′)₂, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope.

As used herein, the modifier “monoclonal” indicates the character of the antibody as being obtained from a substantially homogenous population of antibodies, and is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”. Monoclonal antibodies can be obtained using any technique that provides for the production of antibody molecules by continuous cell lines in culture, such as the hybridoma method described by Kohler, G., et al., Nature 256:495, 1975, or they may be made by recombinant DNA methods (see, e.g., U.S. Pat. No. 4,816,567 to Cabilly). Monoclonal antibodies may also be isolated from phage antibody libraries using the techniques described in Clackson, T., et al., Nature 352:624-628, 1991, and Marks, J. D., et al., J. Mol. Biol. 222:581-597, 1991. Such antibodies can be of any immunoglobulin class including IgG, IgM, IgE, IgA, IgD and any subclass thereof.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids) similarly comprise a variable region (of about 116 amino acids) and one of the aforementioned heavy chain constant regions, e.g., gamma (of about 330 amino acids).

The basic four-chain antibody unit is a heterotetrameric glycoprotein composed of two identical light (L) chains and two identical heavy (H) chains. An IgM antibody consists of 5 of the basic heterotetramer units along with an additional polypeptide called the J chain, and therefore contains 10 antigen binding sites. Secreted IgA antibodies can polymerize to form polyvalent assemblages comprising 2-5 of the basic 4-chain units along with J chain. Each L chain is linked to an H chain by one covalent disulfide bond, while the two H chains are linked to each other by one or more by one or more disulfide bonds, depending on the H chain isotype. Each H and L chain also has regularly spaced intrachain disulfide bridges. The pairing of a VH and VL together forms a single antigen-binding site.

Each H chain has at the N-terminus, a variable domain (VH), followed by three constant domains (CH) for each of the α and γ chains, and four CH domains (CH) for μ and ε isotypes.

Each L chain has at the N-terminus, a variable domain (VL) followed by a constant domain (CL) at its other end. The VL is aligned with the VH and the CL is aligned with the first constant domain of the heavy chain (CH1). The L chain from any vertebrate species can be assigned to one of two clearly distinct types, called kappa (κ) and lambda (λ), based on the amino acid sequences of their constant domains (CL).

Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha (α), delta (δ), epsilon (ε), gamma (γ) and mu (μ), respectively. The γ and α classes are further divided into subclasses on the basis of minor differences in CH sequence and function, for example, humans express the following subclasses: IgG1, IgG2, IgG3, IgG4, IgA1 and IgA2.

For the structure and properties of the different classes of antibodies, see, e.g., Basic and Clinical Immunology, 8th Edition, Daniel P. Stites, Abba I. Terr and Tristram G. Parslow (eds); Appleton and Lange, Norwalk, Conn., 1994, page 71 and Chapter 6.

The term “variable” refers to that fact that certain segments of the V domains differ extensively in sequence among antibodies. The V domain mediates antigen binding and defines specificity of a particular antibody for its particular antigen. However, the variability is not evenly distributed across the 110 amino acid span of the variable domains. Rather, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the n-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md (1991)). The constant domains are not involved directly in binding an antibody to an antigen, but exhibit various effector functions, such as participation of the antibody dependent cellular cytotoxicity (ADCC).

As used herein, the term “hypervariable region” refers to the amino acid residues of an antibody that are responsible for antigen binding. The hypervariable region generally comprises amino acid residues from a “complementary determining region” or “CDR” (i.e., from around about residues 24-34 (L1), 50-56 (L2) and 89-97 (L3) in the light chain variable domain, and around about 31-35 (H1), 50-66 (H2) and 95-102 (H3) in the heavy chain variable domain when numbering in accordance with the Kabat numbering system as described in Kabat, et al., Sequences of Proteins of Immunological Interest, 5th Ed. Public Health Service, National Institutes of Health, Bethesda, Md (1991)); and/or those residues from a “hypervariable loop” (i.e., residues 24-34 (L1), 50-56 (L2) and 89-97 (L3) in the light chain variable domain, and 26-32 (H1), 52-56 (H2) and 95-101 (H3) in the heavy chain variable domain when numbered in accordance with the Chothia numbering system, as described in Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2), and 105-120 (H3) in the VH when numbered in accordance with the IMGT numbering system as described in Lefranc, J. P., et al., Nucleic Acids Res 27:209-212; Ruiz, M., et al., Nucleic Acids Res 28:219-221 (2000)).

As used herein, the term “antibody fragment” refers to a portion derived from or related to a full-length anti-Factor D antibody, generally including the antigen binding or variable region thereof. Illustrative examples of antibody fragments include Fab, Fab′, F(ab)₂, F(ab′)₂ and Fv fragments, scFv fragments, diabodies, linear antibodies, single-chain antibody molecules, bispecific and multispecific antibodies formed from antibody fragments.

As used herein, a “single-chain Fv” or “scFv” antibody fragment comprises the V_{H} and V_{L} domains of an antibody, wherein these domains are present in a single polypeptide chain. Generally, the Fv polypeptide further comprises a polypeptide linker between the V_{H} and V_{L} domains, which enables the scFv to form the desired structure for antigen binding. See Pluckthun in The Pharmacology of Monoclonal Antibodies, Vol. 113, Rosenburg and Moore eds., Springer-Verlag, New York, pp. 269-315 (1994). “Fv” is the minimum antibody fragment that contains a complete antigen-recognition and binding site. This fragment consists of a dimer of one heavy and one light chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, a “humanized antibody” is a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable regions fused onto constant domains or only the CDRs grafted onto appropriate framework regions in the variable domains. Epitope binding sites may be wild type or may be modified by one or more amino acid substitutions. Another approach focuses not only on providing human-derived constant regions, but also on modifying the variable regions as well so as to reshape them as closely as possible to human form. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

As used herein, the term “specific binding” refers to the ability of an antibody to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (dissociation constant) of less than about 100 nM, or less than about 50 nM, or less than about 25 nM, or less than about 10 nM, or less than about 5 nM, or less than about 1 nM.

As used herein, the term “variant” antibody refers to a molecule, which differs in amino acid sequence from a “parent” or reference antibody amino acid sequence by virtue of addition, deletion, and/or substitution of one or more amino acid residue(s) in the parent antibody sequence. In one embodiment, a variant anti-Factor D antibody refers to a molecule which contains variable regions that are identical to the parent variable domains, except for a combined total of 1, 2, 3, 4, 5, 6, 7, 8 9 or 10 amino acid substitutions within the CDR regions of the heavy chain variable region, and/or up to a combined total of 1, 2, 3, 4, 5, 6, 7, 8, 9, or 10 amino acid substitutions with said CDR regions of the light chain variable region. In some embodiments, the amino acid substitutions are conservative sequence modifications.

As used herein, the term “parent antibody” refers to an antibody, which is encoded by an amino acid sequence used for the preparation of the variant. Preferably, the parent antibody has a human framework region and, if present, has human antibody constant region(s). For example, the parent antibody may be a humanized or fully human antibody.

As used herein, the term “isolated antibody” refers to an antibody that has been identified and separated and/or recovered from a component of its natural environment. Contaminant components of its natural environment are materials, which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “epitope” refers to the portion of an antigen to which a monoclonal antibody specifically binds. Epitopic determinants usually consist of chemically active surface groupings of molecules such as amino acids or sugar side chains and usually have specific three-dimensional structural characteristics, as well as specific charge characteristics. More specifically, the term “Pro-Factor D epitope” as used herein refers to a portion of the corresponding polypeptide (SEQ ID NO:4) to which an antibody immunospecifically binds as determined by any method well known in the art, for example, by immunoassays. The term “mature Factor D epitope” as used herein refers to an epitope encompassing the amino-terminal portion of the corresponding polypeptide (SEQ ID NO:3), e.g., an epitope at the N-terminus of mature Factor D comprising or consisting of “ILGGREA,” set forth as SEQ ID NO:5, to which an antibody immunospecifically binds as determined by any method well known in the art, for example, by immunoassays. The term “epitope shared by Pro-Factor D and mature Factor D” as used herein refers to an epitope in the C-terminal region shared by Pro-Factor D and mature Factor D (e.g., amino acids 8 to 228 of SEQ ID NO:3) to which an antibody immunospecifically binds as determined by any method well known in the art, for example, by immunoassays. Antigenic epitopes need not necessarily be immunogenic. Such epitopes can be linear in nature or can be a discontinuous epitope. Thus, as used herein, the term “conformational epitope” refers to a discontinuous epitope formed by a spatial relationship between amino acids of an antigen other than an unbroken series of amino acids.

As used herein, “a mammalian subject” includes, without limitation, humans, non-human primates, dogs, cats, horses, sheep, goats, cows, rabbits, pigs, and rodents.

As used herein, the term “biological sample” includes, without limitation, blood, plasma, serum, sputum, amniotic fluid, cerebrospinal fluid, cell lysate, ascites, urine, saliva, and tissue.

As used herein, the term “contacting” refers to a combining action that brings an antibody of the invention into contact with the biological sample in a manner that a binding interaction will occur between the antibody and the target protein (e.g., Pro-Factor D or mature Factor D) in the biological sample.

As used herein, the term “detecting antibody” or “detection antibody” refers to antibodies that are capable of being discovered. The detecting antibody may be directly or indirectly (e.g. through another antibody) conjugated to a detectable label or signal or to a signal-generating moiety. The signal may be can be radioactive (e.g., radioactive iodine, tritium, carbon, sulfur, or the like), colorimetric, fluorescent signal and the like. Signal-generating moieties that act on signal-generating substrates include, but are not limited to, horseradish peroxidase (HRP) [suitable substrates include 3,3′,5,5′-tetramethylbenzidine (TMB); OPD; 2,2′-azinobis(3-ethylbenzothiazoline)-6-sulfonic acid diammonium salt]; alkaline phosphatase [suitable substrates include p-nitrophenyl phosphate disodium salt]; and beta-galactosidase [suitable substrates include O-nitrophenyl-beta-D-galactopyranoside]. The signal may be amplified by using an Avidin-Biotin conjugation system. A detectable label or signal-generating moiety may be coupled either directly and/or indirectly to the anti-Factor D antibodies and antigen binding fragments thereof of the present invention. For example, the immunoconjugate may comprise an anti-Factor D antibody that is labeled with a radioactive isotope or enzymatic activity which permits detection in an immunoassay.

As used herein, the term “MASP-3 inhibitory agent” refers to any agent that binds to MASP-3 and inhibits the conversion of Pro-Factor D to mature Factor D, thereby inhibiting the alternative pathway of complement activation (APC), including anti-MASP-3 antibodies and MASP-3 binding fragments thereof, natural and synthetic peptides, competitive substrates, small molecules, and expression inhibitors. Exemplary MASP-3 inhibitory antibodies are disclosed in WO2018/026722, hereby incorporated herein by reference. In some embodiments, the MASP-3 inhibitory agent is a MASP-3 inhibitory antibody, such as a MASP-3 inhibitory monoclonal antibody selected from the group consisting of 4D5, 10D12 and 13B1.

As used herein, the amino acid residues are abbreviated as follows: alanine (Ala;A), asparagine (Asn;N), aspartic acid (Asp;D), arginine (Arg;R), cysteine (Cys;C), glutamic acid (Glu;E), glutamine (Gln;Q), glycine (Gly;G), histidine (His;H), isoleucine (Ile;I), leucine (Leu;L), lysine (Lys;K), methionine (Met;M), phenylalanine (Phe;F), proline (Pro;P), serine (Ser;S), threonine (Thr;T), tryptophan (Trp;W), tyrosine (Tyr;Y), and valine (Val;V).

In the broadest sense, the naturally occurring amino acids can be divided into groups based upon the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His. This grouping of amino acids can be further subclassed as follows. By “uncharged hydrophilic” amino acid is meant either Ser, Thr, Asn or Gln. By “acidic” amino acid is meant either Glu or Asp. By “basic” amino acid is meant either Lys, Arg or His.

As used herein the term “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

As used herein, an “isolated nucleic acid molecule” is a nucleic acid molecule (e.g., a polynucleotide) that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

As used herein, a “nucleic acid molecule construct” is a nucleic acid molecule, either single- or double-stranded, that has been modified through human intervention to contain segments of nucleic acid combined and juxtaposed in an arrangement not existing in nature.

As used herein, an “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

As used herein, the terms “approximately” or “about” in reference to a number are generally taken to include numbers that fall within a range of 5% in either direction (greater than or less than) of the number unless otherwise stated or otherwise evident from the context (except where such number would exceed 100% of a possible value). Where ranges are stated, the endpoints are included within the range unless otherwise stated or otherwise evident from the context.

As used herein the singular forms “a”, “an” and “the” include plural aspects unless the context clearly dictates otherwise. Thus, for example, reference to “a cell” includes a single cell, as well as two or more cells; reference to “an agent” includes one agent, as well as two or more agents; reference to “an antibody” includes a plurality of such antibodies and reference to “a framework region” includes reference to one or more framework regions and equivalents thereof known to those skilled in the art, and so forth.

Percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

Each embodiment in this specification is to be applied mutatis mutandis to every other embodiment unless expressly stated otherwise.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y.; Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or other relevant Current Protocol publications and other like references. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

It is contemplated that any embodiment discussed in this specification can be implemented with respect to any method, kit, reagent, or composition of the invention, and vice versa. Furthermore, compositions of the invention can be used to achieve methods of the invention.

### II. Overview

As described in Examples 1-3 herein, the present invention provides monoclonal anti-Factor D antibodies that specifically bind to human mature Factor D (also referred to as mature Factor-D specific antibodies) and do not bind to Pro-Factor D. As described in Examples 8-9, the present invention also provides monoclonal anti-Factor D antibodies that specifically bind to Pro-Factor D (also referred to as Pro-Factor-D-specific antibodies) and do not bind to mature Factor D. As described in Examples 4-5 herein, the present invention also provides monoclonal anti-Factor D antibodies that bind to both Pro- and mature-Factor D. As described in Examples 6 and 7, the mature-Factor D-specific monoclonal antibodies and the Pro-Factor D-specific monoclonal antibodies are useful for detection of the mature and/or the pro-form of Factor D in biological samples and may also be used to determine the status of the Alternative Pathway of Complement (APC) in a mammalian subject. As further described in Examples 10-12, the mature-Factor D specific monoclonal antibodies and/or the Pro-Factor D-specific antibodies may also be used to determine the status of Factor D after treatment with a MASP-3 inhibitory agent, such as a MASP-3 inhibitory antibody which inhibits the conversion of Pro-Factor D to mature Factor D.

Accordingly, in one embodiment, the present invention is directed to monoclonal antibodies that specifically bind to the N-terminal region of human mature Factor D and the use of these antibodies in methods of detecting the presence or amount of mature Factor D in a biological sample. In another embodiment, the present invention is directed to monoclonal antibodies that specifically bind to the activation (pro) peptide of Pro-Factor D and the use of these antibodies in methods of detecting the presence or amount of Pro-Factor D in a biological sample. In another embodiment, the present invention is directed to the use of mature-Factor-D specific monoclonal antibodies and/or the use of Pro-Factor-D-specific monoclonal antibodies to measure the presence or amount of mature-Factor D and/or Pro-Factor D in a mammalian subject before and after treatment with a MASP-3 inhibitory agent, such as a high affinity MASP-3 inhibitory antibody, wherein the MASP-3 inhibitory antibody is capable of inhibiting the conversion of Pro-Factor D to mature Factor D and thereby inhibit the APC.

Therefore, the subject antibodies can be used in diagnostic methods to detect the presence or amount of mature Factor D and/or Pro-Factor D in a biological sample obtained from a subject. In one embodiment, the presence or amount of mature Factor D and/or Pro-Factor D is useful as a biomarker for the determination of efficacy of a MASP-3 inhibitory agent for inhibiting the APC and/or monitoring the dosing in a subject undergoing treatment with a MASP-3 inhibitory agent, such as a MASP-3 inhibitory antibody (e.g., MASP-3 inhibitory antibodies 4D5, 10D12 or 13B1) in the subject.

### III. Overview of the Complement System

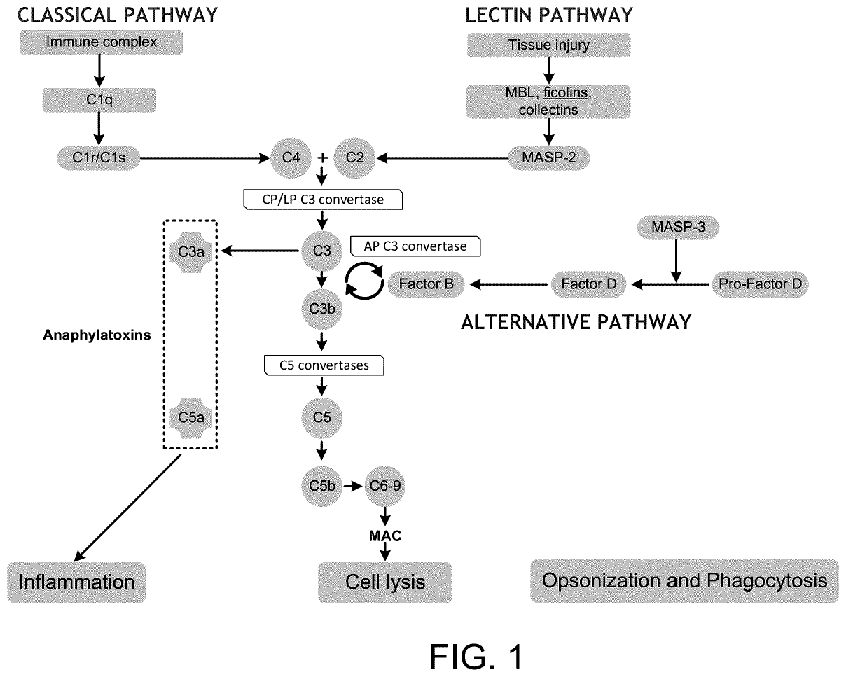

FIG. 1 illustrates the three pathways that drive complement activity in response to distinct initiating events: the classical, lectin, and alternative pathways (Noris M., Semin Nephrol 33 (6): 479-92, 2013). The classical pathway is triggered by immune complexes and mediates important immune effector functions through the early activity of two proteases, C1r and C1s. The lectin pathway can be activated by specific types of cell-surface carbohydrate patterns that are usually found on microbes or on injured host tissue, but not on healthy host cell surfaces. Lectin pathway activation is initiated by a group of enzymes known as mannan-binding lectin-associated serine protease-1 and 2 (MASP-1 and MASP-2) (Yongqing T. et al., Biochim Biophys Acta 1824 (1): 253-62, 2012). These proteases form complexes with lectins, such as the mannan-binding lectin (MBL), ficolins, and collectins 10 and 11. The lectins bind carbohydrate patterns on foreign or injured host cells, thus targeting the proteolytic activity of the MASPs to specific surfaces. Complement Factor D (CFD) is a serine protease that is essential for activation of the alternative pathway. As shown in FIG. 1, Factor D (CFD) is expressed as an inactive zymogen (referred to herein as “Pro-Factor D) and it circulates in plasma predominantly as a cleaved, mature serine protease (referred to herein as “mature Factor D”).). As described in WO2013/180834 and WO2013/192240, it has recently been determined that MASP-3 is responsible for the conversion of complement factor D (CFD) from the zymogen form of the protein (Pro-Factor D) to the mature form (mature Factor D), thus placing the MASP-3 protein at a key upstream regulatory step for the alternative pathway. As further described in WO2018/026722, hereby incorporated herein by reference, numerous high affinity anti-MASP-3 inhibitory antibodies have been generated that bind the serine protease domain of MASP-3 and inhibit its catalytic activity, thereby inhibiting the conversion of Pro-Factor D to mature Factor D and blocking activation of the alternative pathway.

The primary function of the complement system, a part of the innate immune response, is to protect the host against infectious agents (Ricklin et al., Nat Immunol 11 (9): 785-97, 2010). Through the coordinated action of protein complex assembly and proteolytic cascades, this intricate physiological system targets immune and inflammatory responses to surfaces that display molecular patterns not usually present on healthy host cells. Complement system activation culminates in targeted cell destruction by the formation of the membrane attack complex (MAC), which directly disrupts the membranes of the pathogen causing cell lysis, or by opsonization, which facilitates the uptake of the infectious agent by phagocytic cells as shown in FIG. 1. In addition, substrate cleavage by complement proteases releases cytokine-like peptides, called anaphylatoxins, that trigger several important biological activities such as leukocyte recruitment and immune cell activation.

The alternative pathway of complement (APC) is typically described as a downstream amplifier of complement activity, increasing the host immune response following activation of complement via the classical and lectin pathways. However, the ability of the APC to create a positive feedback loop of protease complexes with activity that drives the formation of new complexes of the same type is unique within the complement pathways (Lachmann et al., Adv Immunol 104:115-49, 2009). The self-propagating complex, the APC C3 convertase, is composed of 2 proteins: C3b and Bb. Newly formed C3b can covalently attach to local surfaces via a thioester bond and function as a potent opsonin, targeting the engulfment and destruction of marked cells. In addition, C3b provides the scaffold for binding and activation of complement factor B (CFB) (Lachmann et al., Adv Immunol 104:115-49, 2009; Noris et al., Semin Nephrol 33 (6): 479-92, 2013). In complex with C3b, CFB, adopts an appropriate configuration for cleavage by complement factor D (CFD). This cleavage event converts the single chain polypeptide into noncatalytic (Ba) and catalytic fragments (Bb). The Ba fragment is released from the complex; however, the Bb fragment remains associated with C3b, producing the active APC C3 convertase, C3bBb Lachmann et al., Adv Immunol 104:115-49, 2009; Noris et al., Semin Nephrol 33 (6): 479-92, 2013). It is the ability of C3bBb to cleave additional C3 and produce multiple new convertases that provides the mechanism for rampant signal amplification.

While the complement system supports innate host defense against pathogens, dysregulated and unabated complement activity can also function as a major driver of disease, causing unchecked propagation of inflammation and tissue destruction. In many contexts, the APC and the C3b amplification loop play an important role in determining the magnitude of the complement response and its downstream consequences. Thus, the therapeutic modulation of APC by inhibiting Bb activity or blocking the activation of the CFB through cleavage by CFD are well characterized potential control points for treating many autoimmune and inflammatory diseases mediated by the APC.

As noted above, it has been demonstrated that MASP-3 is responsible for the conversion of CFD from the zymogen form of the protein to the mature form, thus placing the MASP-3 protein at a key upstream regulatory step for the alternative pathway. In a wild-type animal or human plasma, the large majority of systemic CFD has already been processed to the mature form by in vivo MASP-3 activity, making in vitro assessment of APC inhibition by MASP-3 inhibitors using traditional assays impossible. Therefore, a need exists for detection reagents and assays for measuring the presence and amount of Pro-Factor D and/or mature Factor D in a biological sample, which can be used as a biomarker of APC status and also can be used for in vitro and/or in vivo assessment of APC inhibition by MASP-3 inhibitors.

### IV. Anti-Factor D Antibodies

As described above, Complement Factor D (CFD) is a serine protease that is essential for activation of the APC. As shown in FIG. 1, Factor D (CFD) is expressed as an inactive zymogen (referred to herein as “Pro-Factor D”) and it circulates in plasma predominantly as a cleaved, mature serine protease (referred to herein as “mature Factor D”).

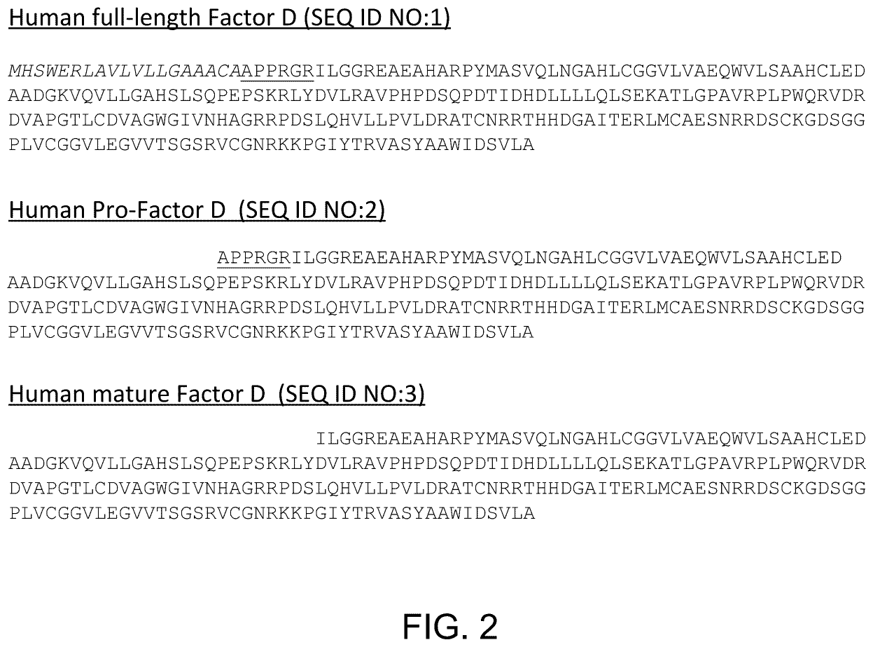

FIG. 2 provides the amino acid sequences of (i) human full-length Factor D (SEQ ID NO:1), including the signal sequence aa 1-19 (shown in italics) with the activation (pro) peptide underlined; (ii) human Pro-Factor D (SEQ ID NO:2), with the pro-peptide underlined; and (iii) human mature Factor D (SEQ ID NO:3). As shown in FIG. 2, the pro-peptide of human Pro-Factor D is (“APPRGR” (SEQ ID NO:4).

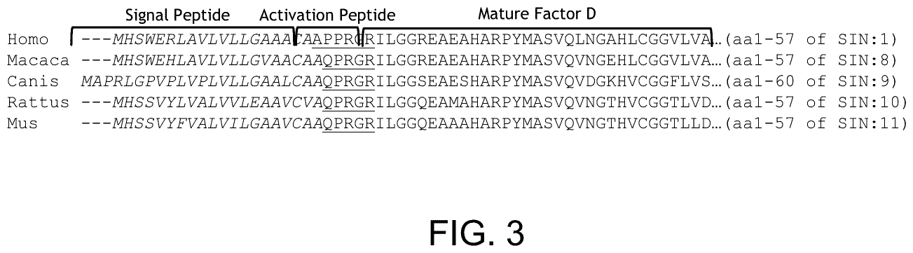

FIG. 3 provides an alignment of the amino acid sequences of complement Factor D (full-length) from various species including Homo sapiens (SEQ ID NO:1); Macaca (SEQ ID NO: 8); Canis (SEQ ID NO:9); Rattus (SEQ ID NO:10); and Mus musculus (SEQ ID NO: 11). The italicized portion of each sequence depicts the signal sequence and the underlined portion depicts the activation “pro” peptide sequence.

As shown in FIGS. 2 and 3, the Factor D protein comprises an N-terminal Pro region, an activation peptide region, and the remaining C-terminal region. Mature Factor D has a unique amino-terminus as compared to pro-Factor D in each species (e.g., an N-terminus starting at residue 26 of human full-length Factor D (SEQ ID NO:1)). Mature and Pro-Factor D have a shared sequence in the C-terminal portion of the respective proteins (e.g., from amino acids 27 to 253 of SEQ ID NO:1).

### A. Anti-Human Mature Factor D-Specific Monoclonal Antibodies

As described in Examples 1 and 2 herein, the inventors have used a peptide “ILGGREA” (SEQ ID NO:5), corresponding to amino acid residues 1 to 7 of the amino-terminal region of human mature Factor D (SEQ ID NO:3) as an antigen to generate anti-mature Factor D-specific antibodies suitable for use in the detection assays and methods described herein. As shown in FIG. 3, the N-terminal sequence “ILLGGREA” (SEQ ID NO: 5) is conserved between human (Homo sapiens) and macaque (macaca) mature Factor D proteins.

As described in Example 2, the variable heavy and light chain fragments of several representative anti-mature Factor D-specific monoclonal antibodies have been cloned and sequenced.

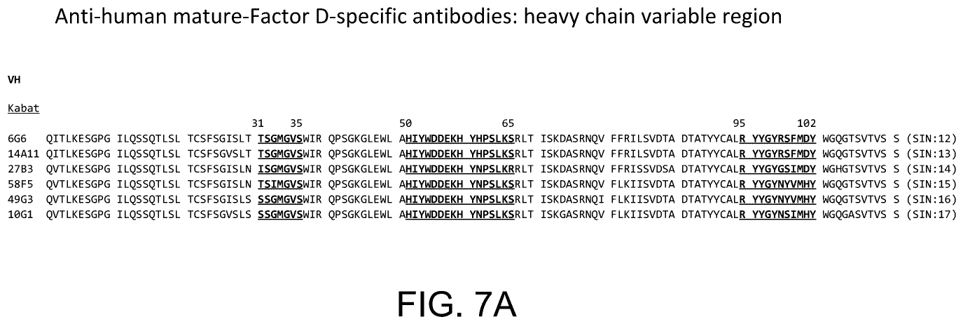

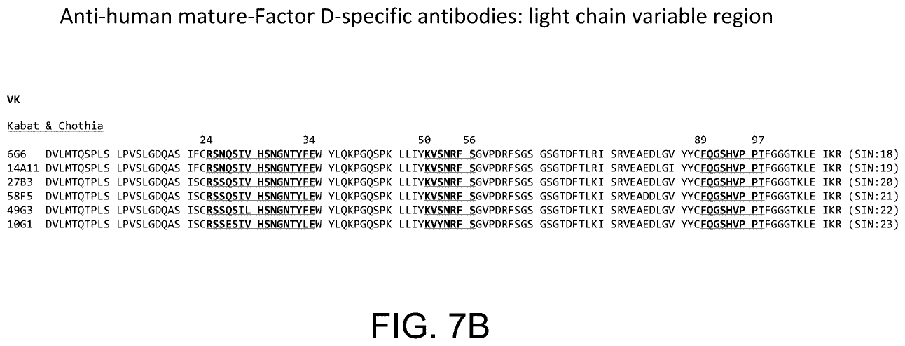

FIG. 7A is an amino acid sequence alignment of the variable heavy chain regions of six anti-mature Factor D-specific clones that were identified as having high binding affinity to the N-terminal peptide “ILGGREA” (SEQ ID NO:5) of mature Factor D.

FIG. 7B is an amino acid sequence alignment of the variable light chain regions of six anti-mature Factor D-specific clones that were identified as having high binding affinity to the N-terminal peptide “ILGGREA,” SEQ ID NO:5, of mature Factor D.

The heavy chain and light chain variable regions and CDRs therein of the six mature Factor D-specific antibodies are provided below in TABLES 1 and 2.

TABLE 1
anti-human mature-Factor D-specific Antibody Sequences: mouse parental

| Anti-human | | | | |
|---|---|---|---|---|
| active-Factor D | Heavy Chain | Light Chain | Heavy chain | Light chain |
| Antibody | Variable Region | Variable Region | variable region | variable region |
| Reference No | (amino acid) | (amino acid) | (DNA) | (DNA) |
| 6G6 | SEQ ID NO: 12 | SEQ ID NO: 18 | SEQ ID NO: 73 | SEQ ID NO: 79 |
| 14A11 | SEQ ID NO: 13 | SEQ ID NO: 19 | SEQ ID NO: 74 | SEQ ID NO: 80 |
| 27B3 | SEQ ID NO: 14 | SEQ ID NO: 20 | SEQ ID NO: 75 | SEQ ID NO: 81 |
| 58F5 | SEQ ID NO: 15 | SEQ ID NO: 21 | SEQ ID NO: 76 | SEQ ID NO: 82 |
| 49G3 | SEQ ID NO: 16 | SEQ ID NO: 22 | SEQ ID NO: 77 | SEQ ID NO: 83 |
| 10G1 | SEQ ID NO: 17 | SEQ ID NO: 23 | SEQ ID NO: 78 | SEQ ID NO: 84 |

TABLE 2
anti-human mature-Factor D-specific antibodies: CDRs

| | | | Heavy Chain: | Light Chain |
|---|---|---|---|---|
| | Heavy Chain: | Light Chain: | consensus | consensus |
| Anti-human | CDR1; | CDR1; | CDR1; | CDR1; |
| active-Factor D | CDR2; | CDR2; | CDR2; | CDR2; |
| Antibody | CDR3 | CDR3 | CDR3 | CDR3 |
| Reference No. | (SEQ ID NOs) | (SEQ ID NOs) | (SEQ ID NOs) | (SEQ ID NOs) |
| 6G6 | 25; 27; 29 | 50; 52; 54 | 65; 66; 67 | 68; 69; 54 |
| 14A11 | 25; 27; 29 | 50; 52; 54 | 65; 66; 67 | 68; 69; 54 |
| 27B3 | 33, 34, 36 | 58, 52, 54 | 65; 66; 67 | 68; 69; 54 |
| 58F5 | 38, 39, 41 | 60, 52, 54 | 65; 66; 67 | 68; 69; 54 |
| 49G3 | 43, 39, 41 | 62, 52, 54 | 65; 66; 67 | 68; 69; 54 |
| 10G1 | 43, 39, 47 | 63, 64, 54 | 65; 66; 67 | 68; 69; 54 |

Accordingly, in one aspect, the present invention provides an isolated antibody, or antigen-binding fragment thereof, that specifically binds to an epitope in the amino-terminal (N-terminal) region of human mature Factor D, wherein the epitope comprises or consists of the amino acid sequence “ILGGREA” (SEQ ID NO:5). In one embodiment, the mature Factor D-specific antibody or fragment thereof specifically binds to human mature Factor D (SEQ ID NO:3) and does not bind to human Pro-Factor D (SEQ ID NO:2). In one embodiment, the mature Factor D-specific antibody is a monoclonal antibody. In one embodiment the mature Factor D-specific antibody is a humanized, chimeric, or fully human antibody. In one embodiment the mature Factor D-specific antibody fragment is selected from the group consisting of Fv, Fab, Fab′, F(ab)₂ and F(ab′)₂. In one embodiment, the mature Factor D-specific antibody is a single-chain molecule. In one embodiment, the mature Factor D-specific antibody is an IgG molecule selected from the group consisting of IgG1, IgG2 and IgG4. In one embodiment, the mature Factor D-specific antibody or antigen-binding fragment thereof binds to human mature Factor D with a K_{D} of less than 10 nM. In one embodiment, the mature Factor D-specific antibody or antigen-binding fragment thereof is labeled with a detectable moiety, for example a detectable moiety suitable for use in an immunoassay as further described herein. In one embodiment, the mature Factor D-specific antibody or fragment thereof is immobilized on a substrate, such as a substrate suitable for use in an immunoassay, as further described herein.

In one embodiment, the mature Factor D-specific antibody or fragment thereof (i.e., an antibody or fragment thereof that specifically binds to human mature Factor D) comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23, wherein the CDRs are numbered according to the Kabat numbering system. In one embodiment, the mature Factor D-specific antibody or fragment thereof comprises a binding domain comprising the following six CDRs: (a) an HC-CDR1 comprising the amino acid sequence XSXMGVS (SEQ ID NO:65), wherein X at position 1 is T, I or S and X at position 3 is G or I; (b) an HC-CDR2 comprising the amino acid sequence HIYWDDEKHYXPSLKX (SEQ ID NO:66), wherein X at position 11 is H or N and X at position 16 is S or R; (c) an HC-CDR3 comprising the amino acid sequence RYYGYXXXMXY (SEQ ID NO:67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is N or S, X at position 4 is Q or E, X at position 7 is V or L, and X at position 15 is F or L; (e) a LC-CDR2 comprising the amino acid sequence KVXNRFS (SEQ ID NO: 69), wherein: X at position 3 is S or Y; and (f) a LC-CDR3 comprising the amino acid sequence FQGSHVPPT (SEQ ID NO:54).

In one embodiment, the mature Factor D-specific antibody or fragment thereof comprises a binding domain comprising the following six CDRs: (a) an HC-CDR1 comprising SEQ ID NO:25, (b) an HC-CDR2 comprising SEQ ID NO:27; (c) an HC-CDR3 comprising SEQ ID NO: 29; (d) a LC-CDR-1 comprising SEQ ID NO:50, (e) a LC-CDR2 comprising SEQ ID NO:52 and (f) a LC-CDR3 comprising SEQ ID NO:54. In one embodiment the mature Factor D-specific antibody or fragment thereof comprises a VH domain having at least 95% sequence identity (such as at least 96%, at least 97%, at least 98%, or at least 99% identity) to the amino acid sequence of SEQ ID NO:12 or SEQ ID NO: 13. In one embodiment, the mature Factor D-specific antibody or fragment thereof comprises a VL domain having at least 95% sequence identity (such as at least 96%, at least 97%, at least 98%, or at least 99% identity) to the amino acid sequence of SEQ ID NO:18 or SEQ ID NO:19. In one embodiment, the mature Factor D-specific antibody or fragment thereof comprises a VH comprising SEQ ID NO:12 and a VL comprising SEQ ID NO:18. In one embodiment, the mature Factor D-specific antibody or fragment thereof comprises a VH comprising SEQ ID NO:13 and a VL comprising SEQ ID NO:19.

In one embodiment, the mature Factor D-specific antibody or fragment thereof comprises a binding domain comprising the following six CDRs: (a) an HC-CDR1 comprising SEQ ID NO:33, (b) an HC-CDR2 comprising SEQ ID NO:34; (c) an HC-CDR3 comprising SEQ ID NO: 36; (d) a LC-CDR1 comprising SEQ ID NO:58, (e) a LC-CDR2 comprising SEQ ID NO:52 and (f) a LC-CDR3 comprising SEQ ID NO:54. In one embodiment, the mature Factor D-specific antibody or fragment thereof comprises a VH domain having at least 95% sequence identity (such as at least 96%, at least 97%, at least 98%, or at least 99% identity) to the amino acid sequence of SEQ ID NO:14. In one embodiment, the mature Factor D-specific antibody or fragment thereof comprises a VL domain having at least 95% sequence identity (such as at least 96%, at least 97%, at least 98%, or at least 99% identity) to the amino acid sequence of SEQ ID NO:20. In one embodiment, the mature Factor D-specific antibody or fragment thereof comprises a VH comprising SEQ ID NO: 14 and a VL comprising SEQ ID NO:20.

In one embodiment, the mature Factor D-specific antibody or fragment thereof comprises a binding domain comprising the following six CDRs: (a) an HC-CDR1 comprising SEQ ID NO:38, (b) an HC-CDR2 comprising SEQ ID NO:39; (c) an HC-CDR3 comprising SEQ ID NO: 41; (d) a LC-CDR1 comprising SEQ ID NO:60, (e) a LC-CDR2 comprising SEQ ID NO:52 and (f) a LC-CDR3 comprising SEQ ID NO:54. In one embodiment the mature Factor D-specific antibody or fragment thereof comprises a VH domain having at least 95% sequence identity (such as at least 96%, at least 97%, at least 98%, or at least 99% identity) to the amino acid sequence of SEQ ID NO:15. In one embodiment, the mature Factor D-specific antibody or fragment thereof comprises a VL domain having at least 95% sequence identity (such as at least 96%, at least 97%, at least 98%, or at least 99% identity) to the amino acid sequence of SEQ ID NO:21. In one embodiment, the mature Factor D-specific antibody or fragment thereof comprises a VH comprising SEQ ID NO: 15 and a VL comprising SEQ ID NO:21.

In one embodiment, the mature Factor D-specific antibody or fragment thereof comprises a binding domain comprising the following six CDRs: (a) an HC-CDR1 comprising SEQ ID NO:43, (b) an HC-CDR2 comprising SEQ ID NO:39; (c) an HC-CDR3 comprising SEQ ID NO: 41; (d) a LC-CDR1 comprising SEQ ID NO:62, (e) a LC-CDR2 comprising SEQ ID NO:52 and (f) a LC-CDR3 comprising SEQ ID NO:54. In one embodiment the mature Factor D-specific antibody or fragment thereof comprises a VH domain having at least 95% sequence identity (such as at least 96%, at least 97%, at least 98%, or at least 99% identity) to the amino acid sequence of SEQ ID NO:16. In one embodiment, the mature Factor D-specific antibody or fragment thereof comprises a VL domain having at least 95% sequence identity (such as at least 96%, at least 97%, at least 98%, or at least 99% identity) to the amino acid sequence of SEQ ID NO:22. In one embodiment, the mature Factor D-specific antibody or fragment thereof comprises a VH comprising SEQ ID NO: 16 and a VL comprising SEQ ID NO:22.

In one embodiment, the mature Factor D-specific antibody or fragment thereof comprises a binding domain comprising the following six CDRs: (a) an HC-CDR1 comprising SEQ ID NO:43, (b) an HC-CDR2 comprising SEQ ID NO:39; (c) an HC-CDR3 comprising SEQ ID NO: 47; (d) a LC-CDR1 comprising SEQ ID NO:63, (e) a LC-CDR2 comprising SEQ ID NO:64 and (f) a LC-CDR3 comprising SEQ ID NO:54. In one embodiment the mature Factor D-specific antibody or fragment thereof comprises a VH domain having at least 95% sequence identity (such as at least 96%, at least 97%, at least 98%, or at least 99% identity) to the amino acid sequence of SEQ ID NO:17. In one embodiment, the mature Factor D-specific antibody or fragment thereof comprises a VL domain having at least 95% sequence identity (such as at least 96%, at least 97%, at least 98%, or at least 99% identity) to the amino acid sequence of SEQ ID NO:23. In one embodiment, the mature Factor D-specific antibody or fragment thereof comprises a VH comprising SEQ ID NO:17 and a VL comprising SEQ ID NO:23.

In certain embodiments, the mature Factor D-specific antibody or fragment thereof that specifically binds to human mature Factor D has a heavy chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the heavy chain variable domain sequences set forth in TABLE 1. In certain embodiments, the mature Factor D-specific antibody or fragment thereof that specifically binds to human mature Factor D has a light chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the light chain variable domain sequences set forth in TABLE 1.

In another embodiment, the present disclosure provides a nucleic acid encoding the complementarity determining regions (CDRs) of a heavy chain variable region of a mature Factor D-specific antibody, or antigen-binding fragment thereof, that specifically binds to human mature Factor D, wherein the heavy chain variable region comprises an amino acid sequence set forth in SEQ ID NOs: 12-17, and wherein the CDRs are numbered according to the Kabat numbering system. In another embodiment, the present disclosure provides a nucleic acid encoding the complementarity determining regions (CDRs) of a light chain variable region of a mature Factor D-specific antibody, or antigen-binding fragment thereof that specifically binds to human mature Factor D, wherein the light chain variable region comprises an amino acid sequence set forth in SEQ ID NOs: 18-23, and wherein the CDRs are numbered according to the Kabat numbering system.

In another embodiment, the present disclosure provides a cloning or expression vector comprising a nucleic acid encoding complementarity determining regions (CDRs) of heavy and/or light chain variable regions of an antibody, or antigen-binding fragment thereof, that specifically binds to human mature Factor D, wherein the heavy chain variable region comprises the amino acid sequence set forth as any of SEQ ID NOs: 12-17 and the light chain variable region comprises the amino acid sequence set forth as any of SEQ ID NOs: 18-23, wherein the CDRs are numbered according to the Kabat numbering system.

In another embodiment, the present disclosure provides a cell containing a cloning or expression vector comprising a nucleic acid encoding complementarity determining regions (CDRs) of heavy and/or light chain variable regions of an antibody, or antigen-binding fragment thereof, that specifically binds to human mature Factor D, wherein the heavy chain variable region comprises the amino acid sequence set forth as any of SEQ ID NOs: 12-17 and the light chain variable region comprises the amino acid sequence set forth as any of SEQ ID NOs: 18-23, wherein the CDRs are numbered according to the Kabat numbering system.

In another embodiment, the present disclosure provides a method for producing a human mature Factor-D-specific antibody comprising culturing a cell containing an expression vector which contains a nucleic acid that encodes one or both of the heavy and light chain polypeptides of any of the mature Factor-D specific antibodies or antigen-binding fragments disclosed herein. The cell or culture of cells is cultured under conditions and for a time sufficient to allow expression by the cell (or culture of cells) of the antibody or antigen-binding fragment thereof encoded by the nucleic acid. The method can also include isolating the antibody or antigen binding fragment thereof from the cell (or culture of cells) or from the media in which the cell or cells were cultured.

In one embodiment, the present disclosure provides a composition comprising any of the mature Factor-D-specific antibodies, or antigen-binding fragments disclosed herein.

In one embodiment, the present disclosure provides a substrate for use in an immunoassay comprising at least one or more of any of the mature Factor-D-specific antibodies, or antigen-binding fragments disclosed herein.

In one embodiment, the present disclosure provides a kit for detecting the presence or amount of mature Factor D in a test sample, such as a biological sample, said kit comprising (a) at least one container, and (b) at least one or more of any of the mature Factor-D-specific antibodies, or antigen-binding fragments disclosed herein.

### B. Anti-Human Pro-Factor D-Specific Monoclonal Antibodies

As described in Examples 8 and 9 herein, the inventors have used the human pro peptide “APPRGR” (SEQ ID NO:4), corresponding to residues 20-25 of human full-length Factor D, as an antigen to generate anti-Pro-Factor D-specific antibodies suitable for use in the detection assays and methods described herein.

As described in Example 9, the variable heavy and light chain fragments of several representative anti-Pro-Factor D-specific monoclonal antibodies have been cloned and sequenced.

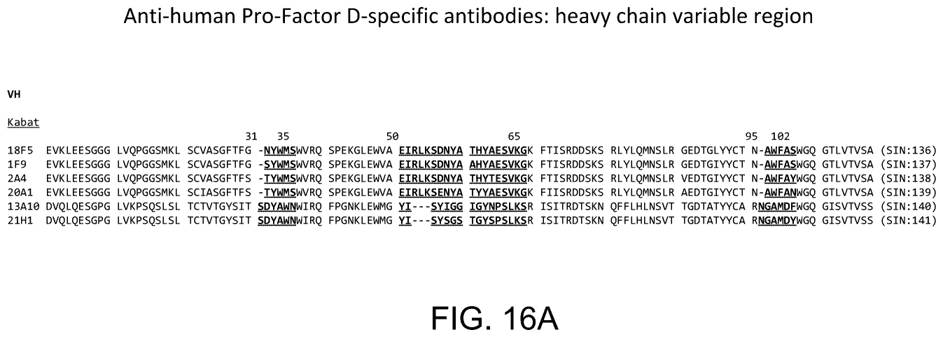

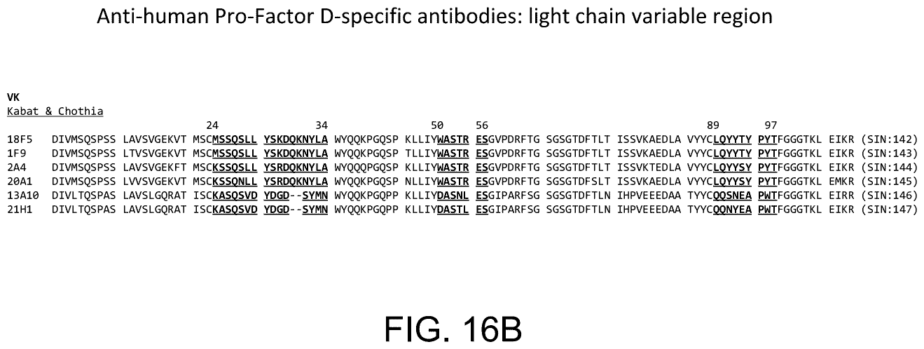

FIG. 16A is an amino acid sequence alignment of the variable heavy chain regions of six anti-Pro-Factor D clones that were identified as having high binding affinity to the human Factor D pro peptide “APPRGR” (SEQ ID NO:4).

FIG. 16B is an amino acid sequence alignment of the variable light chain regions of six anti-Pro-Factor D clones that were identified as having high binding affinity to the human Factor D pro peptide “APPRGR” (SEQ ID NO:4).

The heavy chain and light chain variable regions and CDRs therein of the six pro-Factor D-specific monoclonal antibodies are provided below in TABLES 3 and 4.

TABLE 3
anti-human Pro-Factor D-specific Antibody Sequences: mouse parental

| Anti-human | | | | |
|---|---|---|---|---|
| pro-Factor D | Heavy Chain | Light Chain | Heavy chain | Light chain |
| Antibody | Variable Region | Variable Region | variable region | variable region |
| Reference No | (amino acid) | (amino acid) | (DNA) | (DNA) |
| 18F5 | SEQ ID NO: 136 | SEQ ID NO: 142 | SEQ ID NO: 206 | SEQ ID NO: 212 |
| 1F9 | SEQ ID NO: 137 | SEQ ID NO: 143 | SEQ ID NO: 207 | SEQ ID NO: 213 |
| 2A4 | SEQ ID NO: 138 | SEQ ID NO: 144 | SEQ ID NO: 208 | SEQ ID NO: 214 |
| 20A1 | SEQ ID NO: 139 | SEQ ID NO: 145 | SEQ ID NO: 209 | SEQ ID NO: 215 |
| 13A10 | SEQ ID NO: 140 | SEQ ID NO: 146 | SEQ ID NO: 210 | SEQ ID NO: 216 |
| 21H1 | SEQ ID NO: 141 | SEQ ID NO: 147 | SEQ ID NO: 211 | SEQ ID NO: 217 |

TABLE 4
anti-human Pro-Factor D-specific antibodies: CDRs

| | | | Heavy Chain: | Light Chain |
|---|---|---|---|---|
| | Heavy Chain: | Light Chain: | consensus | consensus |
| Anti-human | CDR1; | CDR1; | CDR1; | CDR1; |
| pro-Factor | CDR2; | CDR2; | CDR2; | CDR2; |
| D Antibody | CDR3 | CDR3 | CDR3 | CDR3 |
| Reference No. | (SEQ ID NOs) | (SEQ ID NOs) | (SEQ ID NOs) | (SEQ ID NOs) |
| 18F5 | 149, 151, 153 | 176, 178, 180 | 201, 202, 203 | 204, 178, 205 |
| 1F9 | 155, 156, 153 | 176, 178, 180 | 201, 202, 203 | 204, 178, 205 |
| 2A4 | 158, 159, 161 | 184, 178, 187 | 201, 202, 203 | 204, 178, 205 |
| 20A1 | 158, 163, 165 | 189, 178, 187 | 201, 202, 203 | 204, 178, 205 |
| 13A10 | 167, 169, 171 | 194, 196, 198 | 167, 169, 171 | 194, 196, 198 |
| 21H1 | 167, 173, 174 | 194, 199, 200 | 167, 173, 174 | 194, 199, 200 |

According, in one aspect, the present invention provides an isolated antibody or fragment thereof that specifically binds to an epitope in the activation (“Pro”) peptide of human Factor D set forth as “APPRGR” (SEQ ID NO:4), wherein the antibody or fragment specifically binds to human Pro-Factor D (SEQ ID NO:2) and does not bind to mature-Factor D (SEQ ID NO:3). In one embodiment, the Pro-Factor D-specific antibody is a monoclonal antibody. In one embodiment the Pro-Factor D-specific antibody is a humanized, chimeric, or fully human antibody. In one embodiment the Pro-Factor D-specific antibody fragment is selected from the group consisting of Fv, Fab, Fab′, F(ab)₂ and F(ab′)₂. In one embodiment, the Pro-Factor D-specific antibody is a single-chain molecule. In one embodiment, the Pro-Factor D-specific antibody is an IgG molecule selected from the group consisting of IgG1, IgG2 and IgG4. In one embodiment, the Pro-Factor D-specific antibody or antigen-binding fragment thereof binds to human Pro-Factor D with a K_{D} of less than 10 nM. In one embodiment, the Pro-Factor D-specific antibody or antigen-binding fragment thereof is labeled with a detectable moiety, for example a detectable moiety suitable for use in an immunoassay as further described herein. In one embodiment, the Pro-Factor D-specific antibody or fragment thereof is immobilized on a substrate, such as a substrate suitable for use in an immunoassay, as further described herein.

In one embodiment, the Pro-Factor D-specific antibody or fragment thereof (i.e., an antibody or fragment thereof that specifically binds to human Pro-Factor D) comprises a binding domain comprising HC-CDR1, HC-CDR-2 and HC-CDR-3 of a heavy chain variable region selected from the group consisting of SEQ ID NO:s 136-141 and comprising LC-CDR-1, LC-CDR2 and LC-CDR3 in a light chain variable region selected from the group consisting of SEQ ID NO:s 142-147, wherein the CDRs are numbered according to the Kabat numbering system.

In one embodiment, the Pro-Factor D-specific antibody or fragment thereof that specifically binds to human Pro-Factor D comprises a binding domain comprising HC-CDR1, HC-CDR-2 and HC-CDR-3 in a heavy chain variable region selected from the group consisting of SEQ ID NO:s 136-139 and comprising LC-CDR1, LC-CDR2 and LC-CDR3 in a light chain variable region selected from the group consisting of SEQ ID NO:s 142-145, wherein the CDRs are numbered according to the Kabat numbering system. In one embodiment the Pro-Factor D-specific antibody or fragment thereof comprises a binding domain comprising the following six CDRs: (a) an HC-CDR1 comprising the amino acid sequence XYWMS (SEQ ID NO:201), wherein X at position 1 is N, S or T; (b) an HC-CDR2 comprising the amino acid sequence EIRLKSXNYAXXYXESVKG (SEQ ID NO: 202), wherein: X at position 7 is D or E, X at position 11 is T or A, X at position 12 is H or Y and X at position 14 is A or T; (c) an HC-CDR3 comprising the amino acid sequence AWFAX (SEQ ID NO:203), wherein X at position 5 is S, Y or N; (d) a LC-CDR1 comprising the amino acid sequence XSSQXLLYSXDQKNYLA (SEQ ID NO:204), wherein X at position 1 is M or K, X at position 5 is S or N, and X at position 10 is K or R; (e) a LC-CDR2 comprising the amino acid sequence WASTRES (SEQ ID NO:178); and (f) a LC-CDR3 comprising the amino acid sequence LQYYXYPYT (SEQ ID NO:205), wherein X at position 5 is T or S. In one embodiment, the Pro-Factor D-specific antibody or fragment thereof comprises a binding domain comprising the following six CDRs: (a) an HC-CDR1 comprising SEQ ID NO:149 or SEQ ID NO:155, (b) an HC-CDR2 comprising SEQ ID NO:151 or SEQ ID NO:156; (c) an HC-CDR3 comprising SEQ ID NO:153; (d) a LC-CDR1 comprising SEQ ID NO:176, (e) a LC-CDR2 comprising SEQ ID NO:178 and (f) a LC-CDR3 comprising SEQ ID NO:180. In one embodiment, Pro-Factor D-specific antibody or fragment thereof comprises a VH domain having at least 95% sequence identity (such as at least 96%, at least 97%, at least 98%, or at least 99% identity) to the amino acid sequence of SEQ ID NO:136. In one embodiment, the Pro-Factor D-specific antibody or fragment thereof comprises a VH domain having at least 95% sequence identity (such as at least 96%, at least 97%, at least 98%, or at least 99% identity) to the amino acid sequence of SEQ ID NO:137. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO: 142. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO:143. In one embodiment, the Pro-Factor D-specific antibody or fragment thereof comprises a VH comprising SEQ ID NO:136 and a VL comprising SEQ ID NO:142. In one embodiment, the Pro-Factor D-specific antibody or fragment thereof comprises a VH comprising SEQ ID NO: 137 and a VL comprising SEQ ID NO:143.

In one embodiment, the Pro-Factor D-specific antibody or fragment thereof that specifically binds to human Pro-Factor D comprises a binding domain comprising the following six CDRs: (a) an HC-CDR1 comprising SEQ ID NO:158, (b) an HC-CDR2 comprising SEQ ID NO:159 or SEQ ID NO:163; (c) an HC-CDR3 comprising SEQ ID NO: 161 or SEQ ID NO:165; (d) a LC-CDR1 comprising SEQ ID NO:184 or SEQ ID NO: 189, (e) a LC-CDR2 comprising SEQ ID NO:178 and (f) a LC-CDR3 comprising SEQ ID NO: 187. In one embodiment the Pro-Factor D-specific antibody or fragment thereof comprises a VH domain having at least 95% sequence identity (such as at least 96%, at least 97%, at least 98%, or at least 99% identity) to the amino acid sequence of SEQ ID NO:138. In one embodiment the Pro-Factor D-specific antibody or fragment thereof comprises a VH domain having at least 95% sequence identity (such as at least 96%, at least 97%, at least 98%, or at least 99% identity) to the amino acid sequence of SEQ ID NO:139. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO:144. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO:145. In one embodiment, the Pro-Factor D-specific antibody or fragment thereof comprises a VH comprising SEQ ID NO:138 and a VL comprising SEQ ID NO:144. In one embodiment, the Pro-Factor D-specific antibody or fragment thereof comprises a VH comprising SEQ ID NO:139 and a VL comprising SEQ ID NO: 145.

In one embodiment, the Pro-Factor D-specific antibody or fragment thereof that specifically binds to human Pro-Factor D comprises a binding domain comprising HC-CDR1, HC-CDR2 and HC-CDR3 in a heavy chain variable region selected from the group consisting of SEQ ID NO:140 and SEQ ID NO:141 and comprising LC-CDR1, LC-CDR2 and LC-CDR3 in a light chain variable region selected from the group consisting of SEQ ID NO: 146 and SEQ ID NO:147. In one embodiment, the Pro-Factor D-specific antibody or fragment thereof comprises a binding domain comprising the following six CDRs: (a) an HC-CDR1 comprising SEQ ID NO:167, (b) an HC-CDR2 comprising SEQ ID NO:169 or SEQ ID NO:173; (c) an HC-CDR3 comprising SEQ ID NO:171 or SEQ ID NO:174; (d) a LC-CDR1 comprising SEQ ID NO:194, (e) a LC-CDR2 comprising SEQ ID NO:196 or SEQ ID NO:199 and (f) a LC-CDR3 comprising SEQ ID NO:198 or SEQ ID NO:200. In one embodiment, the Pro-Factor D-specific antibody or fragment thereof comprises a VH domain having at least 95% sequence identity (such as at least 96%, at least 97%, at least 98%, or at least 99% identity) to the amino acid sequence of SEQ ID NO:140. In one embodiment, the Pro-Factor D-specific antibody or fragment thereof comprises a VH domain having at least 95% sequence identity (such as at least 96%, at least 97%, at least 98%, or at least 99% identity) to the amino acid sequence of SEQ ID NO:141. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO:146. In one embodiment, the Pro-Factor D-specific antibody or fragment thereof comprises a VL domain having at least 95% sequence identity (such as at least 96%, at least 97%, at least 98%, or at least 99% identity) to the amino acid sequence of SEQ ID NO:147. In one embodiment, the Pro-Factor D-specific antibody or fragment thereof comprises a VH comprising SEQ ID NO:140 and a VL comprising SEQ ID NO:146. In one embodiment, the Pro-Factor D-specific antibody or fragment thereof comprises a VH comprising SEQ ID NO:141 and a VL comprising SEQ ID NO: 147.

In certain embodiments, the Pro-Factor D-specific antibody or fragment thereof has a heavy chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the heavy chain variable domain sequences set forth in TABLE 3. In certain embodiments, the Pro-Factor D-specific antibody or fragment thereof has a light chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the light chain variable domain sequences set forth in TABLE 3.

In another embodiment, the present disclosure provides a nucleic acid encoding the complementarity determining regions (CDRs) of a heavy chain variable region of a Pro-Factor D-specific antibody, or antigen-binding fragment thereof, that specifically binds to human Pro-Factor D, wherein the heavy chain variable region comprises an amino acid sequence set forth in SEQ ID NOs: 136-141 and wherein the CDRs are numbered according to the Kabat numbering system. In another embodiment, the present disclosure provides a nucleic acid encoding complementarity determining regions (CDRs) of a light chain variable region of a Pro-Factor D-specific antibody, or antigen-binding fragment thereof, that specifically binds to human Pro-Factor D, wherein the light chain variable region comprises an amino acid sequence set forth in SEQ ID NOs: 142-147 and wherein the CDRs are numbered according to the Kabat numbering system.

In another embodiment, the present disclosure provides a cloning or expression vector comprising a nucleic acid encoding complementarity determining regions (CDRs) of heavy and/or light chain variable regions of an antibody, or antigen-binding fragment thereof, that specifically binds to human Pro-Factor D, wherein the heavy chain variable region comprises the amino acid sequence set forth as any of SEQ ID NOs: 136-141 and the light chain variable region comprises the amino acid sequence set forth as any of SEQ ID NOs: 142-147 wherein the CDRs are numbered according to the Kabat numbering system.

In another embodiment, the present disclosure provides a cell containing a cloning or expression vector comprising a nucleic acid encoding complementarity determining regions (CDRs) of heavy and/or light chain variable regions of a Pro-Factor D-specific antibody, or antigen-binding fragment thereof that specifically binds to human Pro-Factor D, wherein the heavy chain variable region comprises the amino acid sequence set forth as any of SEQ ID NOs: 136-141 and the light chain variable region comprises the amino acid sequence set forth as any of SEQ ID NOs: 142-147 wherein the CDRs are numbered according to the Kabat numbering system.

In another embodiment, the present disclosure provides a method for producing a human Pro-Factor-D-specific antibody comprising culturing a cell containing an expression vector which contains a nucleic acid that encodes one or both of the heavy and light chain polypeptides of any of the Pro-Factor-D specific antibodies or antigen-binding fragments disclosed herein. The cell or culture of cells is cultured under conditions and for a time sufficient to allow expression by the cell (or culture of cells) of the antibody or antigen-binding fragment thereof encoded by the nucleic acid. The method can also include isolating the antibody or antigen binding fragment thereof from the cell (or culture of cells) or from the media in which the cell or cells were cultured.

In one embodiment, the present disclosure provides a composition comprising any of the Pro-Factor-D-specific antibodies, or antigen-binding fragments disclosed herein.

In one embodiment, the present disclosure provides a substrate for use in an immunoassay comprising at least one or more of any of the Pro-Factor-D-specific antibodies, or antigen-binding fragments disclosed herein.

In one embodiment, the present disclosure provides a kit for detecting the presence or amount of Pro-Factor D in a test sample, such as a biological sample, said kit comprising (a) at least one container, and (b) at least one or more of any of the Pro-Factor-D-specific antibodies, or antigen-binding fragments disclosed herein.

C. Anti-Human Factor D Monoclonal Antibodies that Bind to Both Pro and Mature Forms of Factor D

As described in Examples 4-5 herein, the present invention also provides anti-Factor D antibodies that bind to an epitope that is present in both human Pro- and human mature-Factor D. As described in Example 4, the inventors have used the human mature Factor D (SEQ ID NO:3) as an antigen to generate anti-Factor D antibodies which were screened and selected for the ability to detect both the Pro- and mature forms of Factor D and are suitable for use in combination with the mature-Factor D-specific antibodies and the Pro-Factor D-specific antibodies disclosed herein in the detection assays and methods described herein.

As described in Example 5, the variable heavy and light chain fragments of several representative anti-Factor D monoclonal antibodies that bind to both Pro- and mature-Factor D have been cloned and sequenced.

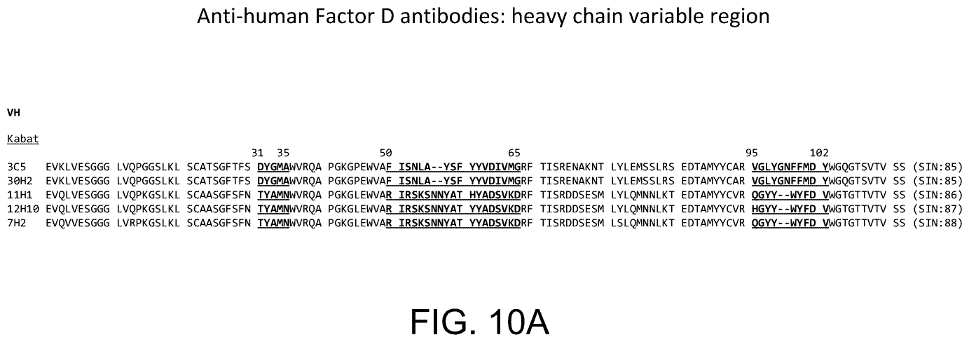

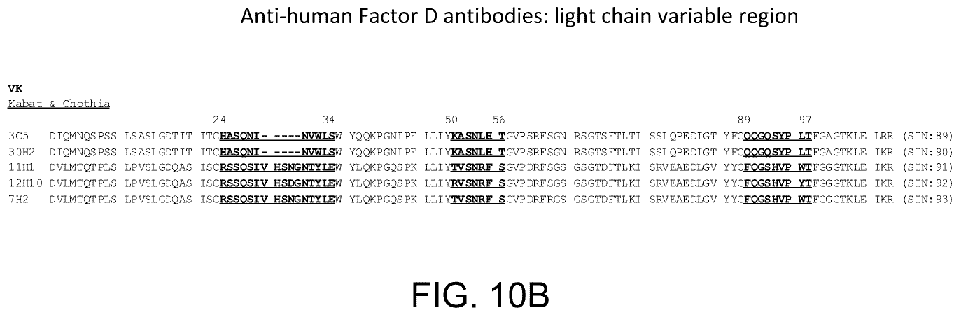

FIG. 10A is an amino acid sequence alignment of the variable heavy chain regions of five anti-Factor D clones that were identified as having high binding affinity to both mature and pro-Factor D.

FIG. 10B is an amino acid sequence alignment of the variable light chain regions of five anti-Factor D clones that were identified as having high binding affinity to both mature and pro-Factor D.

The heavy chain and light chain variable regions and CDRs therein of the five anti-Factor D monoclonal antibodies that bind to both mature and pro-Factor D are provided below in TABLES 5 and 6.

TABLE 5
anti-human Factor D Antibody Sequences: mouse parental

| Anti-human | Heavy Chain | Light Chain | | |
|---|---|---|---|---|
| Factor D | Variable | Variable | Heavy chain | Light chain |
| Antibody | Region | Region | variable region | variable region |
| Reference No | (amino acid) | (amino acid) | (DNA) | (DNA) |
| 3C5 | SEQ ID NO: 85 | SEQ ID NO: 89 | SEQ ID NO: 127 | SEQ ID NO: 131 |
| 30H2 | SEQ ID NO: 85 | SEQ ID NO: 90 | SEQ ID NO: 127 | SEQ ID NO: 132 |
| 11H1 | SEQ ID NO: 86 | SEQ ID NO: 91 | SEQ ID NO: 128 | SEQ ID NO: 133 |
| 12H10 | SEQ ID NO: 87 | SEQ ID NO: 92 | SEQ ID NO: 129 | SEQ ID NO: 134 |
| 7H2 | SEQ ID NO: 88 | SEQ ID NO: 93 | SEQ ID NO: 130 | SEQ ID NO: 135 |

TABLE 6
anti-human Factor D antibodies: CDRs

| | Heavy Chain: | Light Chain: |
|---|---|---|
| | CDR1; | CDR1; |
| Anti-human Factor | CDR2; | CDR2; |
| D Antibody | CDR3 | CDR3 |
| Reference No. | (SEQ ID NOs) | (SEQ ID NOs) |
| 3C5 | 95; 97; 99 | 111; 113; 115 |
| 30H2 | 95; 97; 99 | 111; 113; 115 |
| 11H1 | 101; 103; 105 | 60; 119; 121 |
| 12H10 | 101; 107; 108 | 123; 124; 125 |
| 7H2 | 101; 107; 105 | 60; 126; 121 |

According, in one aspect, the present invention provides an isolated antibody or fragment thereof that specifically binds to an epitope present in both human Pro-Factor D (SEQ ID NO:2), and human mature Factor D (SEQ ID NO:3), wherein the antibody comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

In one embodiment, the anti-Factor D antibody that binds to an epitope present in both Pro-Factor D and mature Factor D is a monoclonal antibody. In one embodiment the anti-Factor D antibody is a humanized, chimeric, or fully human antibody. In one embodiment the anti-Factor D antibody fragment is selected from the group consisting of Fv, Fab, Fab′, F(ab)₂ and F(ab′)₂. In one embodiment, the anti-Factor D antibody is a single-chain molecule. In one embodiment, the anti-Factor D antibody is an IgG molecule selected from the group consisting of IgG1, IgG2 and IgG4. In one embodiment, the anti-Factor D antibody or antigen-binding fragment thereof binds to both human Pro-Factor D and human mature Factor D with a K_{D} of less than 10 nM. In one embodiment, the anti-Factor D antibody or antigen-binding fragment thereof that binds to an epitope present in both Pro-Factor D and mature Factor D is labeled with a detectable moiety, for example a detectable moiety suitable for use in an immunoassay as further described herein. In one embodiment, the anti-Factor D antibody or fragment thereof that binds to an epitope present in both Pro-Factor D and mature Factor D is immobilized on a substrate, such as a substrate suitable for use in an immunoassay, as further described herein.

In one embodiment, the anti-Factor D antibody or fragment thereof that binds to an epitope shared by both human mature Factor D and human Pro-Factor D comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

In one embodiment, the anti-Factor D antibody or fragment thereof that binds to an epitope shared by both human mature Factor D and human Pro-Factor D comprises a binding domain comprising the following six CDRs: (a) an HC-CDR1 comprising the amino acid sequence SEQ ID NO:95 (b) an HC-CDR2 comprising the amino acid sequence SEQ ID NO: 97 (c) an HC-CDR3 comprising the amino acid sequence SEQ ID NO:99 (d) a LC-CDR1 comprising the amino acid sequence SEQ ID NO:111; (e) a LC-CDR2 comprising the amino acid sequence SEQ ID NO:113); and (f) a LC-CDR3 comprising the amino acid sequence SEQ ID NO:115.

In one embodiment, the anti-Factor D antibody or fragment thereof that binds to an epitope shared by both human mature Factor D and human Pro-Factor D comprises a binding domain comprising the following six CDRs: (a) an HC-CDR1 comprising the amino acid sequence SEQ ID NO:101 (b) an HC-CDR2 comprising the amino acid sequence SEQ ID NO: 103 or 107 (c) an HC-CDR3 comprising the amino acid sequence SEQ ID NO:105 or 108, (d) a LC-CDR1 comprising the amino acid sequence SEQ ID NO:60 or 123; (e) a LC-CDR2 comprising the amino acid sequence SEQ ID NO:119, 124 or 126 and (f) a LC-CDR3 comprising the amino acid sequence SEQ ID NO:121 or 125.

In one embodiment, the anti-Factor D antibody or fragment thereof that binds to an epitope shared by both human mature Factor D and human Pro-Factor D comprises a VH domain having at least 95% sequence identity (such as at least 96%, at least 97%, at least 98%, or at least 99% identity) to the amino acid sequence of SEQ ID NO:85. In one embodiment, the anti-Factor D antibody or fragment thereof comprises a VL domain having at least 95% sequence identity (such as at least 96%, at least 97%, at least 98%, or at least 99% identity) to the amino acid sequence of SEQ ID NO:89. In one embodiment, the anti-Factor D antibody or fragment thereof comprises a VL domain having at least 95% sequence identity (such as at least 96%, at least 97%, at least 98%, or at least 99% identity) to the amino acid sequence of SEQ ID NO:90. In one embodiment, the anti-Factor D antibody or fragment thereof comprises a VH comprising SEQ ID NO:85 and a VL comprising SEQ ID NO: 89. In one embodiment, the anti-Factor D antibody or fragment thereof comprises a VH comprising SEQ ID NO:85 and a VL comprising SEQ ID NO:90.

In one embodiment the anti-Factor D antibody or fragment thereof that binds to an epitope shared by both human mature Factor D and human Pro-Factor D comprises a VH domain having at least 95% sequence identity (such as at least 96%, at least 97%, at least 98%, or at least 99% identity) to the amino acid sequence of SEQ ID NO:86. In one embodiment the anti-Factor D antibody or fragment thereof comprises a VH domain having at least 95% sequence identity (such as at least 96%, at least 97%, at least 98%, or at least 99% identity) to the amino acid sequence of SEQ ID NO:87. In one embodiment the anti-Factor D antibody or fragment thereof comprises a VH domain having at least 95% sequence identity (such as at least 96%, at least 97%, at least 98%, or at least 99% identity) to the amino acid sequence of SEQ ID NO:88.

In one embodiment, the anti-Factor D antibody or fragment thereof that binds to an epitope shared by both human mature Factor D and human Pro-Factor D comprises a VL domain having at least 95% sequence identity (such as at least 96%, at least 97%, at least 98%, or at least 99% identity) to the amino acid sequence of SEQ ID NO:91. In one embodiment, the anti-Factor D antibody or fragment thereof comprises a VL domain having at least 95% sequence identity (such as at least 96%, at least 97%, at least 98%, or at least 99% identity) to the amino acid sequence of SEQ ID NO:92. In one embodiment, the anti-Factor D antibody or fragment thereof comprises a VL domain having at least 95% sequence identity (such as at least 96%, at least 97%, at least 98%, or at least 99% identity) to the amino acid sequence of SEQ ID NO:93. In one embodiment, the anti-Factor D antibody or fragment thereof comprises a VH comprising SEQ ID NO:86 and a VL comprising SEQ ID NO: 91. In one embodiment, the anti-Factor D antibody or fragment thereof comprises a VH comprising SEQ ID NO:87 and a VL comprising SEQ ID NO:92. In one embodiment, the anti-Factor D antibody or fragment thereof comprises a VH comprising SEQ ID NO:88 and a VL comprising SEQ ID NO:93.

In certain embodiments, the anti-Factor D antibody or fragment thereof that binds to an epitope shared by both human mature Factor D and human Pro-Factor D has a heavy chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the heavy chain variable domain sequences set forth in TABLE 5.

In certain embodiments, the anti-Factor D antibody or fragment thereof that binds to an epitope shared by both human mature Factor D and human Pro-Factor D has a light chain variable domain that is substantially identical (e.g., at least about 70%, at least 75%, at least about 80%, at least about 85%, at least about 90%, at least about 95%, or at least about 96% identical, or at least about 97% identical, or at least about 98% identical, or at least 99% identical), to that of any of the light chain variable domain sequences set forth in TABLE 5.

In another embodiment, the present disclosure provides a nucleic acid encoding the complementarity determining regions (CDRs) of a heavy chain variable region of an anti-Factor D antibody, or antigen-binding fragment thereof, that binds to an epitope shared by both human mature Factor D and human Pro-Factor D, wherein the heavy chain variable region comprises an amino acid sequence set forth in SEQ ID NOs: 85-88 and wherein the CDRs are numbered according to the Kabat numbering system. In another embodiment, the present disclosure provides a nucleic acid encoding complementarity determining regions (CDRs) of a light chain variable region of an antibody, or antigen-binding fragment thereof, that binds to an epitope shared by both human mature Factor D and human Pro-Factor D, wherein the light chain variable region comprises an amino acid sequence set forth in SEQ ID NOs: 89-93 and wherein the CDRs are numbered according to the Kabat numbering system.

In another embodiment, the present disclosure provides a cloning or expression vector comprising a nucleic acid encoding complementarity determining regions (CDRs) of heavy and/or light chain variable regions of an anti-Factor D antibody, or antigen-binding fragment thereof, that binds to an epitope shared by both human mature Factor D and human Pro-Factor D, wherein the heavy chain variable region comprises the amino acid sequence set forth as any of SEQ ID NOs: 85-88 and the light chain variable region comprises the amino acid sequence set forth as any of SEQ ID NOs: 89-93 wherein the CDRs are numbered according to the Kabat numbering system.

In another embodiment, the present disclosure provides a cell containing a cloning or expression vector comprising a nucleic acid encoding complementarity determining regions (CDRs) of heavy and/or light chain variable regions of an anti-Factor D antibody, or antigen-binding fragment thereof, that binds to an epitope shared by both human mature Factor D and human Pro-Factor D, wherein the heavy chain variable region comprises the amino acid sequence set forth as any of SEQ ID NOs: 85-88 and the light chain variable region comprises the amino acid sequence set forth as any of SEQ ID NOs: 89-93 wherein the CDRs are numbered according to the Kabat numbering system.

In another embodiment, the present disclosure provides a method for producing an anti-Factor D antibody that binds to an epitope shared by both human mature Factor D and human Pro-Factor D comprising culturing a cell containing an expression vector which contains a nucleic acid that encodes one or both of the heavy and light chain polypeptides of any of the antibodies or antigen-binding fragments disclosed herein. The cell or culture of cells is cultured under conditions and for a time sufficient to allow expression by the cell (or culture of cells) of the anti-Factor D antibody or antigen-binding fragment thereof encoded by the nucleic acid. The method can also include isolating the antibody or antigen binding fragment thereof from the cell (or culture of cells) or from the media in which the cell or cells were cultured.

In one embodiment, the present disclosure provides a composition comprising any of the anti-Factor D antibodies, or antigen-binding fragments thereof, that bind to an epitope shared by both human mature Factor D and human Pro-Factor D disclosed herein.

In one embodiment, the present disclosure provides a substrate for use in an immunoassay comprising at least one or more of any of the anti-Factor D antibodies, or antigen-binding fragments thereof, that bind to an epitope shared by both human mature Factor D and human Pro-Factor D disclosed herein.

In one embodiment, the present disclosure provides a kit for detecting the presence of Factor D in a test sample, such as a biological sample, said kit comprising (a) at least one container, and (b) at least one or more of any of the anti-Factor D antibodies, or antigen-binding fragments thereof, that bind to an epitope shared by both human mature Factor D and human Pro-Factor D disclosed herein.

### Single-Chain Anti-Factor D Antibodies

In one embodiment of the present invention, the anti-Factor D antibodies (i.e., any of the mature Factor-D specific antibodies, the Pro-Factor-D-specific antibodies or the anti-Factor-D antibodies that bind both the mature and Pro-forms of Factor D) are single-chain antibodies, defined as a genetically engineered molecule containing the variable region of the light chain, the variable region of the heavy chain, linked by a suitable polypeptide linker as a genetically fused single-chain molecule. Such single-chain antibodies are also referred to as “single-chain Fv” or “scFv” antibody fragments. Generally, the Fv polypeptide further comprises a polypeptide linker between the VH and VL domains that enables the scFv to form the desired structure for antigen binding. The scFv antibodies that bind Factor D can be oriented with the variable light region either amino terminal to the variable heavy region or carboxyl terminal to it.

### Humanized Anti-Factor D Antibodies

The anti-Factor D antibodies disclosed herein (i.e., any of the mature Factor-D specific antibodies, the Pro-Factor-D-specific antibodies or the anti-Factor-D antibodies that bind both the mature and Pro-forms of Factor D) can be modified without changing their ability to be used for the purposes described herein. As an initial matter, it is noted that the antibodies described herein originated from immunized mice. The antibodies thus have framework regions (regions outside the complementarity determining regions, or “CDRs”) which contain the amino acid residues usually found in the framework regions in murine antibodies, and which may be immunogenic when administered to a human patient. To reduce immunogenicity of murine antibodies when used in humans, it is common in the art to engineer the framework regions by replacing residues found at particular positions in the antibodies of mice with the residues more typically found at the same position in human antibodies. Antibodies engineered in these ways are referred to as “humanized antibodies” and are typically preferred for in vivo use, since they have a lower risk of inducing side effects and typically can remain in the circulation longer. Methods of humanizing antibodies are known in the art and are set forth in detail in, for example, U.S. Pat. Nos. 6,180,377; 6,407,213; 5,693,762; 5,585,089; and 5,530,101.

Further, since the CDRs of the variable regions determine antibody specificity, the anti-Factor D antibody CDRs set forth in TABLES 2, 4, 6-10 and 12-17 can be grafted or engineered into an antibody of choice to confer specificity for binding to Factor D upon that antibody. For example, the CDRs from mature Factor-D-specific clones 6G6, 14A11, 27B3, 58F5, 49G3 and 10G1 as set forth in TABLE 2 and/or from Pro-Factor D-specific clones 18F5, 1F9, 2A4, 20A1, 13A10 and 21H1 as set forth in TABLE 4 can be grafted onto a human antibody framework of known three dimensional structure (see e.g., WO98/45322; Jones et al., Nature 321:522 (1986); Verhoeyen et al., Science 239:1534 (1988); Riechmann et al., Nature 332:323 (1988) and Winter & Milstein, Nature 349:293 (1991) to generate an anti-mature Factor D-specific or anti-Pro-Factor D specific antibody with reduced or no immunogenic responses when administered to humans.

### Methods for Producing Anti-Factor D Antibodies

In another aspect, the present invention provides a method of producing an antibody specifically recognizing and binding human Factor D, such as mature Factor-D specific antibodies, the Pro-Factor-D-specific antibodies or anti-Factor-D antibodies that bind both the mature and Pro-forms of Factor D comprising culturing a cell containing an expression vector which contains a nucleic acid that encodes one or both of the heavy and light chain polypeptides of any of the antibodies or antigen-binding fragments disclosed herein. The cell or culture of cells is cultured under conditions and for a time sufficient to allow expression by the cell (or culture of cells) of the antibody or antigen-binding fragment thereof encoded by the nucleic acid. The method can also include isolating the antibody or antigen binding fragment thereof from the cell (or culture of cells) or from the media in which the cell or cells were cultured.

In one embodiment, the present disclosure features a cell containing a cloning or expression vector comprising a nucleic acid encoding complementarity determining regions (CDRs) of heavy and/or light chain variable regions of an antibody, or antigen-binding fragment thereof, that specifically binds to human mature Factor D, wherein the heavy chain variable region comprises the amino acid sequence set forth as any of SEQ ID NOs: 12-17 and the light chain variable region comprises the amino acid sequence set forth as any of SEQ ID NOs: 18-23.

In another embodiment, the present disclosure features a cell containing a cloning or expression vector comprising a nucleic acid encoding complementarity determining regions (CDRs) of heavy and/or light chain variable regions of an antibody, or antigen-binding fragment thereof, that specifically binds to human Pro-Factor D, wherein the heavy chain variable region comprises the amino acid sequence set forth as any of SEQ ID NOs: 136-141 and the light chain variable region comprises the amino acid sequence set forth as any of SEQ ID NOs: 142-147.

In some embodiments, the invention provides a nucleic acid molecule encoding an anti-Factor D antibody, or fragment thereof, of the invention, such as an antibody or antigen-binding fragment thereof that specifically binds to human mature Factor D (e.g., as set forth in TABLE 1), an antibody or antigen-binding fragment thereof that specifically binds to human Pro-Factor D (e.g., as set forth in TABLE 3) or an antibody or antigen-binding fragment thereof that binds to an epitope shared by both human mature Factor D and human Pro-Factor D (e.g., as set forth in TABLE 5). In some embodiments the invention provides a nucleic acid molecule comprising a nucleic acid sequence encoding an anti-Factor D antibody, such as encoding an antibody or antigen-binding fragment thereof that specifically binds to human mature Factor D (e.g., SEQ ID NOs: 73-84), or encoding an antibody or antigen-binding fragment thereof that specifically binds to human Pro-Factor D (e.g., SEQ ID NOs: 206-217) or encoding an antibody or antigen-binding fragment thereof that binds to an epitope shared by both human mature Factor D and human Pro-Factor D (e.g., SEQ ID NOs: 127-135).

In some embodiments, the invention provides a cell comprising a nucleic acid molecule encoding a Factor D-specific monoclonal antibody of the invention (including mature Factor-D specific antibodies, the Pro-Factor-D-specific antibodies and anti-Factor-D antibodies that bind both the mature and Pro-forms of Factor D).

In some embodiments, the invention provides an expression cassette comprising a nucleic acid molecule encoding a Factor D-specific monoclonal antibody of the invention (including mature Factor-D specific antibodies, the Pro-Factor-D-specific antibodies and anti-Factor-D antibodies that bind both the mature and Pro-forms of Factor D).

In some embodiments, the invention provides a method of producing Factor D-specific monoclonal antibodies comprising culturing a cell comprising a nucleic acid molecule encoding a Factor D-specific antibody of the invention (including mature Factor-D specific antibodies, the Pro-Factor-D-specific antibodies and anti-Factor-D antibodies that bind both the mature and Pro-forms of Factor D).

In many embodiments, the nucleic acids encoding a subject monoclonal antibody are introduced directly into a host cell, and the cell incubated under conditions sufficient to induce expression of the encoded antibody.

In one embodiment, the method of producing a Factor D-specific monoclonal antibody (including mature Factor-D specific antibodies, the Pro-Factor-D-specific antibodies or the anti-Factor-D antibodies that bind both the mature and Pro-forms of Factor D) comprises culturing a cell comprising a nucleic acid molecule encoding a Factor D-specific antibody of the invention.

According to certain related embodiments there is provided a recombinant host cell which comprises one or more constructs as described herein; a nucleic acid encoding any anti-Factor D antibody, CDR, VH or VL domain, or antigen-binding fragment thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

For example, any cell suitable for expression of expression cassettes may be used as a host cell, for example, yeast, insect, plant, etc., cells. In many embodiments, a mammalian host cell line that does not ordinarily produce antibodies is used, examples of which are as follows: monkey kidney cells (COS cells), monkey kidney CVI cells transformed by SV40 (COS-7, ATCC CRL 165 1); human embryonic kidney cells (HEK-293, Graham et al., J. Gen Virol. 36:59 (1977)); baby hamster kidney cells (BHK, ATCC CCL 10); Chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

Methods of introducing nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e., in vitro, ex vivo, or in vivo). A general discussion of these methods can be found in Ausubel, et al., Short Protocols in Molecular Biology, 3d ed., Wiley & Sons, 1995. In some embodiments, lipofectamine and calcium mediated gene transfer technologies are used.

After the subject nucleic acids have been introduced into a cell, the cell is typically incubated, normally at 37° C., sometimes under selection, for a suitable time to allow for the expression of the antibody. In most embodiments, the antibody is typically secreted into the supernatant of the media in which the cell is growing in.

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule, may be engineered. Rather than using expression vectors, which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci, which in turn can be cloned and expanded into cell lines. Such engineered cell lines may be particularly useful in screening and evaluation of compounds that interact directly or indirectly with the antibody molecule.

Once an antibody molecule of the invention has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In many embodiments, antibodies are secreted from the cell into culture medium and harvested from the culture medium. For example, a nucleic acid sequence encoding a signal peptide may be included adjacent the coding region of the antibody or fragment. Such a signal peptide may be incorporated adjacent to the 5′ end of the amino acid sequences set forth herein for the subject antibodies in order to facilitate production of the subject antibodies.

Anti-Factor D Antibodies Labeled with a Detectable Moiety

In another aspect, the invention provides anti-Factor D antibodies (including mature Factor-D-specific antibodies, Pro-Factor-D-specific antibodies and anti-Factor-D antibodies that bind both the mature and Pro-forms of Factor D) that are labeled with a detectable moiety (i.e., a moiety that permits detection and/or quantitation). In various embodiments, the antibodies described herein are conjugated to a detectable label that may be detected directly or indirectly. In this regard, an antibody “conjugate” refers to an anti-Factor D antibody that is covalently linked to a detectable label. In the present invention, monoclonal antibodies, antigen-binding fragments thereof, and antibody derivatives thereof, such as a single-chain-variable-fragment antibody or an epitope tagged antibody, may all be covalently linked to a detectable label. In “direct detection”, only one detectable antibody is used, i.e., a primary detectable antibody. Thus, direct detection means that the antibody that is conjugated to a detectable label may be detected, per se, without the need for the addition of a second antibody (secondary antibody).

A “detectable label” is a molecule or material that can produce a detectable (such as visually, electronically, or otherwise) signal that indicates the presence and/or concentration of the label in a sample. When conjugated to an antibody, the detectable label can be used to locate and/or quantify the target to which the specific antibody is directed. Thereby, the presence and/or concentration of the target in a sample can be detected by detecting the signal produced by the detectable label. A detectable label can be detected directly or indirectly, and several different detectable labels conjugated to different specific antibodies can be used in combination to detect one or more targets.

Examples of detectable labels, which may be detected directly, include fluorescent dyes and radioactive substances and metal particles. In contrast, indirect detection requires the application of one or more additional antibodies, i.e., secondary antibodies, after application of the primary antibody. Thus, the detection is performed by the detection of the binding of the secondary antibody or binding agent to the primary detectable antibody. Examples of primary detectable binding agents or antibodies requiring addition of a secondary binding agent or antibody include enzymatic detectable binding agents and hapten detectable binding agents or antibodies.

Examples of detectable labels which may be conjugated to antibodies of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes, or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), .alpha.-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b--d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to the antibodies described herein (i.e., any of the mature Factor-D specific antibodies, the Pro-Factor-D-specific antibodies or the anti-Factor-D antibodies that bind both the mature and Pro-forms of Factor D) or to any other molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biological marker of interest may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

In another embodiment, the anti-Factor D antibody is not labeled (i.e., is naked), and the presence thereof can be detected using a labeled antibody which binds to the anti-Factor D antibody (i.e., any of the mature Factor-D specific antibodies, the Pro-Factor-D-specific antibodies or the anti-Factor-D antibodies that bind both the mature and Pro-forms of Factor D).

### V. Compositions and Kits Comprising Anti-Factor D Antibodies

### Compositions

In another aspect, the present disclosure provides a substrate, such as a solid support (e.g., an insoluble substrate, such as non-aqueous matrix, such as a plate or slide made of glass, polysaccharides (e.g., agarose), polyacrylamides, polystyrene, plastic or metal, a polymer-coated bead, a tube, or a ceramic or metal chip) that comprises immobilized (or otherwise deposited) monoclonal anti-Factor D antibodies disclosed herein (such as mature Factor-D specific antibodies, Pro-Factor-D-specific antibodies and anti-Factor-D antibodies that bind both the mature and Pro-forms of Factor D). In some embodiments, the anti-Factor D antibodies are immobilized (or deposited) at discrete locations (e.g., in the wells of a multiwall plate, or deposited in an array on a biochip). In some embodiments, the substrate comprising the anti-Factor D antibodies may be part of a kit for detecting Factor D (such as mature Factor D, Pro-Factor D, or total Factor D (mature and Pro-Factor D) in a biological sample obtained from a mammalian subject.

### Kits

In another aspect, the present disclosure provides kits for use in performing one or more assays disclosed herein.

In one embodiment, the present disclosure provides a kit (i.e., a packaged combination of reagents in predetermined amounts) with reagents and instructions for detecting the presence of Factor D (such as mature Factor D, Pro-Factor D, or total Factor D (mature and Pro-Factor D)) in a test sample, such as a biological sample. Exemplary kits may contain at least one anti-Factor D monoclonal antibody or antigen binding fragment thereof as described herein (i.e., any of the mature Factor-D specific antibodies, the Pro-Factor-D-specific antibodies or the anti-Factor-D antibodies that bind both the mature and Pro-forms of Factor D). Where the anti-Factor D antibody is labeled with a detectable moiety, such as an enzyme, the kit will include substrates and cofactors required by the enzyme (e.g., a substrate precursor which provides the detectable chromophore or fluorophore). In addition, other additives may be included such as stabilizers, buffers (e.g., a blocking buffer or lysis buffer) and the like. The relative amounts of the various reagents may be varied widely to provide for concentrations in solution of the reagents, which substantially optimize the sensitivity of the assay. Particularly, the reagents may be provided as dry powders, usually lyophilized, including excipients which on dissolution will provide a reagent solution having the appropriate concentration.

In addition, kits may include instructional materials disclosing means of use of an antibody of the present invention (e.g., for detection of mature Factor D or Pro-Factor D as a biomarker for the level of Alternative Pathway Complement (APC) activation, or absence thereof). The kits may also include additional components to facilitate the particular application for which the kit is designed. For example, the kit may additionally contain means of detecting a label (e.g., enzyme substrates for enzymatic labels, filter sets to detect fluorescent labels, appropriate secondary labels such as a sheep anti-mouse-HRP or the like). The kits may additionally include buffers and other reagents routinely used for the practice of a particular immunoassay, as is well known in the art.

Certain embodiments provide kits for detecting the presence or amount of mature Factor D in a sample, wherein the kits contain at least one mature Factor D-specific antibody as described herein, such as an antibody or fragment comprising the CDRs from mature Factor-D-specific clones 6G6, 14A11, 27B3, 58F5, 49G3 and 10G1 as set forth in TABLE 2. In certain embodiments, a kit may comprise buffers, enzymes, labels, substrates, beads, or other surfaces to which the antibodies of the invention are attached, and the like, and instructions for use.

Certain embodiments provide kits for detecting the presence or amount of Pro-Factor D in a sample, wherein the kits contain at least one Pro-Factor D-specific antibody as described herein, such an antibody or fragment comprising the CDRs from Pro-Factor D-specific clones 18F5, 1F9, 2A4, 20A1, 13A10 and 21H1 as set forth in TABLE 4. The subject anti-Factor D antibodies and antigen-binding fragments thereof can be labeled with any appropriate detectable moiety as described herein. In certain embodiments, a kit may comprise buffers, enzymes, labels, substrates, beads, or other surfaces to which the antibodies of the invention are attached, and the like, and instructions for use.

Items in a kit may be individually wrapped or packaged in individual receptacles, which are provided together in a larger container (e.g., a cardboard or styrofoam box).

In accordance with the foregoing, in one embodiment, the present disclosure provides a kit comprising at least one monoclonal antibody that specifically detects or quantitates human mature Factor D (SEQ ID NO:3) and/or Pro-Factor D (SEQ ID NO:2) in an immunoassay, wherein the at least one monoclonal antibody comprises: (i) a mature Factor D-specific monoclonal antibody, or antigen-binding fragment thereof, that specifically binds to an epitope encompassing the amino-terminus of human mature Factor D, wherein the epitope comprises or consists of the amino acids ILGGREA (SEQ ID NO:5) and wherein said antibody does not bind to human Pro-Factor D (SEQ ID NO:2); and/or (ii) a Pro-Factor D-specific monoclonal antibody, or antigen-binding fragment thereof, that specifically binds to an epitope on the activation (“Pro”) peptide of human Factor D, wherein the epitope comprises or consists of “APPRGR” (SEQ ID NO:4) and wherein said antibody does not bind to mature Factor D (SEQ ID NO:3). In one embodiment, the mature Factor D-specific antibody or fragment thereof comprises a binding domain comprising HC-CDR-1, HC-CDR-2 and HC-CDR-3 in a heavy chain variable region selected from the group consisting of SEQ ID NO:s 12-17 and comprising LC-CDR-1, LC-CDR2 and LC-CDR3 in a light chain variable region selected from the group consisting of SEQ ID NO:s 18-23, wherein the CDRs are numbered according to the Kabat numbering system. In one embodiment the Pro-Factor D-specific antibody or fragment thereof comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

In some embodiments, the kit further comprises an anti-Factor D antibody, or fragment thereof, that binds to an epitope shared by both human mature Factor D (SEQ ID NO: 3) and human Pro-Factor D (SEQ ID NO:2). In some embodiments, the anti-Factor D antibody or fragment thereof comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

In some embodiments, the kit further comprises at least one container.

In some embodiments, the kit is for carrying out an enzyme-linked immunosorbent assay (ELISA). In one embodiment, the mature Factor D-specific antibody or fragment thereof is a coating antibody. In one embodiment, the mature Factor D-specific antibody or fragment thereof is a detecting antibody. In one embodiment, the Pro-Factor D-specific antibody or fragment thereof is a coating antibody. In one embodiment, the Pro-Factor D-specific antibody or fragment thereof is a detecting antibody.

In various embodiments of the kits of the invention, the subject anti-Factor D antibodies and antigen-binding fragments thereof (i.e., mature Factor D-specific antibodies, Pro-Factor D-specific antibodies and/or anti-Factor D antibodies) can be labeled with any appropriate detectable moiety as described herein. In certain embodiments, the kit further comprises buffers, enzymes, labels, substrates, beads, or other surfaces to which the antibodies of the invention are attached, and the like, and instructions for use.

### VI. Methods of Detecting Factor D Using Anti-Factor D Antibodies

As described herein, the inventors have generated anti-Factor D antibodies that are suitable for use in an immunoassay for detecting the presence and/or amount of Factor D (such as mature Factor D, Pro-Factor D and total Factor D (both mature and pro forms of Factor D) in a test sample, such as a biological sample obtained from a mammalian subject.

In one aspect, the anti-Factor D antibodies (including mature Factor-D specific antibodies, the Pro-Factor-D-specific antibodies and the anti-Factor-D antibodies that bind both the mature and Pro-forms of Factor D) of the present invention are used in an in vitro immunoassay for analyzing a test sample, such as a biological sample obtained from a test subject, for the presence or amount of Pro-Factor D, mature Factor D, and/or total Factor D. In such in vitro immunoassays, the anti-Factor D antibody, or antigen-binding fragment thereof, may be naked or may be labeled with a detectable moiety, as described herein, and may be utilized in liquid phase or bound to a substrate, as described below. For purposes of in vitro assays, any type of antibody such as murine, chimeric, humanized or human may be utilized, since there is no host immune response to consider.

The antibodies of the present disclosure may be employed in any known immunoassay method, such as competitive binding assays, direct and indirect sandwich assays, lateral flow assays (e.g., dipstick format) and immunoprecipitation assays (see e.g., Zola, Monoclonal Antibodies: A Manual of Techniques, pp. 147-158 (CRC Press. Inc., 1987).

Sandwich assays involve the use of two antibodies, each capable of binding to a different immunogenic portion, or epitope, of the protein to be detected (e.g., Factor D). In a sandwich assay, the test sample analyte is bound by a first antibody (e.g., an anti-Factor D antibody, such as a mature Factor D-specific antibody, a Pro-Factor D-specific antibody and/or an antibody that binds to both mature and pro Factor D), which is immobilized on a solid support (e.g., substrate), and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assay).

For example, one preferable type of sandwich assay is an ELISA assay, in which case the detectable moiety is an enzyme. ELISA assays, regardless of the detection system employed, generally include the immobilization of an antigen or antibody to a substrate (e.g., a solid support), as well as the use of an appropriate detecting reagent. In an ELISA assay, the protein antigen-antibody reaction takes place on a substrate (e.g., a solid support), typically in wells on microtiter plates. Antigen and this first antibody, also called the coating or capture antibody, react and produce a stable complex, which can be visualized by addition of a second antibody, called the detection antibody, which may be directly or indirectly linked to an enzyme. Addition of a substrate for that enzyme results in a color formation, which can be measured photometrically.

In one embodiment, the anti-Factor D antibodies (including mature Factor-D specific antibodies, the Pro-Factor-D-specific antibodies and the anti-Factor-D antibodies that bind both the mature and Pro-forms of Factor D) of the invention are used to detect the presence of the mature or Pro-forms of the Factor D antigen in a biological sample using an enzyme-linked immunosorbent assay (ELISA) (see e.g., Gold et al. J Clin Oncol. 24:252-58, 2006).

In the direct competitive ELISA, a pure or semipure antigen preparation is bound to a substrate that is insoluble in the fluid or cellular extract being tested and a quantity of detectably labeled soluble antibody is added to permit detection and/or quantitation of the binary complex formed between substrate-bound antigen and labeled antibody.

In contrast, a “double-determinant” ELISA, also known as a “two-site ELISA” or “sandwich assay,” requires small amounts of antigen and the assay does not require extensive purification of the antigen. Thus, the double-determinant ELISA is preferred to the direct competitive ELISA for the detection of an antigen in a clinical sample. See, for example, the use of the double-determinant ELISA for quantitation of the c-myc oncoprotein in biopsy specimens. Field et al., Oncogene 4:1463 (1989); Spandidos et al., AntiCancer Res. 9:821 (1989). In a double-determinant ELISA, a quantity of unlabeled monoclonal antibody or antibody fragment (the “capture antibody”) is bound to a substrate (e.g., a solid support), the test sample is brought into contact with the capture antibody, and a quantity of detectably labeled soluble antibody (or antibody fragment) is added to permit detection and/or quantitation of the ternary complex formed between the capture antibody, antigen, and labeled antibody.

In one embodiment, the capture antibody bound to a substrate (e.g., solid support) is an anti-Factor D antibody or antigen-binding fragment thereof as disclosed herein that binds to an epitope that is shared by both the Pro- and mature-Factor D (i.e., in the C-terminal portion of Factor D). In one embodiment, the capture antibody bound to a substrate (e.g., solid support) is a mature Factor D-specific antibody or antigen-binding fragment thereof as disclosed herein. In one embodiment, the capture antibody bound to a substrate (e.g., solid support) a Pro-Factor D-specific antibody or antigen-binding fragment thereof as disclosed herein.

Methods of performing a double-determinant ELISA are well-known by those of skill in the art. See, for example, Field et al., Oncogene 4:1463 (1989); Spandidos et al., AntiCancer Res. 9:821 (1989); and Moore et al., Methods in Molecular Biology Vol 10:273-281 (The Humana Press, Inc. 1992).

In the double-determinant ELISA, the soluble antibody or antibody fragment must bind to a Factor D epitope that is distinct from the epitope recognized by the capture antibody. The double-determinant ELISA can be performed to ascertain whether the Factor D antigen (i.e., mature Factor D or Pro-Factor D) is present in a test biological sample, such as a body fluid (e.g., blood, plasma or serum) or a biopsy sample. Alternatively, the assay can be performed to quantitate the amount of Factor D antigen that is present in a clinical sample of body fluid. The quantitative assay can be performed by including dilutions of purified Factor D antigen.

In vitro immunoassays can be performed in which at least one anti-Factor D antibody or antigen-binding fragment thereof (e.g., a mature Factor-D specific antibody, a Pro-Factor-D-specific antibody and/or an anti-Factor-D antibody that binds both the mature and Pro-forms of Factor D) is bound to a substrate (e.g., a solid-phase carrier). For example, anti-Factor D monoclonal antibodies or fragments thereof can be attached to a polymer, such as aminodextran, in order to link the monoclonal antibody to an insoluble substrate such as a polymer-coated bead, a plate, a tube, or a ceramic or metal chip. In one embodiment, the substrate is suitable for use in an ELISA method (e.g., a multiwell microtitre plate). Accordingly, the determination of the level of Factor D (such as mature Factor D, pro-Factor D, or both mature and pro-Factor D) in the sample may be determined by commercially available methods such as an ELISA based assay, chemical or enzymatic protein determination.

Other suitable in vitro assays will be readily apparent to those of skill in the art. The specific concentrations of detectably labeled anti-anti-Factor D antibody, the temperature and time of incubation, as well as other assay conditions may be varied, depending on various factors including the concentration of the Factor D antigen in the sample, the nature of the sample, and the like. The binding activity of a sample of anti-Factor D antibody may be determined according to well-known methods. Those skilled in the art will be able to determine operative and optimal assay conditions for each determination by employing routine experimentation.

In another embodiment, the subject antibodies and antigen-binding fragments thereof can be used to detect the presence of the Factor D antigen in tissue sections prepared from a histological specimen (e.g., a biopsy sample). Such in situ detection can be used to determine the presence of the Factor D antigen and to determine the distribution of the Factor D antigen in the examined tissue. In situ detection can be accomplished by applying a detectably labeled anti-Factor D antibody to tissue sections. General techniques of in situ detection are well-known to those of ordinary skill. See, for example, Ponder, “Cell Marking Techniques and Their Application,” in Mammalian Development: A Practical Approach 113-38 Monk (ed.) (IRL Press 1987).

### A. Assays to Detect Mature Factor D

In accordance with the foregoing, in one aspect, the present invention provides a method of determining the presence or amount of mature Factor D in a test sample, such as a biological sample, the method comprising (a) contacting a test sample with a mature Factor D-specific monoclonal antibody or antigen-binding fragment thereof in an in vitro immunoassay and (b) detecting the presence or absence of binding of said antibody, wherein the presence of binding indicates the presence or amount of mature Factor D in the sample. In one embodiment, the mature Factor D-specific antibody or fragment thereof binds to an epitope in the amino-terminal region of human mature Factor D, wherein said epitope comprises or consists of the amino acids ILGGREA (SEQ ID NO:5) and wherein the antibody does not bind to Pro-Factor D.

In one embodiment, the anti-human mature Factor D-specific antibody or antigen-binding fragment thereof comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23. In one embodiment, the anti-human mature Factor D-specific antibody or antigen-binding fragment thereof comprises a binding domain comprising the following six CDRs: a) an HC-CDR1 comprising the amino acid sequence XSXMGVS (SEQ ID NO:65), wherein X at position 1 is T, I or S and X at position 3 is G or I; (b) an HC-CDR2 comprising the amino acid sequence HIYWDDEKHYXPSLKX (SEQ ID NO:66), wherein X at position 11 is H or N and X at position 16 is S or R; (c) an HC-CDR3 comprising the amino acid sequence RYYGYXXXMXY (SEQ ID NO:67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is N or S, X at position 4 is Q or E, X at position 7 is V or L, and X at position 15 is F or L; (e) a LC-CDR2 comprising the amino acid sequence KVXNRFS (SEQ ID NO: 69), wherein: X at position 3 is S or Y; and (f) a LC-CDR3 comprising the amino acid sequence FQGSHVPPT (SEQ ID NO:54). In one embodiment, the anti-human mature Factor D-specific antibody or antigen-binding fragment thereof comprises a binding domain comprising the following six CDRs: (a) an HC-CDR1 comprising SEQ ID NO:25, (b) an HC-CDR2 comprising SEQ ID NO:27; (c) an HC-CDR3 comprising SEQ ID NO: 29; (d) a LC-CDR1 comprising SEQ ID NO:50, (e) a LC-CDR2 comprising SEQ ID NO:52 and (f) a LC-CDR3 comprising SEQ ID NO:54.

In some embodiments, the anti-human mature Factor D-specific antibody or fragment thereof is a monoclonal antibody comprising the CDRs from mature Factor-D-specific clones 6G6, 14A11, 27B3, 58F5, 49G3 and 10G1 as set forth in TABLE 2.

In one embodiment, the method further comprises comparing the amount of mature-Factor D detected in accordance with step (b) with a reference standard or control sample to determine the level of mature-Factor D in the test sample.

In one embodiment, the control sample is an individual or pooled sample of subjects suffering from an alternative pathway disease or disorder (e.g., paroxysmal nocturnal hemoglobinuria (PNH), C3 glomerulopathy, or other alternative pathway disease or disorder). In one embodiment, the control sample is an individual or pooled sample of normal healthy volunteers. In one embodiment, the control sample is a baseline sample of a subject prior to treatment with a complement inhibitor (e.g., a MASP-3 inhibitory agent or other complement inhibitor). In one embodiment, the reference standard is a ratio of at least one of: Pro-Factor D versus mature Factor D or mature Factor D versus total Factor D, wherein the ratio is obtained from a test sample or a control sample (e.g., an individual or pooled sample of normal healthy volunteers, or a baseline sample of a subject prior to treatment with a complement inhibitor, or an individual or pooled sample of subject(s) suffering from an alternative pathway disease or disorder). In one embodiment, the anti-human mature Factor D-specific antibody or antigen-binding fragment thereof is immobilized on a substrate. In one embodiment, the immunoassay is an ELISA assay.

In one embodiment, the anti-human mature Factor D-specific antibody is labeled with a detectable moiety and step (b) comprises detecting the presence of said detectable moiety. In one embodiment, said anti-human mature Factor D-specific antibody or antigen-binding fragment thereof is naked (i.e., not labeled), and the presence or amount of the antibody or fragment thereof bound to mature Factor D is detected using a labeled antibody which binds to the anti-mature Factor D antibody. In one embodiment, said anti-human mature Factor D-specific antibody or antigen-binding fragment thereof is immobilized on a substrate (i.e., capture/coating) and the bound mature Factor D is detected with a second antibody that binds to a different epitope of Factor D (e.g., an anti-Factor D antibody that binds to an epitope shared by mature Factor D and Pro-Factor D as described herein).

In one embodiment, the test sample is a biological sample obtained from a mammalian subject. In various embodiments, the biological sample is selected from the group consisting of whole blood, serum, plasma, sputum, amniotic fluid, cerebrospinal fluid, cell lysate, ascites, urine, saliva, and tissue. In one embodiment, the biological sample is selected from the group consisting of blood, serum, plasma, urine, and cerebrospinal fluid.

In one embodiment, the mammalian subject (e.g., human) is suffering from, or at risk for developing an alternative pathway disease or disorder. In one embodiment, the mammalian subject is suffering from, or for developing, a renal disease in which complement Factor D removal is impaired due to a decrease in kidney function.

In one embodiment, the mammalian subject (e.g., human) has been treated with a complement inhibitor, such an alternative pathway complement inhibitor, such as a MASP-3 inhibitory agent (e.g. a MASP-3 inhibitory antibody), as further described herein.

As described herein, the methods of detecting mature Factor D according to various embodiments of the present disclosure may be used to define a pharmacodynamic endpoint or therapeutic threshold of a complement inhibitor, such as an alternative pathway complement inhibitor, such as a MASP-3 inhibitory agent, (e.g., a MASP-3 inhibitory antibody).

Although the details of an immunoassay may vary with the particular format employed, the method of detecting mature Factor D in a test sample comprises the steps of contacting the test sample with an antibody that specifically binds to mature Factor D. The antibody is allowed to bind to mature Factor D in the sample under immunologically reactive conditions, and the presence of the bound antibody is detected directly or indirectly. The mature Factor D-specific antibodies may be used, for example, as the capture antibody of an ELISA, or as a second antibody to bind to mature Factor D captured by the capture antibody. As is known in the art, the presence of the second antibody is typically then detected. In some embodiments, the immunoassay is performed on a solid support. In some embodiments, the immunoassay is an ELISA assay.

### B. Pro-Factor D Assays

In accordance with the foregoing, in another aspect, the present invention provides a method of detecting the presence or amount of Pro-Factor D in a test sample, the method comprising (a) contacting a test sample with a Pro-Factor D-specific antibody or antigen-binding fragment thereof in an in vitro immunoassay and (b) detecting the presence or absence of binding of said antibody, wherein the presence of binding indicates the presence of Pro-Factor D in the sample. In one embodiment, the Pro-Factor D-specific antibody or fragment thereof specifically binds to an epitope in the activation (“Pro”) peptide of human Factor D “APPRGR” (SEQ ID NO:4), wherein the antibody or fragment thereof specifically binds human Pro-Factor D (SEQ ID NO:2) and does not bind to human mature-Factor D (SEQ ID NO:3).

In one embodiment, the Pro-Factor D-specific antibody or fragment thereof that specifically binds to human Pro-Factor D comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147 wherein the CDRs are numbered according to the Kabat numbering system. In one embodiment, the Pro-Factor D-specific antibody or antigen-binding fragment thereof comprises a binding domain comprising HC-CDR1, HC-CDR2 and HC-CDR3 in a heavy chain variable region selected from the group consisting of SEQ ID NO:s 136-139 and comprising LC-CDR1, LC-CDR2 and LC-CDR3 in a light chain variable region selected from the group consisting of SEQ ID NO:s 142-145, wherein the CDRs are numbered according to the Kabat numbering system. In one embodiment, the Pro-Factor D-specific antibody or fragment thereof comprises a binding domain comprising HC-CDR1, HC-CDR2 and HC-CDR3 in a heavy chain variable region selected from the group consisting of SEQ ID NO: 140 and SEQ ID NO:141 and comprising LC-CDR-1, LC-CDR2 and LC-CDR3 in a light chain variable region selected from the group consisting of SEQ ID NO: 146 and SEQ ID NO: 147 wherein the CDRs are numbered according to the Kabat numbering system.

In one embodiment, the Pro-Factor D-specific antibody or antigen-binding fragment thereof comprises a binding domain comprising the following six CDRs: (a) a CDR-H1 comprising SEQ ID NO:167, (b) a CDR-H2 comprising SEQ ID NO:169 or SEQ ID NO: 173; (c) a CDR-H3 comprising SEQ ID NO:171 or SEQ ID NO:174; (d) a CDR-L1 comprising SEQ ID NO:194, (e) a CDR-L2 comprising SEQ ID NO:196 or SEQ ID NO:199 and (f) a CDR-L3 comprising SEQ ID NO:198 or SEQ ID NO:200.

In some embodiments, the Pro-Factor D-specific antibody or fragment thereof is a monoclonal antibody comprising the CDRs from Pro-Factor D-specific clones 18F5, 1F9, 2A4, 20A1, 13A10 and 21H1 as set forth in TABLE 4.

In one embodiment, the method further comprises comparing the amount of Pro-Factor D detected in accordance with step (b) with a reference standard or control sample to determine the level of Pro-Factor D in the test sample.

In one embodiment, the control sample is an individual or pooled sample of subjects suffering from an alternative pathway disease or disorder (e.g., paroxysmal nocturnal hemoglobinuria (PNH), C3 glomerulopathy, or other alternative pathway disease or disorder). In one embodiment, the control sample is an individual or pooled sample of normal healthy volunteers. In one embodiment, the control sample is a baseline sample of a subject prior to treatment with a complement inhibitor (e.g., a MASP-3 inhibitory agent or other complement inhibitor). In one embodiment, the reference standard is a ratio of at least one of: Pro-Factor D versus mature Factor D or Pro-Factor D versus total Factor D, wherein the ratio is obtained from a test sample or a control sample (e.g., an individual or pooled sample of normal healthy volunteers, or a baseline sample of a subject prior to treatment with a complement inhibitor, or an individual or pooled sample of subject(s) suffering from an alternative pathway disease or disorder).

In one embodiment, the anti-human Pro-Factor D-specific antibody or antigen-binding fragment thereof is immobilized on a substrate. In one embodiment, the immunoassay is an ELISA assay.

In one embodiment, the anti-human Pro-Factor D-specific antibody is labeled with a detectable moiety and step (b) comprises detecting the presence of said detectable moiety. In one embodiment, the anti-human Pro-Factor D-specific antibody or antigen-binding fragment thereof is naked (i.e., not labeled), and the presence or amount of the antibody or fragment thereof bound to Pro-Factor D is detected using a labeled antibody which binds to the anti-human Pro-Factor D antibody. In one embodiment, said anti-human Pro-Factor D-specific antibody or antigen-binding fragment thereof is immobilized on a substrate (i.e., capture/coating) and the bound Pro-Factor D is detected with a second antibody that binds to a different epitope of Factor D (e.g., an anti-Factor D antibody that binds to an epitope shared by mature Factor D and Pro-Factor D as described herein).

In one embodiment, the test sample is a biological sample obtained from a mammalian subject. In various embodiments, the biological sample is selected from the group consisting of whole blood, serum, plasma, sputum, amniotic fluid, cerebrospinal fluid, cell lysate, ascites, urine, saliva, and tissue. In one embodiment, the biological sample is selected from the group consisting of blood, serum, plasma, urine, and cerebrospinal fluid.

In one embodiment, the mammalian subject (e.g., human) is suffering from, or at risk for developing an alternative pathway disease or disorder. In one embodiment, the mammalian subject is suffering from, or for developing, a renal disease in which complement Factor D removal is impaired due to a decrease in kidney function.

In one embodiment, the mammalian subject (e.g., human) has been treated with a complement inhibitor, such an alternative pathway complement inhibitor, such as a MASP-3 inhibitory agent (e.g. a MASP-3 inhibitory antibody), as further described herein.

As described herein, the methods of detecting Pro-Factor D according to various embodiments of the present disclosure may be used to define a pharmacodynamic endpoint or therapeutic threshold of a complement inhibitor, such as an alternative pathway complement inhibitor, such as a MASP-3 inhibitory agent, (e.g., a MASP-3 inhibitory antibody). In one embodiment, the mammalian subject (e.g., human) has been treated with a MASP-3 inhibitory agent such as a MASP-3 inhibitory antibody as further described herein.

Although the details of an immunoassay may vary with the particular format employed, the method of detecting Pro-Factor D in a test sample comprises the steps of contacting the test sample with an antibody that specifically binds to Pro-Factor D. The antibody is allowed to bind to Pro-Factor D in the sample under immunologically reactive conditions, and the presence of the bound antibody is detected directly or indirectly. The Pro-Factor D-specific antibodies may be used, for example, as the capture antibody of an ELISA, or as a second antibody to bind to Pro-Factor D captured by the capture antibody. As is known in the art, the presence of the second antibody is typically then detected. In some embodiments, the immunoassay is performed on a solid support. In some embodiments, the immunoassay is an ELISA assay.

### VII. Methods of Diagnosis, Monitoring and Treatment a Subject Suffering from, or at Risk for Developing an Alternative Pathway Disease or Disorder

The inventive anti-Factor D antibodies, methods, reagents, and kits may be used in a number of applications. For example, in certain embodiments, an assay of this invention may be used to assess the level of mature Factor D and/or Pro-Factor D in a subject and/or to assess the extent to which a complement pathway inhibitor, such as an alternative pathway complement inhibitor, such as a MASP-3 inhibitory agent (e.g., a MASP-3 inhibitory antibody) affects the level of mature Factor D and/or Pro-Factor D in a biological sample obtained from the subject and thereby assess the extent of APC activation in said subject. In some embodiments, an assay of this invention may be used to assess the extent to which a complement pathway inhibitor (e.g., a MASP-3 inhibitory agent) decreases alternative complement pathway activation in vivo or in vitro. In some embodiments, an inventive method is performed on a biological sample obtained from a subject. In some embodiments, the level of mature Factor D and/or Pro-Factor D detected in an assay of this invention is compared with a suitable reference value. The reference value may be, e.g., a value measured from a sample obtained from a healthy patient (or a pool of healthy patients), or a value measured from a sample obtained from a patient undergoing treatment with a MASP-3 inhibitory agent (e.g., obtained prior to treatment or at a time point in a sequence of treatments), or the reference value may be a predetermined threshold. In one embodiment, the control sample is an individual or pooled sample of subjects suffering from an alternative pathway disease or disorder (e.g., paroxysmal nocturnal hemoglobinuria (PNH), C3 glomerulopathy, or other alternative pathway disease or disorder). In one embodiment, the control sample is an individual or pooled sample of normal healthy volunteers. In one embodiment, the control sample is a baseline sample of a subject prior to treatment with a complement inhibitor (e.g., a MASP-3 inhibitory agent or other complement inhibitor). In one embodiment, the reference standard is a ratio of at least one of: Pro-Factor D versus mature Factor D, mature Factor D versus total Factor D, or Pro-Factor D versus total Factor D, wherein the ratio is obtained from a test sample or a control sample (e.g., an individual or pooled sample of normal healthy volunteers, or a baseline sample of a subject prior to treatment with a complement inhibitor, or an individual or pooled sample of subject(s) suffering from an alternative pathway disease or disorder).

As described herein, the methods of detecting mature Factor D and/or Pro-Factor D according to various embodiments of the present disclosure may be used assess the extent of alternative pathway complement activation and thereby used to define a pharmacodynamic endpoint or therapeutic threshold of a complement inhibitor, such as an alternative pathway complement inhibitor, such as a MASP-3 inhibitory agent, (e.g., a MASP-3 inhibitory antibody).

### A. Methods of Assessing the Extent of Alternative Pathway Complement Activation in a Mammalian Subject

In one aspect, the present disclosure provides methods of assessing the extent of alternative pathway complement (APC) activation in a test sample and performing an immunoassay comprising capturing and detecting mature Factor D in the test sample and/or capturing and detecting pro-Factor D in the test sample, wherein the level of mature Factor D and/or the level of Pro-Factor D detected in the test sample is indicative of the extent of alternative pathway complement activation in the test sample. In one embodiment, the test sample is a biological sample obtained from a mammalian subject and the method comprises the steps of: (a) providing a biological sample obtained from the mammalian subject; and (b) assessing the extent of APC activation in the subject by performing an immunoassay comprising at least one of capturing and detecting mature Factor D in the biological sample; and/or capturing and detecting Pro-Factor D in the biological sample according to an inventive methods described herein. For example, in one embodiment, the immunoassay comprises capturing and detecting mature Factor D in the test sample, wherein mature Factor D is either captured or detected with a mature Factor D-specific monoclonal antibody or fragment thereof that specifically binds to an epitope in “ILGGREA” (SEQ ID NO:5) present in mature Factor D, but does not bind to Pro-Factor D. In one embodiment, the immunoassay comprises capturing and detecting Pro-Factor D in the test sample, wherein Pro-Factor D is either captured or detected with a Pro-Factor D-specific monoclonal antibody or fragment thereof that specifically binds to an epitope on the activation (“Pro”) peptide “APPRGR” (SEQ ID NO:4) present in Pro-Factor D, but does not bind to mature Factor D. In various embodiments, the method comprises comparing the level of mature Factor D detected in the test sample (e.g., biological sample) with a predetermined level or control sample and/or comparing the level of Pro-Factor D detected in the test sample with a predetermined level or control sample, wherein the level of mature Factor D and/or Pro-Factor D detected in the test sample is indicative of the extent of alternative pathway complement activation in the test sample (e.g., biological sample). In some embodiments, the method further comprises using the result of the comparative analysis to provide diagnostic, prognostic or treatment-related information regarding the mammalian subject from which the biological sample was obtained. In some embodiments, the present disclosure provides a method of assessing the effect on alternative pathway complement activation in vivo of an inhibitor of human complement. Any compound which binds to or otherwise blocks the generation and/or activity of any of the human complement components may be utilized in accordance with the present disclosure. For example, an inhibitor of complement can be, e.g., a small molecule, a nucleic acid or nucleic acid analog, a peptidomimetic, or a macromolecule that is not a nucleic acid or a protein, such as an antibody, or fragment thereof. In some embodiments, the present disclosure provides a method of assessing the effect on alternative complement pathway activation in vivo of an inhibitor (e.g., an antibody or small molecule) specific to a human complement component, such as, for example an inhibitor of a complement component selected from the group consisting of C1 (C1q, C1r, C1s), C2, C3, C4, C5, C6, C7, C8, C9, Factor D, Factor B, Factor P, MBL, MASP-1, MASP-2, and MASP-3. In some embodiments, the present disclosure provides a method of assessing the effect of an alternative complement pathway inhibitor on alternative pathway complement activation. In some embodiments, the present disclosure provides a method of assessing the effect of an inhibitor of Pro-Factor D maturation on alternative pathway complement activation.

In some embodiments, the present disclosure provides a method of assessing the effect on alternative pathway complement activation in vivo of a MASP-3 inhibitory agent that has been administered to a mammalian subject. In various embodiments, a MASP-3 inhibitory agent (e.g., a MASP-3 inhibitory antibody) is administered to a mammalian subject, and a biological sample is subsequently obtained. The extent of alternative pathway complement (APC) activation in the biological sample is then assessed by performing an immunoassay comprising at least one of capturing and detecting mature Factor D in the biological sample; and/or capturing and detecting Pro-Factor D in the biological sample according to an inventive methods described herein.

### B. Methods of Monitoring the Efficacy of a MASP-3 Inhibitory Antibody in a Mammalian Subject

In one embodiment, the present disclosure provides a method for monitoring the efficacy of treatment with a MASP-3 inhibitory antibody in a mammalian subject, the method comprising the steps of (a) administering a dose of a MASP-3 inhibitory antibody to a mammalian subject at a first point in time; (b) assessing a first concentration of mature Factor D and/or Pro-Factor D in a biological sample obtained from the subject after step (a); (c) treating the subject with the MASP-3 inhibitory antibody at a second point in time; (d) assessing a second concentration of mature Factor D and/or Pro-Factor D in a biological sample obtained from the subject after step (c); and (e) comparing the level of mature Factor D and/or Pro-Factor D assessed in step (b) with the level of mature Factor D and/or Pro-Factor D assessed in step (d) to determine the efficacy of the MASP-3 inhibitory antibody in the mammalian subject. In one embodiment, the extent of APC activation in the subject is assessed in an immunoassay, wherein the immunoassay comprises capturing and detecting the level of mature Factor D in the biological sample. Optionally the level of mature Factor D detected in the biological sample is compared with a suitable reference value. The reference value may be, e.g., a value of mature Factor D measured from a biological sample obtained from the subject prior to administration of the MASP-3 inhibitory antibody, an average value measured from samples obtained from a group of healthy control subjects, a value that represents a desired extent of APC activation (e.g., a level of mature Factor D corresponding to 90% inhibition of APC, or 80% inhibition, or 70% inhibition, or 60% inhibition, or 50% inhibition of APC). For example, a first biological sample is obtained from a subject before administration of a MASP-3 inhibitory antibody and a second biological sample is obtained after administration of the MASP-3 inhibitory antibody and the level of mature Factor D is measured in the samples. If the level of mature Factor D in the second biological sample is less than the level of mature Factor D in the first biological sample, or is lower than a control value (e.g. a threshold value corresponding to a percent inhibition of APC), it can be concluded that the MASP-3 inhibitory antibody inhibited APC activation to a desired extent. Alternatively, if the level of mature Factor D in the second biological sample is higher than the level of mature Factor D in the first biological sample, or is higher than a control value (e.g., a threshold value corresponding to a percent inhibition of APC), it can be concluded that the dosage of the MASP-3 inhibitory antibody should be increased, and optionally, the method further comprises administering an increased dosage of the MASP-3 inhibitory antibody to the subject. In some embodiments, if the subject is administered an increased dose of the MASP-3 inhibitory antibody, steps (b) to (e) are repeated to determine whether the increased dose of the MASP-3 inhibitory antibody is sufficient to adjust the level of mature Factor D to the desired level as compared to the respective control or reference standard.

In another embodiment, the extent of APC activation in the mammalian subject is assessed in an immunoassay, wherein the immunoassay comprises capturing and detecting the level of Pro-Factor D in the biological sample. Optionally, the level of Pro-Factor D detected in the biological sample is compared with a suitable reference value. The reference value may be, e.g., a value of Pro-Factor D measured from a biological sample obtained from the subject prior to administration of the MASP-3 inhibitory antibody, an average value measured from samples obtained from a group of healthy control subjects, a value that represents a desired extent of APC activation (e.g., a level of Pro-Factor D corresponding to 90% inhibition of APC, or 80% inhibition, or 70% inhibition, or 60% inhibition, or 50% inhibition of APC). For example, a first biological sample is obtained from a subject before administration of a MASP-3 inhibitory antibody and a second biological sample is obtained after administration of the MASP-3 inhibitory antibody and the level of Pro-Factor D are measured in the samples. If the level of Pro-Factor D in the second biological sample is greater than the level of Pro-Factor D in the first biological sample, or is higher than a control value (e.g., a threshold value corresponding to a percent inhibition of APC), it can be concluded that the MASP-3 inhibitory antibody inhibited APC activation to a desired extent. Alternatively, if the level of Pro-Factor D in the second biological sample is lower than the level of Pro-Factor D in the first biological sample, or is lower than a control value (e.g., a threshold value corresponding to a percent inhibition of APC), it can be concluded that the dosage of the MASP-3 inhibitory antibody should be increased, and optionally, the method further comprises administering an increased dosage of the MASP-3 inhibitory antibody to the subject. In some embodiments, if the subject is administered an increased dose of the MASP-3 inhibitory antibody, steps (b) to (e) are repeated to determine whether the increased dose of the MASP-3 inhibitory antibody is sufficient to adjust the level of Pro-Factor D to the desired level as compared to the respective control or reference standard.

In some embodiments, the methods are used to monitor the efficacy of a MASP-3 inhibitory antibody that is administered to a human subject suffering from or at risk of developing an alternative pathway disease or disorder, such as wherein the alternative pathway disease or disorder is selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

C. Methods of Treating a Mammalian Subject Suffering from, or at Risk of Developing an Alternative Pathway Disease or Disorder

In another aspect, the present disclosure provides a method of treating a mammalian subject suffering from, or at risk of developing an alternative-pathway disease or disorder, comprising administering a MASP-3 inhibitory antibody to the subject if the subject is determined to have: (i) a lower or decreased level of Pro-Factor D in one or more samples taken from the subject compared to a predetermined Pro-Factor D level or compared to the Pro-Factor D level in one or more control samples: and/or (ii) a higher or increased level of mature Factor D in one or more samples taken from the subject compared to a predetermined mature Factor D level or compared to the mature Factor D level in one or more control samples. In one embodiment, the level of mature Factor D in one or more samples taken from the subject is determined by performing an immunoassay comprising the use of a mature Factor D-specific monoclonal antibody. In one embodiment, the level of mature Pro-Factor D in one or more samples taken from the subject is determined by performing an immunoassay comprising the use of a Pro-Factor D-specific monoclonal antibody.

In some embodiments, the methods are used to treat a human subject suffering from or at risk of developing an alternative pathway disease or disorder, such as wherein the alternative pathway disease or disorder is selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopeniarpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

### VIII. MASP-3 Inhibitory Agents

The human MASP-3 polypeptide (SEQ ID NO:7, from Genbank AAK84071.1) has 728 amino acid residues, which includes a leader peptide of 19 residues. As noted above, it has been demonstrated that MASP-3 is responsible for the conversion of complement Factor D from the zymogen form of the protein (i.e., Pro-Factor D) to the mature form (i.e., mature Factor D), thus placing the MASP-3 protein at a key upstream regulatory step for the alternative pathway. Accordingly, in the practice of various aspects and embodiments of the present disclosure, representative MASP-3 inhibitory agents include an agent that binds to or directly interacts with MASP-3 set forth as SEQ ID NO:7, including anti-MASP-3 antibodies and MASP-3 binding fragments thereof, small-molecules and expression inhibitors that inhibit alternative pathway complement activation. In preferred embodiments, the MASP-3 inhibitory agent is specific to MASP-3, and does not bind to MASP-1 or MASP-2. An example of a MASP-3 inhibitory agent is a MASP-3 specific inhibitory agent, such as a MASP-3 inhibitory agent that specifically binds to a portion of human MASP-3 (SEQ ID NO: 7) with a binding affinity of at least 10 times greater than to other components in the complement system. In one embodiment, the MASP-3 inhibitory agent is a high affinity MASP-3 antibody that specifically binds to the serine protease domain of human MASP-3 (SEQ ID NO:7), with an affinity of less than 500 pM. In a preferred embodiment, a MASP-3 inhibitory agent, such as an antibody or antigen-binding fragment thereof or antigen binding peptide inhibits MASP-3-mediated maturation of factor D. MASP-3 inhibitory agents useful in the method of the invention may reduce MASP-3-dependent alternative pathway complement activation by greater than 10%, such as greater than 20%, greater than 50%, or greater than 90%. In one embodiment, the MASP-3 inhibitory agent reduces MASP-3-dependent alternative pathway complement activation by greater than 90% (i.e., resulting in MASP-3 complement activation of only 10% or less).

In one embodiment, the MASP-3 inhibitory agent useful in the methods of the invention is an isolated monoclonal antibody or antigen-binding fragment thereof that specifically binds to the serine protease domain of human MASP-3 (amino acid residues 450 to 728 of SEQ ID NO:7) with high affinity (having a K_{D} of less than 500 pM), wherein the antibody or antigen-binding fragment thereof inhibits alternative pathway complement activation. For example, as described in WO2018/026722, hereby incorporated herein by reference, and as further described in Example 10 and TABLES 18-20 herein, numerous high affinity anti-MASP-3 inhibitory antibodies have been generated that bind the serine protease domain of MASP-3 and inhibit its catalytic activity. As further described in WO2018/026722, several representative MASP-3 inhibitory antibodies (e.g., 4D5, 10D12 and 13B1) were humanized. Representative humanized MASP-3 inhibitory antibodies are described below.

Accordingly, in one embodiment, a MASP-3 inhibitory agent for use in the compositions and methods of the claimed invention comprises a monoclonal antibody that binds a polypeptide consisting of human MASP-3 (SEQ ID NO:7), wherein the monoclonal antibody, or antigen-binding fragment thereof binds to MASP-3 and comprises: at least one of:
(i) a heavy chain variable region comprising a HC-CDR1 comprising SEQ ID NO: 229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO:178 (WASTRES) and a LC-CDR3 comprising SEQ ID NO: 242 (KQSYNLYT);
(ii) a heavy chain variable region comprising a HC-CDR1 comprising SEQ ID NO: 230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO:241 (LVSKLDS) and a LC-CDR3 comprising SEQ ID NO:243 (WQGTHFPWT); or
(iii) a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT);

In one embodiment, the MASP-3 monoclonal antibody comprises a heavy chain variable region comprising at least 80%, 85%, 90%, 95%, 98%, 99% or 100% identical to at least one of SEQ ID NO:220, SEQ ID NO:222, SEQ ID NO:223, SEQ ID NO:225, SEQ ID NO: 226, or SEQ ID NO:228 and a light chain variable region comprising at least 80%, 85%, 90%, 95%, 98%, 99% or 100% identical to at least one of SEQ ID NO:221, SEQ ID NO:224 or SEQ ID NO:227. In one embodiment, the MASP-3 monoclonal antibody comprises a heavy chain comprising at least 95% identical to SEQ ID NO:220 or SEQ ID NO:222 and a light chain comprising at least 95% identical to SEQ ID NO:221. In one embodiment, the MASP-3 monoclonal antibody comprises a heavy chain comprising at least 95% identical to SEQ ID NO:223 or SEQ ID NO:225 and a light chain comprising at least 95% identical to SEQ ID NO:224. In one embodiment, the MASP-3 monoclonal antibody comprises a heavy chain comprising at least 95% identical to SEQ ID NO:226 or SEQ ID NO:228 and a light chain comprising at least 95% identical to SEQ ID NO:227.

### XIV. Pharmaceutical Compositions and Articles of Manufacture

In another aspect, the present disclosure provides a pharmaceutical composition comprising a MASP-3 inhibitory antibody in an aqueous solution comprising a buffer system having a pH of 6.0±5%, 20±5% mM histidine, 100±5% mg/mL sucrose, and 0.035%+5%, polysorbate 80, wherein said MASP-3 inhibitory antibody is included at a concentration of 110 mg/mL+5%, and wherein said MASP-3 inhibitory antibody comprises a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT). In one embodiment, the pharmaceutical composition is sterile. In one embodiment, the MASP-3 inhibitory antibody or antigen-binding fragment thereof comprises a heavy chain variable region comprising at least 80%, 85%, 90%, 95%, 98%, 99% or 100% identical to SEQ ID NO:226 or SEQ ID NO:227 and a light chain variable region comprising at least 80%, 85%, 90%, 95%, 98%, 99% or 100% identical to SEQ ID NO:227. In one embodiment, the MASP-3 inhibitory antibody or antigen binding fragment thereof is selected from the group consisting of a human antibody, a humanized antibody, a chimeric antibody, a murine antibody, and an antigen-binding fragment of any of the foregoing. In one embodiment, the MASP-3 inhibitory antibody or antigen-binding fragment thereof is selected from the group consisting of a single chain antibody, an ScFv, a Fab fragment, an Fab′ fragment, an F(ab′)2 fragment, a univalent antibody lacking a hinge region and a whole antibody. In one embodiment, the MASP-3 inhibitory antibody further comprises an immunoglobulin constant region. In one embodiment, the MASP-3 inhibitory antibody comprises a human IgG4 constant region. In one embodiment, the MASP-3 inhibitory antibody comprises a human IgG4 constant region with an S228P mutation. In one embodiment, the MASP-3 inhibitory antibody comprises a mutation that promotes FcRn interations at low pH, such as, for example, wherein the MASP-3 inhibitory antibody comprises human IgG4 constant region set forth as SEQ ID NO:245.

In one aspect, the present disclosure provides an article of manufacture containing a pharmaceutical composition comprising a MASP-3 inhibitory antibody in a unit dosage form suitable for therapeutic administration to a human subject, such as a unit dosage in the range of from 10 mg to 1000 mg (such as from 50 mg to 800 mg, or from 75 mg to 500, such as from 100 mg to 300 mg, such as 125 to 275 mg, such as 150 to 200 mg, such as 150±5% mg, 155±5% mg, 160±5% mg, 165±5% mg, 170±5% mg, 175±5% mg, 180±5% mg, 185±5% mg or 190±5% mg) of MASP-3 inhibitory antibody, wherein said MASP-3 inhibitory antibody comprises a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT).

In some embodiments, the article of manufacture comprises a container and a label or package insert on or associated with the container. Suitable containers include, for example, bottles, ampoules, pouches (e.g. an intravenous infusion bag), vials, syringes, cartridges, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is the MASP-3 inhibitory antibody or antigen binding fragment thereof of the invention. The label or package insert indicates that the composition is used for treating the particular condition. The label or package insert will further comprise instructions for administering the antibody composition to the patient.

In some embodiments, the pharmaceutical compositions and the articles of manufacture described herein are for use in the treatment of a subject suffering from, or at risk of developing an alternative pathway disease or disorder. In some embodiments, the alternative pathway disease or disorder is from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopeniaurpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

### EXEMPLARY EMBODIMENTS

A. Mature Factor D-Specific mAbs:

1. An isolated antibody, or antigen-binding fragment thereof, that specifically binds to an epitope in the N-terminal region of human mature Factor D, wherein the epitope comprises or consists of the amino acids ILGGREA (SEQ ID NO:5).

2. The isolated antibody or antigen-binding fragment thereof of paragraph 1, wherein the antibody specifically binds human mature Factor D (SEQ ID NO:3) and does not bind to human Pro-Factor D (SEQ ID NO:2).

3. The isolated antibody or antigen-binding fragment thereof of paragraph 1 or paragraph 2, wherein the antibody is a monoclonal antibody.

4. The isolated antibody or antigen-binding fragment thereof of any of paragraphs 1 to 3, wherein said antibody is a humanized, chimeric, or fully human antibody.

5. The isolated antibody or antigen-binding fragment thereof of any of paragraphs 1 to 4, wherein said antigen-binding fragment selected from the group consisting of Fv, Fab, Fab′, F(ab)2 and F(ab′)2.

6. The isolated antibody or antigen-binding fragment thereof of any of paragraphs 1 to 4, wherein said antibody is a single chain molecule.

7. The isolated antibody or antigen-binding fragment thereof of any of paragraphs 1 to 4, wherein said antibody is an IgG molecule selected from the group consisting of IgG1, IgG2 and IgG4.

8. The isolated antibody or antigen-binding fragment thereof of any of paragraphs 1 to 7, wherein said antibody or antigen-binding fragment thereof binds to human mature Factor D with a K_{D} of less than 10 nM.

9. The isolated antibody or antigen-binding fragment thereof of any of paragraphs 1 to 8, wherein, said antibody or antigen-binding fragment thereof is labeled with a detectable moiety.

10. The isolated antibody or antigen-binding fragment thereof of any of paragraphs 1 to 9, wherein said antibody or antigen-binding fragment thereof is immobilized on a substrate.

11. The isolated antibody or antigen-binding fragment thereof of any of paragraphs 1 to 10, wherein the isolated antibody or antigen-binding fragment thereof that specifically binds to human mature Factor D comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23, wherein the CDRs are numbered according to the Kabat numbering system.

12. The isolated antibody or antigen-binding fragment thereof of any of paragraphs 1 to 10, wherein the antibody or antigen-binding fragment thereof that specifically binds to human mature Factor D comprises a binding domain comprising the following six CDRs: a) an HC-CDR1 comprising the amino acid sequence XSXMGVS (SEQ ID NO:65), wherein X at position 1 is T, I or S and X at position 3 is G or I; (b) an HC-CDR2 comprising the amino acid sequence HIYWDDEKHYXPSLKX (SEQ ID NO:66), wherein X at position 11 is H or N and X at position 16 is S or R; (c) an HC-CDR3 comprising the amino acid sequence RYYGYXXXMXY (SEQ ID NO:67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is N or S, X at position 4 is Q or E, X at position 7 is V or L, and X at position 15 is F or L; (e) a LC-CDR2 comprising the amino acid sequence KVXNRFS (SEQ ID NO: 69), wherein: X at position 3 is S or Y; and (f) a LC-CDR3 comprising the amino acid sequence FQGSHVPPT (SEQ ID NO:54).

13. The isolated antibody or antigen-binding fragment thereof of paragraph 12, wherein the binding domain comprises the following six CDRs: (a) an HC-CDR-1 comprising SEQ ID NO: 25, (b) an HC-CDR2 comprising SEQ ID NO:27; (c) an HC-CDR3 comprising SEQ ID NO: 29; (d) a LC-CDR1 comprising SEQ ID NO:50, (e) a LC-CDR2 comprising SEQ ID NO: 52 and (f) a LC-CDR3 comprising SEQ ID NO:54.

14. The isolated antibody or antigen-binding fragment thereof of paragraph 13, wherein the isolated antibody or fragment thereof comprises at least one of:
(a) a VH domain having at least 95% sequence identity to the amino acid sequence of SEQ ID NO:12 or SEQ ID NO:13;
(b) a VL domain having at least 95% sequence identity to the amino acid sequence of SEQ ID NO:18 or SEQ ID NO:19;
(c). a VH comprising SEQ ID NO: 12 and a VL comprising SEQ ID NO:18; and/or
(d) a VH domain comprising SEQ ID NO:13 and a VL domain comprising SEQ ID NO: 19.

15. The isolated antibody or antigen-binding fragment thereof of paragraph 12, wherein the binding domain comprises the following six CDRs: (a) an HC-CDR1 comprising SEQ ID NO: 33, (b) an HC-CDR2 comprising SEQ ID NO:34; (c) an HC-CDR3 comprising SEQ ID NO: 36; (d) a LC-CDR1 comprising SEQ ID NO:58, (e) a LC-CDR2 comprising SEQ ID NO: 52 and (f) a LC-CDR3 comprising SEQ ID NO:54.

16. The isolated antibody or antigen-binding fragment thereof of paragraph 15, wherein the isolated antibody or antigen-binding fragment thereof comprises at least one of:
(a) a VH domain having at least 95% sequence identity to the amino acid sequence of SEQ ID NO:14;
(b) a VL domain having at least 95% sequence identity to the amino acid sequence of SEQ ID NO:20; and/or
(c) a VH domain comprising SEQ ID NO:14 and a VL domain comprising SEQ ID NO: 20.

17. The isolated antibody or antigen-binding fragment thereof of paragraph 12, wherein the binding domain comprises the following six CDRs: (a) aHC-CDR1 comprising SEQ ID NO: 38, (b) an HC-CDR2 comprising SEQ ID NO:39; (c) an HC-CDR3 comprising SEQ ID NO: 41; (d) a LC-CDR1 comprising SEQ ID NO:60, (e) a LC-CDR2 comprising SEQ ID NO: 52 and (f) a LC-CDR3 comprising SEQ ID NO:54.

18. The isolated antibody or antigen-binding fragment thereof of paragraph 17, wherein the isolated antibody or antigen-binding fragment thereof comprises at least one of:
(a) a VH domain having at least 95% sequence identity to the amino acid sequence of SEQ ID NO:15;
(b) a VL domain having at least 95% sequence identity to the amino acid sequence of SEQ ID NO:21; and/or
(c) a VH domain comprising SEQ ID NO:15 and a VL domain comprising SEQ ID NO: 21.

19. The isolated antibody or antigen-binding fragment thereof of paragraph 12, wherein the binding domain comprises the following six CDRs: (a) an HC-CDR1 comprising SEQ ID NO: 43, (b) an HC-CDR2 comprising SEQ ID NO:39; (c) an HC-CDR3 comprising SEQ ID NO: 41; (d) a LC-CDR1 comprising SEQ ID NO:62, (e) a LC-CDR22 comprising SEQ ID NO: 52 and (f) a LC-CDR3 comprising SEQ ID NO:54.

20. The isolated antibody or antigen-binding fragment thereof of paragraph 19, wherein the isolated antibody or antigen-binding fragment thereof comprises at least one of:
(a) a VH domain having at least 95% sequence identity to the amino acid sequence of SEQ ID NO:16;
(b) a VL domain having at least 95% sequence identity to the amino acid sequence of SEQ ID NO:22; and/or
(c) a VH domain comprising SEQ ID NO:16 and a VL comprising SEQ ID NO:22.

21. The isolated antibody or antigen-binding fragment thereof of paragraph 12, wherein the binding domain comprises the following six CDRs: (a) an HC-CDR1 comprising SEQ ID NO: 43, (b) an HC-CDR2 comprising SEQ ID NO:39; (c) an HC-CDR3 comprising SEQ ID NO: 47; (d) a LC-CDR1 comprising SEQ ID NO:63, (e) a LC-CDR2 comprising SEQ ID NO: 64 and (f) a LC-CDR3 comprising SEQ ID NO:54.

22. The isolated antibody or antigen-binding fragment thereof of paragraph 21, wherein the isolated antibody or antigen-binding fragment thereof comprises at least one of:
(a) a VH domain having at least 95% sequence identity to the amino acid sequence of SEQ ID NO:17;
(b) a VL domain having at least 95% sequence identity to the amino acid sequence of SEQ ID NO:23; and/or
(c) a VH domain comprising SEQ ID NO:17 and a VL domain comprising SEQ ID NO: 23.

23. A nucleic acid molecule encoding the amino acid sequence of an antibody, or antigen-binding fragment thereof, that specifically binds human mature Factor D as set forth in any of paragraphs 11 to 22.

24. An expression cassette comprising a nucleic acid molecule encoding an antibody, or antigen-binding fragment thereof, that specifically binds human mature Factor D of the invention according to paragraph 23.

25. A cell comprising at least one of the nucleic acid molecules encoding an antibody, or antigen-binding fragment thereof, that specifically binds human mature Factor D of the invention according to paragraph 23 or paragraph 24.

26. A method of generating an isolated antibody, or antigen-binding fragment thereof, that specifically binds human mature Factor D comprising culturing the cell of paragraph 25 under conditions allowing for expression of the nucleic acid molecules encoding the antibody, or antigen-binding fragment thereof, that specifically binds human mature Factor D and isolating said anti-mature-Factor-D specific antibody, or antigen-binding fragment thereof.

27. A composition comprising an antibody, or antigen-binding fragment thereof, that specifically binds human mature Factor D as set forth in any of paragraphs 1 to 22.

28. A substrate for use in an immunoassay comprising at least one antibody, or antigen-binding fragment thereof, that specifically binds human mature Factor D as set forth in any of paragraphs 1 to 22

29. A kit for detecting the presence or amount of mature Factor D in a test sample, said kit comprising (a) at least one container, and (b) at least one antibody, or antigen-binding fragment thereof, that specifically binds human mature Factor D as set forth in any of paragraphs 1 to 22.

B. Pro-Factor D-Specific mAbs:

1. An isolated antibody, or antigen-binding fragment thereof, that specifically binds to an epitope on the activation (“Pro”) peptide of human Factor D, wherein the epitope comprises or consists of “APPRGR” (SEQ ID NO:4).

2. The antibody or antigen-binding fragment of paragraph 1, wherein the antibody or antigen-binding fragment thereof specifically binds to human Pro-Factor D (SEQ ID NO:2) and does not bind to mature Factor D (SEQ ID NO:3).

3. The isolated antibody or antigen-binding fragment thereof of paragraph 1 or paragraph 2, wherein the antibody is a monoclonal antibody.

4. The isolated antibody or antigen-binding fragment thereof of any of paragraphs 1 to 3, wherein said antibody is a humanized, chimeric, or fully human antibody.

5. The isolated antibody or antigen-binding fragment thereof of any of paragraphs 1 to 4, wherein said antigen-binding fragment is selected from the group consisting of Fv, Fab, Fab′, F(ab)₂ and F(ab′)₂.

6. The isolated antibody or antigen-binding fragment thereof of any of paragraphs 1 to 4, wherein said antibody is a single chain molecule.

7. The isolated antibody or antigen-binding fragment thereof of any of paragraphs 1 to 4, wherein said antibody is an IgG molecule selected from the group consisting of IgG1, IgG2 and IgG4.

8. The isolated antibody or antigen-binding fragment thereof of any of paragraphs 1 to 7, wherein said antibody or antigen-binding fragment thereof binds to human Pro-Factor D with a K_{D} of less than 10 nM.

9. The isolated antibody or antigen-binding fragment thereof of any of paragraphs 1 to 8, wherein said antibody or antigen-binding fragment thereof is labeled with a detectable moiety.

10. The isolated antibody or antigen-binding fragment thereof of any of paragraphs 1 to 9, wherein said antibody or antigen-binding fragment thereof is immobilized on a substrate.

11. The isolated antibody or antigen-binding fragment thereof that specifically binds to human Pro-Factor D of any of paragraphs 1 to 10, wherein the antibody or antigen-binding fragment thereof comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

12. The isolated antibody or antigen-binding fragment thereof of paragraph 11, wherein the isolated antibody or antigen-binding fragment thereof comprises a binding domain comprising HC-CDR1, HC-CDR2 and HC-CDR3 in a heavy chain variable region selected from the group consisting of SEQ ID NO:s 136-139 and comprising LC-CDR1, LC-CDR2 and LC-CDR3 in a light chain variable region selected from the group consisting of SEQ ID NO:s 142-145.

13. The isolated antibody or antigen-binding fragment thereof of paragraph 12, wherein the isolated antibody or antigen-binding fragment thereof comprises a binding domain comprising the following six CDRs: (a) an HC-CDR1 comprising the amino acid sequence XYWMS (SEQ ID NO:201), wherein X at position 1 is N, S or T; (b) an HC-CDR2 comprising the amino acid sequence EIRLKSXNYAXXYXESVKG (SEQ ID NO:202), wherein: X at position 7 is D or E, X at position 11 is T or A, X at position 12 is H or Y and X at position 14 is A or T; (c) an HC-CDR3 comprising the amino acid sequence AWFAX (SEQ ID NO:203), wherein X at position 5 is S, Y or N; (d) a LC-CDR1 comprising the amino acid sequence XSSQXLLYSXDQKNYLA (SEQ ID NO:204), wherein X at position 1 is M or K, X at position 5 is S or N, and X at position 10 is K or R; (e) a LC-CDR2 comprising the amino acid sequence WASTRES (SEQ ID NO:178); and (f) a LC-CDR3 comprising the amino acid sequence LQYYXYPYT (SEQ ID NO:205), wherein X at position 5 is T or S.

14. The isolated antibody or antigen-binding fragment thereof of paragraph 13, wherein the isolated antibody or antigen-binding fragment thereof comprises a binding domain comprising the following six CDRs: (a) an HC-CDR1 comprising SEQ ID NO:149 or SEQ ID NO: 155, (b) a HC-CDR2 comprising SEQ ID NO:151 or SEQ ID NO:156; (c) an HC-CDR3 comprising SEQ ID NO:153; (d) a LC-CDR1 comprising SEQ ID NO:176, (e) a LC-CDR2 comprising SEQ ID NO:178 and (f) a LC-CDR3 comprising SEQ ID NO:180.

15. The isolated antibody or antigen-binding fragment thereof of paragraph 14, wherein the isolated antibody or antigen-binding fragment thereof comprises at least one of:
(a) a VH domain having at least 95% sequence identity to the amino acid sequence of SEQ ID NO:136;
(b) a VH domain having at least 95% sequence identity to the amino acid sequence of SEQ ID NO:137;
(c) a VL domain having at least 95% sequence identity to the amino acid sequence of SEQ ID NO:142;
(d) a VL domain having at least 95% sequence identity to the amino acid sequence of SEQ ID NO:143;
(e) a VH domain comprising SEQ ID NO:136 and a VL domain comprising SEQ ID NO: 142; and/or
(f) a VH domain comprising SEQ ID NO:137 and a VL domain comprising SEQ ID NO: 143.

16. The isolated antibody or antigen-binding fragment thereof of paragraph 13, wherein the isolated antibody or antigen-binding fragment thereof comprises a binding domain comprising the following six CDRs: (a) an HC-CDR1 comprising SEQ ID NO:158, (b) an HC-CDR2 comprising SEQ ID NO:159 or SEQ ID NO:163; (c) an HC-CDR3 comprising SEQ ID NO:161 or SEQ ID NO:165; (d) a LC-CDR-1 comprising SEQ ID NO:184 or SEQ ID NO: 189, (e) a LC-CDR2 comprising SEQ ID NO:178 and (f) a LC-CDR3 comprising SEQ ID NO:187.

17. The isolated antibody or antigen-binding fragment thereof of paragraph 16, wherein the isolated antibody or antigen-binding fragment thereof comprises at least one of:
(a) a VH domain having at least 95% sequence identity to the amino acid sequence of SEQ ID NO:138; (b) a VH domain having at least 95% sequence identity to the amino acid sequence of SEQ ID NO:139;
(c) a VL domain having at least 95% sequence identity to the amino acid sequence of SEQ ID NO:144;
(d) a VL domain having at least 95% sequence identity to the amino acid sequence of SEQ ID NO:145;
(e) a VH domain comprising SEQ ID NO:138 and a VL domain comprising SEQ ID NO: 144; and/or
(f) a VH domain comprising SEQ ID NO:139 and a VL domain comprising SEQ ID NO: 145.

18. The isolated antibody or antigen-binding fragment thereof of paragraph 11, wherein the isolated antibody or antigen-binding fragment thereof comprises a binding domain comprising HC-CDR1, HC-CDR2 and HC-CDR3 in a heavy chain variable region selected from the group consisting of SEQ ID NO: 140 and SEQ ID NO:141 and comprising LC-CDR1, LC-CDR2 and LC-CDR3 in a light chain variable region selected from the group consisting of SEQ ID NO: 146 and SEQ ID NO:147.

19. The isolated antibody or antigen-binding fragment thereof of paragraph 18, wherein the isolated antibody or antigen-binding fragment thereof comprises a binding domain comprising the following six CDRs: (a) a CDR-H1 comprising SEQ ID NO:167, (b) a CDR-H2 comprising SEQ ID NO:169 or SEQ ID NO:173; (c) a CDR-H3 comprising SEQ ID NO: 171 or SEQ ID NO:174; (d) a CDR-L1 comprising SEQ ID NO:194, (e) a CDR-L2 comprising SEQ ID NO:196 or SEQ ID NO:199 and (f) a CDR-L3 comprising SEQ ID NO: 198 or SEQ ID NO:200.

20. The isolated antibody or antigen-binding fragment thereof of paragraph 19, wherein the isolated antibody or antigen-binding fragment thereof comprises at least one of:
(a) a VH domain having at least 95% sequence identity to the amino acid sequence of SEQ ID NO:140;
(b) a VH domain having at least 95% sequence identity to the amino acid sequence of SEQ ID NO:141;
(c) a VL domain having at least 95% sequence identity to the amino acid sequence of SEQ ID NO:146;
(d) a VL domain having at least 95% sequence identity to the amino acid sequence of SEQ ID NO:147;
(e) a VH domain comprising SEQ ID NO:140 and a VL domain comprising SEQ ID NO: 146; and/or
(f) a VH domain comprising SEQ ID NO:141 and a VL domain comprising SEQ ID NO: 147.

21. A nucleic acid molecule encoding the amino acid sequence of an antibody, or antigen-binding fragment thereof, that specifically binds human Pro-Factor D as set forth in any of paragraphs 11 to 20.

22. An expression cassette comprising a nucleic acid molecule encoding an antibody, or antigen-binding fragment thereof, that specifically binds human Pro-Factor D according to paragraph 21.

23. A cell comprising at least one of the nucleic acid molecules encoding an antibody, or antigen-binding fragment thereof, that specifically binds human Pro-Factor D according to paragraph 21 or paragraph 22.

24. A method of generating an isolated antibody, or antigen-binding fragment thereof, that specifically binds human Pro-Factor D comprising culturing the cell of paragraph 23 under conditions allowing for expression of the nucleic acid molecules encoding the antibody, or antigen-binding fragment thereof, that specifically binds human Pro-Factor D and isolating said anti-Pro-Factor-D specific antibody, or antigen-binding fragment thereof.

25. A composition comprising an antibody, or antigen-binding fragment thereof, that specifically binds human Pro-Factor D as set forth in any of paragraphs 1 to 20.

26. A substrate for use in an immunoassay comprising at least one antibody, or antigen-binding fragment thereof, that specifically binds human Pro-Factor D as set forth in any of paragraphs 1 to 20.

27. A kit for detecting the presence or amount of Pro-Factor D in a test sample, said kit comprising (a) at least one container, and (b) at least one antibody, or antigen-binding fragment thereof, that specifically binds human mature Factor D as set forth in any of paragraphs 1 to 20.

C. Anti-Factor D mAbs (Detect Pro and Mature Factor D Via Binding to a Shared Epitope)

1. An isolated antibody or antigen-binding fragment thereof that binds to an epitope shared by both human mature Factor D and human Pro-Factor D, wherein the antibody or antigen-binding fragment thereof comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

2. The isolated antibody or antigen-binding fragment thereof of paragraph 1, wherein the antibody is a monoclonal antibody.

3. The isolated antibody or antigen-binding fragment thereof of any of paragraphs 1 to 2, wherein said antibody is a humanized, chimeric, or fully human antibody.

4. The isolated antibody or antigen-binding fragment thereof of any of paragraphs 1 to 3, wherein said antigen-binding fragment is selected from the group consisting of Fv, Fab, Fab′, F(ab)2 and F(ab′)2.

5. The isolated antibody or antigen-binding fragment thereof of any of paragraphs 1 to 4, wherein said antibody or antigen-binding fragment is a single chain molecule.

6. The isolated antibody or antigen-binding fragment thereof of any of paragraphs 1 to 4, wherein said antibody is an IgG molecule selected from the group consisting of IgG1, IgG2 and IgG4.

7. The isolated antibody or antigen-binding fragment thereof of any of paragraphs 1 to 7, wherein said antibody or antigen-binding fragment thereof binds to human Factor D with a K_{D} of less than 10 nM.

8. The isolated antibody or antigen-binding fragment thereof of any of paragraphs 1 to 7, wherein, said antibody or antigen-binding fragment thereof is labeled with a detectable moiety.

9. The isolated antibody or antigen-binding fragment thereof of any of paragraphs 1 to 8, wherein said antibody or antigen-binding fragment thereof is immobilized on a substrate.

10. The isolated antibody or antigen-binding fragment thereof that specifically binds to human Pro Factor D of any of paragraphs 1 to 9, wherein the antibody or antigen-binding fragment thereof comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

11. The isolated antibody or antigen-binding fragment thereof of any of paragraphs 1 to 10, wherein the antibody or antigen-binding fragment thereof comprises a binding domain comprising the following six CDRs: (a) an HC-CDR1 comprising the amino acid sequence SEQ ID NO:95, (b) a HC-CDR2 comprising the amino acid sequence SEQ ID NO:97 (c) an HC-CDR3 comprising the amino acid sequence SEQ ID NO:99 (d) a LC-CDR1 comprising the amino acid sequence SEQ ID NO:111; (e) a LC-CDR2 comprising the amino acid sequence SEQ ID NO:113); and (f) a LC-CDR3 comprising the amino acid sequence SEQ ID NO: 115.

12. The isolated antibody or antigen-binding fragment thereof of any of paragraphs 1 to 10, wherein the antibody or antigen-binding fragment thereof comprises a binding domain comprising the following six CDRs: (a) an HC-CDR1 comprising the amino acid sequence SEQ ID NO:101 (b) an HC-CDR2 comprising the amino acid sequence SEQ ID NO:103 or 107 (c) an HC-CDR3 comprising the amino acid sequence SEQ ID NO:105 or 108, (d) a LC-CDR1 comprising the amino acid sequence SEQ ID NO:60 or 123; (e) a LC-CDR2 comprising the amino acid sequence SEQ ID NO:119, 124 or 126 and (f) a LC-CDR3 comprising the amino acid sequence SEQ ID NO:121 or 125.

13. The isolated antibody or antigen-binding fragment thereof of paragraph 10, wherein the isolated antibody or antigen-binding fragment thereof comprises at least one of:
(a) a VH domain having at least 95% sequence identity to the amino acid sequence of SEQ ID NO:85;
(b) a VH domain having at least 95% sequence identity to the amino acid sequence of SEQ ID NO:86;
(c) a VH domain having at least 95% sequence identity to the amino acid sequence of SEQ ID NO:87;
(d) a VH domain having at least 95% sequence identity to the amino acid sequence of SEQ ID NO:88;
(e) a VL domain having at least 95% sequence identity to the amino acid sequence of SEQ ID NO:89;
(f) a VL domain having at least 95% sequence identity to the amino acid sequence of SEQ ID NO:90;
(g) a VL domain having at least 95% sequence identity to the amino acid sequence of SEQ ID NO:91;
(h) a VL domain having at least 95% sequence identity to the amino acid sequence of SEQ ID NO:92;
(i) a VL domain having at least 95% sequence identity to the amino acid sequence of SEQ ID NO:93;
(j) a VH domain comprising SEQ ID NO:85 and a VL domain comprising SEQ ID NO: 89 or SEQ ID NO:90;
(k) a VH domain comprising SEQ ID NO:86 and a VL domain comprising SEQ ID NO: 91;
(l) a VH domain comprising SEQ ID NO:87 and a VL domain comprising SEQ ID NO: 92; and/or
(m) a VH domain comprising SEQ ID NO:88 and a VL domain comprising SEQ ID NO: 93.

14. A nucleic acid molecule encoding the amino acid sequence of an antibody, or antigen-binding fragment thereof, that binds an epitope shared by both human mature Factor D and human Pro-Factor D as set forth in any of paragraphs 10 to 13.

15. An expression cassette comprising a nucleic acid molecule encoding an antibody, or antigen-binding fragment thereof, that binds an epitope shared by both human mature Factor D and human Pro-Factor D according to paragraph 14.

16. A cell comprising at least one of the nucleic acid molecules encoding an antibody, or antigen-binding fragment thereof, that binds an epitope shared by both human mature Factor D and human Pro-Factor D according to paragraph 14 or paragraph 15.

17. A method of generating an isolated antibody, or antigen-binding fragment thereof, that binds an epitope shared by both human mature Factor D and human Pro-Factor D comprising culturing the cell of paragraph 16 under conditions allowing for expression of the nucleic acid molecules encoding the antibody, or antigen-binding fragment thereof, that binds human Factor D and isolating said anti-Factor-D antibody, or antigen-binding fragment thereof.

18. A composition comprising an antibody, or antigen-binding fragment thereof, that specifically binds an epitope shared by both human mature Factor D and human Pro-Factor D as set forth in any of paragraphs 1 to 13.

19. A substrate for use in an immunoassay comprising at least one antibody, or antigen-binding fragment thereof, that binds an epitope shared by both human mature Factor D and human Pro-Factor D as set forth in any of paragraphs 1 to 13.

20. A kit for detecting the presence of Factor D in a biological sample, said kit comprising (a) at least one container, and (b) at least one antibody, or antigen-binding fragment thereof, that binds an epitope shared by both human mature Factor D and human Pro-Factor D as set forth in any of paragraphs 1 to 13.

D. Kits for Detecting Mature Factor D and/or Pro-Factor D in an Immunoassay

1. A kit comprising at least one monoclonal antibody or antigen-binding fragment thereof that specifically detects or quantitates human mature Factor D (SEQ ID NO:3) and/or Pro-Factor D (SEQ ID NO:2) in an immunoassay, wherein the at least one monoclonal antibody or antigen-binding fragment thereof comprises:
(i) a monoclonal antibody, or antigen-binding fragment thereof, that specifically binds to an epitope encompassing the amino-terminus of human mature Factor D, wherein the epitope comprises or consists of the amino acids ILGGREA (SEQ ID NO:5) and wherein said antibody does not bind to human Pro-Factor D (SEQ ID NO:2); or
(ii) a monoclonal antibody, or antigen-binding fragment thereof, that specifically binds to an epitope on the activation (“Pro”) peptide of human Factor D, wherein the epitope comprises or consists of “APPRGR” (SEQ ID NO:4) and wherein said antibody does not bind to mature Factor D (SEQ ID NO:3).

2. The kit of paragraph 1, wherein the kit further comprises an antibody, or fragment thereof, that binds to an epitope shared by both human mature Factor D (SEQ ID NO:3) and human Pro-Factor D (SEQ ID NO:2).

3. The kit of paragraph 1 or 2, wherein the kit further comprises at least one container.

4. The kit of any of paragraphs 1-3, wherein the antibody or antigen-binding fragment thereof of paragraph 1 subpart (i) that specifically binds to human mature Factor D comprises a binding domain comprising HC-CDR-1, HC-CDR-2 and HC-CDR-3 in a heavy chain variable region selected from the group consisting of SEQ ID NO:s 12-17 and comprising LC-CDR-1, LC-CDR2 and LC-CDR3 in a light chain variable region selected from the group consisting of SEQ ID NO:s 18-23, wherein the CDRs are numbered according to the Kabat numbering system.

5. The kit of any of paragraphs 1-3, wherein the antibody or antigen-binding fragment thereof of paragraph 1 subpart (ii) that specifically binds to an epitope on the activation (“Pro”) peptide of human Factor D comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

6. The kit of any of paragraphs 1-5, wherein the immunoassay is an enzyme-linked immunosorbent assay (ELISA).

7. The kit of any of paragraphs 1-6, wherein the antibody or antigen-binding fragment thereof of paragraph 1 subpart (i) is a coating antibody.

8. The kit of any of paragraphs 1-6, wherein the antibody or antigen-binding fragment thereof of paragraph 1 subpart (i) is a detecting antibody.

9. The kit of any of paragraphs 1-6, wherein the antibody or antigen-binding fragment thereof of paragraph 1 subpart (ii) is a coating antibody.

10. The kit of any of paragraphs 1-6, wherein the antibody or antigen-binding fragment thereof of paragraph 1 subpart (ii) is a detecting antibody.

11. The kit of any of paragraphs 1-10, wherein the kit further comprises an anti-Factor D antibody, or antigen-binding fragment thereof, that binds to an epitope shared by both human mature Factor D (SEQ ID NO:3) and human Pro-Factor D (SEQ ID NO:2).

12. The kit of paragraph 11, wherein the anti-Factor D antibody or antigen-binding fragment thereof comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

### E. Assays for Detecting Mature Factor D

1. A method of determining the presence or amount of mature Factor D in a test sample, the method comprising:
(a) contacting a test sample with an anti-human mature Factor D-specific monoclonal antibody or antigen-binding fragment thereof, in an in vitro immunoassay; and
(b) detecting the presence or absence or amount of the antibody or antigen-binding fragment thereof bound to mature Factor D, wherein the presence of binding indicates the presence or amount of mature Factor D in the sample;
wherein the anti-human mature Factor D-specific antibody or antigen-binding fragment thereof binds to an epitope in the N-terminal region of mature Factor D, set forth as amino acids ILGGREA (SEQ ID NO:5).

2. The method of paragraph 1, wherein the antibody or antigen-binding fragment thereof specifically binds human mature Factor D (SEQ ID NO:3) and does not bind to human Pro-Factor D (SEQ ID NO:2).

3. The method of any of paragraphs 1 or 2, wherein the anti-human mature Factor D-specific antibody or antigen-binding fragment thereof is immobilized on a substrate.

4. The method of any of paragraphs 1 to 3, wherein the immunoassay is an ELISA assay.

5. The method of any of paragraphs 1 to 4, wherein said anti-human mature Factor D-specific antibody or antigen-binding fragment thereof is labeled with a detectable moiety and step (b) comprises detecting the presence or amount of said detectable moiety.

6. The method of any of paragraphs 1 to 4, wherein said anti-human mature Factor D-specific antibody or antigen-binding fragment thereof is naked (i.e., not labeled), and the presence or amount of the antibody or antigen-binding fragment thereof bound to mature Factor D is detected using a labeled antibody which binds to the anti-mature Factor D antibody.

7. The method of any of paragraphs 1 to 4, wherein said anti-human mature Factor D-specific antibody or antigen-binding fragment thereof is immobilized on a substrate (i.e., capture/coating) and the bound mature Factor D is detected with a second antibody that binds to a different epitope of Factor D.

8. The method of any of paragraphs 1 to 7, wherein the test sample is a biological sample obtained from a mammalian subject, such as wherein the biological sample is selected from the group consisting of blood, serum, plasma, urine and cerebrospinal fluid.

9. The method of any of paragraphs 1 to 8, wherein the sample is obtained from a mammalian subject that is suffering from, or at risk for developing an Alternative Pathway related disease.

10. The method of any of paragraphs 1 to 9, wherein the sample is obtained from a mammalian subject after treatment with a complement inhibitory agent, such as an alternative complement pathway inhibitory agent, such as an inhibitor of pro-Factor D maturation, such as a MASP-3 inhibitory antibody.

11. The method of any of paragraphs 1 to 10, wherein the anti-human mature Factor D-specific antibody or antigen-binding fragment thereof comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23.

12. The method of any of paragraphs 1 to 11, wherein the anti-human mature Factor D-specific antibody or antigen-binding fragment thereof comprises a binding domain comprising the following six CDRs: a) an HC-CDR1 comprising the amino acid sequence XSXMGVS (SEQ ID NO:65), wherein X at position 1 is T, I or S and X at position 3 is G or I; (b) an HC-CDR2 comprising the amino acid sequence HIYWDDEKHYXPSLKX (SEQ ID NO: 66), wherein X at position 11 is H or N and X at position 16 is S or R; (c) an HC-CDR3 comprising the amino acid sequence RYYGYXXXMXY (SEQ ID NO:67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is N or S, X at position 4 is Q or E, X at position 7 is V or L, and X at position 15 is F or L; (e) a LC-CDR2 comprising the amino acid sequence KVXNRFS (SEQ ID NO:69), wherein: X at position 3 is S or Y; and (f) a LC-CDR3 comprising the amino acid sequence FQGSHVPPT (SEQ ID NO: 54).

13. The method of any of paragraphs 1 to 11, wherein the anti-human mature Factor D-specific antibody or antigen-binding fragment thereof comprises a binding domain comprising the following six CDRs: (a) an HC-CDR1 comprising SEQ ID NO:25, (b) an HC-CDR2 comprising SEQ ID NO:27; (c) an HC-CDR3 comprising SEQ ID NO: 29; (d) a LC-CDR1 comprising SEQ ID NO:50, (e) a LC-CDR2 comprising SEQ ID NO:52 and (f) a LC-CDR3 comprising SEQ ID NO:54.

### F. Assays for Detecting Pro-Factor D

1. A method of determining the presence or amount of Pro-Factor D in a test sample, the method comprising:
(a) contacting a test sample with an anti-human Pro-Factor D-specific monoclonal antibody or antigen-binding fragment thereof, in an in vitro immunoassay; and
(b) detecting the presence or absence or amount of the antibody or antigen-binding fragment thereof bound to Pro-Factor D, wherein the presence of binding indicates the presence or amount of Pro-Factor D in the sample;
wherein the anti-human mature Pro-Factor D-specific antibody or antigen-binding fragment thereof specifically binds to an epitope in the activation (“Pro”) peptide of human Factor D, set forth as “APPRGR” (SEQ ID NO:4).

2. The method of paragraph 1, wherein the antibody or antigen-binding fragment thereof specifically binds human Pro-Factor D (SEQ ID NO:2) and does not bind to human mature Factor D (SEQ ID NO:3).

3. The method of any of paragraphs 1 or 2, wherein the anti-human Pro-Factor D-specific antibody or antigen-binding fragment thereof is immobilized on a substrate.

4. The method of any of paragraphs 1 to 3, wherein the immunoassay is an ELISA assay.

5. The method of any of paragraphs 1 to 4, wherein said anti-human Pro-Factor D-specific antibody or antigen-binding fragment thereof is labeled with a detectable moiety and step (b) comprises detecting the presence or amount of said detectable moiety.

6. The method of any of paragraphs 1 to 4, wherein said anti-human Pro-Factor D-specific antibody or antigen-binding fragment thereof is naked (i.e., not labeled), and the presence or amount of the antibody or antigen-binding fragment thereof bound to mature Factor D is detected using a labeled antibody which binds to the anti-Pro-Factor D antibody.

7. The method of any of paragraphs 1 to 4, wherein said anti-human Pro-Factor D-specific antibody or antigen-binding fragment thereof is immobilized on a substrate (i.e., capture/coating) and the bound Pro-Factor D is detected with a second antibody or antigen-binding fragment thereof that binds to a different epitope of Factor D.

8. The method of any of paragraphs 1 to 7, wherein the test sample is a biological sample obtained from a mammalian subject, such as wherein the biological sample is selected from the group consisting of blood, serum, plasma, urine and cerebrospinal fluid.

9. The method of any of paragraphs 1 to 8, wherein the sample is obtained from a mammalian subject that is suffering from, or at risk for developing an Alternative Pathway related disease.

10. The method of any of paragraphs 1 to 9, wherein the sample is obtained from a mammalian subject after treatment with a complement inhibitory agent, such as an alternative complement pathway inhibitory agent, such as an inhibitor of pro-Factor D maturation, such as a MASP-3 inhibitory antibody.

11. The method of any of paragraphs 1 to 10, wherein the anti-human Pro-Factor D-specific antibody or antigen-binding fragment thereof comprises a binding domain comprising HC-CDR-1, HC-CDR-2 and HC-CDR-3 of a heavy chain variable region selected from the group consisting of SEQ ID NO:s 136-141 and comprising LC-CDR-1, LC-CDR2 and LC-CDR3 in a light chain variable region selected from the group consisting of SEQ ID NO:s 142-147.

12. The method of any of paragraphs 1 to 11, wherein the anti-human Pro-Factor D-specific antibody or antigen-binding fragment thereof comprises a binding domain comprising HC-CDR-1, HC-CDR-2 and HC-CDR-3 in a heavy chain variable region selected from the group consisting of SEQ ID NO:s 136-139 and comprising LC-CDR-1, LC-CDR2 and LC-CDR3 in a light chain variable region selected from the group consisting of SEQ ID NO:s 142-145.

13. The method of any of paragraphs 1 to 11, wherein the anti-human Pro-Factor D-specific antibody or antigen-binding fragment thereof comprises a binding domain comprising HC-CDR-1, HC-CDR-2 and HC-CDR-3 in a heavy chain variable region selected from the group consisting of SEQ ID NO:140 and SEQ ID NO:141 and comprising LC-CDR-1, LC-CDR2 and LC-CDR3 in a light chain variable region selected from the group consisting of SEQ ID NO:146 and SEQ ID NO:147.

14. The method of any of paragraphs 1 to 11, wherein the anti-human Pro-Factor D-specific antibody or antigen-binding fragment thereof comprises a binding domain comprising the following six CDRs: (a) a CDR-H1 comprising SEQ ID NO:167, (b) a CDR-H2 comprising SEQ ID NO:169 or SEQ ID NO:173; (c) a CDR-H3 comprising SEQ ID NO: 171 or SEQ ID NO:174; (d) a CDR-L1 comprising SEQ ID NO:194, (e) a CDR-L2 comprising SEQ ID NO:196 or SEQ ID NO:199 and (f) a CDR-L3 comprising SEQ ID NO: 198 or SEQ ID NO:200.

### G. Method of Assessing the Extent of Alternative Pathway Activation in a Test Sample

1. A method of assessing the extent of alternative pathway complement (APC) activation in a test sample comprising:
(a) providing a test sample;
(b) performing an immunoassay comprising at least one of:
   (i) capturing and detecting mature Factor D in the test sample, wherein mature Factor D is either captured or detected with a monoclonal antibody or antigen-binding fragment thereof that specifically binds to an epitope in “ILGGREA” (SEQ ID NO:5) present in mature Factor D, but does not bind to Pro-Factor D; and/or
   (ii) capturing and detecting Pro-Factor D in the test sample, wherein Pro-Factor D is either captured or detected with a monoclonal antibody or antigen-binding fragment thereof that specifically binds to an epitope on the activation (“Pro”) peptide “APPRGR” (SEQ ID NO:4) present in Pro-Factor D, but does not bind to mature Factor D; and
(c) comparing the level of mature Factor D detected in accordance with (b) (i) with a predetermined level or control sample and/or comparing the level of Pro-Factor D detected in accordance with (b (ii) with a predetermined level or control sample, wherein the level of mature Factor D and/or Pro-Factor D detected in the test sample is indicative of the extent of Alternative Pathway Complement activation.

2. The method of paragraph 1, wherein step (b) (i) comprises capturing mature Factor D with a monoclonal antibody or antigen-binding fragment thereof that specifically binds to an epitope in “ILGGREA” (SEQ ID NO:5) present in mature Factor D, but does not bind to Pro-Factor D and detecting with an antibody or antigen-binding fragment thereof that binds to an epitope shared by both human mature Factor D and human Pro-Factor D.

3. The method of paragraph 1, wherein step (b) (i) comprises capturing mature Factor D with an antibody or antigen-binding fragment thereof that binds to an epitope shared by both human mature Factor D and human Pro-Factor D and detecting with a monoclonal antibody or antigen-binding fragment thereof that specifically binds to an epitope in “ILGGREA” (SEQ ID NO:5) present in mature Factor D, but does not bind to Pro-Factor D.

4. The method of paragraph 1, wherein step (b) (ii) comprises capturing Pro-Factor D with a monoclonal antibody or antigen-binding fragment thereof that specifically binds to an epitope on the activation (“Pro”) peptide “APPRGR” (SEQ ID NO:4) present in Pro-Factor D, but does not bind to mature Factor D and detecting with an antibody or antigen-binding fragment thereof that binds to an epitope shared by both human mature Factor D and human Pro-Factor D.

5. The method of paragraph 1, wherein step (b) (ii) comprises capturing Pro-Factor D with an antibody or antigen-binding fragment thereof that binds to an epitope shared by both human mature Factor D and human Pro-Factor D and detecting with a monoclonal antibody or antigen-binding fragment thereof that specifically binds to an epitope on the activation (“Pro”) peptide “APPRGR” (SEQ ID NO:4) present in Pro-Factor D, but does not bind to mature Factor D.

6. The method of paragraph 1, wherein the monoclonal antibody or antigen-binding fragment thereof that specifically binds to an epitope in “ILGGREA” (SEQ ID NO:5) present in mature Factor D, but does not bind to Pro-Factor D comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23.

7. The method of paragraph 1, wherein the monoclonal antibody or antigen-binding fragment thereof that specifically binds to an epitope on the activation (“Pro”) peptide “APPRGR” (SEQ ID NO:4) present in Pro-Factor D, but does not bind to mature Factor D comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

8. The method of any of paragraphs 1-7, wherein the test sample is a biological sample obtained from a mammalian subject.

9. The method of paragraph 8, wherein the biological sample comprises whole blood, serum, plasma, urine, or cerebrospinal fluid.

10. The method of any of paragraphs 1-9, wherein the test sample comprises a complement inhibitory agent, such as an alternative complement pathway inhibitory agent, such as an inhibitor of pro-Factor D maturation, such as a MASP-3 inhibitory agent (e.g., a MASP-3 inhibitory antibody or an antigen-binding fragment thereof).

11. The method of paragraph 8, wherein the mammalian subject has been treated with a complement inhibitory agent, such as an alternative complement pathway inhibitory agent, such as an inhibitor of pro-Factor D maturation, such as a MASP-3 inhibitory agent (e.g., a MASP-3 inhibitory antibody or an antigen-binding fragment thereof).

12. The method of paragraph 8, wherein the mammalian subject is a human subject.

13 The method of paragraph 12, wherein the human subject is suffering from, or at risk of developing, or suspected of having an alternative-pathway disease or disorder.

14. The method of paragraph 13, wherein the alternative-pathway disease or disorder is selected from the group consisting of: paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

15. The method of any of paragraphs 11-14, wherein the control sample is a sample taken from the subject prior to treatment with the MASP-3 inhibitory agent, or a sample taken at an earlier point in time during a course of treatment with the MASP-3 inhibitory agent.

16. The method of any of paragraphs 11-15, wherein the MASP-3 inhibitory agent is a MASP-3 inhibitory antibody or antigen-binding fragment thereof.

17. The method of paragraph 16, wherein the MASP-3 inhibitory antibody or antigen-binding fragment thereof is a monoclonal antibody, or antigen-binding fragment thereof, that binds to MASP-3 and comprises at least one of:
(i) a binding domain comprising HC-CDR1, HC-CDR2 and HC-CDR3 of a heavy chain variable region selected from the group consisting of SEQ ID NO:s 220, 222, 223, 225, 226 and 228 and comprising LC-CDR1, LC-CDR2 and LC-CDR3 of a light chain variable region selected from the group consisting of SEQ ID NO:s 221, 224 and 227, wherein the CDRs are numbered according to the Kabat numbering system;
(ii) a heavy chain variable region comprising a HC-CDR1 comprising SEQ ID NO: 229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO:178 (WASTRES) and a LC-CDR3 comprising SEQ ID NO: 242 (KQSYNLYT);
(iii) a heavy chain variable region comprising a HC-CDR1 comprising SEQ ID NO: 230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO:241 (LVSKLDS) and a LC-CDR3 comprising SEQ ID NO:243 (WQGTHFPWT); or
(iv) a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT).
   H. Methods of Monitoring the Efficacy of Treatment with a MASP-3 Inhibitory Agent

1. A method for monitoring the efficacy of treatment with a MASP-3 inhibitory antibody in a mammalian subject, the method comprising:
(a) administering a dose of a MASP-3 inhibitory antibody or antigen-binding fragment thereof to a mammalian subject at a first point in time;
(b) assessing a first concentration of mature Factor D and/or Pro-Factor D in a biological sample obtained from the subject after step (a);
(c) treating the subject with the MASP-3 inhibitory antibody or antigen-binding fragment thereof at a second point in time;
(d) assessing a second concentration of mature Factor D and/or Pro-Factor D in a biological sample obtained from the subject after step (c); and
(e) comparing the level of mature Factor D and/or Pro-Factor D assessed in step (b) with the level of mature Factor D and/or Pro-Factor D assessed in step (d) to determine the efficacy of the MASP-3 inhibitory antibody in the mammalian subject.

2. The method of paragraph 1, wherein the method further comprises adjusting the dose of the MASP-3 inhibitory antibody or antigen-binding fragment thereof.

3. The method of paragraph 2, wherein the dose of MASP-3 inhibitory antibody or antigen-binding fragment thereof administered to the subject is increased if the level of mature Factor D is higher than the control or reference standard.

4. The method of paragraph 2, wherein the dose of MASP-3 inhibitory antibody or antigen-binding fragment administered to the subject is increased if the level of Pro-Factor D is lower than the control or reference standard.

5. The method of paragraph 3 or 4, wherein if the subject is administered an increased dose of the MASP-3 inhibitory antibody or antigen-binding fragment thereof, steps (b) to (e) are repeated to determine whether the increased dose is sufficient to adjust the level of mature Factor D and/or Pro-Factor D to the desired level as compared to the respective control or reference standard.

6. The method of any of paragraphs 1-5, wherein steps (b) and (d) comprise assessing the concentration of mature Factor D in the biological samples in an immunoassay.

7. The method of paragraph 6, wherein the immunoassay comprises (i) a first monoclonal antibody or antigen-binding fragment thereof that specifically binds to an epitope in the N-terminal region of human mature Factor D, wherein the epitope comprises or consists of the amino acids ILGGREA (SEQ ID NO:5) and does not bind to human Pro-Factor D; and (ii) a second antibody or antigen-binding fragment thereof that binds to an epitope shared by both human mature Factor D and human Pro-Factor D, wherein the first and second antibody or antigen-binding fragments thereof function together in the immunoassay to specifically detect or quantitate the amount of mature Factor D protein (SEQ ID NO:3) and not Pro-Factor D protein (SEQ ID NO:2) that may be present in the biological sample.

8. The method of any of paragraphs 1-5, wherein steps (b) and (d) comprise assessing the concentration of Pro-Factor D in the biological samples in an immunoassay.

9. The method of paragraph 8, wherein the immunoassay comprises (i) a first monoclonal antibody or antigen-binding fragment thereof that specifically binds to an epitope in the pro peptide of human Factor D, wherein the epitope comprises or consists of the amino acids APPRGR (SEQ ID NO:4) and does not bind to human mature Factor D; and (ii) a second antibody or antigen-binding fragment thereof that binds to an epitope shared by both human mature Factor D and human Pro-Factor D, wherein the first and second antibody function together in the immunoassay to specifically detect or quantitate the amount of Pro-Factor D protein (SEQ ID NO:2) and not mature-Factor D protein (SEQ ID NO:3) that may be present in the biological sample.

10. The method of any of paragraphs 1-9, wherein the mammalian subject is a human subject.

11. The method of paragraph 10, wherein the human subject is suffering from, or at risk of developing an alternative pathway disease or disorder.

12. The method of paragraph 11, wherein the alternative pathway disease or disorder is selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

13. The method of any of paragraphs 1-12 wherein the MASP-3 inhibitory antibody or antigen-binding fragment thereof is a monoclonal antibody or antigen-binding fragment thereof.

14. The method of paragraph 13, wherein the MASP-3 inhibitory antibody or antigen-binding fragment thereof is a monoclonal antibody, or antigen-binding fragment thereof binds to MASP-3 and comprises at least one of:
(i) a binding domain comprising HC-CDR1, HC-CDR2 and HC-CDR3 of a heavy chain variable region selected from the group consisting of SEQ ID NO:s 220, 222, 223, 225, 226 and 228 and comprising LC-CDR1, LC-CDR2 and LC-CDR3 of a light chain variable region selected from the group consisting of SEQ ID NO:s 221, 224 and 227, wherein the CDRs are numbered according to the Kabat numbering system;
(ii) a heavy chain variable region comprising a HC-CDR1 comprising SEQ ID NO: 229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO:178 (WASTRES) and a LC-CDR3 comprising SEQ ID NO: 242 (KQSYNLYT);
(iii) a heavy chain variable region comprising a HC-CDR1 comprising SEQ ID NO: 230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO:241 (LVSKLDS) and a LC-CDR3 comprising SEQ ID NO:243 (WQGTHFPWT); or
(iv) a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT).
   I. Methods of Treating a Mammalian Subject Suffering from or at Risk of Developing an Alternative Pathway Disease or Disorder

1. A method of treating a mammalian subject suffering from, or at risk of developing an alternative-pathway disease or disorder, comprising administering a MASP-3 inhibitory antibody or antigen-binding fragment thereof to the subject if the subject is determined to have:
(i) a lower or decreased level of Pro-Factor D in one or more samples taken from the subject compared to a predetermined Pro-Factor D level or compared to the Pro-Factor D level in one or more control samples; and/or
(ii) a higher or increased level of mature Factor D in one or more samples taken from the subject compared to a predetermined mature Factor D level or compared to the mature Factor D level in one or more control samples.

2. The method of paragraph 1, wherein the level of Pro-Factor D in one or more samples taken from the subject is determined by performing an immunoassay comprising the use of a Pro-Factor D-specific monoclonal antibody or antigen-binding fragment thereof.

3. The method of paragraph 2, wherein the immunoassay comprises (i) a first monoclonal antibody or antigen-binding fragment thereof that specifically binds to an epitope in the pro peptide of human Factor D, wherein the epitope comprises or consists of the amino acids APPRGR (SEQ ID NO:4) and does not bind to human mature Factor D; and (ii) a second antibody or antigen-binding fragment thereof that binds to an epitope shared by both human mature Factor D and human Pro-Factor D, wherein the first and second antibody or antigen-binding fragments thereof function together in the immunoassay to specifically detect or quantitate the amount of Pro-Factor D protein (SEQ ID NO:2) and not mature-Factor D protein (SEQ ID NO:3) that may be present in the sample.

4. The method of paragraph 1, wherein the level of mature Factor D in one or more samples taken from the subject is determined by performing an immunoassay comprising the use of a mature Factor D-specific monoclonal antibody or antigen-binding fragment thereof.

5. The method of paragraph 4, wherein the immunoassay comprises (i) a first monoclonal antibody or antigen-binding fragment thereof that specifically binds to an epitope in the N-terminal region of human mature Factor D, wherein the epitope comprises or consists of the amino acids ILGGREA (SEQ ID NO:5) and does not bind to human Pro-Factor D; and (ii) a second antibody or antigen-binding fragment thereof that binds to an epitope shared by both human mature Factor D and human Pro-Factor D, wherein the first and second antibody or antigen-binding fragments thereof function together in the immunoassay to specifically detect or quantitate the amount of mature Factor D protein (SEQ ID NO:3) and not Pro-Factor D protein (SEQ ID NO:2) that may be present in the sample.

6. The method of any of paragraphs 1-5, wherein the mammalian subject is a human subject.

7. The method of any of paragraphs 1-6, wherein the mammalian subject is suffering from, or at risk of developing an alternative pathway disease or disorder selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

8. The method of any of paragraphs 1-7 wherein the MASP-3 inhibitory antibody or antigen-binding fragment thereof is a monoclonal antibody or antigen-binding fragment thereof.

9. The method of paragraph 8, wherein the MASP-3 inhibitory antibody or antigen-binding fragment thereof is a monoclonal antibody, or antigen-binding fragment thereof binds to MASP-3 and comprises at least one of:
(i) a binding domain comprising HC-CDR1, HC-CDR2 and HC-CDR3 of a heavy chain variable region selected from the group consisting of SEQ ID NO:s 220, 222, 223, 225, 226 and 228 and comprising LC-CDR1, LC-CDR2 and LC-CDR3 of a light chain variable region selected from the group consisting of SEQ ID NO:s 221, 224 and 227, wherein the CDRs are numbered according to the Kabat numbering system;
(ii) a heavy chain variable region comprising a HC-CDR1 comprising SEQ ID NO: 229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO:178 (WASTRES) and a LC-CDR3 comprising SEQ ID NO: 242 (KQSYNLYT);
(iii) a heavy chain variable region comprising a HC-CDR1 comprising SEQ ID NO: 230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO:241 (LVSKLDS) and a LC-CDR3 comprising SEQ ID NO:243 (WQGTHFPWT); or
(iv) a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT).

### J. Pharmaceutical Compositions and Articles of Manufacture

1. A pharmaceutical composition comprising a MASP-3 inhibitory antibody or an antigen-binding fragment thereof in an aqueous solution comprising a buffer system having a pH of 6.0±5%, 20±5% mM histidine, 100±5% mg/mL sucrose, and 0.035%+5%, polysorbate 80 wherein said MASP-3 inhibitory antibody is included at a concentration of 110 mg/mL±5%, and wherein said MASP-3 inhibitory antibody or antigen-binding fragment thereof comprises a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO: 238 (SEDV); and a light chain variable region comprising a LC-CDR1 comprising SEQ ID NO: 239, a LC-CDR2 comprising SEQ ID NO:178 (WASTRES); and a LC-CDR3 comprising SEQ ID NO:244 (KQSYNIPT).

2. The pharmaceutical composition of paragraph 1, wherein the pharmaceutical composition is sterile.

3. The pharmaceutical composition of paragraph 1 or 2, wherein the MASP-3 inhibitory antibody or antigen-binding fragment thereof comprises a heavy chain variable region comprising at least 80%, 85%, 90%, 95%, 98%, 99% or 100% identical to SEQ ID NO: 226 or SEQ ID NO:227 and a light chain variable region comprising at least 80%, 85%, 90%, 95%, 98%, 99% or 100% identical to SEQ ID NO:227.

4. The pharmaceutical composition of any of paragraphs 1 to 3, wherein the MASP-3 inhibitory antibody or antigen-binding fragment thereof is selected from the group consisting of a human antibody, a humanized antibody, a chimeric antibody, a murine antibody, and an antigen-binding fragment of any of the foregoing.

5. The pharmaceutical composition of any of paragraphs 1 to 3, wherein the MASP-3 inhibitory antibody or antigen-binding fragment thereof is selected from the group consisting of a single chain antibody, an ScFv, a Fab fragment, an Fab′ fragment, an F(ab′)₂ fragment, a univalent antibody lacking a hinge region and a whole antibody.

6. The pharmaceutical composition of any of paragraphs 1 to 3, wherein the MASP-3 inhibitory antibody or antigen-binding fragment thereof further comprises an immunoglobulin constant region.

7. The pharmaceutical composition of paragraph 6, wherein the MASP-3 inhibitory antibody or antigen-binding fragment thereof comprises a human IgG4 constant region.

8. The pharmaceutical composition of paragraph 7, wherein the MASP-3 inhibitory antibody or antigen-binding fragment thereof comprises a human IgG4 constant region with an S228P mutation.

9. The pharmaceutical composition of paragraph 7 or 8, wherein the MASP-3 inhibitory antibody or antigen-binding fragment thereof comprises a mutation that promotes FcRn interactions at low pH.

10. The pharmaceutical composition of any of paragraphs 7-9, wherein the MASP-3 inhibitory antibody or antigen-binding fragment thereof comprises human IgG4 constant region set forth as SEQ ID NO:245.

11. An article of manufacture containing a pharmaceutical composition according to any of paragraphs 1-10.

12. The article of manufacture of paragraph 11, wherein the MASP-3 inhibitory antibody or antigen-binding fragment thereof is in a unit dosage form of from 10 mg to 1000 mg suitable for therapeutic administration to a human subject.

13. The article of manufacture of paragraph 10 or 11, wherein the article of manufacture comprises a container and a label or package insert on or associated with the container.

14. The article of manufacture of paragraph 13, wherein the container is selected from the group consisting of a bottle, an ampoule, a pouch (e.g. an intravenous infusion bag), a vial, a syringe, and a cartridge.

15. The pharmaceutical composition of any of paragraphs 1 to 10 or the article of manufacture of any of paragraphs 11 to 14, wherein the composition and/or article of manufacture is for use in the treatment of a subject suffering from, or at risk of developing an alternative pathway disease or disorder.

16. The pharmaceutical composition or article of manufacture of paragraph 15, wherein the alternative pathway disease or disorder is selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

The following examples merely illustrate the best mode now contemplated for practicing the invention, but should not be construed to limit the invention.

### Example 1

This Example describes the generation of monoclonal antibodies that specifically bind to human mature Factor D.

### Background

This Example describes the generation of anti-human mature Factor D-specific antibodies suitable for use as detection reagents for use in assays to measuring the presence and/or amount of mature Factor D in a biological sample for use as a biomarker of APC status. The antibodies described in this Example specifically bind to human mature Factor D (SEQ ID NO: 3) and do not bind to human pro Factor D (SEQ ID NO:2).

### Methods:

### 1. Expression of a Synthetic Mature Factor D Peptide Antigen

The amino acid sequences of human full-length Factor D (SEQ ID NO:1), human pro-Factor D (SEQ ID NO:2) and human mature Factor D (SEQ ID NO:3) are shown in FIG. 2. As shown in FIG. 2, the pro-peptide of human Pro-Factor D is “APPRGR” (SEQ ID NO: 4). FIG. 3 provides an alignment of the amino acid sequences of complement Factor D (full-length) from various species including Homo sapiens (SEQ ID NO: 1); Macaca (SEQ ID NO:8); Canis (SEQ ID NO:9); Rattus (SEQ ID NO:10); and Mus (SEQ ID NO:11). The italicized portion of each sequence depicts the signal sequence and the underlined portion depicts the activation “pro” peptide sequence. As shown in FIGS. 2 and 3, the Factor D protein comprises an activation peptide (Pro peptide, underlined). Once the pro peptide is cleaved, mature Factor D has a unique amino-terminus as compared to pro-Factor D in each species (e.g., having an N-terminus starting at residue 26 of human full-length Factor D (SEQ ID NO:1)).

In order to generate anti-human mature Factor D-specific antibodies, a synthetic peptide was generated corresponding to amino acid residues 26-32 of human complement factor D: “ILGGREA” (SEQ ID NO:5) as follows. A synthetic mature factor D peptide-KLH conjugate construct was generated by inserting the nucleic acid sequence encoding “ILGGREA” (SEQ ID NO:5) separated by a spacer amino acid sequence from the PADRE sequence, a spacer amino acid sequence and a C-terminal cysteine: “ILGGREAGPGPGAKFVAAAWTLKAAAKKC” (SEQ ID NO:6), allowing for conjugation to KLH by Sulfo-SMCC linkage chemistry.

2. Immunization with the Mature Factor D Antigen

C57BL6 mice were immunized with the synthetic mature factor D peptide-KLH conjugate (SEQ ID NO:6) described above. The mice were immunized three times, subcutaneously, with 50 μL of adjuvant-emulsions of peptide conjugate (50-100 μg total protein per injection).

Serum samples from the immunized mice were prepared from retro-orbital sinus bleeds and tested by ELISA for the presence of antigen-specific antibodies capable of binding to plate-immobilized recombinant human pro-factor D (hPro-CFD) (SEQ ID NO:2) and recombinant human mature Factor D (hCFD) (SEQ ID NO:3) as follows:

Recombinant human Pro-CFD-His or recombinant human mature CFD-His were immobilized on Maxisorp™ ELISA plates at 1 μg/mL in PBS, 100 μL/well and incubated overnight at 4° C. Plate wells were then washed three times with 300 μL PBS containing 0.05% Tween 20 (PBST), blocked for 1 hour at room temperature with 250 μL PBS containing 1% bovine serum albumen (BSA) and washed again. Serum from each mouse was diluted in PBST and allowed to bind for 1 hour at room temperature, then washed three times in PBST. A horseradish peroxidase (HRP)-labeled goat anti-mouse IgG Fc antibody (Jackson ImmunoResearch) was then applied (100 μL/well), allowed to bind for 1 hour at room temperature, and then washed three times with PBST. TMB substrate (ThermoFischer) (100 μL/well) was then applied and incubated for 5 minutes at room temperature. The reaction was then stopped with 1N H₂SO₄ (50 μL/well). The plate was read for optical density at 450 nM with a Biotek™ ELISA plate reader.

### Results:

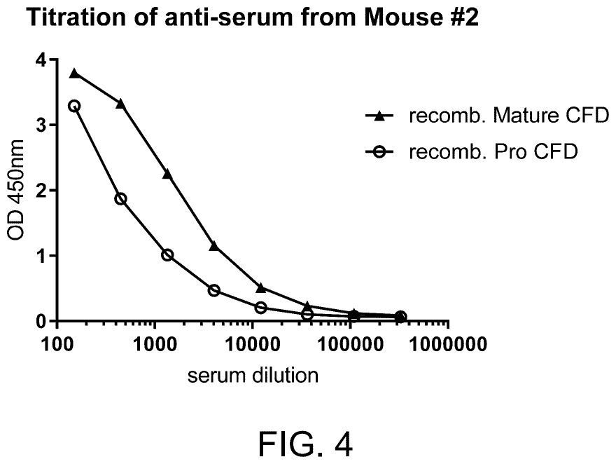

FIG. 4 graphically illustrates a titration of the serum of a representative mouse #2 after immunization with a synthetic peptide corresponding to amino acid residues 26-32 of human complement factor D “ILGGREA” (SEQ ID NO:5) in the presence of recombinant mature Factor D or recombinant pro-Factor D. As shown in FIG. 4, the serum from representative mouse #2 contains antibodies capable of selectively binding to mature Factor D as compared to pro-Factor D.

The mice showing the most favorable binding to mature Factor D and the least favorable binding to pro-Factor D (i.e., mouse #2) were selected for hybridoma fusion. Three days prior to the fusion, mice were treated subcutaneously with 50 μg of an anti-CD40 agonist mAb in PBS (R&D Systems, Minneapolis, MN) to increase B cells numbers (see Rycyzyn et al., Hybridoma 27:25-30, 2008). The mice were sacrificed, and the spleen cells were harvested and fused to a selected murine myeloma cell line P3/NSI/1-AG4-1 (NS-1) (ATCC No. TIB18) using 50% polyethylene glycol or 50% polyethylene glycol plus 10% DMSO. The fusions generated hybridoma cells which were plated in 96 well Nunc tissue culture treated plates containing HAT (hypoxanthine, aminopterin and thymidine) medium to inhibit proliferation of non-fused cells, myeloma hybrids and spleen hybrids. Hybridoma wells were fed by replacement of 80% of media with fresh medium containing HAT supplement. After hybridoma selection, the culture supernatants were assayed for binding to recombinant human mature factor D as described below.

### 3. Hybridoma Screening

Hybridoma supernatants were first screened for binding to immobilized recombinant human mature Factor D-His. 10 hybridomas were identified (n=10) which were then tested for their ability to detect recombinant human mature Factor D or recombinant human pro-Factor D when captured by a polyclonal goat anti-human factor D antibody AF1824 (R&D Systems) as follows. ELISA plates were coated with polyclonal anti-human factor D antibody AF1824 (R&D Systems). Hybridoma supernatants were diluted two-fold in PBS, 0.05% Tween 20 (PBST). Supernatant from NS-1 myeloma cell line (NS-1 sup) was included as a matrix control to determine the level of assay background.

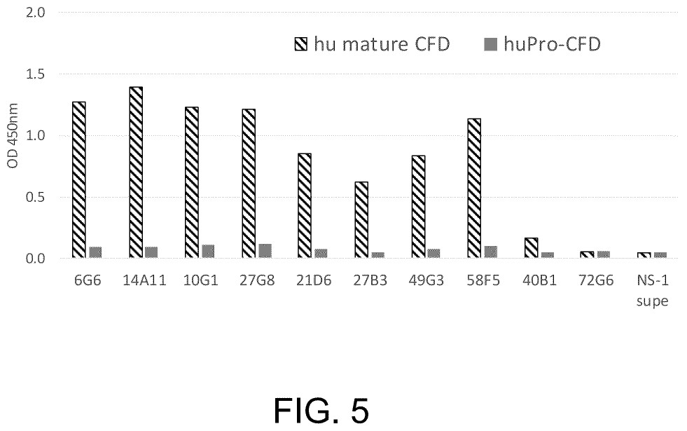

FIG. 5 graphically illustrates the results of a capture ELISA assay in which hybridoma supernatants were screened for binding to human mature-Factor D or human Pro-Factor D when captured by a polyclonal anti-Factor D antibody AF1824 (R&D Systems). As shown in FIG. 5, out of 10 hybridomas tested, the supernatants from 8 hybridomas (6G6, 14A11, 10G1, 27G8, 21D6, 27B3, 49G3, 58F5) showed preferential binding to recombinant human mature factor D as compared to recombinant human pro-Factor D. Hybridomas 6G6, 14A11, 10G1, 27B3, 49G3 and 58GF5 were selected for DNA cloning and recombinant antibody production.

### Hybridoma Supernatant Specificity

The specificity of the supernatants of hybridomas 14A11 and 6G6 were further analyzed by measuring detection of captured or endogenous proteins in human serum or plasma matrix as follows. ELISA plates were coated overnight at 4° C. with polyclonal goat anti-human CFD AF1824 (R&D Systems). Plate wells were washed, blocked, and washed again. Normal human serum pool, normal human plasma pool or Factor D-depleted human serum were diluted 10-fold in assay buffer (PBS with 1% BSA and 0.05% Tween 20, PBST-BSA), either un-spiked or spiked with 2 μg/mL recombinant pro-(pro CFD) or mature-factor D (mature CFD). These matrices, including a buffer control with or without recombinant protein spiked in, were incubated for 60 minutes at room temperature, and then washed. Anti-human Factor D detection antibodies were diluted as follows then applied to the plate: subclones of 6G6 and 14A11 hybridoma supernatants were diluted in half with PBST-BSA. Purified mAb1824 mouse monoclonal antibody was diluted to 0.5 μg/mL in PBST-BSA buffer. The detection antibodies were incubated for one hour at room temperature and washed, then developed with a horseradish peroxidase (HRP)-labeled goat polyclonal to mouse IgG Fc (Jackson ImmunoResearch).

### Results:

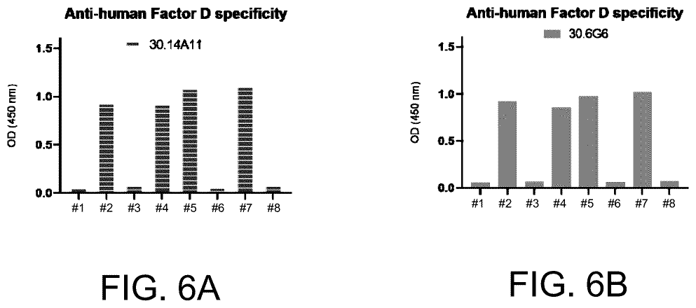

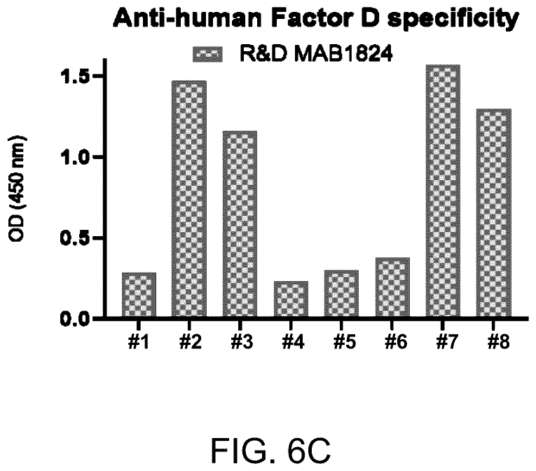

The results are shown in FIG. 6A-C. The following samples #1 to #8 are shown in each of FIGS. 6A-C:
#1: Factor D-depleted serum control
#2: Factor D-depleted serum spiked with recombinant human mature-CFD
#3: Factor D-depleted serum spiked with recombinant human pro-CFD
#4: Normal human serum control
#5: Normal human plasma control
#6: Buffer control
#7: Buffer, spiked with recombinant human mature-CFD
#8: Buffer, spiked with recombinant human pro-CFD

FIG. 6A graphically illustrates the results with samples #1 to #8 described above in an ELISA assay with coated polyclonal goat anti-human CFD 1824 and detected with hybridoma supernatant 14A11 present in each condition. As shown in FIG. 6A, hybridoma supernatant 14A11 is capable of selectively detecting recombinant mature complement factor D and does not detect recombinant pro factor D. It is noted that the signal obtained when Df-Dpl serum is spiked with recombinant mature CFD (#2) is similar to the signal obtained when 10% normal human serum (#4) or normal human plasma (#5) are added, both of which should contain normal levels of mature Factor D.

FIG. 6B graphically illustrates the results with samples #1 to #8 described above in an ELISA assay with coated polyclonal goat anti-human CFD 1824 and detected with hybridoma supernatant 6G6 present in each condition. As shown in FIG. 6B, hybridoma supernatant 6G6 is capable of selectively detecting recombinant mature complement factor D and does not detect recombinant pro factor D. It is noted that the signal obtained when Df-Dpl serum is spiked with recombinant mature CFD (#2) is similar to the signal obtained when 10% normal human serum (#4) or normal human plasma (#5) are added, both of which should contain normal levels of mature CFD.

FIG. 6C graphically illustrates the results with samples #1 to #8 described above in an ELISA assay with coated polyclonal goat anti-human CFD 1824 and detected with mAb 1824 present in each condition. As shown in FIG. 6C, monoclonal antibody MAB1824 (R&D Systems) detects both recombinant mature CFD and recombinant active CFD and therefore is not capable of selectively detecting mature CFD as compared to pro CFD.

Those supernatants showing preferential binding to the mature version of Factor D (i.e., 6G6, 14A11, 10G1, 27B3, 49G3 and 58GF5) were expanded and cloned by limiting dilution until monoclonal.

### Example 2

This Example describes the cloning and sequence analysis of anti-human mature factor D-specific monoclonal antibodies.

### Background/Rationale:

This Example describes the cloning and sequence analysis of antibodies produced by the hybridomas showing preferential binding to the mature version of Factor D (i.e., clones 6G6, 14A11, 10G1, 27B3, 49G3 and 58F5) that were generated as described in Example 1.

### Methods:

### Cloning and Purification of Recombinant Antibodies:

Positive hybridomas 6G6, 14A11, 10G1, 27B3, 49G3, 58F5 were generated and identified as described in Example 1. These hybridomas were subcloned by serial dilution methods. The heavy chain and light chain variable regions were cloned from the hybridomas described in Example 1 using RT-PCR and were sequenced. Antibody-encoding sequences were amplified from total RNA with isotype-specific reverse primers using the SMARTer™ RACE 5′/3′ kit (Takara Bio). After verifying the sequences, the variable (V) regions were re-amplified with designed cloning primers and cloned into expression vectors carrying either the human IgG4 heavy chain (SEQ ID NO:71) and kappa light chain (SEQ ID NO:72) constant regions or the mouse IgG2a (SEQ ID NO:218) and kappa light chain (SEQ ID NO: 219) constant regions using the In-Fusion HD™ cloning kit (Clontech). The expression constructs were co-transfected transiently into Expi293 cells (Life Technologies) and after 5 days of culture, secreted recombinant antibodies were purified from supernatants by protein A chromatography.

The sequences of the heavy chain variable regions and light chain variable regions are shown in FIGS. 7A and 7B, respectively (“SIN”=“SEQ ID NO:” in FIG. 7A and FIG. 7B), and are included below. The complementarity determining regions (CDRs) and framework regions (FRs) of each are provided in TABLES 7-10 below.

### Anti-Human Mature-Factor D-Specific Antibody Heavy Chain Variable Region (VH) Sequences

FIG. 7A shows an amino acid alignment of the heavy chain variable region (VH) sequences for the anti-human mature-Factor D-specific clones: 6G6_VH (SEQ ID NO:12), 14A11_VH (SEQ ID NO:13), 27B3_VH (SEQ ID NO:14), 58F5_VH (SEQ ID NO:15), 49G3_VH (SEQ ID NO:16), and 10G1_VH (SEQ ID NO:17).

Presented below is the heavy chain variable region (VH) sequence for each anti-human mature-factor-D-specific antibody. The Kabat CDRs are underlined.

6G6_VH:
SEQ ID NO: 12
QITLKESGPGILQSSQTLSLTCSFSGISLTTSGMGVSWIRQPSGKGLEW
LAHIYWDDEKHYHPSLKSRLTISKDASRNQVFFRILSVDTADTATYYCA
LRYYGYRSFMDYWGQGTSVTVSS
14A11_VH:
SEQ ID NO: 13
QITLKESGPGILQSSQTLSLTCSFSGVSLTTSGMGVSWIRQPSGKGLEW
LAHIYWDDEKHYHPSLKSRLTISKDASRNQVFFRILSVDTADTATYYCA
LRYYGYRSFMDYWGQGTSVTVSS
27B3_VH:
SEQ ID NO: 14
QVTLKESGPGILQSSQTLSLTCSFSGISLNISGMGVSWIRQPSGKGLEW
LAHIYWDDEKHYNPSLKRRLTISKDASRNQVFFRISSVDSADTATYYCA
LRYYGYGSIMDYWGHGTSVTVSS
58F5_VH:
SEQ ID NO: 15
QVTLKESGPGILQSSQTLSLTCSFSGISLNTSIMGVSWIRQPSGKGLEW
LAHIYWDDEKHYNPSLKSRLTISKDASRNQVFLKIISVDTADTATYYCA
LRYYGYNYVMHYWGQGTSVTVSS
49G3_VH:
SEQ ID NO: 16
QVTLKESGPGILQSSQTLSLTCSFSGISLSSSGMGVSWIRQPSGKGLEW
LAHIYWDDEKHYNPSLKSRLTISKDASRNQIFLKIISVDTADTATYYCA
LRYYGYNYVMHYWGQGTSVTVSS
10G1_VH:
SEQ ID NO: 17
QVTLKESGPGILQSSQTLSLTCSFSGVSLSSSGMGVSWIRQPSGKGLEW
LAHIYWDDEKHYNPSLKSRLTISKGASRNQVFLKIISVDTADTATYYCA
LRYYGYNSIMHYWGQGASVTVSS

TABLE 7
anti-human mature-Factor D-specific Antibody
VH Sequences (CDRs and FR regions, Kabat)

| Antibody | HC FR1 | HC CDR1 |
|---|---|---|
| 6G6 | QITLKESGPGILQSS | TSGMGVS |
| | QTLSLTCSFSGISLT | (SEQ ID NO: 25) |
| | (SEQ ID NO: 24) | |
| 14A11 | QITLKESGPGILQSS | TSGMGVS |
| | QTLSLTCSFSGVSLT | (SEQ ID NO: 25) |
| | (SEQ ID NO: 31) | |
| 27B3 | QVTLKESGPGILQSS | ISGMGVS |
| | QTLSLTCSFSGISLN | (SEQ ID NO: 33) |
| | (SEQ ID NO: 32) | |
| 58F5 | QVTLKESGPGILQSS | TSIMGVS |
| | QTLSLTCSFSGISLN | (SEQ ID NO: 38) |
| | (SEQ ID NO: 32) | |
| 49G3 | QVTLKESGPGILQSS | SSGMGVS |
| | QTLSLTCSFSGISLS | (SEQ ID NO: 43) |
| | (SEQ ID NO: 42) | |
| 10G1 | QVTLKESGPGILQSS | SSGMGVS |
| | QTLSLTCSFSGVSLS | (SEQ ID NO: 43) |
| | (SEQ ID NO: 45) | |
| Antibody | HC FR2 | HC CDR2 |
| 6G6 | WIRQPSGKGLEWLA | HIYWDDEKHYHPSLKS |
| | (SEQ ID NO: 26) | (SEQ ID NO: 27) |
| 14A11 | WIRQPSGKGLEWLA | HIYWDDEKHYHPSLKS |
| | (SEQ ID NO: 26) | (SEQ ID NO: 27) |
| 27B3 | WIRQPSGKGLEWLA | HIYWDDEKHYNPSLKR |
| | (SEQ ID NO: 26) | (SEQ ID NO: 34) |
| 58F5 | WIRQPSGKGLEWLA | HIYWDDEKHYNPSLKS |
| | (SEQ ID NO: 26) | (SEQ ID NO: 39) |
| 49G3 | WIRQPSGKGLEWLA | HIYWDDEKHYNPSLKS |
| | (SEQ ID NO: 26) | (SEQ ID NO: 39) |
| 10G1 | WIRQPSGKGLEWLA | HIYWDDEKHYNPSLKS |
| | (SEQ ID NO: 26) | (SEQ ID NO: 39) |
| Antibody | HC FR3 | HC CDR3 |
| 6G6 | RLTISKDASRNQVFFR | RYYGYRSFMDY |
| | ILSVDTADTATYYCAL | (SEQ ID NO: 29) |
| | (SEQ ID NO: 28) | |
| 14A11 | RLTISKDASRNQVFFR | RYYGYRSFMDY |
| | ILSVDTADTATYYCAL | (SEQ ID NO: 29) |
| | (SEQ ID NO: 28) | |
| 27B3 | RLTISKDASRNQVFFR | RYYGYGSIMDY |
| | ISSVDSADTATYYCAL | (SEQ ID NO: 36) |
| | SEQ ID NO: 35) | |
| 58F5 | RLTISKDASRNQVFLK | RYYGYNYVMHY |
| | IISVDTADTATYYCAL | (SEQ ID NO: 41) |
| | (SEQ ID NO: 40) | |
| 49G3 | RLTISKDASRNQIFLK | RYYGYNYVMHY |
| | IISVDTADTATYYCAL | (SEQ ID NO: 41) |
| | (SEQ ID NO: 44) | |
| 10G1 | RLTISKGASRNQVFLK | RYYGYNSIMHY |
| | IISVDTADTATYYCAL | (SEQ ID NO: 47) |
| | (SEQ ID NO: 46) | |
| Antibody | HC FR4 | |
| 6G6 | WGQGTSVTVSS | |
| | (SEQ ID NO: 30) | |
| 14A11 | WGQGTSVTVSS | |
| | (SEQ ID NO: 30) | |
| 27B3 | WGHGTSVTVSS | |
| | (SEQ ID NO: 37) | |
| 58F5 | WGQGTSVTVSS | |
| | (SEQ ID NO: 30) | |
| 49G3 | WGQGTSVTVSS | |
| | (SEQ ID NO: 30) | |
| 10G1 | WGQGASVTVSS | |
| | (SEQ ID NO: 48) | |

### Anti-Human Mature-Factor D-Specific Antibody Light Chain Variable Region (VL) Sequences

FIG. 7B shows an amino acid alignment of the light chain variable region (VL) sequences for the anti-human mature-Factor D-specific clones: 6G6_VK (SEQ ID NO:18), 14A11_VK: (SEQ ID NO:19), 27B3_VK: (SEQ ID NO:20), 58F5_VK: (SEQ ID NO:21), 49G3_VK: (SEQ ID NO:22), 10G1_VK: (SEQ ID NO:23).

Presented below is the light chain variable region (VL) sequence for each anti-human mature-factor-D-specific antibody. The Kabat CDRs are underlined. These regions are the same whether numbered by the Kabat or Chothia system.

6G6_VK:
SEQ ID NO: 18
DVLMTQSPLSLPVSLGDQASIFCRSNQSIVHSNGNTYFEWYLQKPGQSP
KLLIYKVSNRFSGVPDRFSGSGSGTDFTLRISRVEAEDLGVYYCFQGSH
VPPTFGGGTKLEIKR
14A11_VK:
SEQ ID NO: 19
DVLMTQSPLSLPVSLGDQASIFCRSNQSIVHSNGNTYFEWYLQKPGQSP
KLLIYKVSNRFSGVPDRFSGSGSGTDFTLRISRVEAEDLGIYYCFQGSH
VPPTFGGGTKLEIKR
27B3_VK:
SEQ ID NO: 20
DVLMTQTPLSLPVSLGDQASISCRSSQSIVHSNGNTYFEWYLQKPGQSP
KLLIYKVSNRFSGVPDRFSGSGSGTDFTLKISRVEAEDLGVYYCFQGSH
VPPTFGGGTKLEIKR
58F5_VK:
SEQ ID NO: 21
DVLMTQTPLSLPVSLGDQASISCRSSQSIVHSNGNTYLEWYLQKPGQSP
KLLIYKVSNRFSGVPDRFSGSGSGTDFTLKISRVEADDLGVYYCFQGSH
VPPTFGGGTKLEIKR
49G3_VK:
SEQ ID NO: 22
DVLMTQTPLSLPVSLGDQASISCRSSQSILHSNGNTYFEWYLQKPGQSP
KLLIYKVSNRFSGVPDRFSGSGSGTDFTLKISRVEAEDLGVYYCFQGSH
VPPTFGGGTKLEIKR
10G1_VK:
SEQ ID NO: 23
DVLMTQTPLSLPVSLGDQASISCRSSESIVHSNGNTYLEWYLQKPGQSP
KLLIYKVYNRFSGVPDRFSGSGSGTDFTLKISRVEAEDLGVYYCFQGSH
VPPTFGGGTKLEIKR

TABLE 8
anti-human mature-Factor D-specific Antibody VL
Sequences (CDRs and FR regions, Kabat and Chothia)

| Antibody | LC FR1 | LC CDR1 |
|---|---|---|
| 6G6 | DVLMTQSPLSLPVSLGDQASIFC | RSNQSIVHSNGNTYFE |
| | (SEQ ID NO: 49) | (SEQ ID NO: 50) |
| 14A11 | DVLMTQSPLSLPVSLGDQASIFC | RSNQSIVHSNGNTYFE |
| | (SEQ ID NO: 49) | (SEQ ID NO: 50) |
| 27B3 | DVLMTQTPLSLPVSLGDQASISC | RSSQSIVHSNGNTYFE |
| | (SEQ ID NO: 57) | (SEQ ID NO: 58) |
| 58F5 | DVLMTQTPLSLPVSLGDQASISC | RSSQSIVHSNGNTYLE |
| | (SEQ ID NO: 57) | (SEQ ID NO: 60) |
| 49G3 | DVLMTQTPLSLPVSLGDQASISC | RSSQSILHSNGNTYFE |
| | (SEQ ID NO: 57) | (SEQ ID NO: 62) |
| 10G1 | DVLMTQTPLSLPVSLGDQASISC | RSSESIVHSNGNTYLE |
| | (SEQ ID NO: 57) | (SEQ ID NO: 63) |
| Antibody | LC FR2 | LC CDR2 |
| 6G6 | WYLQKPGQSPKLLIY | KVSNRFS |
| | (SEQ ID NO: 51) | (SEQ ID NO: 52) |
| 14A11 | WYLQKPGQSPKLLIY | KVSNRFS |
| | (SEQ ID NO: 51) | (SEQ ID NO: 52) |
| 27B3 | WYLQKPGQSPKLLIY | KVSNRFS |
| | (SEQ ID NO: 51) | (SEQ ID NO: 52) |
| 58F5 | WYLQKPGQSPKLLIY | KVSNRFS |
| | (SEQ ID NO: 51) | (SEQ ID NO: 52) |
| 49G3 | WYLQKPGQSPKLLIY | KVSNRFS |
| | (SEQ ID NO: 51) | (SEQ ID NO: 52) |
| 10G1 | WYLQKPGQSPKLLIY | KVYNRFS |
| | (SEQ ID NO: 51) | (SEQ ID NO: 64) |
| Antibody | LC FR3 | LC CDR3 |
| 6G6 | GVPDRFSGSGSGTDFT | FQGSHVPPT |
| | LRISRVEAEDLGVYYC | (SEQ ID NO: 54) |
| | (SEQ ID NO: 53) | |
| 14A11 | GVPDRFSGSGSGTDFT | FQGSHVPPT |
| | LRISRVEAEDLGIYYC | (SEQ ID NO: 54) |
| | (SEQ ID NO: 56) | |
| 27B3 | GVPDRFSGSGSGTDFT | FQGSHVPPT |
| | LKISRVEAEDLGVYYC | (SEQ ID NO: 54) |
| | (SEQ ID NO: 59) | |
| 58F5 | GVPDRFSGSGSGTDFT | FQGSHVPPT |
| | LKISRVEADDLGVYYC | (SEQ ID NO: 54) |
| | (SEQ ID NO: 61) | |
| 49G3 | GVPDRFSGSGSGTDFT | FQGSHVPPT |
| | LKISRVEAEDLGVYYC | (SEQ ID NO: 54) |
| | (SEQ ID NO: 59) | |
| 10G1 | GVPDRFSGSGSGTDFT | FQGSHVPPT |
| | LKISRVEAEDLGVYYC | (SEQ ID NO: 54) |
| | (SEQ ID NO: 59) | |
| Antibody | LC FR4 | |
| 6G6 | FGGGTKLEIKR | |
| | (SEQ ID NO: 55) | |
| 14A11 | FGGGTKLEIKR | |
| | (SEQ ID NO: 55) | |
| 27B3 | FGGGTKLEIKR | |
| | (SEQ ID NO: 55) | |
| 58F5 | FGGGTKLEIKR | |
| | (SEQ ID NO: 55) | |
| 49G3 | FGGGTKLEIKR | |
| | (SEQ ID NO: 55) | |
| 10G1 | FGGGTKLEIKR | |
| | (SEQ ID NO: 55) | |

TABLE 9
Consensus Sequences for anti-human mature-Factor
D-specific HC CDRs:

| Antibody | Region | Sequence |
|---|---|---|
| 6G6 | HC-CDR1 | TSGMGVS (SEQ ID NO: 25) |
| 14A11 | HC-CDR1 | TSGMGVS (SEQ ID NO: 25) |
| 27B3 | HC-CDR1 | ISGMGVS (SEQ ID NO: 33) |
| 58F5 | HC-CDR1 | TSIMGVS (SEQ ID NO: 38) |
| 49G3 | HC-CDR1 | SSGMGVS (SEQ ID NO: 43) |
| 10G1 | HC-CDR1 | SSGMGVS (SEQ ID NO: 43) |
| Con- | HC-CDR1 | XSXMGVS (SEQ ID NO: 65) |
| sensus | | Wherein |
| | | X at position 1 is T, I or S; |
| | | X at position 3 is G or I |
| 6G6 | HC-CDR2 | HIYWDDEKHYHPSLKS (SEQ ID NO: 27) |
| 14A11 | HC-CDR2 | HIYWDDEKHYHPSLKS (SEQ ID NO: 27) |
| 27B3 | HC-CDR2 | HIYWDDEKHYNPSLKR (SEQ ID NO: 34) |
| 58F5 | HC-CDR2 | HIYWDDEKHYNPSLKS (SEQ ID NO: 39) |
| 49G3 | HC-CDR2 | HIYWDDEKHYNPSLKS (SEQ ID NO: 39) |
| 10G1 | HC-CDR2 | HIYWDDEKHYNPSLKS (SEQ ID NO: 39) |
| Con- | HC-CDR2 | HIYWDDEKHYXPSLKX (SEQ ID NO: 66) |
| sensus | | Wherein |
| | | X at position 11 is H or N; |
| | | X at position 16 is S or R |
| 6G6 | HC-CDR3 | RYYGYRSFMDY (SEQ ID NO: 29) |
| 14A11 | HC-CDR3 | RYYGYRSFMDY (SEQ ID NO: 29) |
| 27B3 | HC-CDR3 | RYYGYGSIMDY (SEQ ID NO: 36) |
| 58F5 | HC-CDR3 | RYYGYNYVMHY (SEQ ID NO: 41) |
| 49G3 | HC-CDR3 | RYYGYNYVMHY (SEQ ID NO: 41) |
| 10G1 | HC-CDR3 | RYYGYNSIMHY (SEQ ID NO: 47) |
| Con- | HC-CDR3 | RYYGYXXXMXY (SEQ ID NO: 67) |
| sensus | | Wherein |
| | | X at position 6 is R, G or N; |
| | | X at position 7 is S or Y; |
| | | X at position 8 is F, I or V; |
| | | X at position 10 is D or H |

TABLE 10
Consensus Sequences for mature-Factor D-specific
LC CDRs:

| Antibody | Region | Sequence |
|---|---|---|
| 6G6 | LC-CDR1 | RSNQSIVHSNGNTYFE (SEQ ID NO: 50) |
| 14A11 | LC-CDR1 | RSNQSIVHSNGNTYFE (SEQ ID NO: 50) |
| 27B3 | LC-CDR1 | RSSQSIVHSNGNTYFE (SEQ ID NO: 58) |
| 58F5 | LC-CDR1 | RSSQSIVHSNGNTYLE (SEQ ID NO: 60) |
| 49G3 | LC-CDR1 | RSSQSILHSNGNTYFE (SEQ ID NO: 62) |
| 10G1 | LC-CDR1 | RSSESIVHSNGNTYLE (SEQ ID NO: 63) |
| Con- | LC-CDR1 | RSXXSIXHSNGNTYXE (SEQ ID NO: 68) |
| sensus | | Wherein: |
| | | X at position 3 is N or S; |
| | | X at position 4 is Q or E; |
| | | X at position 7 is V or L; |
| | | X at position 15 is F or L |
| 6G6 | LC-CDR2 | KVSNRFS (SEQ ID NO: 52) |
| 14A11 | LC-CDR2 | KVSNRFS (SEQ ID NO: 52) |
| 27B3 | LC-CDR2 | KVSNRFS (SEQ ID NO: 52) |
| 58F5 | LC-CDR2 | KVSNRFS (SEQ ID NO: 52) |
| 49G3 | LC-CDR2 | KVSNRFS (SEQ ID NO: 52) |
| 10G1 | LC-CDR2 | KVYNRFS (SEQ ID NO: 64) |
| Con- | LC-CDR2 | KVXNRFS (SEQ ID NO: 69) |
| sensus | | Wherein: |
| | | X at position 3 is S or Y |
| 6G6 | LC-CDR3 | FQGSHVPPT (SEQ ID NO: 54) |
| 14A11 | LC-CDR3 | FQGSHVPPT (SEQ ID NO: 54) |
| 27B3 | LC-CDR3 | FQGSHVPPT (SEQ ID NO: 54) |
| 58F5 | LC-CDR3 | FQGSHVPPT (SEQ ID NO: 54) |
| 49G3 | LC-CDR3 | FQGSHVPPT (SEQ ID NO: 54) |
| 10G1 | LC-CDR3 | FQGSHVPPT (SEQ ID NO: 54) |
| Consensus | LC-CDR3 | FQGSHVPPT (SEQ ID NO: 54) |

SEQ ID NO: 70: human IgG4 constant region:
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 71: human IgG4 constant region with
S228P mutation
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
SEQ ID NO: 245: human IgG4 constant region with
S228P mutation and also a mutation that promotes
FcRn interactions at low pH
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVLHEALHSHYTQKSLSLSLGK
SEQ ID NO: 72: human IgK constant region
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC
SEQ ID NO: 218: mouse IgG2a constant region:
AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVS
EDDPDVQISWFVNNVEVHTAQTQTHREDYNSTLRVVSALPIQHQDWMSGK
EFKCKVNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNW
VERNSYSCSVVHEGLHNHHTTKSFSRTPGK
SEQ ID NO: 219: mouse IgK constant region:
ADAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKS
FNRNEC

### DNA Encoding Mouse Anti-Human Mature-Factor D-Specific Antibody mAb Heavy and Light Chains:

SEQ ID NO: 73: nucleic acid encoding 6G6 HC variable region
CAGATTACTCTGAAAGAGTCTGGCCCTGGGATATTGCAGTCCTCCCAGACCCTCA
GTCTGACTTGTTCTTTCTCTGGGATTTCACTGACTACTTCTGGTATGGGTGTGAGCTGGAT
TCGTCAGCCTTCAGGAAAGGGTCTGGAATGGCTGGCACACATTTATTGGGATGATGAGA
AACACTATCATCCATCCCTGAAGAGCCGGCTCACAATCTCCAAGGATGCCTCCAGAAAC
CAGGTTTTCTTCAGGATCCTTAGTGTGGACACTGCAGATACTGCCACATACTACTGTGCT
CTCCGTTACTACGGTTATAGGTCTTTTATGGACTACTGGGGTCAAGGAACCTCAGTCACC
GTCTCCTCA
SEQ ID NO: 74: nucleic acid encoding 14A11 HC variable region
CAGATTACTCTGAAAGAGTCTGGCCCTGGGATATTGCAGTCCTCCCAGACCCTCA
GTCTGACTTGTTCTTTCTCTGGGGTTTCACTGACTACTTCTGGTATGGGTGTGAGCTGGAT
TCGTCAGCCTTCAGGAAAGGGTCTGGAATGGCTGGCACACATTTATTGGGATGATGAGA
AACACTATCATCCATCCCTGAAGAGCCGGCTCACAATCTCCAAGGATGCCTCCAGAAAC
CAGGTTTTCTTCAGGATCCTTAGTGTGGACACTGCAGATACTGCCACATATTACTGTGCT
CTCCGTTACTACGGTTATAGGTCTTTTATGGACTATTGGGGTCAAGGAACCTCAGTCACC
GTCTCCTCA
SEQ ID NO: 75: nucleic acid encoding 27B3 HC variable region
CAGGTTACTCTGAAAGAGTCTGGCCCTGGGATATTGCAGTCCTCCCAGACCCTCA
GTCTGACTTGTTCTTTCTCTGGGATTTCACTGAATATTTCCGGTATGGGTGTGAGCTGGAT
TCGTCAGCCTTCAGGAAAGGGTCTGGAGTGGCTGGCACACATTTACTGGGATGATGAAA
AACACTATAATCCATCCCTGAAGAGACGGCTCACTATCTCCAAGGATGCCTCCAGAAAC
CAGGTTTTCTTCAGGATCAGTAGTGTGGACTCTGCAGATACTGCCACATACTACTGTGCG
CTCCGTTACTACGGTTATGGTTCTATTATGGACTATTGGGGTCATGGAACCTCAGTCACC
GTCTCCTCA
SEQ ID NO: 76: nucleic acid encoding 58F5 HC variable region
CAGGTTACTCTGAAAGAGTCTGGCCCTGGGATATTGCAGTCCTCCCAGACCCTCA
GTCTGACTTGTTCTTTCTCTGGGATTTCATTGAATACTTCTATTATGGGTGTGAGCTGGAT
TCGTCAGCCTTCAGGAAAGGGTCTGGAGTGGCTGGCACACATTTACTGGGATGATGAGA
AACACTATAACCCATCCCTGAAGAGCCGACTCACAATCTCCAAGGATGCCTCCAGAAAC
CAGGTATTCCTCAAGATCATTAGTGTGGACACTGCAGATACTGCCACATACTACTGTGCT
CTCCGTTACTACGGTTATAACTATGTTATGCACTACTGGGGTCAAGGAACCTCAGTCACC
GTCTCCTCA
SEQ ID NO: 77: nucleic acid encoding 49G3 HC variable region
CAGGTTACTCTGAAAGAGTCTGGCCCTGGGATATTGCAGTCCTCCCAGACCCTCA
GTCTGACTTGTTCTTTCTCTGGGATTTCACTGAGTTCTTCTGGTATGGGTGTGAGCTGGAT
TCGTCAGCCTTCAGGAAAGGGTCTGGAGTGGCTGGCACACATTTACTGGGATGATGAGA
AACACTATAACCCATCCCTGAAGAGCCGGCTCACAATCTCCAAGGATGCCTCCAGAAAC
CAGATATTCCTCAAGATCATTAGTGTGGACACTGCAGATACTGCCACATATTATTGTGCT
CTCCGTTACTACGGTTATAACTATGTTATGCACTACTGGGGTCAAGGAACCTCAGTCACC
GTCTCCTCA
SEQ ID NO: 78: nucleic acid encoding 10G1 HC variable region
CAGGTTACTCTGAAAGAGTCTGGCCCTGGGATATTGCAGTCCTCCCAGACCCTCA
GTCTGACTTGTTCTTTCTCTGGGGTTTCACTGAGTTCTTCTGGTATGGGTGTGAGCTGGAT
TCGTCAGCCTTCAGGAAAGGGTCTGGAGTGGCTGGCACACATTTACTGGGATGATGAGA
AACACTATAACCCATCCCTGAAGAGCCGGCTCACAATCTCCAAGGGTGCCTCCAGAAAC
CAGGTCTTCCTCAAGATCATTAGTGTGGACACTGCAGATACTGCCACATACTACTGTGCT
CTCCGTTACTACGGTTATAACTCTATTATGCACTACTGGGGTCAAGGAGCCTCAGTCACC
GTCTCCTCA
SEQ ID NO: 79: nucleic acid encoding 6G6 LC variable region
GATGTTTTGATGACCCAATCTCCACTCTCCCTGCCTGTCAGTCTTGGAGATCAAG
CCTCCATCTTTTGCAGATCTAATCAGAGCATTGTACATAGTAATGGAAACACCTATTTCG
AATGGTACCTGCAGAAACCAGGCCAGTCTCCAAAGCTCCTGATCTACAAAGTTTCCAAC
CGATTTTCTGGGGTCCCAGACAGGTTCAGTGGCAGTGGATCAGGGACAGATTTCACACT
CAGGATCAGCAGAGTGGAGGCTGAGGATCTGGGAGTTTATTACTGCTTTCAAGGTTCAC
ATGTTCCTCCGACGTTCGGTGGAGGCACCAAGCTGGAAATCAAACGG
SEQ ID NO: 80: nucleic acid encoding 14A11 LC variable region
GATGTTTTGATGACCCAATCTCCACTCTCCCTGCCTGTCAGTCTTGGAGATCAAG
CCTCCATCTTTTGCAGATCTAATCAGAGCATTGTTCATAGTAATGGAAACACCTATTTCG
AATGGTACCTGCAGAAACCAGGCCAGTCTCCAAAGCTCCTGATCTACAAAGTTTCCAAC
CGATTTTCTGGGGTCCCAGACAGGTTCAGTGGCAGTGGATCAGGGACAGATTTCACACT
CAGGATCAGCAGAGTGGAGGCTGAGGATCTGGGAATTTATTACTGCTTTCAAGGTTCAC
ATGTTCCTCCGACGTTCGGTGGAGGCACCAAGCTGGAAATCAAACGG
SEQ ID NO: 81: nucleic acid encoding 27B3 LC variable region
GATGTTTTGATGACCCAAACTCCACTCTCCCTGCCTGTCAGTCTTGGAGATCAAG
CCTCCATCTCTTGCAGATCTAGTCAGAGCATTGTTCATAGTAATGGAAATACCTATTTTG
AATGGTACCTCCAGAAACCAGGCCAGTCTCCAAAGCTCCTGATCTACAAGGTTTCCAAC
CGATTTTCTGGGGTCCCAGACAGGTTCAGTGGCAGTGGATCAGGGACAGATTTCACACT
CAAGATCAGCAGAGTGGAGGCTGAGGATCTGGGAGTTTATTACTGCTTTCAAGGTTCAC
ATGTTCCTCCGACGTTCGGTGGAGGCACCAAGCTGGAGATCAAACGG
SEQ ID NO: 82: nucleic acid encoding 58F5 LC variable region
GATGTTTTGATGACCCAAACTCCACTCTCCCTGCCTGTCAGTCTTGGAGATCAAG
CCTCCATCTCTTGCAGATCTAGTCAGAGCATTGTACATAGTAATGGAAACACCTATTTAG
AATGGTACCTGCAGAAACCAGGCCAGTCTCCAAAGCTCCTGATCTACAAAGTTTCCAAC
CGATTTTCTGGGGTCCCAGACAGATTCAGTGGCAGTGGATCAGGGACAGATTTCACACT
CAAGATCAGCAGAGTGGAGGCTGATGATCTGGGAGTTTATTACTGCTTTCAAGGTTCAC
ATGTTCCTCCGACGTTCGGTGGAGGCACCAAGCTGGAAATCAAACGG
SEQ ID NO: 83: nucleic acid encoding 49G3 LC variable region
GATGTTTTGATGACCCAAACTCCACTCTCCCTGCCTGTCAGTCTTGGAGATCAAG
CCTCCATCTCTTGCAGATCTAGTCAGAGCATTCTACATAGTAATGGAAACACCTATTTTG
AATGGTACCTGCAGAAACCAGGCCAGTCTCCAAAGCTCCTGATCTACAAAGTTTCCAAC
CGATTTTCTGGGGTCCCAGACAGGTTCAGTGGCAGTGGATCAGGGACAGATTTCACACT
CAAGATCAGCAGAGTGGAGGCTGAGGATCTGGGAGTTTATTACTGCTTTCAAGGTTCAC
ATGTTCCTCCGACGTTCGGTGGAGGCACCAAGCTGGAAATCAAACGG
SEQ ID NO: 84: nucleic acid encoding 10G1 LC variable region
GATGTTTTGATGACCCAAACTCCACTCTCCCTGCCTGTCAGTCTTGGAGATCAAG
CCTCCATCTCTTGCAGATCTAGTGAGAGCATTGTACATAGTAATGGAAACACCTATTTAG
AATGGTACCTGCAGAAACCAGGCCAGTCTCCAAAGCTCCTGATCTACAAAGTTTACAAC
CGATTTTCTGGGGTCCCAGACAGGTTCAGTGGCAGTGGATCAGGGACAGATTTCACACT
CAAGATCAGCAGAGTGGAGGCTGAGGATCTGGGAGTTTATTACTGCTTTCAAGGTTCAC
ATGTTCCTCCGACGTTCGGTGGAGGCACCAAGCTGGAGATCAAACGG

### Example 3

This Example describes the functional characterization of recombinant purified anti-human mature factor D-specific antibodies in several in vitro assays.

### Background/Rationale:

This Example describes the functional characterization of recombinant anti-human mature factor D-specific monoclonal antibodies that were generated as described in Examples 1 and 2 for binding to human mature Factor D and binding to human pro-Factor D.

### Methods:

### Sandwich ELISA Assay

Purified, recombinant anti-human mature-Factor D-specific antibodies 6G6, 14A11, 10G1, 49G3, 27B3 and 58F5 (human IgG4 Fc) that were generated as described in Examples 1 and 2 were tested in a sandwich ELISA format as detection antibodies. Recombinant human pro-factor D protein (SEQ ID NO:2), referred to as “pro” and recombinant human mature-factor D protein (SEQ ID NO:3), referred to as “mature” were captured by plate-bound goat anti-human CFD polyclonal AF1824 (R&D systems). Purified recombinant antibodies 6G6, 14A11, 10G1, 49G3, 27B3 and 58F5 as well as a control human IgG4 were added to the washed plate and incubated. An HRP-tagged anti-mouse secondary antibody followed by TMB substrate was used to develop the assay.

### Results:

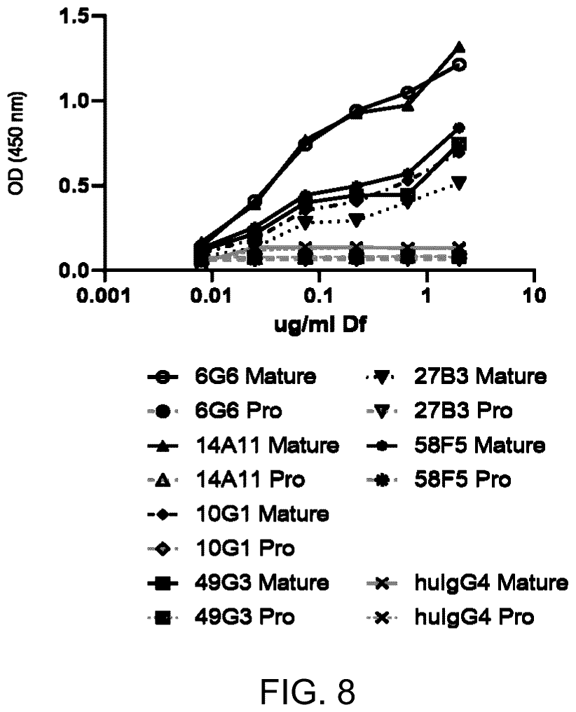

FIG. 8 graphically illustrates the detection of recombinant human pro-Factor D or mature-Factor D with numerous candidate anti-human mature-Factor-D-specific antibodies. As shown in FIG. 8, all the purified antibodies tested, namely 6G6, 14A11, 10G1, 49G3, 27B3 and 58F5, were found to be specific for the mature form of Factor D as compared to pro-Factor D in an ELISA assay format.

### Affinity Assay

Affinities of candidate antibodies to human mature-Factor-D versus human pro-Factor D were determined as follows.

Association and dissociation constants were determined by Octet Fortebio. 20 nM recombinant human IgG4 candidate antibodies 6G6, 14A11, 10G1, 49G3, 27B3 and 58F5 were loaded onto anti-human sensors and allowed to associate and dissociate over 5 minute time periods with recombinant human mature-Factor-D (111 nM) or recombinant human pro-Factor D (111 nM). The results are shown below in TABLE 11.

TABLE 11
Affinities of Candidate Antibodies to human
mature-factor D versus human pro-factor D

| Candidate | KD (M) mature- | KD (M) |
|---|---|---|
| antibody | Factor D | pro-Factor D |
| 6G6 | 1.43E−08 | NC |
| 14A11 | 9.77E−09 | NC |
| 10G1 | 7.31E−10 | NC |
| 49G3 | 2.30E−09 | NC |
| 58F5 | <1.0E−12 | NC |
| 27B3 | 2.41E−08 | NC |
| blank | NC | NC |

NC = instrument software could not calculate

Based on the results described in this Example, the antibodies 6G6 and 14A11 were chosen for further analysis and development due to their superior sensitivity and specificity for mature human factor D versus human pro-factor D.

### Conclusion:

As described in Example 1-3, the inventors have generated mature-Factor D-specific monoclonal antibodies that specifically bind to mature Factor D and do not bind to Pro-Factor D. As further described in Examples 10-12, the level of mature Factor D correlates with alternative pathway activity, therefore, mature Factor D-specific monoclonal antibodies may be used to measure the level of mature Factor D as a surrogate endpoint in a diagnostic assay to assess the level of alternative pathway activation in a mammalian subject. As further described herein in Example 12, the mature-Factor D-specific monoclonal antibodies may be used as a pharmacodynamic (PD) measurement of MASP-3 inhibition in a subject treated with a MASP-3 inhibitor, which may be used to determine efficacious dosing of a MASP-3 inhibitor.

### Example 4

This Example describes the generation of monoclonal antibodies raised against mature human factor D and selected for the ability to detect both mature- and pro-Factor D proteins (i.e., antibodies that bind to an epitope of Factor D that is common to both mature and pro-Factor D proteins).

### Background/Rationale:

This Example describes the generation of anti-human factor D antibodies capable of binding both the pro and mature form of human Factor D. The antibodies described in this Example bind to both pro-factor D and mature factor D and are useful as coating antibodies in an ELISA assay to assess the status of Factor D in a biological sample.

### Methods:

Immunization with the Mature Factor D Antigen

C57BL/6, MASP-1/3 knockout mice were immunized with recombinant human mature Factor D-His tagged protein. The mice were immunized two times, subcutaneously, with 50 μL of adjuvant-emulsions of protein (50-100 μg total protein per injection). Serum samples from the immunized mice were prepared from tail bleeds and tested by ELISA for the presence of antigen-specific antibodies capable of binding to plate-immobilized recombinant human pro-factor D (SEQ ID NO:2) and recombinant human mature Factor D (SEQ ID NO:3), both strep-tagged, as follows.

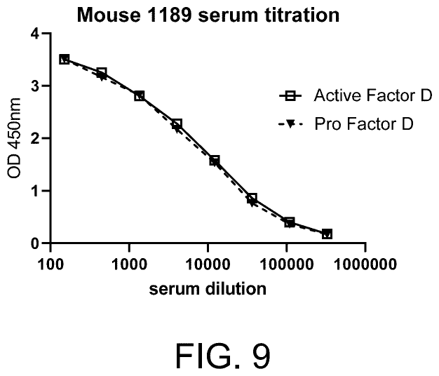

Recombinant human Pro-CFD-Strep tagged or recombinant human mature CFD-Strep tagged were immobilized on Maxisorp™ ELISA plates at 1 μg/mL in PBS, 100 μL/well, overnight at 4° C. Plate wells were washed three times with 300 μL PBS containing 0.05% Tween 20 (PBST), blocked for 1 hour at room temperature with 250 μL PBS containing 1% BSA and washed again. Serum from representative mouse #1189 was diluted in PBST and allowed to bind for 1 hour at room temperature, then washed three times in PBST. An HRP-labeled goat anti-mouse IgG Fc antibody was then applied (100 μL/well), allowed to bind for 1 hour at room temperature, and then washed three times with PBST. TMB substrate (ThermoFisher) (100 μL/well) was then applied and incubated for 5 minutes at room temperature. The reaction was then stopped with 1N H₂SO₄ (50 μL/well). The plate was read for optical density at 450 nM with a Biotek™ ELISA plate reader. The results from the serum from a representative mouse (mouse #1189) are shown in FIG. 9.

### Results:

FIG. 9 graphically illustrates a titration of the serum of a representative mouse #1189 after immunization with human mature Factor D in the presence of recombinant mature Factor D or recombinant pro-Factor D. As shown in FIG. 9, the serum from representative mouse #1189 contains antibodies capable of binding to both mature Factor D and pro-Factor D. Based on these results, mouse #1189 was selected for hybridoma fusion which was carried out as follows.

A final injection of 50 μg total protein in 50 μL was delivered subcutaneously to mouse #1189 four days prior to hybridoma fusion. Three days prior to the fusion, mouse #1189 was treated subcutaneously with 50 μg of an anti-CD40 agonizing antibody in PBS (R&D Systems, Minneapolis, MN) to increase B cells numbers (see Rycyzyn et al., Hybridoma 27:25-30, 2008). The mouse was sacrificed, and the spleen cells were harvested and fused to a selected murine myeloma cell line P3/NSI/1-AG4-1 (NS-1) (ATCC No. TIB18) using 50% polyethylene glycol or 50% polyethylene glycol plus 10% DMSO. The fusions generated hybridoma cells which were plated in 96 well Nunc tissue culture treated plates containing HAT (hypoxanthine, aminopterin and thymidine) medium to inhibit proliferation of non-fused cells, myeloma hybrids and spleen hybrids. Hybridoma wells were fed by replacement of 80% of media with fresh medium containing HAT supplement.

After hybridoma selection, the culture supernatants were assayed for binding to recombinant human pro-factor D and mature factor D as follows.

### Hybridoma Screening

Hybridoma supernatants were first screened for binding to immobilized recombinant human mature Factor D-Streptavidin. 54 hybridomas were identified and were then tested for their ability to bind to recombinant human pro-Factor D-Strep when captured by a polyclonal goat anti-human factor D antibody AF1824 (R&D Systems) as follows. Hybridoma supernatants were diluted two-fold in PBS, 0.05% Tween 20 (PBST). Supernatant from NS-1 myeloma cell line (NS-1 sup) was included as a control. ELISA plates were coated with polyclonal anti-human factor D antibody AF1824. Out of 54 hybridomas tested, the supernatants from 5 hybridomas (3C5, 30H2, 11H1, 12H10 and 7H2) showed equal binding affinity to recombinant human pro-factor D and recombinant human mature Factor D and were selected for DNA cloning and recombinant antibody production, as further described in Example 5.

### Example 5

This Example describes the cloning and sequence analysis of anti-human Factor D antibodies that bind to both pro-Factor D and mature-Factor D.

### Background/Rationale:

This Example describes the cloning and sequence analysis of antibodies produced by the hybridomas selected for the ability to detect both mature- and pro-Factor D proteins (i.e., clones 3C5, 30H2, 11H1, 12H10 and 7H2 that bind to an epitope of Factor D that is common to both mature and pro-Factor D proteins) that were generated as described in Example 4.

### Methods:

### Cloning and Purification of Recombinant Antibodies:

Hybridoma clones 3C5, 30H2, 11H1, 12H10 and 7H2 were generated and selected for the ability to detect both mature- and pro-Factor D proteins as described in Example 4. These hybridomas were subcloned by serial dilution methods. The heavy chain and light chain variable regions were cloned using RT-PCR and were sequenced. Antibody-encoding sequences were amplified from total RNA with isotype-specific reverse primers using the SMARTer™ RACE 5′/3′ kit (Takara Bio). After verifying the sequences, the variable (V) regions were re-amplified with designed cloning primers and cloned into expression vectors carrying either the human IgG4 heavy chain (SEQ ID NO:71) and kappa light chain (SEQ ID NO: 72) constant regions or the mouse IgG2a (SEQ ID NO:218) and kappa light chain (SEQ ID NO: 219) constant regions using the In-Fusion HD™ cloning kit (Clontech). The expression constructs were co-transfected transiently into Expi293 cells (Life Technologies), and after 5 days of culture, secreted recombinant antibodies were purified from supernatants by protein A chromatography.

The sequences of the heavy chain variable regions and light chain variable regions are shown in FIGS. 10A and 10B, respectively (“SIN”=“SEQ ID NO:” in FIG. 10A and FIG. 10B), and are included below. The complementarity determining regions (CDRs) and framework regions (FRs) of each are provided in TABLES 12-13 below.

### Anti-Human Factor D (C-Term) Antibody Heavy Chain Variable Regions

FIG. 10A shows an amino acid alignment of the heavy chain variable region (VH) sequences for the anti-human Factor D clones: 3C5_VH (SEQ ID NO:85), 30H2_VH (SEQ ID NO:85), 11H1_VH (SEQ ID NO:86), 12H10_VH (SEQ ID NO:87), and 7H2_VH (SEQ ID NO:88).

Presented below is the heavy chain variable region (VH) sequence for each anti-human factor-D antibody. The Kabat CDRs are underlined.

3C5 VH:
SEQ ID NO: 85
EVKLVESGGGLVQPGGSLKLSCATSGFTFSDYGMAWVRQAPGKGPEWVA
FISNLAYSFYYVDIVMGRFTISRENAKNTLYLEMSSLRSEDTAMYYCAR
VGLYGNFFMDYWGQGTSVTVSS
30H2 VH:
SEQ ID NO: 85
EVKLVESGGGLVQPGGSLKLSCATSGFTFSDYGMAWVRQAPGKGPEWVA
FISNLAYSFYYVDIVMGRFTISRENAKNTLYLEMSSLRSEDTAMYYCAR
VGLYGNFFMDYWGQGTSVTVSS
11H1 VH:
SEQ ID NO: 86
EVQLVESGGGLVQPKGSLKLSCAASGFSFNTYAMNWVRQAPGKGLEWVA
RIRSKSNNYATHYADSVKDRFTISRDDSESMLYLQMNNLKTEDTAMYYC
VRQGYYWYFDVWGTGTTVTVSS
12H10 VH:
SEQ ID NO: 87
EVQLVESGGGLVQPKGSLKLSCAASGFSFNTYAMNWVRQAPGKGLEWVA
RIRSKSNNYATYYADSVKDRFTISRDDSESMLYLQMNNLKTEDTAMYYC
VRHGYYWYFDVWGTGTTVTVSS
7H2 VH:
SEQ ID NO: 88
EVQVVESGGGLVRPKGSLKLSCAASGFSFNTYAMNWVRQAPGKGLEWVA
RIRSKSNNYATYYADSVKDRFTISRDDSESMLSLQMNNLKTEDTAMYYC
VRQGYYWYFDVWGTGTTVTVSS

TABLE 12
anti-human Factor D (C-term) Antibody VH Sequences
(CDRs and FR regions, Kabat)

| Antibody | HC FR1 | HC CDR1 |
|---|---|---|
| 3C5 | EVKLVESGGGLVQPG | DYGMA |
| | GSLKLSCATSGFTFS | (SEQ ID NO: 95) |
| | (SEQ ID NO: 94) | |
| 30H2 | EVKLVESGGGLVQPG | DYGMA |
| | GSLKLSCATSGFTFS | (SEQ ID NO: 95) |
| | (SEQ ID NO: 94) | |
| 11H1 | EVQLVESGGGLVQPK | TYAMN |
| | GSLKLSCAASGFSFN | (SEQ ID NO: 101) |
| | (SEQ ID NO: 100) | |
| 12H10 | EVQLVESGGGLVQPK | TYAMN |
| | GSLKLSCAASGFSFN | (SEQ ID NO: 101) |
| | (SEQ ID NO: 100) | |
| 7H2 | EVQVVESGGGLVRPK | TYAMN |
| | GSLKLSCAASGFSFN | (SEQ ID NO: 101) |
| | (SEQ ID NO: 109) | |
| Antibody | HC FR2 | HC CDR2 |
| 3C5 | WVRQAPGKGPEWVA | FISNLAYSFYYVDIVMG |
| | (SEQ ID NO: 96) | (SEQ ID NO: 97) |
| 30H2 | WVRQAPGKGPEWVA | FISNLAYSFYYVDIVMG |
| | (SEQ ID NO: 96) | (SEQ ID NO: 97) |
| 11H1 | WVRQAPGKGLEWVA | RIRSKSNNYATHYADSVKD |
| | (SEQ ID NO: 102) | (SEQ ID NO: 103) |
| 12H10 | WVRQAPGKGLEWVA | RIRSKSNNYATYYADSVKD |
| | (SEQ ID NO: 102) | (SEQ ID NO: 107) |
| 7H2 | WVRQAPGKGLEWVA | RIRSKSNNYATYYADSVKD |
| | (SEQ ID NO: 102) | (SEQ ID NO: 107) |
| Antibody | HC FR3 | HC CDR3 |
| 3C5 | RFTISRENAKNTLYLE | VGLYGNFFMDY |
| | MSSLRSEDTAMYYCAR | (SEQ ID NO: 99) |
| | (SEQ ID NO: 98) | |
| 30H2 | RFTISRENAKNTLYLE | VGLYGNFFMDY |
| | MSSLRSEDTAMYYCAR | (SEQ ID NO: 99) |
| | (SEQ ID NO: 98) | |
| 11H1 | RFTISRDDSESMLYLQ | QGYYWYFDV |
| | MNNLKTEDTAMYYCVR | (SEQ ID NO: 105) |
| | (SEQ ID NO: 104) | |
| 12H10 | RFTISRDDSESMLYLQ | HGYYWYFDV |
| | MNNLKTEDTAMYYCVR | (SEQ ID NO: 108) |
| | (SEQ ID NO: 104) | |
| 7H2 | RFTISRDDSESMLSLQ | QGYYWYFDV |
| | MNNLKTEDTAMYYCVR | (SEQ ID NO: 105) |
| | (SEQ ID NO: 246) | |
| Antibody | HC FR4 | |
| 3C5 | WGQGTSVTVSS | |
| | (SEQ ID NO: 30) | |
| 30H2 | WGQGTSVTVSS | |
| | (SEQ ID NO: 30) | |
| 11H1 | WGTGTTVTVSS | |
| | (SEQ ID NO: 106) | |
| 12H10 | WGTGTTVTVSS | |
| | (SEQ ID NO: 106) | |
| 7H2 | WGTGTTVTVSS | |
| | (SEQ ID NO: 106) | |

### Anti-Human Factor D Antibody Light Chain Variable Regions:

FIG. 10B shows an amino acid alignment of the light chain variable region (VL) sequences for the anti-human Factor D clones: 3C5_VL (SEQ ID NO:89), 30H2_VL (SEQ ID NO: 90), 11H1_VL (SEQ ID NO:91), 12H10_VL (SEQ ID NO:92) and 7H2_VL (SEQ ID NO: 93).

Presented below are the light chain variable region (VL) sequences for the anti-human Factor D antibodies. The Kabat CDRs are underlined. These regions are the same whether numbered by the Kabat or Chothia system.

3C5_VL:
SEQ ID NO: 89
DIQMNQSPSSLSASLGDTITITCHASQNINVWLSWYQQKPGNIPELLIY
KASNLHTGVPSRFSGNRSGTSFTLTISSLQPEDIGTYFCQQGQSYPLTF
GAGTKLELRR
30H2_VL:
SEQ ID NO: 90
DIQMNQSPSSLSASLGDTITITCHASQNINVWLSWYQQKPGNIPELLIY
KASNLHTGVPSRFSGNRSGTSFTLTISSLQPEDIGTYFCQQGQSYPLTF
GAGTKLEIKR
11H1_VL:
SEQ ID NO: 91
DVLMTQTPLSLPVSLGDQASISCRSSQSIVHSNGNTYLEWYLQKPGQSP
KLLIYTVSNRFSGVPDRFSGSGSGTDFTLKISRVEAEDLGVYYCFQGSH
VPWTFGGGTKLEIKR
12H10_VL:
SEQ ID NO: 92
DVLMTQTPLSLPVSLGDQASISCRSSQSIVHSDGNTYLEWYLQKPGQSP
KLLIYRVSNRFSGVPDRFSGSGSGTDFTLKISRVEAEDLGVYYCFQGSH
VPYTFGGGTKLEIKR
7H2_VL:
SEQ ID NO: 93
DVLMTQTPLSLPVSLGDQASISCRSSQSIVHSNGNTYLEWYLQKPGQSP
KLLIYTVSNRFSGVPDRFRGSGSGTDFTLKISRVEAEDLGVYYCFQGSH
VPWTFGGGTKLEIKR

TABLE 13
anti-human Factor D (C-term) Antibody VL Sequences
(CDRs and FR regions, Kabat and Chothia)

| Antibody | LC FR1 | LC CDR1 |
|---|---|---|
| 3C5 | DIQMNQSPSSLSASLGDTI | HASQNINVWLS |
| | TITC | (SEQ ID NO: 111) |
| | (SEQ ID NO: 110) | |
| 30H2 | DIQMNQSPSSLSASLGDTI | HASQNINVWLS |
| | TITC | (SEQ ID NO: 111) |
| | (SEQ ID NO: 110) | |
| 11H1 | DVLMTQTPLSLPVSLGDQA | RSSQSIVHSNGNTYLE |
| | SISC | (SEQ ID NO: 60) |
| | (SEQ ID NO: 118) | |
| 12H10 | DVLMTQTPLSLPVSLGDQA | RSSQSIVHSDGNTYLE |
| | SISC | (SEQ ID NO: 123) |
| | (SEQ ID NO: 118) | |
| 7H2 | DVLMTQTPLSLPVSLGDQA | RSSQSIVHSNGNTYLE |
| | SISC | (SEQ ID NO: 60) |
| | (SEQ ID NO: 118) | |
| Antibody | LC FR2 | LC CDR2 |
| 3C5 | WYQQKPGNIPELLIY | KASNLHT |
| | (SEQ ID NO: 112) | (SEQ ID NO: 113) |
| 30H2 | WYQQKPGNIPELLIY | KASNLHT |
| | (SEQ ID NO: 112) | (SEQ ID NO: 113) |
| 11H1 | WYLQKPGQSPKLLIY | TVSNRFS |
| | (SEQ ID NO: 51) | (SEQ ID NO: 119) |
| 12H10 | WYLQKPGQSPKLLIY | RVSNRFS |
| | (SEQ ID NO: 51) | (SEQ ID NO: 124) |
| 7H2 | WYLQKPGQSPKLLIY | TVSNRFS |
| | (SEQ ID NO: 51) | (SEQ ID NO: 119) |
| Antibody | LC FR3 | LC CDR3 |
| 3C5 | GVPSRFSGNRSGTSFT | QQGQSYPLT |
| | LTISSLQPEDIGTYFC | (SEQ ID NO: 115) |
| | (SEQ ID NO: 114) | |
| 30H2 | GVPSRFSGNRSGTSFT | QQGQSYPLT |
| | LTISSLQPEDIGTYFC | (SEQ ID NO: 115) |
| | (SEQ ID NO: 114) | |
| 11H1 | GVPDRFSGSGSGTDFT | FQGSHVPWT |
| | LKISRVEAEDLGVYYC | (SEQ ID NO: 121) |
| | (SEQ ID NO: 120) | |
| 12H10 | GVPDRFSGSGSGTDFT | FQGSHVPYT |
| | LKISRVEAEDLGVYYC | (SEQ ID NO: 125) |
| | (SEQ ID NO: 120) | |
| 7H2 | GVPDRFRGSGSGTDFT | FQGSHVPWT |
| | LKISRVEAEDLGVYYC | (SEQ ID NO: 121) |
| | (SEQ ID NO: 126) | |
| Antibody | LC FR4 | |
| 3C5 | FGAGTKLELRR | |
| | (SEQ ID NO: 116) | |
| 30H2 | FGAGTKLEIKR | |
| | (SEQ ID NO: 117) | |
| 11H1 | FGGGTKLEIKR | |
| | (SEQ ID NO: 122) | |
| 12H10 | FGGGTKLEIKR | |
| | (SEQ ID NO: 122) | |
| 7H2 | FGGGTKLEIKR | |
| | (SEQ ID NO: 122) | |

DNA Encoding Mouse Anti-Human Factor D Antibodies (that Bind to Both Pro- and Mature-Factor D) Heavy and Light Chains:

SEQ ID NO: 127: 3C5_VH
GAGGTGAAGCTGGTGGAGTCTGGGGGAGGCTTAGTGCAGCCTGGAGGGT
CCCTGAAACTCTCCTGTGCAACCTCTGGATTCACTTTCAGTGACTACGG
AATGGCGTGGGTTCGACAGGCTCCAGGGAAGGGGCCTGAGTGGGTAGCA
TTCATTAGTAATTTGGCATATAGTTTCTACTATGTAGACATTGTGATGG
GCCGATTCACCATCTCTAGAGAGAATGCCAAGAACACCCTGTACCTGGA
AATGAGCAGTCTGAGGTCTGAGGACACGGCCATGTATTACTGTGCAAGA
GTGGGGCTCTATGGTAACTTTTTTATGGACTACTGGGGTCAAGGAACCT
CAGTCACCGTCTCCTCA
SEQ ID NO: 127: 30H2_VH
GAGGTGAAGCTGGTGGAGTCTGGGGGAGGCTTAGTGCAGCCTGGAGGGT
CCCTGAAACTCTCCTGTGCAACCTCTGGATTCACTTTCAGTGACTACGG
AATGGCGTGGGTTCGACAGGCTCCAGGGAAGGGGCCTGAGTGGGTAGCA
TTCATTAGTAATTTGGCATATAGTTTCTACTATGTAGACATTGTGATGG
GCCGATTCACCATCTCTAGAGAGAATGCCAAGAACACCCTGTACCTGGA
AATGAGCAGTCTGAGGTCTGAGGACACGGCCATGTATTACTGTGCAAGA
GTGGGGCTCTATGGTAACTTTTTTATGGACTACTGGGGTCAAGGAACCT
CAGTCACCGTCTCCTCA
SEQ ID NO: 128: 11H1_VH
GAGGTGCAGCTTGTTGAGTCTGGTGGAGGATTGGTGCAGCCTAAAGGGT
CATTGAAACTCTCATGTGCAGCCTCTGGATTCAGCTTCAATACCTACGC
CATGAACTGGGTCCGCCAGGCTCCAGGAAAGGGTTTGGAATGGGTTGCT
CGCATAAGAAGTAAAAGTAATAATTATGCAACACATTATGCCGATTCAG
TGAAAGACAGATTCACCATCTCCAGAGATGATTCAGAAAGCATGCTCTA
TCTGCAAATGAACAACTTGAAAACTGAGGACACAGCCATGTATTACTGT
GTGAGACAGGGTTACTACTGGTACTTCGATGTCTGGGGCACAGGGACCA
CGGTCACCGTCTCCTCA
SEQ ID NO: 129: 12H10_VH
GAGGTGCAGCTTGTTGAGTCTGGTGGAGGATTGGTGCAGCCTAAAGGGT
CATTGAAACTCTCATGTGCAGCCTCTGGATTCAGCTTCAATACCTACGC
CATGAACTGGGTCCGCCAGGCTCCAGGAAAGGGTTTGGAATGGGTTGCT
CGCATAAGAAGTAAAAGTAATAATTATGCAACATATTATGCCGATTCAG
TGAAAGACAGATTCACCATCTCCAGAGATGATTCAGAAAGCATGCTCTA
TCTGCAAATGAACAACTTGAAAACTGAGGACACAGCCATGTATTACTGT
GTGAGACATGGTTACTACTGGTACTTCGATGTCTGGGGCACAGGGACCA
CGGTCACCGTCTCCTCA
SEQ ID NO: 130: 7H2_VH
GAGGTGCAGGTTGTTGAGTCTGGTGGAGGATTGGTGCGGCCTAAAGGGT
CATTGAAACTCTCATGTGCAGCCTCTGGATTCAGCTTCAATACCTACGC
CATGAACTGGGTCCGCCAGGCTCCAGGAAAGGGTTTGGAATGGGTTGCT
CGCATAAGAAGTAAAAGTAATAATTATGCAACATATTATGCCGATTCAG
TGAAAGACAGATTCACCATCTCCAGAGATGATTCAGAAAGCATGCTCTC
TCTGCAAATGAACAACTTGAAAACTGAGGACACAGCCATGTATTACTGT
GTGAGACAGGGTTACTACTGGTACTTCGATGTCTGGGGCACAGGGACCA
CGGTCACCGTCTCCTCA
SEQ ID NO: 131: 3C5_VL
GACATCCAGATGAACCAGTCTCCATCCAGTCTGTCTGCATCCCTTGGAG
ACACAATTACCATCACTTGCCATGCCAGTCAGAACATTAATGTTTGGTT
AAGCTGGTACCAGCAGAAACCAGGAAATATTCCTGAACTTTTGATCTAT
AAGGCTTCCAACTTGCACACAGGCGTCCCTTCTAGGTTTAGTGGCAATA
GATCTGGAACAAGTTTCACATTAACCATCAGCAGCCTGCAGCCTGAAGA
CATTGGCACTTACTTCTGTCAACAGGGTCAAAGTTATCCGCTCACGTTC
GGTGCTGGGACCAAGCTGGAGCTGAGACGG
SEQ ID NO: 132: 30H2_VL
GACATCCAGATGAACCAGTCTCCATCCAGTCTGTCTGCATCCCTTGGAG
ACACAATTACCATCACTTGCCATGCCAGTCAGAACATTAATGTTTGGTT
AAGCTGGTACCAGCAGAAACCAGGAAATATTCCTGAACTTTTGATCTAT
AAGGCTTCCAACTTGCACACAGGCGTCCCTTCTAGGTTTAGTGGCAATA
GATCTGGAACAAGTTTCACATTAACCATCAGCAGCCTGCAGCCTGAAGA
CATTGGCACTTACTTCTGTCAACAGGGTCAAAGTTATCCGCTCACGTTC
GGTGCGGGGACCAAGCTGGAAATAAAACGG
SEQ ID NO: 133: 11H1_VL
GATGTTTTGATGACCCAAACTCCACTCTCCCTGCCTGTCAGTCTTGGAG
ATCAAGCCTCCATCTCTTGCAGATCTAGTCAGAGCATTGTACATAGTAA
TGGAAACACCTATTTAGAATGGTACCTGCAGAAACCAGGCCAGTCTCCA
AAGCTCCTGATCTACACAGTTTCCAACCGATTTTCTGGGGTCCCAGACA
GGTTCAGTGGCAGTGGATCAGGGACAGATTTCACACTCAAGATCAGCAG
AGTGGAGGCTGAGGATCTGGGAGTTTATTACTGCTTTCAAGGTTCACAT
GTTCCGTGGACGTTCGGTGGAGGCACCAAGCTGGAAATCAAACGG
SEQ ID NO: 134: 12H10_VL
GATGTTTTGATGACCCAAACTCCACTCTCCCTGCCTGTCAGTCTTGGAG
ATCAAGCCTCCATCTCTTGCAGATCTAGTCAGAGCATTGTACATAGTGA
TGGAAACACCTATTTAGAATGGTACCTGCAGAAACCAGGCCAGTCTCCA
AAGCTCCTGATCTACAGAGTTTCCAACCGATTTTCTGGGGTCCCAGACA
GGTTCAGTGGCAGTGGATCAGGGACAGATTTCACACTCAAGATCAGCAG
AGTGGAGGCTGAGGATCTGGGAGTTTATTACTGCTTTCAAGGTTCACAT
GTTCCGTACACGTTCGGAGGAGGCACCAAGCTGGAAATCAAACGG
SEQ ID NO: 135: 7H2_VL
GATGTTTTGATGACCCAAACTCCACTCTCCCTGCCTGTCAGTCTTGGAG
ATCAAGCCTCCATCTCTTGCAGATCTAGTCAGAGCATTGTACATAGTAA
TGGAAACACCTATTTAGAATGGTACCTGCAGAAACCAGGCCAGTCTCCA
AAGCTCCTGATCTACACAGTTTCCAACCGATTTTCTGGGGTCCCAGACA
GGTTCCGTGGCAGTGGATCAGGGACAGATTTCACACTCAAGATCAGCAG
AGTGGAGGCTGAGGATCTGGGAGTTTATTACTGCTTTCAAGGTTCACAT
GTTCCGTGGACGTTCGGTGGAGGCACCAAGCTGGAAATCAAACGG

### Binding Titers of Anti-Human Factor D Antibodies

Recombinant purified monoclonal antibody IgG2a Fc clones 3C5, 30H2, 11H1, 12H10 and 7H2 were analyzed in a binding assay for the ability to bind to human mature Factor D and human pro-Factor D as follows:

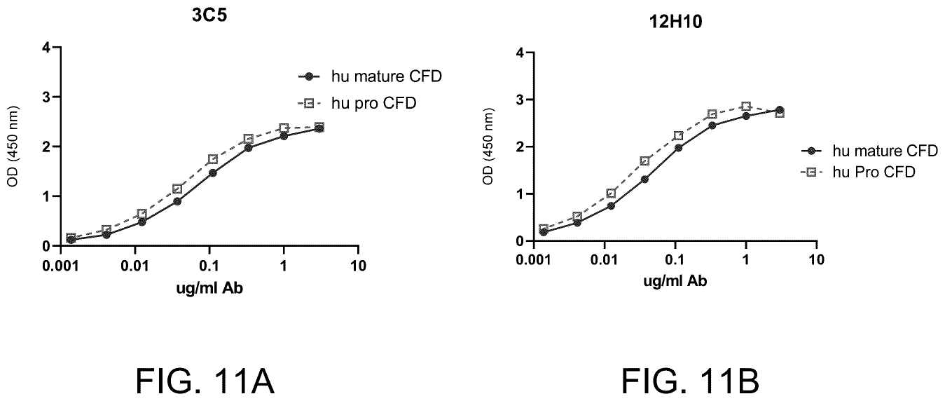

The candidate antibodies were titrated starting at 3 μg/mL antibody binding to 1 μg/mL plate-immobilized recombinant human mature Factor D-His or recombinant human Pro-Factor D-His proteins. Bound antibodies were detected by a labeled goat polyclonal antibody specific for mouse IgG Fc (Jackson ImmunoResearch). The results of representative antibodies 3C5 and 12H10 are shown in FIGS. 11A and 11B, respectively.

FIG. 11A graphically illustrates the binding of recombinant human pro-Factor D or mature-Factor D with candidate anti-human Factor D antibody 3C5, demonstrating that antibody 3C5 binds to both human pro-Factor D and mature-Factor D.

FIG. 11B graphically illustrates the binding of recombinant human pro-Factor D or mature-Factor D with candidate anti-human Factor D antibody 12H10, demonstrating that antibody 12H10 binds to both human pro-Factor D and mature-Factor D.

### Example 6

This Example describes the development of an ELISA assay capable of detecting the presence and amount of mature-Factor D in human and cynomolgus monkey serum.

### Background/Rationale:

Purified, recombinant antibodies were generated against a unique N-terminal epitope “ILGGREA” (SEQ ID NO:5) present on both mature human Factor D and mature cynomolgus monkey Factor D as described in Examples 1-3 herein. As described in Examples 4 and 5 herein, purified recombinant antibodies were also generated against mature Factor D which were selected for the ability to detect both pro-Factor D and mature Factor D (i.e., bind to an epitope common to the mature and pro forms of Factor D) are were determined to be suitable for use in an immunoassay. This Example describes the analysis of several representative anti-Factor D antibodies (3C5, 12H10 and others) as coating antibodies in combination with a representative anti-human mature factor D-specific antibody 14A11 in an ELISA assay.

### Methods:

1. Testing the Use of Anti-Human Factor D Antibodies 3C5, 11H1, 12H10 and 30H2 for Use as Coating Antibodies in an ELISA Assay with Detection by Anti-Human Mature Factor D-Specific mAb 14A11

Human IgG4 Fc recombinant purified anti-Factor D antibodies (3C5, 11H1, 12H10 and 30H2) were coated onto ELISA plates and allowed to capture recombinant human and cynomolgus mature and pro-Factor D (huMat CFD, cy Mat CFD, huProCFD and cyPro CFD). Also captured was Factor D-depleted human serum (CFD Dpl serum) and a sample of pooled normal cynomolgus plasma (NCP). Captured Factor D was detected with a mouse IgG2a Fc version of anti-human mature Factor D-specific mAb 14A11. An HRP-labeled F(ab′)₂ fragment donkey anti-mouse IgG H&L antibody (Jackson ImmunoResearch) was used to signal the detection antibody, followed by development with TMB substrate (ThermoFisher).

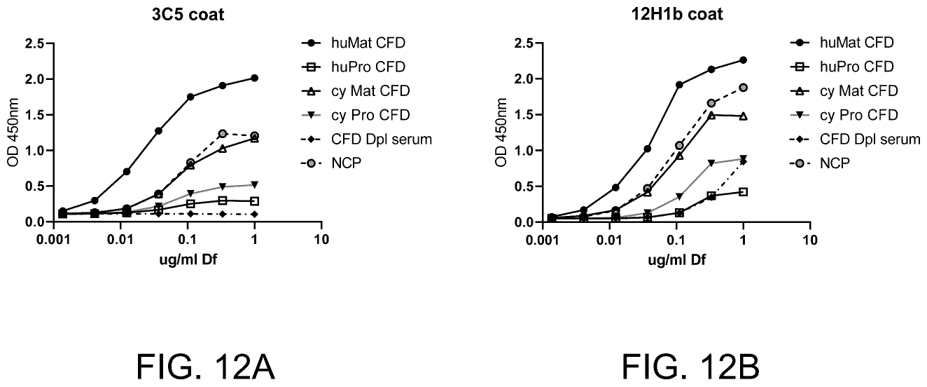

The results of the ELISA assay with representative antibodies 3C5 and 12H10 are shown in FIGS. 12A and 12B, respectively.

### Results:

FIG. 12A graphically illustrates the results of an ELISA assay in which the recombinant anti-Factor D antibody 3C5 was coated onto the ELISA plate and allowed to capture recombinant human and cynomolgus mature and pro-Factor D (huMat CFD, cy Mat CFD, huProCFD and cyPro CFD). Also captured was Factor D-depleted human serum (CFD Dpl serum) and a sample of pooled normal cynomolgus plasma (NCP). Captured Factor D was detected with a mouse IgG2a Fc version of anti-human mature Factor D-specific mAb 14A11.

FIG. 12B graphically illustrates the results of an ELISA assay in which the recombinant anti-Factor D antibody 12H10 was coated onto the ELISA plate and allowed to capture recombinant human and cynomolgus mature and pro-Factor D (huMat CFD, cy Mat CFD, huProCFD and cyPro CFD). Also captured was Factor D-depleted human serum (CFD Dpl serum) and a sample of pooled normal cynomolgus plasma (NCP). Capture Factor D was detected with a mouse IgG2a Fc version of anti-human mature Factor D-specific mAb 14A11.

As shown in FIGS. 12A and 12B, both anti-Factor D antibodies 3C5 and 12H10 are suitable for use as coating antibodies in combination with the anti-human mature-Factor D-specific antibody 14A11 in an ELISA assay for detecting mature Factor D in human and cynomolgus plasma.

2. ELISA Assay to Detect Mature Factor D with a Combination of Coating Antibody 3C5 (Anti-Human/Cyno Factor D) and Detection Antibody 14A11 (Anti-Human/Cyno Mature-Factor D-Specific)

### Methods:

Human IgG4 Fc recombinant purified antibody 3C5 was coated onto an ELISA plate and was tested with the following samples (3-fold serial dilutions):
#1: Cynomolgus recombinant mature Factor D (cy Mat CFD, 1 μg/mL to 1.4 pg/mL)
#2: Human recombinant mature Factor D (hu Mat CFD 1 μg/mL to 1.4 pg/mL)
#3: Cynomolgus recombinant Pro-Factor D (cy Pro CFD 1 μg/mL to 1.4 pg/mL)
#4: Human recombinant Pro-Factor D (hu Pro CFD 1 μg/mL to 1.4 pg/mL)
#5: Purified (from human plasma) human Factor D (CFD) (1 μg/mL to 1.4 pg/mL)
#6: Human Factor D-depleted serum (CFD-Dpl serum, 50% to 0.07%)
#7: Normal human serum (NHS, 50% to 0.07%)
#8: Serum from a human 3MC patient (3MC, 25% to 0.03%)

Captured Factor D was detected with a mouse IgG2a Fc version of anti-human mature Factor D-specific mAb 14A11. An HRP-labeled F(ab′)₂ fragment donkey anti-mouse IgG H&L antibody (Jackson ImmunoResearch) was used to signal the detection antibody, followed by development with TMB substrate (ThermoFisher).

Micro-titer ELISA plates (Maxisorb, Nunc), were coated with antibody clone 3C5 at a concentration of 3 μg/mL using coating buffer (PBS: 1.06 mM potassium phosphate monobasic KH₂PO₄, 155 mM sodium chloride NaCl, 8.97 mM sodium phosphate dibasic Na₂HPO₄-7H20, pH7.4 (ThermoFisher). The plate was incubated overnight at 4° C. The next day, the plate was washed 3 times with PBS buffer with 0.05% and Tween20 (PBST). Residual protein binding sites were blocked by adding 250 μL of 1% bovine serum albumen (BSA) in PBST (PBST-BSA) to each well in the plate and incubated at room temperature for 1 hour. The plates were then washed three times with PBST buffer. 3-fold Serial dilutions of samples #1-6 in a concentration range as shown above were added to the plates and incubated for one hour at room temperature. Wells were then washed three times. 100 μL of antibody clone 14A11 (diluted to 3 μg/mL in PBST-BSA) was then added to each well and incubated for one hour at room temperature. The plates were washed three times. 100 μL of Horseradish Peroxidase labeled F(ab′)2 Fragment Donkey anti-Mouse IgG H&L antibody (Jackson ImmunoResearch), diluted 1:30,000, was added to each well and incubated for one hour at room temperature. Wells were then washed three times. 100 μL of room temperature TMB substrate solution (ThermoFisher) was then added and incubated at room temperature for 5 minutes. 50 μL of 1N H₂SO₄ was added to stop the reaction. The absorbance was measured at 450 nm using Biotek Synergy HT ELISA micro-titre plate reader.

### Results:

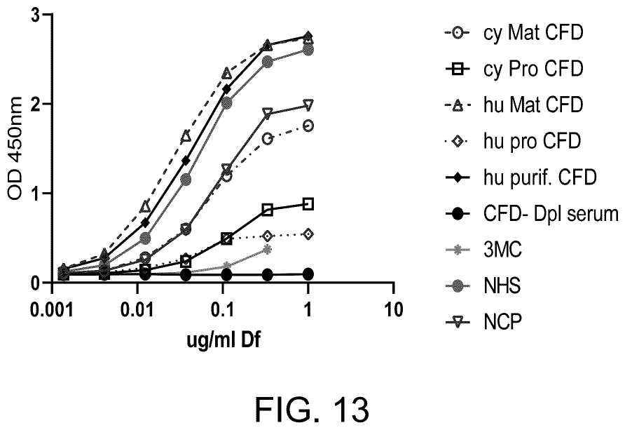

FIG. 13 graphically illustrates the detection of human and cynomolgus monkey mature Factor D and Pro-Factor D with a combination of capture antibody 3C5 (anti-human/cyno Factor D) and detection antibody 14A11 (anti-human/cyno mature-Factor D-specific) in an ELISA assay. As shown in FIG. 13, binding of recombinant human mature factor D (hu Mat CFD), Normal Human Serum (NHS) and purified human complement factor D (hu purif. CFD, Complement Technologies) far exceed binding of recombinant human and cynomolgus pro factor D (hu pro CFD, cy Pro CFD), as well as the plasma from a patient with 3MC syndrome (3MC). Human factor D depleted serum (CFD-Dpl serum) is non-binding. Recombinant cynomolgus mature factor D (cy Mat CFD) and normal cynomolgus plasma (NCP) give less robust readouts than their human corollaries.

### Example 7

This Example describes the generation of monoclonal antibodies that specifically bind to human Pro-Factor D

### Background/Rationale:

This Example describes the generation of anti-human Pro-Factor D-specific antibodies. The antibodies described in this Example specifically bind to human Pro-Factor D and do not bind to human mature Factor D.

### Methods:

### 1. Construction of the Pro-Factor D Antigen

As shown in FIGS. 2 and 3, the pro-peptide of human Factor D is “APPRGR” (SEQ ID NO: 4), corresponding to residues 20-25 of human full-length Factor D. A synthetic peptide-KLH conjugate was generated comprising the amino acids 20-25 of human complement

Factor D “APPRGR” (SEQ ID NO:4), with the addition of a C-terminal Cysteine to allow for conjugation to KLH by Sulfo-SMCC linkage chemistry.
1. Immunization with the Pro-Factor D Antigen

BALB/c mice were immunized with the synthetic peptide-KLH conjugate comprising the amino acids 20-25 of human complement Factor D “APPRGR” (SEQ ID NO:4), with the addition of a C-terminal Cysteine to allow for conjugation to KLH by Sulfo-SMCC linkage chemistry. The mice were immunized four times, subcutaneously, with 100 to 200 μL of adjuvant emulsions of peptide conjugate (50-100 μg total protein per injection.)

Serum samples from the immunized mice were prepared from retro-orbital bleeds and tested by ELISA assay for the presence of antigen-specific antibodies capable of binding to plate-immobilized recombinant human Pro-Factor D (SEQ ID NO:2) and recombinant human mature Factor D (SEQ ID NO:3) as follows.

Recombinant human Pro-CFD-His or recombinant human mature CFD-His were immobilized on Maxisorp™ ELISA plates at 1 μg/mL in PBS, 100 μL/well, incubated overnight at 4° C. Plate wells were then washed three times with 300 μL PBS containing 0.05% Tween 20 (PBST), blocked for 1 hour at room temperature with 250 μL PBS containing 1% BSA and washed again. Serum from mouse #2, taken after the third boost, was diluted in PBST and allowed to bind for 1 hour at room temperature, then washed three times in PBST. An HRP-labeled goat anti-mouse IgG Fc antibody (Jackson ImmunoResearch) was then applied (100 μL/well), allowed to bind for 1 hour at room temperature, and then washed three times with PBST. TMB substrate (Thermo Fisher) (100 μL/well) was then applied and incubated for 5 minutes at room temperature. The reaction was then stopped with 1N H₂SO₄ (50 μL/well). The plate was read for optical density at 450 nM with a Biotek™ ELISA plate reader.

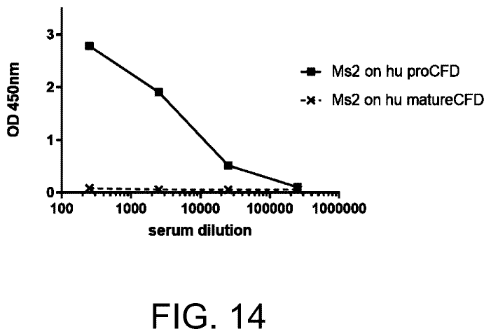

FIG. 14 graphically illustrates a titration of the serum of a representative mouse #2 (Ms2) after immunization with a synthetic peptide comprising “APPRGR” (SEQ ID NO: 4) corresponding to amino acid residues 20-25 of human complement factor D in the presence of recombinant mature Factor D (hu mature CFD) or recombinant pro-Factor D (hu proCFD). As shown in FIG. 14, the serum from representative mouse #2 contains antibodies capable of selectively binding to pro-Factor D as compared to mature Factor D.

The mice showing the most favorable binding to pro-Factor D and the least favorable binding to mature-Factor D (i.e., mouse #2) were selected for hybridoma fusion. Three days prior to the fusion, mice were treated subcutaneously with 50 μg of an anti-CD40 agonist mAb in PBS (R&D Systems, Minneapolis, MN) to increase B cells numbers (see Rycyzyn et al., Hybridoma 27:25-30, 2008). The mice were sacrificed, and the spleen cells were harvested and fused to a selected murine myeloma cell line P3/NSI/1-AG4-1 (NS-1) (ATCC No. TIB18) using 50% polyethylene glycol or 50% polyethylene glycol plus 10% DMSO. The fusions generated hybridoma cells which were plated in 96 well Nunc tissue culture treated plates containing HAT (hypoxanthine, aminopterin and thymidine) medium to inhibit proliferation of non-fused cells, myeloma hybrids and spleen hybrids. Hybridoma wells were fed by replacement of 80% of media with fresh medium containing HAT supplement. After hybridoma selection, the culture supernatants were assayed for binding to recombinant human Pro-Factor D and mature Factor D as described below.

### 2. Hybridoma Screening

Hybridoma supernatants were first screening for binding to immobilized recombinant human pro-Factor D. Hybridomas testing positive by ELISA for binding to immobilized recombinant human pro-Factor D-His (n=6) (1F9, 2A4, 13A10, 18F5, 20A1, 21H1) were then tested for binding to recombinant human mature Factor D.

The specificity of anti-human pro-CFD hybridomas were further analyzed as follows. Hybridoma supernatants (18F5, 1F9, 2A4, 20A1, 13A10, 21H1) were tested for their ability to detect recombinant pro-Factor D or mature Factor D when captured by a polyclonal goat anti-human factor D antibody AF1824 (R&D Systems) as follows. ELISA plates were coated with polyclonal anti-human factor D antibody AF1824 (R&D Systems). Hybridoma supernatants were diluted two-fold in PBS, 0.05% Tween 20 (PBST). Control samples included: Control supernatant from a hybridoma known to bind to human MASP3 (aM3 35C1) was similarly diluted. Control samples also included: PBST, 1 μg/ml of Mouse IgG (Jackson Immunoresearch 015-000-003), and 1 μg/ml mouse monoclonal anti-human CFD (R&D Systems MAB1824). Hybridoma supernatants and control samples were allowed to bind to captured pro or mature Factor D for one hour at room temperature. After washing three times with PBST, 100 μL/well of HRP-goat anti-mouse IgG Fc gamma secondary antibody (Jackson ImmunoResearch) diluted in PBST was added and incubated for 1 hour at room temperature. After washing three times with PBST, TMB substrate (ThermoFisher), 100 μL/well, was added. After 4 minutes the reaction was stopped with 50 μL/well of 1N H2SO₄, then read at 450 nm on a Biotek™ ELISA plate reader.

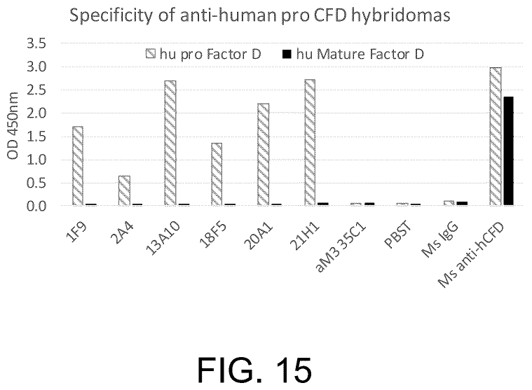

FIG. 15 graphically illustrates the results of a capture ELISA assay in which hybridoma supernatants were screened for binding to human pro-Factor D or human mature-Factor D when captured by a polyclonal anti-Factor D antibody AF1824 (R&D Systems).

As shown in FIG. 15, out of 6 hybridomas tested, the supernatants from all 6 hybridomas (18F5, 1F9, 2A4, 20A1, 13A10, 21H1) showed preferential binding to recombinant human pro-Factor D as compared to recombinant human mature-Factor D.

Hybridomas 18F5, 1F9, 2A4, 20A1, 13A10, 21H1 were selected for DNA cloning and recombinant antibody production.

### Example 8

This Example describes the cloning and sequence analysis of anti-human pro-Factor D-specific monoclonal antibodies.

### Background/Rationale:

This Example describes the cloning and sequence analysis of antibodies produced by the hybridomas showing preferential binding to the pro form of Factor D (i.e., 18F5, 1F9, 2A4, 20A1, 13A10, 21H1) described in Example 7.

### Methods:

### 1. Cloning and Purification of Recombinant Antibodies

Positive hybridomas 18F5, 1F9, 2A4, 20A1, 13A10, 21H1 were generated and identified as described in Example 7. These hybridomas were subcloned by serial dilution methods.

The heavy chain and light chain variable regions were cloned from the hybridomas 18F5, 1F9, 2A4, 20A1, 13A10, 21H1 using RT-PCR and were sequenced. Antibody-encoding sequences were amplified from total RNA with isotype-specific reverse primers using the SMARTer™ RACE 5′/3′ kit (Takara Bio). After verifying the sequences, the variable (V) regions were re-amplified with designed cloning primers and cloned into expression vectors carrying either the human IgG4 heavy chain (SEQ ID NO:71) and kappa light chain (SEQ ID NO:72) constant regions or the mouse IgG2a (SEQ ID NO:218) and kappa light chain (SEQ ID NO:219) constant regions using the In-Fusion HD™ cloning kit (Clontech). The expression constructs were co-transfected transiently into Expi293 cells (Life Technologies), and after 5 days of culture, secreted recombinant antibodies were purified from supernatants by protein A chromatography.

The sequences of the heavy chain variable regions and light chain variable regions are shown in FIGS. 16A and 16B, respectively (“SIN”=“SEQ ID NO:” in FIG. 16A and FIG. 16B), and are included below. The complementarity regions (CDRs) and framework regions (FRs) of each are provided in TABLES 13-16 below.

### Anti-Human Pro-Factor D-Specific Antibody Heavy Chain Variable Region (VH) Sequences

FIG. 16A shows an amino acid alignment of the heavy chain variable region (VH) sequences for the anti-human pro-Factor D-specific clones: 18F5_VH (SEQ ID NO: 136), 1F9_VH (SEQ ID NO:137), 2A4_VH (SEQ ID NO:138), 20A1_VH (SEQ ID NO: 139), 13A10_VH (SEQ ID NO:140) and 21H1_VH (SEQ ID NO:141).

Presented below is the heavy chain variable region (VH) sequence for each anti-human Pro-Factor-D-specific antibody. The Kabat CDRs are underlined.

18F5_VH:
SEQ ID NO: 136
EVKLEESGGGLVQPGGSMKLSCVASGFTFGNYWMSWVRQSPEKGLEWVA
EIRLKSDNYATHYAESVKGKFTISRDDSKSRLYLQMNSLRGEDTGLYYC
TNAWFASWGQGTLVTVSA
1F9_VH:
SEQ ID NO: 137
EVKLEESGGGLVQPGGSMKLSCVASGFTFGSYWMSWVRQSPEKGLEWVA
EIRLKSDNYAAHYAESVKGKFTISRDDSKSRLYLQMNSLRGEDTGIYYC
TNAWFASWGQGTLVTVSA
2A4_VH:
SEQ ID NO: 138
EVKLEESGGGLVQPGGSMKLSCVASGFTFSTYWMSWVRQSPEKGLEWVA
EIRLKSDNYATHYTESVKGKFTISRDDSKSRLYLQMNSLRVEDTGIYYC
TNAWFAYWGQGTLVTVSA
20A1_VH:
SEQ ID NO: 139
EVKLEESGGGLVQPGGSMKLSCIASGFTFSTYWMSWVRQSPEKGLEWVA
EIRLKSENYATYYAESVKGKFIISRDDSKSRLYLQMNSLRAEDTGIYYC
TNAWFANWGQGTLVTVSA
13A10_VH:
SEQ ID NO: 140
DVQLQESGPGLVKPSQSLSLTCTVTGYSITSDYAWNWIRQFPGNKLEWM
GYISYIGGIGYNPSLKSRISITRDTSKNQFFLHLNSVTTGDTATYYCAR
NGAMDFWGQGISVTVSS
21H1_VH:
SEQ ID NO: 141
DVQLQESGPGLVKPSQSLSLTCTVTGYSITSDYAWNWIRQFPGNKLEWM
GYISYSGSTGYSPSLKSRISITRDTSKNQFFLHLNSVTTGDTATYYCAR
NGAMDYWGQGISVTVSS

TABLE 14
anti-human pro-Factor D-specific Antibody VH
Sequences (CDRs and FR regions, Kabat)

| Antibody | HC FR1 | HC CDR1 |
|---|---|---|
| 18F5 | EVKLEESGGGLVQPG | NYWMS |
| | GSMKLSCVASGFTFG | (SEQ ID NO: 149) |
| | (SEQ ID NO: 148) | |
| 1F9 | EVKLEESGGGLVQPG | SYWMS |
| | GSMKLSCVASGFTFG | (SEQ ID NO: 155) |
| | (SEQ ID NO: 148) | |
| 2A4 | EVKLEESGGGLVQPG | TYWMS |
| | GSMKLSCVASGFTFS | (SEQ ID NO: 158) |
| | (SEQ ID NO: 247) | |
| 20A1 | EVKLEESGGGLVQPG | TYWMS |
| | GSMKLSCIASGFTFS | (SEQ ID NO: 158) |
| | (SEQ ID NO: 162) | |
| 13A10 | DVQLQESGPGLVKPS | SDYAWN |
| | QSLSLTCTVTGYSIT | (SEQ ID NO: 167) |
| | (SEQ ID NO: 166) | |
| 21H1 | DVQLQESGPGLVKPS | SDYAWN |
| | QSLSLTCTVTGYSIT | (SEQ ID NO: 167) |
| | (SEQ ID NO: 166) | |
| Antibody | HC FR2 | HC CDR2 |
| 18F5 | WVRQSPEKGLEWVA | EIRLKSDNYATHYAESVKG |
| | (SEQ ID NO: 150) | (SEQ ID NO: 151) |
| 1F9 | WVRQSPEKGLEWVA | EIRLKSDNYAAHYAESVKG |
| | (SEQ ID NO: 150) | (SEQ ID NO: 156) |
| 2A4 | WVRQSPEKGLEWVA | EIRLKSDNYATHYTESVKG |
| | (SEQ ID NO: 150) | (SEQ ID NO: 159) |
| 20A1 | WVRQSPEKGLEWVA | EIRLKSENYATYYAESVKG |
| | (SEQ ID NO: 150) | (SEQ ID NO: 163) |
| 13A10 | WIRQFPGNKLEWMG | YISYIGGIGYNPSLKS |
| | (SEQ ID NO: 168) | (SEQ ID NO: 169) |
| 21H1 | WIRQFPGNKLEWMG | YISYSGSTGYSPSLKS |
| | (SEQ ID NO: 168) | (SEQ ID NO: 173) |
| Antibody | HC FR3 | HC CDR3 |
| 18F5 | KFTISRDDSKSRLYLQ | AWFAS |
| | MNSLRGEDTGLYYCTN | (SEQ ID NO: 153) |
| | (SEQ ID NO: 152) | |
| 1F9 | KFTISRDDSKSRLYLQ | AWFAS |
| | MNSLRGEDTGIYYCTN | (SEQ ID NO: 153) |
| | (SEQ ID NO: 157) | |
| 2A4 | KFTISRDDSKSRLYLQ | AWFAY |
| | MNSLRVEDTGIYYCTN | (SEQ ID NO: 161) |
| | (SEQ ID NO: 160) | |
| 20A1 | KFIISRDDSKSRLYLQ | AWFAN |
| | MNSLRAEDTGIYYCTN | (SEQ ID NO: 165) |
| | (SEQ ID NO: 164) | |
| 13A10 | RISITRDTSKNQFFLH | NGAMDF |
| | LNSVTTGDTATYYCAR | (SEQ ID NO: 171) |
| | (SEQ ID NO: 170) | |
| 21H1 | RISITRDTSKNQFFLH | NGAMDY |
| | LNSVTTGDTATYYCAR | (SEQ ID NO: 174) |
| | (SEQ ID NO: 170) | |
| Antibody | HC FR4 | |
| 18F5 | WGQGTLVTVSA | |
| | (SEQ ID NO: 154) | |
| 1F9 | WGQGTLVTVSA | |
| | (SEQ ID NO: 154) | |
| 2A4 | WGQGTL VTVSA | |
| | (SEQ ID NO: 154) | |
| 20A1 | WGQGTL VTVSA | |
| | (SEQ ID NO: 154) | |
| 13A10 | WGQGISVTVSS | |
| | (SEQ ID NO: 172) | |
| 21H1 | WGQGISVTVSS | |
| | (SEQ ID NO: 172) | |

### Anti-Human Pro-Factor D-Specific Antibody Light Chain Variable Region (VL) Sequences

FIG. 16B shows an amino acid alignment of the light chain variable region (VL) sequences for the anti-human mature-Factor D-specific clones: 18F5_VL (SEQ ID NO:142), 1F9_VL (SEQ ID NO:143), 2A4_VL (SEQ ID NO:144), 20A1_VL (SEQ ID NO:145), 13A10_VL (SEQ ID NO:146), and 21H1_VL (SEQ ID NO:147).

Presented below is the light chain variable region (VL) sequence for each anti-human pro-factor-D-specific antibody. The Kabat CDRs are underlined. These regions are the same whether numbered by the Kabat or Chothia system.

18F5_VL:
SEQ ID NO: 142
DIVMSQSPSSLAVSVGEKVTMSCMSSQSLLYSKDQKNYLAWYQQKPGQS
PKLLIYWASTRESGVPDRFTGSGSGTDFTLTISSVKAEDLAVYYCLQYY
TYPYTFGGGTKLEIKR
1F9_VL:
SEQ ID NO: 143
DIVMSQSPSSLTVSVGEKVTMSCMSSQSLLYSKDQKNYLAWYQQKPGQS
PTLLIYWASTRESGVPDRFTGSGSGTDFTLTISSVKAEDLAVYYCLQYY
TYPYTFGGGTKLEIKR
2A4_VL:
SEQ ID NO: 144
DIVMSQSPSSLAVSVGEKFTMSCKSSQSLLYSRDQKNYLAWYQQQPGQS
PKLLIYWASTRESGVPDRFTGSGSGTDFTLTISSVKTEDLAVYYCLQYY
SYPYTFGGGTKLEIKR
20A1_VL:
SEQ ID NO: 145
DIVMSQSPSSLVVSVGEKVTMSCKSSQNLLYSRDQKNYLAWYQQKPGQS
PNLLIYWASTRESGVPDRFTGSGSGTDFSLTISSVKAEDLAVYYCLQYY
SYPYTFGGGTKLEMKR
13A10_VL:
SEQ ID NO: 146
DIVLTQSPASLAVSLGQRATISCKASQSVDYDGDSYMNWYQQKPGQPPK
LLIYDASNLESGIPARFSGSGSGTDFTLNIHPVEEEDAATYYCQQSNEA
PWTFGGGTKLEIKR
21H1_VL:
SEQ ID NO: 147
DIVLTQSPASLAVSLGQRATISCKASQSVDYDGDSYMNWYQQKPGQPPK
LLIYDASTLESGIPARFSGSGSGTDFTLNIHPVEEEDAATYYCQQNYEA
PWTFGGGTKLEIKR

TABLE 15
anti-human pro-Factor D-specific Antibody VL
Sequences (CDRs and FR regions, Kabat and Chothia)

| Antibody | LC FR1 | LC CDR1 |
|---|---|---|
| 18F5 | DIVMSQSPSSLAVSVGEKV | MSSQSLLYSKDQKNYLA |
| | TMSC | (SEQ ID NO: 176) |
| | (SEQ ID NO: 175) | |
| 1F9 | DIVMSQSPSSLTVSVGEKV | MSSQSLLYSKDQKNYLA |
| | TMSC | (SEQ ID NO: 176) |
| | (SEQ ID NO: 181) | |
| 2A4 | DIVMSQSPSSLAVSVGEKF | KSSQSLLYSRDQKNYLA |
| | TMSC | (SEQ ID NO: 184) |
| | (SEQ ID NO: 183) | |
| 20A1 | DIVMSQSPSSLVVSVGEKV | KSSQNLLYSRDQKNYLA |
| | TMSC | (SEQ ID NO: 189) |
| | (SEQ ID NO: 188) | |
| 13A10 | DIVLTQSPASLAVSLGQRA | KASQSVDYDGDSYMN |
| | TISC | (SEQ ID NO: 194) |
| | (SEQ ID NO: 193) | |
| 21H1 | DIVLTQSPASLAVSLGQRA | KASQSVDYDGDSYMN |
| | TISC | (SEQ ID NO: 194) |
| | (SEQ ID NO: 193) | |
| Antibody | LC FR2 | LC CDR2 |
| 18F5 | WYQQKPGQSPKLLIY | WASTRES |
| | (SEQ ID NO: 177) | (SEQ ID NO: 178) |
| 1F9 | WYQQKPGQSPTLLIY | WASTRES |
| | (SEQ ID NO: 182) | (SEQ ID NO: 178) |
| 2A4 | WYQQQPGQSPKLLIY | WASTRES |
| | (SEQ ID NO: 185) | (SEQ ID NO: 178) |
| 20A1 | WYQQKPGQSPNLLIY | WASTRES |
| | (SEQ ID NO: 190) | (SEQ ID NO: 178) |
| 13A10 | WYQQKPGQPPKLLIY | DASNLES |
| | (SEQ ID NO: 195) | (SEQ ID NO: 196) |
| 21H1 | WYQQKPGQPPKLLIY | DASTLES |
| | (SEQ ID NO: 195) | (SEQ ID NO: 199) |
| Antibody | LC FR3 | LC CDR3 |
| 18F5 | GVPDRFTGSGSGTDFT | LQYYTYPYT |
| | LTISSVKAEDLAVYYC | (SEQ ID NO: 180) |
| | (SEQ ID NO: 179) | |
| 1F9 | GVPDRFTGSGSGTDFT | LQYYTYPYT |
| | LTISSVKAEDLAVYYC | (SEQ ID NO: 180) |
| | (SEQ ID NO: 179) | |
| 2A4 | GVPDRFTGSGSGTDFT | LQYYSYPYT |
| | LTISSVKTEDLAVYYC | (SEQ ID NO: 187) |
| | (SEQ ID NO: 186) | |
| 20A1 | GVPDRFTGSGSGTDFS | LQYYSYPYT |
| | LTISSVKAEDLAVYYC | (SEQ ID NO: 187) |
| | (SEQ ID NO: 191) | |
| 13A10 | GIPARFSGSGSGTDFT | QQSNEAPWT |
| | LNIHPVEEEDAATYYC | (SEQ ID NO: 198) |
| | (SEQ ID NO: 197) | |
| 21H1 | GIPARFSGSGSGTDFT | QQNYEAPWT |
| | LNIHPVEEEDAATYYC | (SEQ ID NO: 200) |
| | (SEQ ID NO: 197) | |
| Antibody | LC FR4 | |
| 18F5 | FGGGTKLEIKR | |
| | (SEQ ID NO: 55) | |
| 1F9 | FGGGTKLEIKR | |
| | (SEQ ID NO: 55) | |
| 2A4 | FGGGTKLEIKR | |
| | (SEQ ID NO: 55) | |
| 20A1 | FGGGTKLEMKR | |
| | (SEQ ID NO: 192) | |
| 13A10 | FGGGTKLEIKR | |
| | (SEQ ID NO: 55) | |
| 21H1 | FGGGTKLEIKR | |
| | (SEQ ID NO: 55) | |

TABLE 16
Consensus Sequences for anti-human pro-Factor
D-specific HC CDRs:

| Antibody | Region | Sequence |
|---|---|---|
| 18F5 | HC-CDR1 | NYWMS (SEQ ID NO: 149) |
| 1F9 | HC-CDR1 | SYWMS (SEQ ID NO: 155) |
| 2A4 | HC-CDR1 | TYWMS (SEQ ID NO: 158) |
| 20A1 | HC-CDR1 | TYWMS (SEQ ID NO: 158) |
| Consensus | HC-CDR1 | XYWMS (SEQ ID NO: 201) |
| | | Wherein: |
| | | X at position 1 is N, S or T |
| 18F5 | HC-CDR2 | EIRLKSDNYATHYAESVKG |
| | | (SEQ ID NO: 151) |
| 1F9 | HC-CDR2 | EIRLKSDNYAAHYAESVKG |
| | | (SEQ ID NO: 156) |
| 2A4 | HC-CDR2 | EIRLKSDNYATHYTESVKG |
| | | (SEQ ID NO: 159) |
| 20A1 | HC-CDR2 | EIRLKSENYATYYAESVKG |
| | | (SEQ ID NO: 163) |
| Consensus | HC-CDR2 | EIRLKSXNYAXXYXESVKG |
| | | (SEQ ID NO: 202) |
| | | Wherein: |
| | | X at position 7 is D or E: |
| | | X at position 11 is T or A; |
| | | X at position 12 is H or Y; |
| | | X at position 14 is A or T |
| 18F5 | HC-CDR3 | AWFAS (SEQ ID NO: 153) |
| 1F9 | HC-CDR3 | AWFAS (SEQ ID NO: 153) |
| 2A4 | HC-CDR3 | AWFAY (SEQ ID NO: 161) |
| 20A1 | HC-CDR3 | AWFAN (SEQ ID NO: 165) |
| Consensus | HC-CDR3 | AWFAX (SEQ ID NO: 203) |
| | | Wherein X at position 5 is |
| | | S, Y or N |

TABLE 17
Consensus Sequences for pro-Factor D-specific
LC CDRs:

| Antibody | Region | Sequence |
|---|---|---|
| 18F5 | LC-CDR1 | MSSQSLLYSKDQKNYLA |
| | | (SEQ ID NO: 176) |
| 1F9 | LC-CDR1 | MSSQSLLYSKDQKNYLA |
| | | (SEQ ID NO: 176) |
| 2A4 | LC-CDR1 | KSSQSLLYSRDQKNYLA |
| | | (SEQ ID NO: 184) |
| 20A1 | LC-CDR1 | KSSQNLLYSRDQKNYLA |
| | | (SEQ ID NO: 189) |
| Consensus | LC-CDR1 | XSSQXLLYSXDQKNYLA |
| | | (SEQ ID NO: 204) |
| | | Wherein: |
| | | X at position 1 is M or K; |
| | | X at position 5 is S or N; |
| | | X at position 10 is K or R |
| 18F5 | LC-CDR2 | WASTRES (SEQ ID NO: 178) |
| 1F9 | LC-CDR2 | WASTRES (SEQ ID NO: 178) |
| 2A4 | LC-CDR2 | WASTRES (SEQ ID NO: 178) |
| 20A1 | LC-CDR2 | WASTRES (SEQ ID NO: 178) |
| Consensus | LC-CDR2 | WASTRES (SEQ ID NO: 178) |
| 18F5 | LC-CDR3 | LQYYTYPYT (SEQ ID NO: 180) |
| 1F9 | LC-CDR3 | LQYYTYPYT (SEQ ID NO: 180) |
| 2A4 | LC-CDR3 | LQYYSYPYT (SEQ ID NO: 187) |
| 20A1 | LC-CDR3 | LQYYSYPYT (SEQ ID NO: 187) |
| Consensus | LC-CDR3 | LQYYXYPYT (SEQ ID NO: 205) |
| | | Wherein X at position 5 is |
| | | T or S |

### Nucleic Acid Sequences Encoding Pro-Factor D-Specific Monoclonal Antibodies:

18F5_VH:
SEQ ID NO: 206
GAAGTGAAGCTTGAGGAGTCTGGAGGAGGCCTGGTGCAACCTGGAGGAT
CCATGAAACTCTCCTGTGTAGCCTCTGGATTTACTTTCGGTAACTACTG
GATGTCTTGGGTCCGCCAGTCTCCAGAGAAGGGGCTTGAGTGGGTTGCT
GAAATTAGATTGAAATCTGATAATTATGCAACACATTATGCGGAGTCTG
TGAAAGGGAAGTTCACCATCTCAAGAGATGATTCCAAAAGTCGTCTCTA
CCTGCAAATGAACAGCTTAAGAGGTGAAGACACTGGACTTTATTACTGT
ACGAATGCCTGGTTTGCTTCCTGGGGCCAAGGGACTCTGGTCACTGTCT
CTGCA
1F9_VH:
SEQ ID NO: 207
GAAGTGAAGCTTGAGGAGTCTGGAGGAGGCTTGGTGCAACCTGGAGGAT
CCATGAAACTCTCCTGTGTTGCCTCTGGATTTACTTTCGGTAGCTACTG
GATGTCTTGGGTCCGCCAGTCTCCAGAGAAGGGGCTTGAGTGGGTTGCT
GAAATTAGATTGAAATCTGATAATTATGCAGCACATTATGCGGAGTCTG
TGAAAGGGAAGTTCACCATCTCAAGAGATGATTCCAAAAGTCGTCTCTA
CCTGCAAATGAACAGCTTAAGAGGCGAAGACACTGGAATTTATTACTGT
ACGAATGCCTGGTTTGCTTCCTGGGGCCAAGGGACTCTGGTCACTGTTT
CTGCA
2A4_VH:
SEQ ID NO: 208
GAAGTGAAGCTTGAGGAGTCTGGAGGAGGCTTGGTGCAACCTGGAGGAT
CCATGAAACTCTCCTGTGTTGCCTCTGGATTTACTTTCAGCACTTATTG
GATGTCTTGGGTCCGCCAGTCTCCAGAGAAGGGGCTTGAGTGGGTTGCT
GAAATTAGATTGAAATCTGATAATTATGCAACACATTATACGGAGTCTG
TGAAAGGGAAGTTCACCATCTCAAGAGATGATTCCAAAAGTCGTCTCTA
CCTGCAAATGAACAGTTTAAGAGTTGAAGACACTGGAATTTATTATTGT
ACGAATGCCTGGTTTGCTTACTGGGGCCAAGGGACTCTGGTCACTGTCT
CTGCA
20A1_VH:
SEQ ID NO: 209
GAAGTGAAGCTTGAGGAGTCTGGAGGAGGCTTGGTGCAACCTGGAGGAT
CCATGAAACTCTCCTGTATTGCCTCTGGATTTACTTTCAGTACCTACTG
GATGTCTTGGGTCCGCCAGTCTCCAGAGAAGGGGCTTGAGTGGGTTGCT
GAAATTAGATTGAAATCTGAAAATTATGCAACATATTATGCGGAGTCTG
TGAAAGGGAAGTTCATCATCTCAAGAGATGATTCCAAAAGTCGTCTCTA
CCTGCAAATGAACAGCTTAAGAGCTGAAGACACTGGAATTTATTACTGT
ACGAATGCCTGGTTTGCTAACTGGGGCCAAGGGACTCTGGTCACTGTCT
CTGCA
13A10_VH:
SEQ ID NO: 210
GATGTGCAGCTTCAGGAGTCGGGACCTGGCCTGGTGAAACCTTCTCAGT
CTCTGTCCCTCACCTGCACTGTCACTGGCTACTCAATCACCAGTGATTA
TGCCTGGAACTGGATCCGGCAGTTTCCAGGAAACAAACTGGAGTGGATG
GGCTACATAAGCTACATTGGTGGCATTGGCTACAACCCATCTCTCAAAA
GTCGAATCTCTATCACTCGAGACACATCCAAGAACCAGTTCTTCCTGCA
CTTGAATTCTGTGACTACTGGGGACACAGCCACATATTACTGTGCAAGA
AACGGGGCTATGGACTTCTGGGGTCAAGGAATCTCAGTCACCGTCTCCT
CA
21H1_VH:
SEQ ID NO: 211
GATGTGCAGCTTCAGGAGTCGGGACCTGGCCTGGTGAAACCTTCTCAGT
CTCTGTCCCTCACCTGCACTGTCACTGGCTACTCAATCACCAGTGATTA
TGCCTGGAACTGGATCCGGCAGTTTCCAGGAAACAAACTGGAGTGGATG
GGCTACATAAGTTACAGTGGTAGCACTGGCTATAGCCCATCTCTCAAAA
GTCGAATCTCTATCACTCGAGACACATCCAAGAACCAGTTCTTCCTGCA
CTTGAATTCTGTGACTACTGGAGACACAGCCACATATTACTGTGCACGA
AACGGGGCTATGGACTACTGGGGTCAAGGAATCTCAGTCACCGTCTCCT
CA
18F5_VK:
SEQ ID NO: 212
GACATTGTGATGTCACAGTCTCCATCCTCCCTAGCTGTGTCAGTTGGAG
AGAAGGTTACTATGAGCTGCATGTCCAGTCAGAGCCTTTTATATAGTAA
AGATCAAAAGAACTACTTGGCCTGGTACCAACAGAAACCAGGGCAGTCT
CCTAAACTGCTGATTTACTGGGCATCCACTAGGGAATCTGGGGTCCCTG
ATCGCTTCACAGGCAGTGGATCTGGGACAGATTTCACTCTCACCATCAG
CAGTGTGAAGGCTGAAGACCTGGCAGTTTATTACTGTCTGCAATATTAT
ACCTATCCGTACACGTTCGGAGGGGGGACCAAGCTGGAAATAAAACGG
1F9_VK:
SEQ ID NO: 213
GACATTGTGATGTCACAGTCTCCATCCTCCCTAACTGTGTCAGTTGGAG
AGAAGGTTACTATGAGCTGCATGTCCAGTCAGAGCCTTTTATATAGTAA
AGATCAAAAGAACTACTTGGCCTGGTACCAACAGAAACCAGGGCAGTCT
CCTACACTGCTGATTTACTGGGCATCCACTAGGGAATCTGGGGTCCCTG
ATCGCTTCACAGGCAGTGGATCTGGGACAGATTTCACTCTCACCATCAG
CAGTGTGAAGGCTGAAGACCTGGCAGTTTATTACTGTCTGCAATATTAT
ACCTATCCGTACACGTTCGGAGGGGGGACCAAGCTGGAAATAAAACGG
2A4_VK:
SEQ ID NO: 214
GACATTGTGATGTCACAGTCTCCATCCTCCCTAGCTGTGTCAGTTGGAG
AGAAGTTTACTATGAGCTGCAAGTCCAGTCAGAGCCTTTTATATAGTCG
CGATCAAAAGAACTACTTGGCCTGGTACCAGCAGCAACCAGGGCAGTCT
CCTAAACTTCTGATTTACTGGGCATCCACTAGGGAGTCTGGGGTCCCTG
ATCGCTTCACAGGCAGTGGATCTGGGACAGATTTCACTCTCACCATCAG
CAGTGTGAAGACTGAAGACCTGGCAGTTTATTACTGTCTCCAATATTAT
AGCTATCCGTACACTTTCGGAGGGGGGACCAAGCTGGAAATAAAACGG
20A1_VK:
SEQ ID NO: 215
GACATTGTGATGTCACAGTCTCCATCCTCCCTAGTTGTGTCAGTTGGAG
AGAAGGTTACTATGAGCTGTAAGTCCAGTCAGAACCTTTTATATAGTAG
GGATCAAAAGAACTACTTGGCCTGGTACCAGCAGAAACCAGGGCAGTCT
CCTAACTTGCTGATTTACTGGGCATCCACTAGGGAATCTGGGGTCCCTG
ATCGCTTCACAGGCAGTGGATCTGGGACAGATTTCTCTCTCACCATCAG
CAGTGTGAAGGCTGAAGACCTGGCAGTTTATTACTGTCTCCAATATTAT
AGCTATCCGTACACGTTCGGAGGGGGGACCAAGCTGGAAATGAAACGG
13A10_VK:
SEQ ID NO: 216
GACATTGTGCTGACCCAATCTCCAGCTTCTTTGGCTGTGTCTCTAGGGC
AGAGGGCCACCATCTCCTGCAAGGCCAGCCAAAGTGTTGATTATGATGG
TGATAGTTATATGAACTGGTACCAACAGAAACCAGGACAGCCACCCAAA
CTCCTCATCTATGATGCATCCAATCTAGAATCTGGGATCCCAGCCAGGT
TTAGTGGCAGTGGGTCTGGGACAGACTTCACCCTCAACATCCATCCTGT
GGAGGAGGAGGATGCTGCAACCTATTACTGTCAGCAAAGTAATGAGGCT
CCGTGGACGTTCGGTGGAGGCACCAAGCTGGAAATCAAACGG
21H1_VK:
SEQ ID NO: 217
GACATTGTGCTGACCCAATCTCCAGCTTCTTTGGCTGTGTCTCTAGGGC
AGAGGGCCACCATCTCCTGCAAGGCCAGCCAAAGTGTTGATTATGATGG
TGATAGTTATATGAACTGGTACCAACAGAAACCAGGACAGCCACCCAAA
CTCCTCATTTATGATGCTTCCACTCTAGAATCTGGGATCCCAGCCAGGT
TTAGTGGCAGTGGGTCTGGGACAGACTTCACCCTCAACATCCATCCTGT
GGAGGAGGAGGATGCTGCAACCTATTACTGTCAGCAAAATTATGAGGCT
CCGTGGACGTTCGGTGGAGGCACCAAGCTGGAAATCAAACGG

### Example 9

This Example describes the functional characterization of recombinant purified anti-human pro-Factor D-specific antibodies in several in vitro assays.

### Background/Rationale:

This Example describes the functional characterization of recombinant anti-human pro-Factor D-specific monoclonal antibodies that were generated as described in Examples 7 and 8 for binding to human pro-Factor D and binding to human mature-Factor D.

### Methods:

### 1. Purified Recombinant Antibodies: Binding to Immobilized Pro-Factor D and Mature Factor D

Monoclonal anti-human pro-CFD antibodies 18F5, 1F9, 2A4, 20A1, 13A10, and 21H1 were made recombinantly on a human IgG4 framework as described in Example 8. These antibodies showed preferential binding by ELISA as hybridoma supernatants on immobilized recombinant human pro-Factor D (as described in Example 7) and were re-tested as recombinant antibodies for their ability to bind to immobilized recombinant human mature Factor D or recombinant human pro-Factor D as described below.

Purified, recombinant antibodies 18F5, 1F9, 2A4, 20A1, 13A10, and 21H1 were serially diluted in PBST from 3 μg/ml in 3-fold dilutions on ELISA plates coated with 1 μg/ml recombinant human pro-Factor D and mature-Factor D and blocked with PBS, 1% BSA. Control purified antibodies were likewise diluted and included: Rat monoclonal anti-mouse CFD (R&D Systems MAB5430), Monoclonal mouse anti-human CFD (R&D MAB18241, Rat IgG (Jackson ImmunoResearch 012-000-003) and Mouse IgG (Jackson ImmunoResearch 015-000-003). After a 1 hour incubation, the plates were washed and an HRP-tagged anti-mouse, rat or human secondary antibody (Southern Biotech 9230-05) was added and incubated for an hour, followed by a wash, then TMB substrate (ThermoFisher). The reaction was stopped with addition of 1N H₂SO₄, and read at 450 nm on a Biotek™ ELISA plate reader.

### Results:

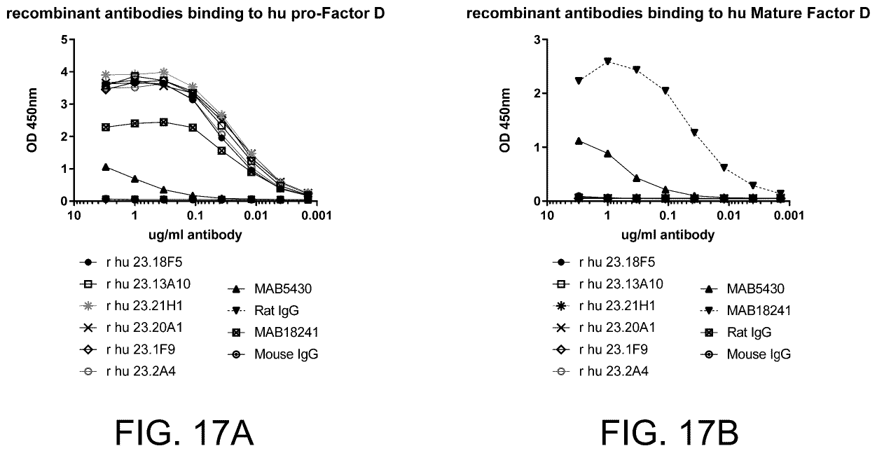

FIG. 17A graphically illustrates the detection of recombinant human pro-Factor D with numerous candidate anti-human pro Factor-D-specific antibodies. As shown in FIG. 17A, all the purified antibodies tested, namely, 18F5, 1F9, 2A4, 20A1, 13A10, and 21H1 were capable of detecting the pro form of Factor D.

FIG. 17B graphically illustrates the detection of recombinant human mature-Factor D with numerous candidate anti-human pro Factor-D-specific antibodies. As shown in FIG. 17B, none of the purified antibodies tested, namely, 18F5, 1F9, 2A4, 20A1, 13A10, or 21H1, were capable of detecting the mature form of Factor D. Control samples included rat IgG and mouse IgG, which bind neither human pro or mature Factor D. Two forms of commercial anti-Factor D antibodies were tested, namely MAB5430 and MAB18241. Both of these commercial antibodies lack specificity for either pro or mature human Factor D.

### 2. Further Analysis of Purified Recombinant Anti-Pro-Factor D-Specific Antibody 21H1: Specificity and Sensitivity

### A. Detection of Recombinant Human Pro-Factor D and Mature Factor D

### Methods:

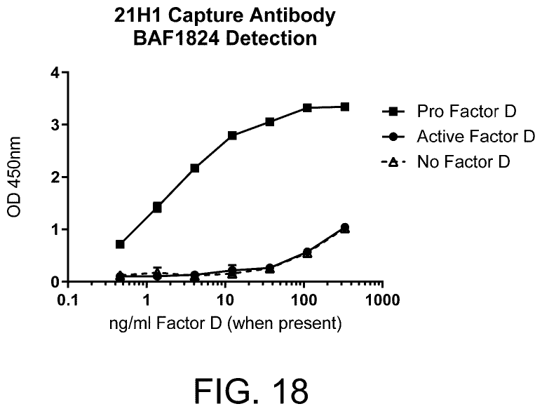

Purified, recombinant anti-pro Factor D-specific antibody 21H1 (human IgG4 Fc) was tested in a sandwich ELISA format as the coating/capturing antibody. After overnight incubation, blocking and washing, recombinant human pro-Factor D and mature-Factor D were diluted to 2 μg/mL normal human plasma, then subsequently in PBST-BSA. A control of no additional Factor D was treated similarly. Assay wells were incubated for 1 hour at room temperature, then washed three times with PBST. Captured molecules were detected with 0.1 μg/mL biotin-labeled, affinity purified, goat polyclonal antibody raised to human mature Factor D (R&D BAF1824) in PBST-BSA. After incubation and washing HRP-tagged Streptavidin was added, incubated, and washed, followed by TMB substrate (ThermoFisher). The results are shown in FIG. 18.

### Results:

FIG. 18 graphically illustrates the detection of recombinant pro-Factor D and mature (active) Factor D in an ELISA assay with recombinant anti-pro-Factor D antibody 21H1 as the coating antibody and goat polyclonal anti-Factor D antibody BAF1824 (R&D Systems) as the detection antibody. As shown in FIG. 18, there is greater signaling in the presence of mature Factor D than for buffer and matrix (No Factor D). mAb 21H1 continues to detect pro-Factor D out to the lowest concentration tested (0.46 ng/ml), while the matrix or mature Factor D signaling is at background levels at 10 ng/ml. These results demonstrate that the specificity of the assay is due to the anti-pro Factor D-specific antibody, 21H1.

### B. Analysis for the Presence of Human Pro-Factor D in Human Serum

### Methods:

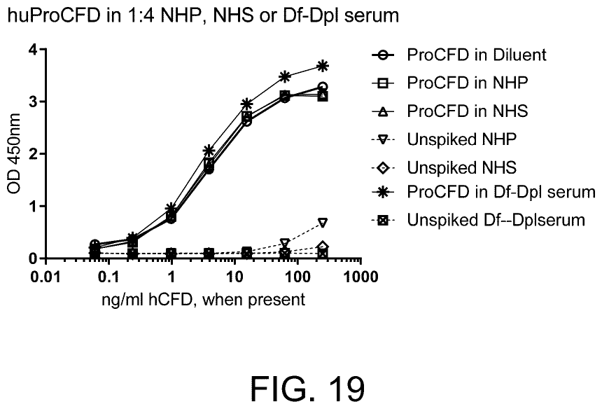

Purified, recombinant anti-pro-Factor D-specific antibody 21H1 (human IgG4 Fc) was tested in a sandwich ELISA format as the coating/capturing antibody. After overnight incubation of 1 μg/ml 21H1 antibody, plates were blocked and washed, and recombinant human pro-Factor D (Pro-CFD) was diluted to 1 μg/ml in 50% Normal Human Plasma (NHP), 50% Normal Human Serum (NHS) or 50% Factor D Depleted Serum (Df-Dpl serum), then subsequently in PBST-BSA. Controls of no additional Factor D in sera were treated similarly (unspiked). Assay wells were incubated for 1 hour at room temperature then washed three times with PBST. Captured molecules were detected with 0.1 μg/ml biotin-labeled, affinity purified, goat polyclonal antibody raised to human mature Factor D (R&D BAF1824) in PBST-BSA. After incubation and washing, HRP-tagged Streptavidin was added, incubated, and washed, followed by TMB substrate. The results are shown in FIG. 19.

### Results:

FIG. 19 graphically illustrates the amount of pro-Factor D present in normal human plasma (NHP), normal human serum (NHS) or Factor-D-depleted serum (Df-Dpl serum) as determined in an ELISA assay with anti-Pro-Factor D antibody 21H1 as the coating antibody and goat polyclonal anti-Factor D antibody AF1824 (R&D Systems) as the detection antibody. As shown in FIG. 19, positive signaling results from the spiking of recombinant pro-Factor D into diluent, normal human plasma, normal human serum, or factor D-depleted serum. Very low to no signaling occurs with unspiked normal human plasma, normal human serum, or factor D-depleted serum.

### C. Analysis for the Presence of Human Pro-Factor D in Test Serum Samples

### Methods:

Purified, recombinant anti-pro-Factor D-specific antibody 21H1 (human IgG4 Fc) was tested in a sandwich ELISA format as the coating/capturing antibody with Normal Human Serum (NHS, Complement Technologies), C1q-Depleted Serum (C1q-Dpl, Complement Technologies A399), Factor D-Depleted Serum (Df-Dpl, Complement Technologies FactorD-Dpl) and 3MC-syndrome patient serum (Patient 3, deficient in MASP-3 activity, kindly provided by Dr. Wilhelm Schwaeble). After overnight incubation of 1 μg/mL 21H1 antibody in PBS, blocking and washing, sera were diluted 1:10 in PBST-BSA, then subsequently 2-fold in PBST-BSA. Assay wells were incubated for 1 hour at room temperature, then washed three times with PBST. Captured molecules were detected with 0.1 μg/mL biotin-labeled, affinity purified, goat polyclonal antibody raised to human mature Factor D (R&D BAF1824) in PBST-BSA. After incubation and washing, HRP-tagged Streptavidin was added, incubated, and washed, followed by TMB substrate (ThermoFisher).

### Results:

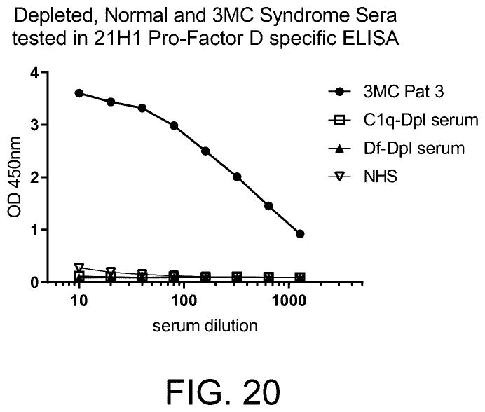

FIG. 20 graphically illustrates the amount of pro-Factor D present in Normal Human Serum (NHS), C1q-Depleted Serum (C1q-Dpl), Factor D-Depleted Serum (Df-Dpl) and 3MC-syndrome patient serum as determined in an ELISA assay with anti-pro-Factor D antibody 21H1 as the coating antibody and goat polyclonal anti-Factor D antibody AF1824 (R&D Systems) as the detection antibody. While Normal Human Serum and human serum depleted of Factor D or C1q have very low signaling when diluted 10-fold and beyond, serum from a 3MC syndrome patient results in strong detection of pro-Factor D.

### Conclusion:

As described in Examples 7-9, the inventors have generated Pro-Factor D-specific monoclonal antibodies that specifically bind to Pro-Factor D and do not bind to mature Factor D. As further described in Examples 10-12, the level of Pro-Factor D correlates with alternative pathway activity, therefore, Pro-Factor D-specific monoclonal antibodies may be used to measure the level of Pro-Factor D as a surrogate endpoint in a diagnostic assay to assess the level of alternative pathway activation in a mammalian subject. As further described herein in Example 12, the Pro-Factor D-specific monoclonal antibodies may be used as a pharmacodynamic (PD) measurement of MASP-3 inhibition in a subject treated with a MASP-3 inhibitor, which may be used to determine efficacious dosing of a MASP-3 inhibitor.

### Example 10

This Example describes the generation of humanized antibodies that bind to MASP-3 and inhibit the maturation of pro-Factor D to factor D and thereby inhibit the Alternative Pathway.

### Background/Rationale:

As shown in FIG. 1 and described herein, it has been determined that MASP-3 is required for the conversion of pro-Factor D to Factor D, therefore MASP-3 is a key regulator of the alternative pathway of complement (APC). Numerous high-affinity anti-MASP-3 inhibitory monoclonal antibodies have been generated as described in WO2018/026722, hereby incorporated herein by reference. As further described in WO2018/026722, several representative MASP-3 inhibitory antibodies (e.g., 4D5, 10D12 and 13B1) were humanized. Representative humanized MASP-3 inhibitory antibodies are described below.

Presented below is the heavy chain variable region (VH) and light chain variable region (VL) sequence for representative humanized MASP-3 inhibitory antibodies. The Kabat CDRs are underlined.

h4D5-14-1-NA_VH
(SEQ ID NO: 220)
QVQLVQSGAEVKKPGASVKVSCKASGYTFTTDDINWVRQAPGQGLEWIG
WIYPRDDRTKYNDKFKDKATLTVDTSSNTAYMELSSLRSEDTAVYYCSS
LEDTYWGQGTLVTVSS
h4D5-14-1-NA_VL
(SEQ ID NO: 221)
DIVMTQSPDSLAVSLGERATINCKSSQSLLASRTRKNYLAWYQQKPGQP
PKLLIYWASTRESGVPDRFSGSGSGTDFTLTISSLQAEDVAVYYCKQSY
NLYTFGQGTKVEIKR
h4D5-19-1-NA_VH
(SEQ ID NO: 222)
QVQLVQSGAEVKKPGASVKVSCKASGYTFTTDDINWVRQAPGQGLEWIG
WIYPRDDRTKYNDKFKDRATLTVDTSSNTAYMELSSLRSEDTAVYYCSS
LEDTYWGQGTLVTVSS
h4D5-19-1-NA_VL
(SEQ ID NO: 221)
DIVMTQSPDSLAVSLGERATINCKSSQSLLASRTRKNYLAWYQQKPGQP
PKLLIYWASTRESGVPDRFSGSGSGTDFTLTISSLQAEDVAVYYCKQSY
NLYTFGQGTKVEIKR
h10D12-45-21-GA_VH
(SEQ ID NO: 223)
QIQLVQSGSELKKPGASVKVSCKASGYIFTSYGMSWVRQAPGKGLKWMG
WINTYSGVPTYADDFKGRFVFSLDTSVRTPYLQISSLKAEDTAVYFCAR
GGEAMDYWGQGTLVTVSS
h10D12-45-21-GA_VL
(SEQ ID NO: 224)
DVLMTQTPLSLSVTPGQPASISCKSSQSLLDSDAKTYLNWLLQRPGQSP
KRLIYLVSKLDSGVPDRFSGSGSGTDFTLKISRVEAEDVGVYYCWQGTH
FPWTFGQGTKVEIKR
h10D12-49-21-GA_VH
(SEQ ID NO: 225)
QIQLVQSGSELKKPGASVKVSCKASGYIFTSYGMSWVRQAPGKGLKWMG
WINTYSGVPTYADDFKGRFVFSLDTSVRTPYLQISSLKAEDTATYFCAR
GGEAMDYWGQGTLVTVSS
h10D12-49-21-GA_VL
(SEQ ID NO: 224)
DVLMTQTPLSLSVTPGQPASISCKSSQSLLDSDAKTYLNWLLQRPGQSP
KRLIYLVSKLDSGVPDRFSGSGSGTDFTLKISRVEAEDVGVYYCWQGTH
FPWTFGQGTKVEIKR
h13B1-9-1-NA_VH
(SEQ ID NO: 226)
QVQLVQSGAEVKKPGASVKVSCKASGYTFTGKWIEWVRQAPGQGLEWIG
EILPGTGSTNYAQKFQGRATFTADSSTSTAYMELSSLRSEDTAVYYCLR
SEDVWGQGTLVTVSS
h13B1-9-1-NA_VL
(SEQ ID NO: 227)
DIVMTQSPDSLAVSLGERATINCKSSQSLLASRTRKNYLAWYQQKPGQP
PKLLIYWASTRESGVPDRFSGSGSGTDFTLTISSLQAEDVAVYYCKQSY
NIPTFGQGTKVEIKR
h13B1-10-1-NA_VH
(SEQ ID NO: 228)
QVQLVQSGAEVKKPGASVKVSCKASGYTFTGKWIEWVRQAPGQGLEWIG
EILPGTGSTNYNEKFKGRATFTADSSTSTAYMELSSLRSEDTAVYYCLR
SEDVWGQGTLVTVSS
h13B1-10-1-NA_VL
(SEQ ID NO: 227)
DIVMTQSPDSLAVSLGERATINCKSSQSLLASRTRKNYLAWYQQKPGQP
PKLLIYWASTRESGVPDRFSGSGSGTDFTLTISSLQAEDVAVYYCKQSY
NIPTFGQGTKVEIKR

TABLE 18
anti-human MASP-3-specific mAb HC CDRs:

| Antibody | Region | Sequence |
|---|---|---|
| h4D5-14-1 NA | HC-CDR1 | TDDIN (SEQ ID NO: 229) |
| h4D5-19-1 NA | HC-CDR1 | TDDIN (SEQ ID NO: 229) |
| h10D12-45-21-GA | HC-CDR1 | SYGMS (SEQ ID NO: 230) |
| h10D12-49-21-GA | HC-CDR1 | SYGMS (SEQ ID NO: 230) |
| h13B1-9-1-NA | HC-CDR1 | GKWIE (SEQ ID NO: 231) |
| h13B1-10-1-NA | HC-CDR1 | GKWIE (SEQ ID NO: 231) |
| h4D5-14-1 NA | HC-CDR2 | WIYPRDDRTKYNDKFKD |
| | | (SEQ ID NO: 232) |
| h4D5-19-1 NA | HC-CDR2 | WIYPRDDRTKYNDKFKD |
| | | (SEQ ID NO: 232) |
| h10D12-45-21-GA | HC-CDR2 | WINTYSGVPTYADDFKG |
| | | (SEQ ID NO: 233) |
| h10D12-49-21-GA | HC-CDR2 | WINTYSGVPTYADDFKG |
| | | (SEQ ID NO: 233) |
| h13B1-9-1-NA | HC-CDR2 | EILPGTGSTNYAQKFQG |
| | | (SEQ ID NO: 234) |
| h13B1-10-1-NA | HC-CDR2 | EILPGTGSTNYNEKFKG |
| | | (SEQ ID NO: 235) |
| h4D5-14-1 NA | HC-CDR3 | LEDTY (SEQ ID NO: 236) |
| h4D5-19-1 NA | HC-CDR3 | LEDTY (SEQ ID NO: 236) |
| h10D12-45-21-GA | HC-CDR3 | GGEAMDY (SEQ ID NO: 237) |
| h10D12-49-21-GA | HC-CDR3 | GGEAMDY (SEQ ID NO: 237) |
| h13B1-9-1-NA | HC-CDR3 | SEDV (SEQ ID NO: 238) |
| h13B1-10-1-NA | HC-CDR3 | SEDV (SEQ ID NO: 238) |

TABLE 19
anti-human MASP-3-specific mAbs LC CDRs:

| Antibody | Region | Sequence |
|---|---|---|
| h4D5-14-1 NA | LC-CDR1 | KSSQSLLASRTRKNYLA |
| | | (SEQ ID NO: 239) |
| h4D5-19-1 NA | LC-CDR1 | KSSQSLLASRTRKNYLA |
| | | (SEQ ID NO: 239) |
| h10D12-45-21-GA | LC-CDR1 | KSSQSLLDSDAKTYLN |
| | | (SEQ ID NO: 240) |
| h10D12-49-21-GA | LC-CDR1 | KSSQSLLDSDAKTYLN |
| | | (SEQ ID NO: 240) |
| h13B1-9-1-NA | LC-CDR1 | KSSQSLLASRTRKNYLA |
| | | (SEQ ID NO: 239) |
| h13B1-10-1-NA | LC-CDR1 | KSSQSLLASRTRKNYLA |
| | | (SEQ ID NO: 239) |
| h4D5-14-1 NA | LC-CDR2 | WASTRES (SEQ ID NO: 178) |
| h4D5-19-1 NA | LC-CDR2 | WASTRES (SEQ ID NO: 178) |
| h10D12-45-21-GA | LC-CDR2 | LVSKLDS (SEQ ID NO: 241) |
| h10D12-49-21-GA | LC-CDR2 | LVSKLDS (SEQ ID NO: 241) |
| h13B1-9-1-NA | LC-CDR2 | WASTRES (SEQ ID NO: 178) |
| h13B1-10-1-NA | LC-CDR2 | WASTRES (SEQ ID NO: 178) |
| h4D5-14-1 NA | LC-CDR3 | KQSYNLYT (SEQ ID NO: 242) |
| h4D5-19-1 NA | LC-CDR3 | KQSYNLYT (SEQ ID NO: 242) |
| h10D12-45-21-GA | LC-CDR3 | WQGTHFPWT (SEQ ID NO: 243) |
| h10D12-49-21-GA | LC-CDR3 | WQGTHFPWT (SEQ ID NO: 243) |
| h13B1-9-1-NA | LC-CDR3 | KQSYNIPT (SEQ ID NO: 244) |
| h13B1-10-1-NA | LC-CDR3 | KQSYNIPT (SEQ ID NO: 244) |

TABLE 20
Representative humanized high afinity MASP-3 inhibitory antibodies

| | Heavy | | | |
|---|---|---|---|---|
| | Chain | Light | | |
| | Variable | Chain | | |
| | Region | Variable | Heavy Chain: | Light Chain: |
| | aa | Region aa | CDR1; CDR2; | CDR1; CDR2; |
| MASP-3 Antibody | (SEQ ID | (SEQ ID | CDR3 | CDR3 |
| Reference No. | NO) | NO) | (SEQ ID NOs) | (SEQ ID NOs) |
| h4D5-14-1-NA | 220 | 221 | 229, 232, 236 | 239, 178, 242 |
| h4D5-19-1-NA | 222 | 221 | 229, 232, 236 | 239, 178, 242 |
| h10D12-45-21-GA | 223 | 224 | 230, 233, 237 | 240, 241, 243 |
| h10D12-49-21-GA | 225 | 224 | 230, 233, 237 | 240, 241, 243 |
| h13B1-9-1-NA | 226 | 227 | 231, 234, 238 | 239, 178, 244 |
| h13B1-10-1-NA | 228 | 227 | 231, 235, 238 | 239, 178, 244 |

In some embodiments, the variable light chain and heavy chain fragments of the MASP-3 inhibitory antibodies were isolated in a full-length IgG4 format as follows: In some embodiments, the chimeric mAbs were fused to the human IgG4 constant region (SEQ ID NO: 70). In some embodiments, the chimeric mAbs were fused to the human IgG4 constant region which contains the stabilizing S228P amino acid substation (SEQ ID NO:71). In some embodiments, the chimeric mAbs were fused to the human IgG4 constant region which contains the S228P amino acid substitution and also a mutation that promotes FcRn interactions at low pH (SEQ ID NO:245).

As further described in WO2018/026722, high affinity MASP-3 inhibitory antibodies 13B1, 10D12 and 4D5 completely inhibit the alternative pathway in mammalian subjects such as rodents and non-primates at molar concentrations less than the concentration of the MASP-3 target (e.g., at a molar ratio of from about 1:1 to about 2.5:1 (MASP-3 target to mAb) (see in Examples 11-21). As described in Example 11, a single dose administration of a high affinity MASP-3 inhibitory antibody, mAb 13B1, to mice led to near-complete ablation of systemic alternative pathway complement activity for at least 14 days. As further described in Example 12, in a study conducted in a well-established animal model associated with PNH it was demonstrated that mAb 13B1 significantly improved the survival of PNH-like red blood cells and protected PNH-like red blood cells significantly better than did C5 inhibition. As described in Example 13, it was further demonstrated that mAb 13B1 reduced the incidence and severity of disease in a mouse model of arthritis. As further described in WO2018/026722, representative high affinity MASP-3 inhibitory mAbs 13B1, 10D12 and 4D5 are highly effective at blocking the alternative pathway in primates. Single dose administration of mAb 13B1, 10D12 or 4D5 to cynomolgus monkeys resulted in sustained ablation of systemic alternative pathway activity lasting for approximately 16 days. The extent of alternative pathway ablation in cynomolgus monkeys treated with high affinity MASP-3 inhibitory antibodies was comparable to that achieved by factor D blockade in vitro and in vivo, indicating complete blockade of factor D conversion by the MASP-3 inhibitory antibodies. Therefore, high affinity MASP-3 inhibitory mAbs have therapeutic utility in the treatment of patients suffering from diseases or disorders related to alternative pathway hyperactivity, such as, for example, wherein the disease or disorder related to alternative pathway hyperactivity (also referred to as alternative-pathway disease or disorder) is selected from the group consisting of: paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

### Example 11

Analysis of Factor D in Cynomolgus Monkey Samples before and after Treatment with a representative MASP-3 inhibitory antibody (13B1) in an immunoassay using mature Factor D-specific antibody 14A11 as a detection antibody.

### Background/Rationale:

As described in Example 10, numerous high affinity MASP-3 inhibitory antibodies have been generated that are capable of inhibiting steady-state (resting) pro-factor D maturation in vivo. This Example describes an analysis of the status of Factor D (i.e., the amount of mature Factor-D) in cynomolgus monkeys after treatment with a representative high affinity MASP-3 inhibitory mAb 13B1, which is known to be capable of inhibiting APC activity in a non-human primate.

### Methods:

In this study, 9 cynomolgus monkeys (3 animals per mAb condition) were given a single 5 mg/kg intravenous dose with one of three representative high affinity MASP-3 inhibitory antibodies: 4D5, 10D12, or 13B1 (IgG4 constant region). Plasma (EDTA) and serum samples were collected at regular intervals over a period of three weeks or longer.

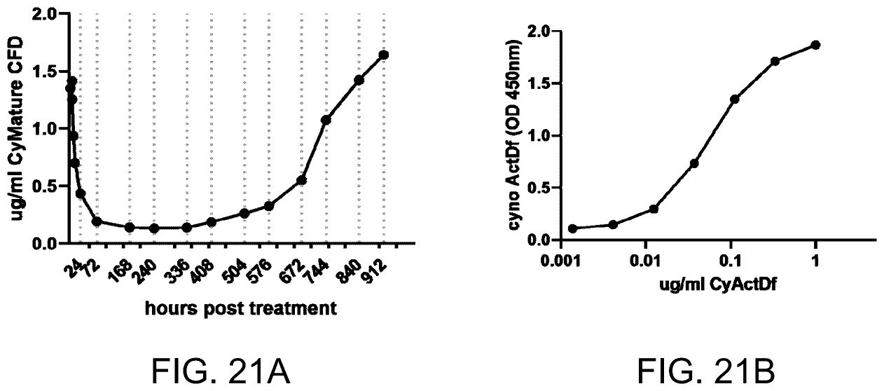

Plasma samples from a single dose of 13B1 in cynomolgus monkey were tested for the amount of mature Factor D as follows. ELISA plates were coated overnight with 3 μg/mL of anti-human/cyno Factor D mAb 3C5 (human IgG4) which binds to both human pro-Factor D and mature-Factor D. The plates were washed, blocked, and loaded with 20-fold dilutions of study samples and incubated at room temperature for 1 hour. After washing, a 3 μg/mL solution of mature Factor D-specific antibody 14A11 was added and incubated for 1 hour. After washing, an HRP-labeled secondary antibody (HRP-labeled F(ab′)2 Fragment Donkey anti-Mouse IgG H&L antibody (Jackson ImmunoResearch)) was used to signal the detection antibody, followed by development with TMB (ThermoFisher). Samples were interpolated from a 4-parameter logistics curve of cynomolgus recombinant mature Factor D dilutions. The results are shown in FIG. 21A and FIG. 21B.

### Results:

FIG. 21A graphically illustrates the amount of mature Factor D in a cynomolgus monkey over a time period after treatment with representative anti-MASP-3 inhibitory mAb 13B1. As shown in FIG. 21A, the level of mature Factor D drops to a low level within 24 hours after administration of the MASP-3 mAb and remains low for about 500 hours post treatment.

FIG. 21B graphically illustrates the standard curve as determined from a 4-parameter logistics curve of cynomolgus recombinant mature Factor D dilutions. The sample readouts were interpolated from this standard curve, and graphed as ug/ml of cynomolgus mature factor D.

The results in this Example demonstrate that an immunoassay utilizing an antibody specific for mature Factor D may be used to monitor the serum level of mature Factor D after treatment with a MASP-3 inhibitory antibody that inhibits the conversion of pro-Factor D to mature Factor D.

### Example 12

Analysis of the pharmacology of representative anti-MASP-3 mAb (13B1) in cynomolgus monkeys as part of a single-dose pharmacokinetic (PK) and pharmacodynamic (PD) study.

### Background/Rationale:

As demonstrated in Example 11, an immunoassay utilizing an antibody specific for mature Factor D may be used to monitor the serum level of mature Factor D after treatment with a MASP-3 inhibitory antibody that inhibits the conversion of pro-Factor D to mature Factor D. This Example describes the use of an immunoassay utilizing an antibody specific for mature Factor D to quantitate the plasma concentration of mature Factor D in a single-dose pharmacokinetic (PK) and pharmacodynamic (PD) study in female cynomolgus monkeys.

### Methods:

The pharmacology of anti-MASP-3 mAb 13B1 in monkeys was explored as part of a single-dose pharmacokinetic (PK) and pharmacodynamic (PD) study in female cynomolgus monkeys. mAb 13B1 was administered by subcutaneous (SC) injection at 0.5 mg/kg, 1.5 mg/kg, or 5 mg/kg; or by intravenous (IV) bolus injection at 5 mg/kg or 100 mg/kg. Each study group contained 3 monkeys. Serial blood samples were collected for the assessment of PK and PD from pre-dose out to 1344 hours (8 weeks) post-dose.

The effect of mAb 13B1 on alternative pathway activity was assessed by the quantitation of mature Factor D in an ELISA assay and also by using an ex vivo Factor Ba activity assay. The concentration of Factor Ba produced upon stimulation was calculated by the subtraction of Factor Ba concentration in unstimulated samples from the Factor Ba concentration in stimulated samples. A decrease in mature (i.e., active) Factor D plasma concentration and a decrease in stimulated Factor Ba concentration are indicative of mAb13B1 PD activity Mature Factor D plasma concentration data and stimulated Factor Ba concentration were summarized by timepoint and dose group using mean, median, standard deviation and coefficient of variation (CV %).

Serum samples were stimulated with zymosan to activate the alternative pathway. The extent of alternative pathway activation by zymosan was determined by the quantitation of Factor Ba as follows. For determining generation of the fluid phase marker Ba, the APC was induced in ex vivo assays by incubating zymosan (1 mg/mL final) in serum (5% final, diluted in GVB+Mg/EGTA) prepared from anti-MASP-3 mAb-treated cynomolgus monkeys. The mixtures were incubated at 37° C. for 40 minutes, and the APC activity was measured by ELISA-based detection of the complement endpoints. Ba was detected in the reaction supernatants using commercially available ELISA kits (Quidel). Absorbance values of all tests were normalized by setting pre-treatment values as 100% activity, and a pre-treatment sample incubated, but not exposed to zymosan, to 0%.

Mature Factor D ELISA assay: ELISA plates were coated overnight with 3 μg/mL of anti-human/cyno Factor D mAb 3C5 (human IgG4) which binds to both human pro-Factor D and mature-Factor D. The plates were washed, blocked, and loaded with 20-fold dilutions of study samples and incubated at room temperature for 1 hour. After washing, a 3 μg/mL solution of mature Factor D-specific antibody 14A11 was added and incubated for 1 hour. After washing, an HRP-labeled secondary antibody (Jackson ImmunoResearch) was added to develop the assay.

### Results:

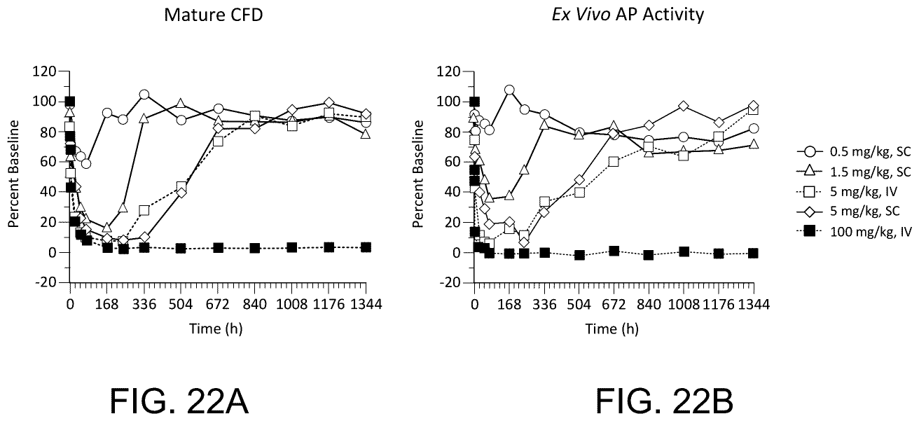

FIG. 22A graphically illustrates the concentration of mature Factor D in monkeys over a time period of 56 days (1344 hours) after s.c. or i.v. administration of anti-MASP-3 mAb 13B1, as determined in an ELISA assay with mature Factor D-specific antibody 14A11 utilized as a detection antibody. As shown in FIG. 22A, administration of mAb 13B1 in monkeys was associated with a dose-dependent decrease in mature Factor D concentration.

FIG. 22B graphically illustrates the ex vivo alternative pathway activity (% baseline) over a time period of 56 days (1344 hours) after administration anti-MASP-3 mAb 13B1, as determined in a Factor Ba assay. As shown in FIG. 22B, following administration of mAb 13B1, ex vivo alternative pathway activity was inhibited in a dose-dependent manner. With increasing dose, the extent and duration of the inhibition of ex vivo activity increased. Following s.c. or i.v. administration of 5 mg/kg mAb 13B1, ex vivo activity decreased to approximately 10% of pre-dose levels for approximately 2 weeks. At the highest dose levels evaluated, ex vivo activity was inhibited for the duration of the sampling period.

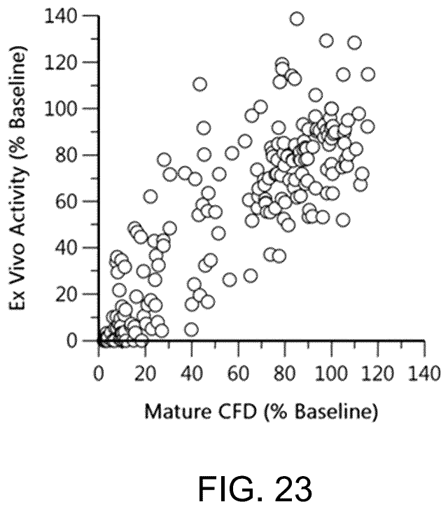

FIG. 23 graphically illustrates the relationship of anti-MASP-3 mAb 13B1 effects on ex vivo alternative pathway activity and mature Factor D concentration following a single intravenous bolus or subcutaneous administration in monkeys. As shown in FIG. 23, the effect of mAb 13B1 on mature Factor D concentration was linearly related to the effect of mAb 13B1 on ex vivo alternative pathway activity.

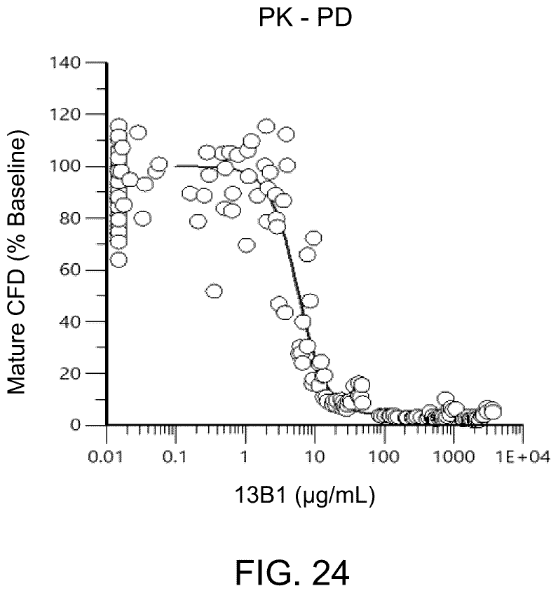

The relationship between mAb 13B1 concentration and PD effect on mature CFD concentration in the monkey study was explored graphically and fit using a sigmoidal concentration-response model. Due to the lag between mAb13B1 serum concentration and decrease in mature CFD concentration, data collected prior to 72 hours post-dose were excluded from the analysis. A plot of the observed data and PD model fit is presented in FIG. 24.

FIG. 24 graphically illustrates the relationship between anti-MASP-3 mAb13B1 serum concentration and pharmacodynamic effect of mature Factor D concentration following a single administration to monkeys.

### Summary of Results:

As expected for MASP-3 inhibition, APC inhibition in mice and non-human primates is associated with a decrease in the activation of systemic CFD. Post-dose concentration measurements of plasma CFD, by enzyme-linked immunosorbent assays (ELISAs) that specifically detect the either the zymogen form (inactive proenzyme) or the mature form, demonstrate that anti-MASP-3 mAb13B1 blocks the maturation of CFD to the active form without a measurable change in the level of total CFD. A single 5 mg/kg dose of mAb13B1 administered s.c. can maintain ≥90% alternative pathway blockade for approximately 2 weeks in nonhuman primates.

Following administration of anti-MASP-3 mAb13B1 in monkeys, ex vivo alternative pathway activity was inhibited in a dose-dependent manner. With increasing dose, the extent and duration of the inhibition of ex vivo activity increased. Following s.c. or i.v. administration of 5 mg/kg mAb13B1, ex vivo activity decreased to approximately 10% of pre-dose levels for approximately 2 weeks. Administration of mAb13B1 in monkeys was also associated with a dose-dependent decrease in mature CFD concentration. The effect of mAb13B1 on mature CFD concentration was generally linearly related to the effect of mAb13B1 on ex vivo alternative pathway activity. These data indicate that mAb13B1 inhibits MASP-3 activity after a single administration in monkeys, and that the inhibition of MASP-3 leads to a decrease in alternative pathway activity. The decrease in mature Factor D plasma concentration and stimulated Factor Ba concentration demonstrated the mAb13B1 dose-dependent inhibition of the alternative pathway in monkeys.

### Example 13

Phase 1 Clinical Trial to assess safety, tolerability, pharmacokinetic (PK) and pharmacodynamics (PD) of mAb13B1

### Background/Rationale:

As described herein, mAb13B1 is a humanized monoclonal antibody that binds to the serine protease domain of MASP-3 and inhibits its activity. This Example describes a Phase 1 first in human study that will be carried out to assess safety, tolerability, pharmacokinetic (PK) and pharmacodynamics (PD) of mAb13B1. The PD analysis in this study comprises the use of immunoassays disclosed herein to assess the extent of alternative pathway complement (APC) inhibition in the subjects treated with mAb13B1 by capturing and detecting mature Factor D in the test sample, wherein mature Factor D is either captured or detected with a mature Factor D-specific monoclonal antibody or fragment thereof that specifically binds to an epitope in “ILGGREA” (SEQ ID NO:5) present in mature Factor D, but does not bind to Pro-Factor D; and/or capturing and detecting Pro-Factor D in the test sample, wherein Pro-Factor D is either captured or detected with a Pro-Factor D-specific monoclonal antibody or fragment thereof that specifically binds to an epitope on the activation (“Pro”) peptide “APPRGR” (SEQ ID NO:4) present in Pro-Factor D, but does not bind to mature Factor D.

### Methods:

The Phase 1 first-in-human study of mAb13B1 will consist of a single ascending-dose study of IV and SC administration of mAb13B1 and a multiple ascending-dose study of SC administration of mAb13B1. In both parts of the study, healthy subjects will be enrolled to assess safety, tolerability, pharmacokinetics (PK), pharmacodynamics (PD), and immunogenicity. The aim of single ascending-dose study will be to establish a dose range and schedule that is well tolerated and provides ≥90% inhibition of MASP-3 activity for approximately 30 days (measured by reduction in mature CFD plasma concentration in an immunoassay as disclosed herein). The multiple ascending-dose portion of the study is designed to determine a dose level and frequency of SC dosing that will sustain ≥90% inhibition of MASP-3. Nonclinical toxicity studies of mAb13B1 indicate that there is an adequate safety margin to conduct initial human testing at the proposed doses in healthy subjects, and it is predicted that dose levels explored in the Phase 1 study will provide efficacy in diseases characterized by APC overactivity for a period of time that would be convenient for patients.

As described herein, mAb13B1 is a humanized monoclonal antibody (mAb) that binds to the serine protease domain of MASP-3 and inhibits its activity. By inhibiting MASP-3, mAb13B1 blocks the proteolytic activation of CFD and thereby disrupts the APC and its associated amplification of complement activity. mAb13B1 comprises a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT). mAb13B1 comprises a humanized variable region of murine origin fused to IgG4 constant region of human origin set forth as SEQ ID NO:245. mAb13B1 is secreted as a disulfide-linked glycosylated tetramer consisting of 2 identical 219-amino-acid kappa light chains and 2 identical 440-amino-acid heavy chains.

The Drug Product used in this Phase 1 trial contains mAb13B1 at a concentration of 110 mg/mL, 20 mM histidine, 100 mg/mL sucrose, and 0.035% polysorbate 80 at a pH of 6.0.

### Dosage Determination

As described in Example 12, the relationship between mAb13B1 concentration and PD effect on mature CFD concentration in the 2 single-dose studies in monkeys was explored graphically and fit using a sigmoidal concentration-response model. Due to the lag between mAb 13B1 serum concentration and decrease in mature CFD concentration, data collected prior to 72 hours post-dose were excluded from the analysis. The plot of the observed data and PD model fit is shown in FIG. 24.

The mAb13B1 PK and PD model parameters were then scaled to predict mAb13B1 PK and PD effect in humans across a wide dose range. The PK model parameters were scaled to human using allometric coefficients typically used for monoclonal antibodies (Deng R. et al., MAbs, 3 (1): 61-6, 2011; Dong J. et al., Clin Pharmacokinet 50 (2): 131-42, 2011). Based on the in vitro potency and binding affinity data, the PD model parameters were assumed to be the same in humans as in monkeys. The mAb13B1 exposure-response relationship model was used to estimate the mAb13B1 concentration associated with a 10%, 50%, and 90% decrease in mature CFD concentrations. Using the mAb13B1 PK model, mAb13B1 serum concentration over time profiles were simulated across a range of IV and SC dose levels in humans. The predicted mAb13B1 exposure values and PD effect across the range of IV and SC dose levels in humans are presented in TABLE 21.

TABLE 21
Predicted mAb13B1 Exposure and Pharmacodynamic Effect Following a
Single Subcutaneous or Intravenous Administration in Healthy Volunteers

| | | | Predicted Pharmacokinetics | | Predicted Pharmacodynamics | |
|---|---|---|---|---|---|---|
| | | | mAb13B1 Exposure | | Reduction in Mature CFD | |
| | Dose and Route | | Cₘₐₓ | AUC_{(0-168 h)} | Eₘₐₓ | |
| Cohort | (mg/kg) | | (μg/mL) | (μg * h/mL) | (%) | Duration > 90% |
| 1 | 0.1 | IV^{a} | 2.07 | 90.3 | ~10 | 0 |
| 2 | 0.3 | IV^{a} | 6.24 | 323 | ~50 | 0 |
| 3 | 1 | IV^{a} | 20.9 | 1420 | ~90 | 0 |
| 4 | 3 | IV^{a} | 62.8 | 5140 | >90 | ~1 week |
| 5 | 3 | SC | 31.0 | 4160 | >90 | ~1 week |
| 6 | 5 | SC | 53.5 | 7310 | >90 | ~2 weeks |
| 7 | 8 | SC | 87.6 | 12100 | >90 | ~4 weeks |
| 8 | 8 | IV^{a} | 168 | 14800 | >90 | ~4 weeks |

AUC_{(0-168 h)} = Area under the concentration curve over time from 0 to 168 hours postdose; Cₘₐₓ = Maximum observed concentration; Eₘₐₓ = Maximum pharmacodynamic effect; IV = Intravenous; CFD = complement factor D; SC = subcutaneous
^{a}30-minute infusion

The predicted mAb13B1 pharmacokinetic (PK) profile and pharmacodynamic (PD) activity suggest that a single IV administration of 0.1 mg/kg in humans would be associated with a maximum 10% decrease in mature CFD plasma concentration. Similarly, a single SC or IV administration of 8 mg/kg is predicted to be associated with a >90% decrease in mature CFD levels for approximately 4 weeks.

### Summary of Results

A total of 80 healthy subjects were administered mAb13B1 intravenously (IV) at dosages of 0.1 mg/kg, 0.3 mg/kg, 1 mg/kg, or 3 mg/kg or subcutaneously (SC) at dosages of 3 mg/kg or 5 mg/kg. At all doses tested, mAb13B1 was well tolerated, with no apparent safety signals.

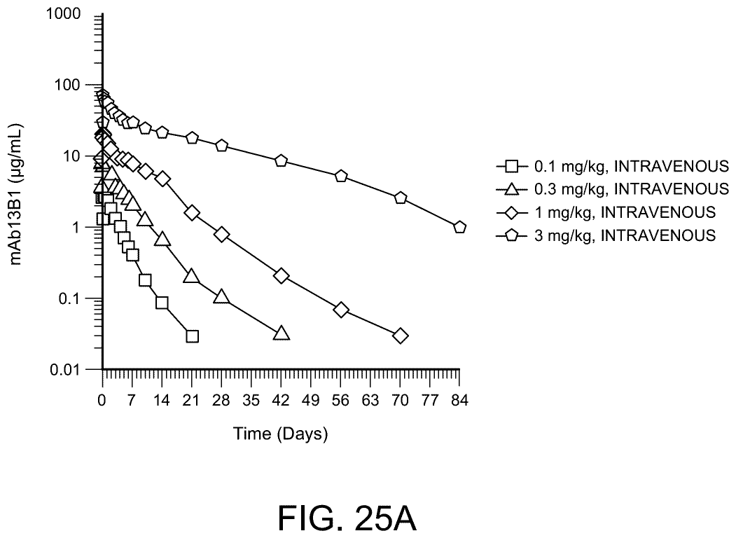

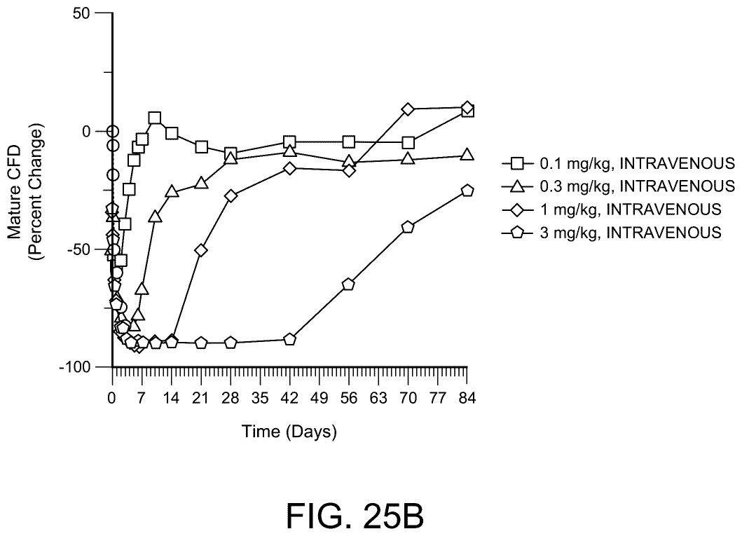

FIG. 25A graphically illustrates the concentration of mAb13B1 in plasma of subjects over a time period of up to 84 days after IV administration of mAb13B1, as determined by ELISA. As shown in FIG. 25A, increased dosing resulted in higher levels of mAb13B1 and a longer period of time during which mAb13B1 was detected in plasma samples.

FIG. 25B graphically illustrates the levels of mature Factor D in subjects over a time period of 84 days after IV administration of anti-MASP-3 mAb 13B1, as determined in an ELISA assay with mature Factor D-specific antibody 14A11 used as a detection antibody. As shown in FIG. 25B, administration of mAb 13B1 was associated with a dose-dependent decrease in mature Factor D concentration as well as a dose-dependent increase in the duration of the effect. At dosages of 3 mg/kg, IV administration resulted in a decrease in mature CFD levels below detectable levels (i.e., a decrease of approximately 90% or more) that persisted for up to four weeks. It was also determined that the single lowest subcutaneous dose of mAb 13B1 was able to suppress mature Factor D concentration to below detectable levels (i.e., a decrease of approximately 90% or more) for four weeks in most subjects.

These data illustrate that the PK and PD profile across all dosages tested is favorable and supports low-dose, once-monthly (or less frequent) dosing for mAb 13B1. Such dosing may be either intravenous or subcutaneous.

The utility of the pro-Factor D and mature Factor D assays to measure a pharmacodynamic response in normal human volunteers who received a single dose of MASP-3 inhibitory antibody 13B1 has been demonstrated in the ongoing Phase 1 study. At multiple dose levels of Ab 13B1, the pro-Factor D and mature Factor D plasma concentrations consistently show an inverse correlation. Relative to pre-dose, baseline levels, pro-Factor D levels increased as mature Factor D levels decreased following Ab 13B1 administration. Furthermore, the extent of the measured increase of pro-Factor D consistently approximated the decrease in the concentration of mature Factor D. This observation is concordant with the expected outcome of inhibition of MASP-3 and Factor D maturation in humans if the clearance rates of pro-Factor D and mature Factor D do not differ dramatically. In summary, the outcomes of the two assays that measure the two different forms of Factor D are supportive of one another and, when utilized together, may provide additional diagnostic value for characterizing therapeutic MASP-3 inhibition.

Accordingly, in one aspect, the present disclosure provides a pharmaceutical composition comprising a MASP-3 inhibitory antibody in an aqueous solution comprising a buffer system having a pH of 6.0±5%, 20±5% mM histidine, 100±5% mg/mL sucrose, and 0.035%+5%, polysorbate 80, wherein said MASP-3 inhibitory antibody is included at a concentration of 110 mg/mL+5%, and wherein said MASP-3 inhibitory antibody comprises a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT). In one embodiment, the pharmaceutical composition is sterile. In one embodiment, the MASP-3 inhibitory antibody or antigen-binding fragment thereof comprises a heavy chain variable region comprising at least 80%, 85%, 90%, 95%, 98%, 99% or 100% identical to SEQ ID NO:226 or SEQ ID NO:227 and a light chain variable region comprising at least 80%, 85%, 90%, 95%, 98%, 99% or 100% identical to SEQ ID NO:227. In one embodiment, the MASP-3 inhibitory antibody or antigen binding fragment thereof is selected from the group consisting of a human antibody, a humanized antibody, a chimeric antibody, a murine antibody, and an antigen-binding fragment of any of the foregoing. In one embodiment, the MASP-3 inhibitory antibody or antigen-binding fragment thereof is selected from the group consisting of a single chain antibody, an ScFv, a Fab fragment, an Fab′ fragment, an F(ab′)2 fragment, a univalent antibody lacking a hinge region and a whole antibody. In one embodiment, the MASP-3 inhibitory antibody further comprises an immunoglobulin constant region. In one embodiment, the MASP-3 inhibitory antibody comprises a human IgG4 constant region. In one embodiment, the MASP-3 inhibitory antibody comprises a human IgG4 constant region with an S228P mutation. In one embodiment, the MASP-3 inhibitory antibody comprises a mutation that promotes FcRn interations at low pH, such as, for example, wherein the MASP-3 inhibitory antibody comprises human IgG4 constant region set forth as SEQ ID NO:245. In one embodiment, the pharmaceutical composition is administered to a subject in need thereof at a dosage in the range of 0.1 to 10 mg of MASP-3 inhibitory antibody per kg of body weight (such as from 0.1 mg/kg to 8 mg/kg, from 0.3 mg/kg to 5 mg/kg, from 0.3 mg/kg to 3 mg/kg, from 1 mg/kg to 3 mg/kg, from 1 mg/kg to 5 mg/kg, from 2 mg/kg to 5 mg/kg, 0.1±5% mg/kg, 0.3±5% mg/kg, 1.0±5% mg/kg, 3.0±5% mg/kg, 5.0±5% mg/kg, or 8.0±5% mg/kg). In one embodiment, the pharmaceutical composition is administered to a subject in need thereof at a dosage in the range of 0.5 to 5 mL per 100 kg of body weight (such as from 0.7 mL to 4.5 mL, from 1.0 mL to 3.5 mL, from 1.5 mL to 3.0 mL, from 2 mL to 2.5 mL, 0.5±5% mL, 0.7±5% mL, 1.1±5% mL, 1.4±5% mL, 2.1±5% mL, 2.3±5% mL, 2.8±5% mL, 3.4±5% mL, or 4.5±5% mL).

In another aspect, the present disclosure provides an article of manufacture containing a pharmaceutical composition comprising a MASP-3 inhibitory antibody in a unit dosage form suitable for therapeutic administration to a human subject, such as a unit dosage in the range of from 10 mg to 1000 mg (such as from 50 mg to 800 mg, or from 75 mg to 500, such as from 100 mg to 300 mg, such as 125 to 275 mg, such as 150 to 200 mg, such as 150±5% mg, 155±5% mg, 160±5% mg, 165±5% mg, 170±5% mg, 175±5% mg 180±5% mg, 185±5% mg, or 190±5% mg) of MASP-3 inhibitory antibody, wherein said MASP-3 inhibitory antibody comprises a heavy chain variable region comprising a HC-CDR1 comprising SEQ ID NO: 231 (GKWIE); a HC-CDR2 comprising SEQ ID NO:234 (EILPGTGSTNYNEKFKG) or SEQ ID NO:235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT).

While the preferred embodiment of the invention has been illustrated and described, it will be appreciated that various changes can be made therein without departing from the spirit and scope of the invention.

1. An isolated antibody, or antigen-binding fragment thereof, that specifically binds to an epitope in the amino-terminal region of human mature Factor D, wherein the epitope comprises or consists of the amino acid sequence ILGGREA (SEQ ID NO:5).

2. The isolated antibody or antigen-binding fragment thereof of claim 1, wherein the antibody or antigen-binding fragment thereof specifically binds human mature Factor D (SEQ ID NO: 3) and does not bind to human Pro-Factor D (SEQ ID NO:2).

3. The isolated antibody or antigen-binding fragment thereof of claim 1, wherein the antibody is a monoclonal antibody.

4. The isolated antibody or antigen-binding fragment thereof of claim 3, wherein said antibody is a humanized, chimeric, or fully human antibody.

5. The isolated antibody or antigen-binding fragment thereof of claim 3, wherein said antigen-binding fragment is selected from the group consisting of Fv, Fab, Fab′, F(ab)₂ and F(ab′)₂.

6. The isolated antibody or antigen-binding fragment thereof of claim 3, wherein said antibody or antigen-binding fragment thereof is a single chain molecule.

7. The isolated antibody or antigen-binding fragment thereof of claim 3, wherein said antibody is an IgG molecule selected from the group consisting of IgG1, IgG2 and IgG4.

8. The isolated antibody or antigen-binding fragment thereof of claim 1, wherein said antibody or antigen-binding fragment thereof binds to human mature Factor D with a K_{D} of less than 10 nM.

9. The isolated antibody or antigen-binding fragment thereof of claim 1, wherein said antibody or antigen-binding fragment thereof is labeled with a detectable moiety.

10. The isolated antibody or antigen-binding fragment thereof of claim 1, wherein said antibody or antigen-binding fragment thereof is immobilized on a substrate.

11. The isolated antibody or antigen-binding fragment thereof of claim 1, wherein the isolated antibody or antigen-binding fragment thereof that specifically binds to human mature Factor D comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23, wherein the CDRs are numbered according to the Kabat numbering system.

12. The isolated antibody or antigen-binding fragment thereof of claim 1, wherein the antibody or antigen-binding fragment thereof that specifically binds to human mature Factor D comprises a binding domain comprising the following six CDRs: a) an HC-CDR1 comprising the amino acid sequence XSXMGVS (SEQ ID NO:65), wherein X at position 1 is T, I or S and X at position 3 is G or I; (b) an HC-CDR2 comprising the amino acid sequence HIYWDDEKHYXPSLKX (SEQ ID NO:66), wherein X at position 11 is H or N and X at position 16 is S or R; (c) an HC-CDR3 comprising the amino acid sequence RYYGYXXXMXY (SEQ ID NO: 67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is N or S, X at position 4 is Q or E, X at position 7 is V or L, and X at position 15 is F or L; (e) a LC-CDR2 comprising the amino acid sequence KVXNRFS (SEQ ID NO:69), wherein: X at position 3 is S or Y; and (f) a LC-CDR3 comprising the amino acid sequence FQGSHVPPT (SEQ ID NO:54).

13. The isolated antibody or antigen-binding fragment thereof of claim 12, wherein the binding domain comprises the following six CDRs: (a) an HC-CDR1 comprising SEQ ID NO: 25, (b) an HC-CDR2 comprising SEQ ID NO:27; (c) an HC-CDR3 comprising SEQ ID NO: 29; (d) a LC-CDR1 comprising SEQ ID NO:50, (e) a LC-CDR2 comprising SEQ ID NO:52 and (f) a LC-CDR3 comprising SEQ ID NO:54.

14. The isolated antibody or antigen-binding fragment thereof of claim 13, wherein the isolated antibody or antigen-binding fragment thereof comprises at least one of:
(a) a VH domain having at least 95% sequence identity to the amino acid sequence of SEQ ID NO: 12 or SEQ ID NO:13;
(b) a VL domain having at least 95% sequence identity to the amino acid sequence of SEQ ID NO: 18 or SEQ ID NO:19;
(c). a VH comprising SEQ ID NO:12 and a VL comprising SEQ ID NO:18; and/or
(d) a VH domain comprising SEQ ID NO:13 and a VL domain comprising SEQ ID NO:19.

15. The isolated antibody or antigen-binding fragment thereof of claim 12, wherein the binding domain comprises the following six CDRs: (a) an HC-CDR1 comprising SEQ ID NO: 33, (b) an HC-CDR2 comprising SEQ ID NO:34; (c) an HC-CDR3 comprising SEQ ID NO: 36; (d) a LC-CDR1 comprising SEQ ID NO:58, (e) a LC-CDR2 comprising SEQ ID NO:52 and (f) a LC-CDR3 comprising SEQ ID NO:54.

16. The isolated antibody or antigen-binding fragment thereof of claim 15, wherein the isolated antibody or antigen-binding fragment thereof comprises at least one of:
(a) a VH domain having at least 95% sequence identity to the amino acid sequence of SEQ ID NO: 14;
(b) a VL domain having at least 95% sequence identity to the amino acid sequence of SEQ ID NO: 20; and/or
(c) a VH domain comprising SEQ ID NO:14 and a VL domain comprising SEQ ID NO:20.

17. The isolated antibody or antigen-binding fragment thereof of claim 12, wherein the binding domain comprises the following six CDRs: (a) an HC-CDR1 comprising SEQ ID NO: 38, (b) an HC-CDR2 comprising SEQ ID NO:39; (c) an HC-CDR3 comprising SEQ ID NO: 41; (d) a LC-CDR1 comprising SEQ ID NO:60, (e) a LC-CDR2 comprising SEQ ID NO:52 and (f) a LC-CDR3 comprising SEQ ID NO:54.

18. The isolated antibody or antigen-binding fragment thereof of claim 17, wherein the isolated antibody or antigen-binding fragment thereof comprises at least one of:
(a) a VH domain having at least 95% sequence identity to the amino acid sequence of SEQ ID NO: 15;
(b) a VL domain having at least 95% sequence identity to the amino acid sequence of SEQ ID NO: 21; and/or
(c) a VH domain comprising SEQ ID NO:15 and a VL domain comprising SEQ ID NO:21.

19. The isolated antibody or antigen-binding fragment thereof of claim 12, wherein the binding domain comprises the following six CDRs: (a) an HC-CDR1 comprising SEQ ID NO: 43, (b) an HC-CDR2 comprising SEQ ID NO:39; (c) an HC-CDR3 comprising SEQ ID NO: 41; (d) a LC-CDR1 comprising SEQ ID NO:62, (e) a LC-CDR22 comprising SEQ ID NO:52 and (f) a LC-CDR3 comprising SEQ ID NO:54.

20. The isolated antibody or antigen-binding fragment thereof of claim 19, wherein the isolated antibody or antigen-binding fragment thereof comprises at least one of:
(a) a VH domain having at least 95% sequence identity to the amino acid sequence of SEQ ID NO: 16;
(b) a VL domain having at least 95% sequence identity to the amino acid sequence of SEQ ID NO: 22; and/or
(c) a VH domain comprising SEQ ID NO:16 and a VL comprising SEQ ID NO:22.

21. The isolated antibody or antigen-binding fragment thereof of claim 12, wherein the binding domain comprises the following six CDRs: (a) an HC-CDR1 comprising SEQ ID NO: 43, (b) an HC-CDR2 comprising SEQ ID NO:39; (c) an HC-CDR3 comprising SEQ ID NO: 47; (d) a LC-CDR1 comprising SEQ ID NO:63, (e) a LC-CDR2 comprising SEQ ID NO:64 and (f) a LC-CDR3 comprising SEQ ID NO:54.

22. The isolated antibody or antigen-binding fragment thereof of claim 21, wherein the isolated antibody or antigen-binding fragment thereof comprises at least one of:
(a) a VH domain having at least 95% sequence identity to the amino acid sequence of SEQ ID NO: 17;
(b) a VL domain having at least 95% sequence identity to the amino acid sequence of SEQ ID NO: 23; and/or
(c) a VH domain comprising SEQ ID NO:17 and a VL domain comprising SEQ ID NO:23.

23. A nucleic acid molecule encoding the CDRs of a heavy chain variable region of an antibody, or antigen-binding fragment thereof, that specifically binds human mature Factor D as set forth claim 11.

24. A nucleic acid molecule encoding the CDRs of a light chain variable region of an antibody, or antigen-binding fragment thereof, that specifically binds human mature Factor D as set forth 11.

25. A cloning vector or expression cassette comprising a nucleic acid molecule encoding the CDRs of a heavy chain variable region and/or the CDRs of a light chain variable region of an antibody, or antigen-binding fragment thereof, that specifically binds human mature Factor D.

26. A cell comprising at least one of the nucleic acid molecules encoding an antibody, or antigen-binding fragment thereof, that specifically binds human mature Factor D according to claim 23 or claim 24.

27. A method of generating an isolated antibody, or antigen-binding fragment thereof, that specifically binds human mature Factor D comprising culturing the cell of claim 26 under conditions allowing for expression of the nucleic acid molecules encoding the antibody, or antigen-binding fragment thereof, that specifically binds human mature Factor D and isolating said anti-mature-Factor-D specific antibody, or antigen-binding fragment thereof.

28. A composition comprising the antibody, or antigen-binding fragment thereof, that specifically binds human mature Factor D of claim 1.

29. A substrate for use in an immunoassay comprising at least one antibody, or antigen-binding fragment thereof, that specifically binds human mature Factor D as set forth in claim 1.

30. A kit for detecting the presence or amount of mature Factor D in a test sample, said kit comprising (a) at least one container, and (b) at least one antibody, or antigen-binding fragment thereof, that specifically binds human mature Factor D as set forth claim 1.

31-79. (canceled)

80. A kit comprising at least one monoclonal antibody that specifically detects or quantitates human mature Factor D (SEQ ID NO:3):2 in an immunoassay, wherein the at least one monoclonal antibody comprises:
a mature Factor D-specific monoclonal antibody, or antigen-binding fragment thereof, that specifically binds to an epitope encompassing the amino-terminus of human mature Factor D, wherein the epitope comprises or consists of the amino acids ILGGREA (SEQ ID NO:5) and wherein said antibody does not bind to human Pro-Factor D (SEQ ID NO:2).

81. The kit of claim 80, wherein the kit further comprises an anti-Factor D antibody, or antigen-binding fragment thereof, that binds to an epitope shared by both human mature Factor D (SEQ ID NO:3) and human Pro-Factor D (SEQ ID NO:2).

82. The kit of claim 80, wherein the kit further comprises at least one container.

83. The kit of claim 80, wherein the antibody or antigen-binding fragment thereof that specifically binds to human mature Factor D comprises a binding domain comprising HC-CDR-1, HC-CDR-2 and HC-CDR-3 in a heavy chain variable region selected from the group consisting of SEQ ID NO:s 12-17 and comprising LC-CDR-1, LC-CDR2 and LC-CDR3 in a light chain variable region selected from the group consisting of SEQ ID NO:s 18-23, wherein the CDRs are numbered according to the Kabat numbering system.

84. (canceled)

85. The kit of claim 80, wherein the immunoassay is an enzyme-linked immunosorbent assay (ELISA).

86. The kit of claim 80, wherein the mature Factor D-specific antibody or antigen-binding fragment thereof of subpart (i) is a coating antibody.

87. The kit of claim 80, wherein the mature Factor D-specific antibody or antigen-binding fragment thereof of subpart (i) is a detecting antibody.

88-89. (canceled)

90. The kit of claim 81, wherein the anti-Factor D antibody or antigen-binding fragment thereof comprises a binding domain comprising HC-CDR1, HC-CDR2 and HC-CDR3 in a heavy chain variable region selected from the group consisting of SEQ ID NO:s 85-88 and comprising LC-CDR1, LC-CDR2 and LC-CDR3 in a light chain variable region selected from the group consisting of SEQ ID NO:s 89-93, wherein the CDRs are numbered according to the Kabat numbering system.

91. A method of determining the presence or amount of mature Factor D in a test sample, the method comprising:
(a) contacting a test sample with a mature Factor D-specific monoclonal antibody or antigen-binding fragment thereof, in an in vitro immunoassay; and
(b) detecting the presence or absence or amount of the antibody or antigen-binding fragment thereof bound to mature Factor D, wherein the presence of binding indicates the presence or amount of mature Factor D in the sample;
wherein the anti-human mature Factor D-specific antibody or antigen binding fragment thereof binds to an epitope in the N-terminal region of mature Factor D, set forth as amino acids ILGGREA (SEQ ID NO:5).

92. The method of claim 91, wherein the antibody, or antigen-binding fragment thereof, specifically binds human mature Factor D (SEQ ID NO:3) and does not bind to human Pro-Factor D (SEQ ID NO:2).

93. The method of claim 91, wherein the anti-human mature Factor D-specific antibody or antigen-binding fragment thereof is immobilized on a substrate.

94. The method of claim 91, wherein the immunoassay is an ELISA assay.

95. The method of claim 91, wherein said anti-human mature Factor D-specific antibody or antigen-binding fragment thereof is labeled with a detectable moiety and step (b) comprises detecting the presence or amount of said detectable moiety.

96. The method of claim 91, wherein said anti-human mature Factor D-specific antibody or antigen-binding fragment thereof is naked (i.e., not labeled), and the presence or amount of the antibody or fragment thereof bound to mature Factor D is detected using a labeled antibody which binds to the anti-mature Factor D antibody.

97. The method of claim 91, wherein said anti-human mature Factor D-specific antibody or antigen-binding fragment thereof is immobilized on a substrate (i.e., capture/coating) and the bound mature Factor D is detected with a second antibody that binds to a different epitope of Factor D.

98. The method of claim 91, wherein the test sample is a biological sample obtained from a mammalian subject, such as wherein the biological sample is selected from the group consisting of blood, serum, plasma, urine, and cerebrospinal fluid.

99. The method of claim 98, wherein the mammalian subject is suffering from, or at risk for developing an alternative pathway disease or disorder.

100. The method of claim 98, wherein the mammalian subject has been treated with a complement inhibitory agent, such as an alternative complement pathway inhibitory agent, such as an inhibitor of pro-Factor D maturation, such as a MASP-3 inhibitory antibody or antigen-binding fragment thereof.

101. The method of claim 91, wherein the anti-human mature Factor D-specific antibody or antigen-binding fragment thereof comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23.

102. The method of claim 101, wherein the anti-human mature Factor D-specific antibody or antigen-binding fragment thereof comprises a binding domain comprising the following six CDRs: a) an HC-CDR1 comprising the amino acid sequence XSXMGVS (SEQ ID NO: 65), wherein X at position 1 is T, I or S and X at position 3 is G or I; (b) an HC-CDR2 comprising the amino acid sequence HIYWDDEKHYXPSLKX (SEQ ID NO:66), wherein X at position 11 is H or N and X at position 16 is S or R; (c) an HC-CDR3 comprising the amino acid sequence RYYGYXXXMXY (SEQ ID NO:67), wherein X at position 6 is R, G or N, X at position 7 is S or Y, X at position 8 is F, I or V, and X at position 10 is D or H; (d) a LC-CDR1 comprising the amino acid sequence RSXXSIXHSNGNTYXE (SEQ ID NO:68), wherein: X at position 3 is Nor S, X at position 4 is Q or E, X at position 7 is V or L, and X at position 15 is F or L; (e) a LC-CDR2 comprising the amino acid sequence KVXNRFS (SEQ ID NO:69), wherein: X at position 3 is S or Y; and (f) a LC-CDR3 comprising the amino acid sequence FQGSHVPPT (SEQ ID NO: 54).

103. The method of claim 102, wherein the anti-human mature Factor D-specific antibody or antigen-binding fragment thereof comprises a binding domain comprising the following six CDRs: (a) an HC-CDR1 comprising SEQ ID NO:25, (b) an HC-CDR2 comprising SEQ ID NO:27; (c) an HC-CDR3 comprising SEQ ID NO: 29; (d) a LC-CDR1 comprising SEQ ID NO: 50, (e) a LC-CDR2 comprising SEQ ID NO:52 and (f) a LC-CDR3 comprising SEQ ID NO: 54.

104-117. (canceled)

118. A method of assessing the extent of alternative pathway complement (APC) activation in a test sample comprising:
(a) providing a test sample;
(b) performing an immunoassay comprising at least one of:
(i) capturing and detecting mature Factor D in the test sample, wherein mature Factor D is either captured or detected with a mature Factor D-specific monoclonal antibody or antigen-binding fragment thereof that specifically binds to an epitope in “ILGGREA” (SEQ ID NO:5) present in mature Factor D, but does not bind to Pro-Factor D; and/or
(ii) capturing and detecting Pro-Factor D in the test sample, wherein Pro-Factor D is either captured or detected with a Pro-Factor D-specific monoclonal antibody or antigen-binding fragment thereof that specifically binds to an epitope on the activation (“Pro”) peptide “APPRGR” (SEQ ID NO: 4) present in Pro-Factor D, but does not bind to mature Factor D; and
(c) comparing the level of mature Factor D detected in accordance with (b) (i) with a predetermined level or control sample and/or comparing the level of Pro-Factor D detected in accordance with (b) (ii) with a predetermined level or control sample, wherein the level of mature Factor D and/or Pro-Factor D detected in the test sample is indicative of the extent of alternative pathway complement activation.

119. The method of claim 118, wherein step (b) (i) comprises capturing mature Factor D with a mature Factor D-specific monoclonal antibody or antigen-binding fragment thereof that specifically binds to an epitope in “ILGGREA” (SEQ ID NO:5) present in mature Factor D, but does not bind to Pro-Factor D and detecting with an antibody or antigen-binding fragment thereof that binds to an epitope shared by both human mature Factor D and human Pro-Factor D.

120. The method of claim 118, wherein step (b) (i) comprises capturing mature Factor D with an anti-Factor D antibody or antigen-binding fragment thereof that binds to an epitope shared by both human mature Factor D and human Pro-Factor D and detecting with a mature Factor D-specific monoclonal antibody or antigen-binding fragment thereof that specifically binds to an epitope in “ILGGREA” (SEQ ID NO:5) present in mature Factor D, but does not bind to Pro-Factor D.

121-122. (canceled)

123. The method of claim 118, wherein the mature Factor D-specific monoclonal antibody or antigen-binding fragment thereof that specifically binds to an epitope in “ILGGREA” (SEQ ID NO: 5) present in mature Factor D, but does not bind to Pro-Factor D comprises a binding domain comprising HC-CDR1, HC-CDR2 and HC-CDR3 in a heavy chain variable region selected from the group consisting of SEQ ID NO:s 12-17 and comprising LC-CDR1, LC-CDR2 and LC-CDR3 in a light chain variable region selected from the group consisting of SEQ ID NO:s 18-23 wherein the CDRs are numbered according to the Kabat numbering system.

124. The method of claim 118, wherein the Pro-Factor D-specific monoclonal antibody or antigen-binding fragment thereof that specifically binds to an epitope on the activation (“Pro”) peptide “APPRGR” (SEQ ID NO:4) present in Pro-Factor D, but does not bind to mature Factor D comprises a binding domain comprising HC-CDR1, HC-CDR2 and HC-CDR3 of a heavy chain variable region selected from the group consisting of SEQ ID NO:s 136-141 and comprising LC-CDR1, LC-CDR2 and LC-CDR3 in a light chain variable region selected from the group consisting of SEQ ID NO:s 142-147, wherein the CDRs are numbered according to the Kabat numbering system.

125. The method of claim 118, wherein the test sample is a biological sample obtained from a mammalian subject.

126. The method of claim 125, wherein the biological sample comprises whole blood, serum, plasma, urine, or cerebrospinal fluid.

127. The method of claim 118, wherein the test sample comprises a complement inhibitory agent, such as an alternative complement pathway inhibitory agent, such as an inhibitor of pro-Factor D maturation, such as a MASP-3 inhibitory agent.

128. The method of claim 125, wherein the mammalian subject has been treated with a complement inhibitory agent, such as an alternative complement pathway inhibitory agent, such as an inhibitor of pro-Factor D maturation, such as a MASP-3 inhibitory agent and the assay is used to measure the extent of alternative pathway inhibition.

129. The method of claim 125, wherein the mammalian subject is a human subject.

130. The method of claim 129 wherein the human subject is suffering from, or at risk of developing, or suspected of having an alternative-pathway disease or disorder.

131. The method of claim 130, wherein the alternative-pathway disease or disorder is selected from the group consisting of: paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopeni purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

132. The method of claim 128, wherein the control sample is a sample taken from the subject prior to treatment with the MASP-3 inhibitory agent, or a sample taken at an earlier point in time during a course of treatment with the MASP-3 inhibitory agent.

133. The method of claim 128, wherein the MASP-3 inhibitory agent is a MASP-3 inhibitory antibody or antigen-binding fragment thereof.

134. The method of claim 133, wherein the MASP-3 inhibitory antibody is a monoclonal antibody, or antigen-binding fragment thereof, that binds to MASP-3 and comprises at least one of:
(i) a binding domain comprising HC-CDR1, HC-CDR2 and HC-CDR3 of a heavy chain variable region selected from the group consisting of SEQ ID NO:s 220, 222, 223, 225, 226 and 228 and comprising LC-CDR1, LC-CDR2 and LC-CDR3 of a light chain variable region selected from the group consisting of SEQ ID NO:s 221, 224 and 227, wherein the CDRs are numbered according to the Kabat numbering system;
(ii) a heavy chain variable region comprising a HC-CDR1 comprising SEQ ID NO:229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO: 178 (WASTRES) and a LC-CDR3 comprising SEQ ID NO:242 (KQSYNLYT);
(iii) a heavy chain variable region comprising a HC-CDR1 comprising SEQ ID NO:230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO: 241 (LVSKLDS) and a LC-CDR3 comprising SEQ ID NO:243 (WQGTHFPWT); or
(iv) a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO: 235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT).

135-148. (canceled)

149. A method of treating a mammalian subject suffering from, or at risk of developing an alternative-pathway disease or disorder, comprising administering a MASP-3 inhibitory antibody or antigen-binding fragment thereof to the subject if the subject is determined to have:
(i) a lower or decreased level of Pro-Factor D in one or more samples taken from the subject compared to a predetermined Pro-Factor D level or compared to the Pro-Factor D level in one or more control samples; and/or
(ii) a higher or increased level of mature Factor D in one or more samples taken from the subject compared to a predetermined mature Factor D level or compared to the mature Factor D level in one or more control samples.

150. The method of claim 149, wherein the level of Pro-Factor D in one or more samples taken from the subject is determined by performing an immunoassay comprising the use of a Pro-Factor D-specific monoclonal antibody.

151. The method of claim 150, wherein the immunoassay comprises (i) a first monoclonal antibody, or antigen-binding fragment thereof. that specifically binds to an epitope in the pro peptide of human Factor D, wherein the epitope comprises or consists of the amino acids APPRGR (SEQ ID NO:4) and does not bind to human mature Factor D; and (ii) a second antibody, or antigen-binding fragment thereof, that binds to an epitope shared by both human mature Factor D and human Pro-Factor D, wherein the first and second antibody or antigen-binding fragments thereof function together in the immunoassay to specifically detect or quantitate the amount of Pro-Factor D protein (SEQ ID NO:2) and not mature-Factor D protein (SEQ ID NO:3) that may be present in the sample.

152. The method of claim 149, wherein the level of mature Factor D in one or more samples taken from the subject is determined by performing an immunoassay comprising the use of a mature Factor D-specific monoclonal antibody or antigen-binding fragment thereof.

153. The method of claim 152, wherein the immunoassay comprises (i) a first monoclonal antibody, or antigen-binding fragment thereof, that specifically binds to an epitope in the N-terminal region of human mature Factor D, wherein the epitope comprises or consists of the amino acids ILGGREA (SEQ ID NO:5) and does not bind to human Pro-Factor D; and (ii) a second antibody, or antigen-binding fragment thereof, that binds to an epitope shared by both human mature Factor D and human Pro-Factor D, wherein the first and second antibody, or antigen-binding fragments thereof, function together in the immunoassay to specifically detect or quantitate the amount of mature Factor D protein (SEQ ID NO:3) and not Pro-Factor D protein (SEQ ID NO:2) that may be present in the sample.

154. The method of claim 149, wherein the mammalian subject is a human subject.

155. The method of claim 154, wherein the human subject is suffering from, or at risk of developing an alternative pathway disease or disorder selected from the group consisting of paroxysmal nocturnal hemoglobinuria (PNH), age-related macular degeneration (AMD, including wet and dry AMD), ischemia-reperfusion injury, arthritis, disseminated intravascular coagulation, thrombotic microangiopathy (including hemolytic uremic syndrome (HUS), atypical hemolytic uremic syndrome (aHUS), thrombotic thrombocytopenia purpura (TTP) or transplant-associated TMA), asthma, dense deposit disease, pauci-immune necrotizing crescentic glomerulonephritis, traumatic brain injury, aspiration pneumonia, endophthalmitis, neuromyelitis optica, Behcet's disease, multiple sclerosis, Guillain Barre Syndrome, Alzheimer's disease, Amylotrophic lateral sclerosis (ALS), lupus nephritis, systemic lupus erythematosus (SLE), Diabetic retinopathy, Uveitis, Chronic obstructive pulmonary disease (COPD), C3 glomerulopathy, transplant rejection, Graft-versus-host disease (GVHD), hemodialysis, sepsis, Systemic inflammatory response syndrome (SIRS), Acute Respiratory Distress Syndrome (ARDS), ANCA vasculitis, Anti-phospholipid syndrome, Atherosclerosis, IgA Nephropathy and Myasthenia Gravis.

156. The method of claim 149, wherein the MASP-3 inhibitory antibody or antigen-binding fragment thereof is a monoclonal antibody or antigen-binding fragment thereof.

157. The method of claim 156, wherein the MASP-3 inhibitory antibody or antigen-binding fragment thereof is a monoclonal antibody, or antigen-binding fragment thereof, that binds to MASP-3 and comprises at least one of:
(i) a binding domain comprising HC-CDR1, HC-CDR2 and HC-CDR3 of a heavy chain variable region selected from the group consisting of SEQ ID NO:s 220, 222, 223, 225, 226 and 228 and comprising LC-CDR1, LC-CDR2 and LC-CDR3 of a light chain variable region selected from the group consisting of SEQ ID NO:s 221, 224 and 227, wherein the CDRs are numbered according to the Kabat numbering system;
(ii) a heavy chain variable region comprising a HC-CDR1 comprising SEQ ID NO:229 (TDDIN), a HC-CDR2 comprising SEQ ID NO:232 (WIYPRDDRTKYNDKFKD), a HC-CDR3 comprising SEQ ID NO:236 (LEDTY); and a light chain variable region comprising a LC-CDR1 comprising SEQ ID NO:239 (KSSQSLLASRTRKNYLA), a LC-CDR2 comprising SEQ ID NO: 178 (WASTRES) and a LC-CDR3 comprising SEQ ID NO:242 (KQSYNLYT);
(iii) a heavy chain variable region comprising a HC-CDR1 comprising SEQ ID NO:230 (SYGMS), a HC-CDR2 comprising SEQ ID NO:233 (WINTYSGVPTYADDFKG) and a HC-CDR3 comprising SEQ ID NO:237 (GGEAMDY); and a light chain variable region comprising a LC-CDR1 comprising SEQ ID NO:240 (KSSQSLLDSDAKTYLN), a LC-CDR2 comprising SEQ ID NO: 241 (LVSKLDS) and a LC-CDR3 comprising SEQ ID NO:243 (WQGTHFPWT); or
(iv) a heavy chain variable region comprising a HC-CDR1 comprising SEQ ID NO:231 (GKWIE); a HC-CDR2 comprising SEQ ID NO:234 (EILPGTGSTNYNEKFKG) or SEQ ID NO: 235 (EILPGTGSTNYAQKFQG); and a HC-CDR3 comprising SEQ ID NO:238 (SEDV); and a light chain variable region comprising a LC-CDR1 comprising SEQ ID NO:239, a LC-CDR2 comprising SEQ ID NO:178 (WASTRES); and a LC-CDR3 comprising SEQ ID NO:244 (KQSYNIPT).

158-174. (canceled)
